(19) United States
(12) Patent Application Publication
(10) Pub. No.: US 2023/0219942 A1
(43) Pub. Date: Jul. 13, 2023

(54) COMPOUNDS COMPRISING A THREE RING CORE AS PD-1/PD-L1 BLOCKERS

(71) Applicant: RELIVE THERAPEUTICS INC., Ottawa (CA)

(72) Inventors: George L. Trainor, Wilmington, DE (US); Perry Molinoff, Johns Island, SC (US); Adel Nefzi, Port Saint Lucie, FL (US); Marcello Giulianotti, Vero Beach, FL (US); Richard Houghten, Port Saint Lucie, FL (US)

(21) Appl. No.: 18/001,442

(22) PCT Filed: Jun. 2, 2021
(86) PCT No.: PCT/CA2021/050747

§ 371 Date: Dec. 9, 2022

Related U.S. Application Data
(60) Provisional application No. 63/036,647, filed on Jun. 9, 2020.
(60) Provisional application No. 63/160,113, filed on Mar. 12, 2021.

(51) Int. Cl. C07D 417/14

(52) U.S. Cl. CPC C07D 417/14

(57) ABSTRACT

The present disclosure relates to compounds of Formula (I): (I), wherein R₁, R₂, L₁, L₂, Y₁, Y₂, Y₃, X₁, Ring A, Ring B and Ring C are as defined herein, as well as to compositions comprising such compounds. The compounds and compositions may be useful for treating diseases and conditions that are amenable to treatment by blocking PD-1, PD-L1 and/or the PD-1/PD-L1 interaction, cancers, sepsis and/or autoimmune diseases.

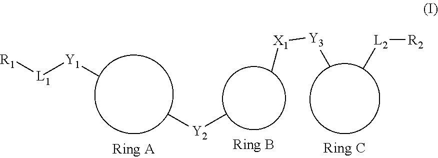

### CROSS-REFERENCE TO RELATED APPLICATIONS

This application claims priority to United States Provisional Patent Application Nos. 63/036,647, entitled “Compounds Comprising a 4-[(1-Piperazinyl)Methyl]-1,3-Thiazole Core”, filed on Jun. 9, 2020 and 63/160,113, entitled “Compounds Comprising a Three Ring Core as PD-1/PD-L1 Blockers”, filed on Mar. 12, 2021, the entire contents of which are incorporated by reference herein.

### FIELD

The present disclosure relates to compounds that may block the PD-1/PD-L1 interaction. Specifically, the present disclosure relates to compounds of Formula (I), compositions comprising such compounds, and uses thereof, such as for the treatment of cancer, sepsis and autoimmune diseases.

### BACKGROUND

PD-1 is an immune checkpoint protein that is expressed as a receptor on the surfaces of various immune cells, including T-cells and B-cells. PD-L1, one of the two ligands for PD-1 (the other being PD-L2), is expressed, for example, on antigen presenting cells and in some non-lymphoid tissue. The binding of PD-L1 to PD-1 on the surface of T-cells down-regulates the immune system and provides T-cell homeostasis.

The interaction of PD-1 with its ligands is believed to play an important role in several disease states. For example, the PD-1 signalling pathway is involved in the inhibition of self-reactive T-cells, which serves to protect against auto-immune diseases. In the cancer disease state, the interaction of PD-L1 expressed on tumor cells-PD-L1 has been found to be overexpressed on the surface of different cancer types, including breast cancer, lung cancer, bladder cancer, lymphoma, glioblastoma and melanoma (Ganesan, Aravindhan, et al. “Comprehensive in vitro characterization of PD-L1 small molecule inhibitors” (2019) Scientific Reports, 9, 12392, https://doi.org/10.1038/s41598-019-48826-6)—with PD-1 receptors on T-cells suppresses the T-cells' activity and prevents the immune system from attacking tumor cells.

The recognition that reagents that block the interaction of PD-L1 with PD-1 (known as an immune checkpoint blockade) may prevent tumor cells from evading the immune system has transformed cancer treatment and spurred the development of PD-1 and PD-L1 blockers, with more than 1500 clinical studies involving PD-1 and PD-L1 blockers as of 2017 (see: Musielak, Bogdan et al. “CA-170—A Potent Small-Molecule PD-L1 Inhibitor or Not?” (1 Aug. 2019) Molecules, 24(15), 2804, doi:10.3390/molecules24152804; and Iwai, Yoshiko et al. “Cancer immunotherapies targeting the PD-1 signaling pathway” (4 Apr. 2017) Journal of Biomedical Science, 24(1), 26, doi:10.1186/s12929-017-0329-9).

Further, at least six PD-1 and PD-L1 blockers have been approved for clinical use by the U.S. Food and Drug Administration (FDA) to treat a range of cancers (see Table 1, below).

TABLE 1
Approved PD-1/PD-L1 Blockers

| Name | Target |
|---|---|
| Nivolumab (Opdivo ™) | PD-1 |
| Pembrolizumab (Keytruda ™) | PD-1 |
| Atezolizumab (Tecentriq ™) | PD-L1 |
| Avelumab (Bavencio ™) | PD-L1 |
| Durvalumab (Imfinzi ™) | PD-L1 |
| Cemiplimab (Libtayo ™) | PD-1 |

Nivolumab has been approved to treat non-small-cell lung cancer (NSCLC), renal cell carcinoma (RCC), bladder cancer (BC), colorectal cancer (CRC) with microsatellite instability or mismatch repair deficiency (MSI-H/dMMR), hepatocellular carcinoma (HCC), classic Hodgkin lymphoma (cHL), melanoma, and head and neck squamous cell carcinoma (HNSCC). Pembrolizumab has been approved to treat melanoma, HNSCC, cervical cancer, cHL, NSCLC, BC, stomach and gastroesophageal cancers, and all advanced solid tumors classified as MSI-H/dMMR. Avelumab has been approved to treat Merkel cell carcinoma and BC. Atezolizumab has been approved to treat NSCLC and BC. Durvalumab has been approved for BC and NSCLC (stage III) (see, Qin, Weiting et al. “The Diverse Function of PD-1/PD-L Pathway Beyond Cancer” (4 Oct. 2019) Frontiers in Immunology, 10, 2298).

However, it is noteworthy that each of the PD-1/PD-L1 blockers listed in Table 1 is a monoclonal antibody. Such immunotherapies have several limitations, including: the high production cost of antibodies; the lack of oral bioavailability of monoclonal antibodies; the poor diffusion and permeation profiles correlated with high molecular weights of monoclonal antibodies; and the unfavorable pharmacokinetic profiles of monoclonal antibodies, which are related to toxicities and immunogenicity leading to severe immune-related adverse events (Guzik, Katarzyna et al. “Development of the Inhibitors that Target the PD-1/PD-L1 Interaction-A Brief Look at Progress on Small Molecules, Peptides and Macrocycles” (30 May 2019) Molecules, 24(11), 2071, doi:10.3390/molecules24112071).

While large molecule approaches continue to be of interest, with alternative large molecule approaches in progress both preclinically and clinically (e.g. bi-specific, nano-body, pro-body, vaccines, etc.), and significant activity in peptide and cyclic-peptide design, small molecule PD-1/PD-L1 blockers have received considerable interest as alternatives to and/or complements for existing therapies (see, for example: Guzik, Katarzyna et al. “Development of the Inhibitors that Target the PD-1/PD-L1 Interaction-A Brief Look at Progress on Small Molecules, Peptides and Macrocycles” (30 May 2019) Molecules, 24(11), 2071, doi:10.3390/molecules24112071; and Huck, Bayard R et al. “Small Molecules Drive Big Improvements in Immuno-Oncology Therapies” (2018) Angewandte Chemie (International ed. in English), 57(16), 4412, doi:10.1002/anie.201707816).

In this regard, WO 2015/033301, WO 2018/119286, WO 2018/195321 and WO 2015/160641, for example, each disclose small molecule PD-1/PD-L1 blockers. Selected examples of specific small molecule PD-1/PD-L1 blockers are shown below.

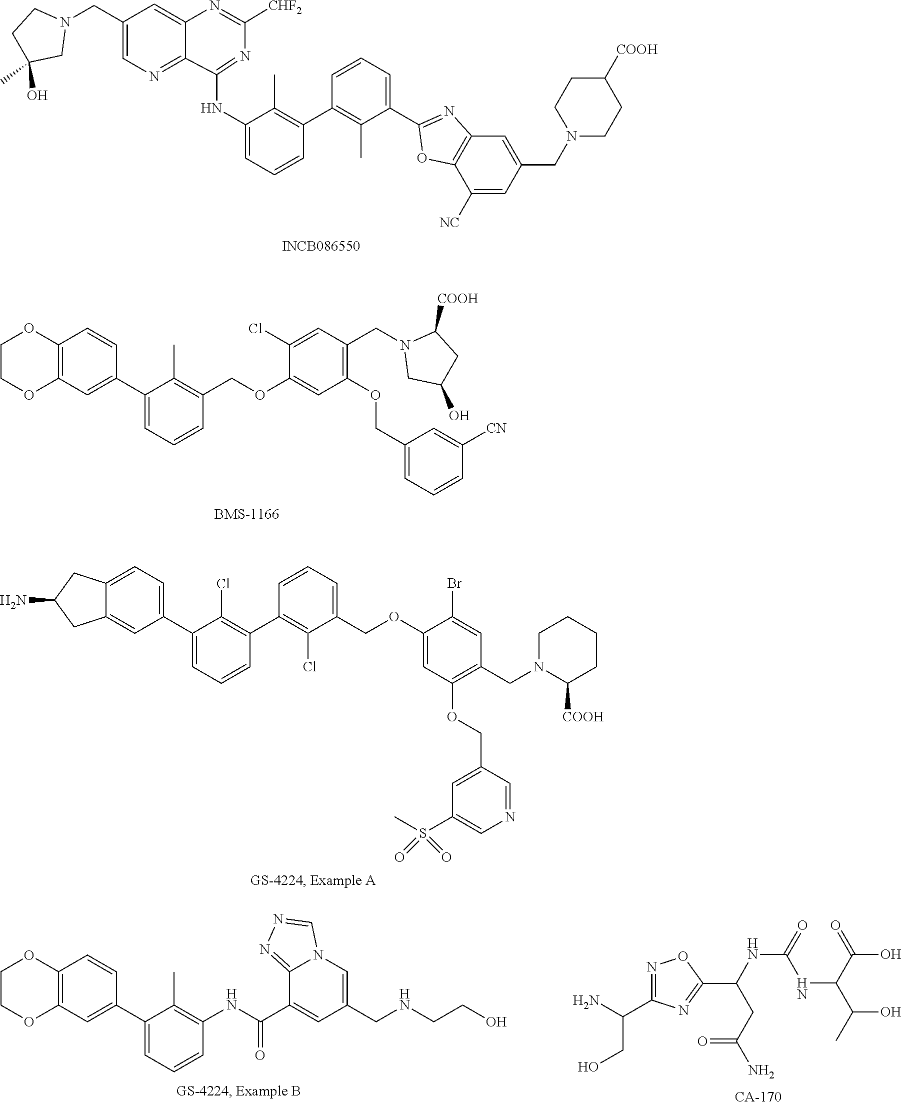 [SMILES: Cc1c(Nc2nc(C(F)F)nc3cc(CN4CC[C@](C)(O)C4)cnc23)cccc1-c1cccc(-c2nc3cc(CN4CCC(C(=O)O)CC4)cc(C#N)c3o2)c1C ; Cc1c(COc2cc(OCc3cccc(C#N)c3)c(CN3C[C@H](O)C[C@@H]3C(=O)O)cc2Cl)cccc1-c1ccc2c(c1)OCCO2 ; CS(=O)(=O)c1cncc(COc2cc(OCc3cccc(-c4cccc(-c5ccc6c(c5)C[C@@H](N)C6)c4Cl)c3Cl)c(Br)cc2CN2CCCC[C@H]2C(=O)O)c1 ; Cc1c(NC(=O)c2cc(CNCCO)cn3cnnc23)cccc1-c1ccc2c(c1)OCCO2 ; CC(O)C(NC(=O)NC(CC(N)=O)c1nc(C(N)CO)no1)C(=O)O]

Despite the interest and potential advantages of small molecule agents-including lower production costs, higher stability, improved tumor penetration, amenability for oral administration and elimination of immunogenicity issues—there remain relatively few small molecule agents currently in clinical development (Musielak, Bogdan et al. “CA-170—A Potent Small-Molecule PD-L1 Inhibitor or Not?” (1 Aug. 2019) Molecules, 24(15), 2804, doi:10.3390/molecules 24152804; Skalniak, Lukasz et al. “Small-molecule inhibitors of PD-1/PD-L1 immune checkpoint alleviate the PD-L1-induced exhaustion of T-cells” (7 Aug. 2017) Oncotarget, 8(42), 72167, doi:10.18632/oncotarget.20050).

The present disclosure reports a group of novel small molecule compounds which may be useful as blockers of the PD-1/PD-L1 interaction. The compounds may be useful in the treatment of diseases and/or conditions that are amenable to treatment by blocking PD-1, PD-L1 or the PD-1/PD-L1 interaction, cancer, sepsis and autoimmune diseases.

### SUMMARY

In a first aspect, the present disclosure relates to a compound of Formula (I):

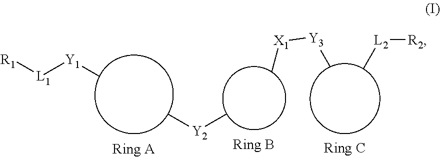

wherein:
R₁ is C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆heteroalkyl or C₅₋₁₄heteroaryl-C₂₋₆heteroalkyl, each of which is unsubstituted or substituted with one or more R^{A};
R₂ is C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆heteroalkyl or C₅₋₁₄heteroaryl-C₂₋₆heteroalkyl, each of which is unsubstituted or substituted with one or more R^{A};
L₁ is optional and if present is C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl or C₅₋₁₄ heterocycloalkenyl, each of which is unsubstituted or substituted with one or more R^{A};
L₂ is optional and if present is C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl or C₅₋₁₄ heterocycloalkenyl, each of which is unsubstituted or substituted with one or more R^{A};
Y₁, Y₂ and Y₃ are independently —CR₅R₆—, —C(CR₅R₆)—, —C(O)—, —C(NR^{B})—, —C(S)—, —NR^{B}—, —O—, —S—, —(O)— or —(O)₂—;
X₁ is —CR^{B}₂—, —C(CR^{B}₂)—, —C(O)—, —C(NR^{B})—, —C(S)—, —NR^{B}—, —O—, —S—, —(O)— or —S(O)₂—;
Ring A is a C₆ cycloalkyl or C₆ heterocycloalkyl, each of which is unsubstituted or substituted with one or more R₃;
Ring B is a C₆ aryl or C₅₋₆ heteroaryl, each of which is unsubstituted or substituted with one or more R^{A};
Ring C is a C₅₋₆ cycloalkyl or C₅₋₆ heterocycloalkyl, each of which is unsubstituted or substituted with one or more R₄;
R₃ and R₄ at each occurrence are independently C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₁₋₆ haloalkyl, C₂₋₆ haloalkenyl, C₂₋₆ haloalkynyl, C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₃₋₁₄ haloheterocycloalkyl, C₅₋₁₄ haloheterocycloalkenyl, C₅₋₁₄ aryl, C₅₋₁₄ heteroaryl, haloC₅₋₁₄ aryl, haloC₅₋₁₄heteroaryl, C₅₋₁₄aryl-C₁₋₆alkyl, C₅₋₁₄heteroaryl-C₁₋₆alkyl, haloC₅₋₁₄aryl-C₁₋₆alkyl, haloC₅₋₁₄heteroaryl-C₁₋₆alkyl, C₅₋₁₄aryl-C₂₋₆heteroalkyl, C₅₋₁₄heteroaryl-C₂₋₆ heteroalkyl, haloC₅₋₁₄aryl-C₂₋₆heteroalkyl, haloC₅₋₁₄heteroaryl-C₂₋₆heteroalkyl, —F, —Cl, —Br, —I, —CN, —NO₂, —SO₂, —N₃, —SCN, —NCS, —OR^{B}, —NR^{C}R^{D}, —NR^{B}NR^{C}R^{D}, —SR^{B}, —C(O)R^{B}, —C(O)OR^{B}, —C(O)NR^{C}R^{D}, —C(NR^{C})R^{B}, —C(NR^{B})NR^{C}R^{D}, —S(O)R^{B}, —S(O)₂R^{B}, —S(O)OR^{B}, —S(O)₂OR^{B}, —NR^{B}NR^{C}R^{D}, —NR^{B}C(O)NR^{C}R^{D} or —NR^{B}C(NR^{B})NR^{C}R^{D}; two or more R₃ or two or more R₄ together with the atom(s) to which they are attached form a cyclic group; and/or two R₃ or two R₄ on the same atom form (═O), (═NR^{B}) or (═S);
R₅ and R₆ at each occurrence are independently —H, C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₁₋₆ haloalkyl, C₂₋₆ haloalkenyl, C₂₋₆ haloalkynyl, C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₃₋₁₄ haloheterocycloalkyl, C₅₋₁₄ haloheterocycloalkenyl, C₅₋₁₄ aryl, C₅₋₁₄ heteroaryl, haloC₅₋₁₄ aryl, haloC₅₋₁₄heteroaryl, C₅₋₁₄aryl-C₁₋₆alkyl, C₅₋₁₄heteroaryl-C₁₋₆alkyl, haloC₅₋₁₄aryl-C₁₋₆alkyl, haloC₅₋₁₄heteroaryl-C₁₋₆alkyl, C₅₋₁₄aryl-C₂₋₆heteroalkyl, C₅₋₁₄heteroaryl-C₂₋₆ heteroalkyl, haloC₅₋₁₄aryl-C₂₋₆heteroalkyl, haloC₅₋₁₄heteroaryl-C₂₋₆heteroalkyl, —F, —Cl, —Br, —I, —CN, —NO₂, —SO₂, —N₃, —SCN, —NCS, —OR^{B}, —NR^{C}R^{D}, —NR^{B}NR^{C}R^{D}, —SR^{B}, —C(O)R^{B}, —C(O)OR^{B}, —C(O)NR^{C}R^{D}, —C(NR^{C})R^{B}, —C(NR^{B})NR^{C}R^{D}, —S(O)R^{B}, —S(O)₂R^{B}, —S(O)OR^{B}, —S(O)₂OR^{B}, —NR^{B}NR^{C}R^{D}, —NR^{B}C(O)NR^{C}R^{D} or —NR^{B}C(NR^{B})NR^{C}R^{D}; or R₅ and R₆ together with the carbon atom to which they are attached form a C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₃₋₁₄ haloheterocycloalkyl, or C₅₋₁₄ haloheterocycloalkenyl;
R^{A} at each occurrence is independently —F, —Cl, —Br, —I, —CN, —NO₂, —SO₂, —N₃, —SCN, —NCS, —OR^{B}, —NR^{C}R^{D}, —NR^{B}NR^{C}R^{D}, —SR^{B}, —C(O)R^{B}, —C(O)OR^{B}, —C(O)NR^{C}R^{D}, —C(NR^{B})R^{B}, —C(NR^{B})NR^{C}R^{D}, —S(O)R^{B}, —S(O)₂R^{B}, —S(O)OR^{B}, —S(O)₂OR^{B}, —NR^{B}NR^{C}R^{D}, —NR^{B}C(O)NR^{C}R^{D}, —NR^{B}C(NR^{B})NR^{C}R^{D}, C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆heteroalkyl or C₅₋₁₄heteroaryl-C₂₋₆ heteroalkyl, wherein each C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₆₋₁₄heteroaryl-C₁₋₆alkyl, C₅₋₁₄aryl-C₂₋₆heteroalkyl or C₅₋₁₄heteroaryl-C₂₋₆heteroalkyl is unsubstituted or substituted with one or more —F, —Cl, —Br, —I, —CN, —NO₂, —SO₂, —N₃, —SCN, —NCS, —OR^{B}, —NR^{C}R^{D}, —NR^{B}NR^{C}R^{D}, —SR^{B}, —C(O)R^{B}, —C(O)OR^{B}, —C(O)NR^{C}R^{D}, —C(NR^{B})R^{B}, —C(NR^{B})NR^{C}R^{D}, —NR^{B}C(NR^{B})NR^{C}R^{D}, —S(O)R^{B}, —S(o)₂R^{B}, —S(O)OR^{B}, —S(O)₂OR^{B}, —NR^{B}NR^{C}R^{D} or —NR^{B}C(O)NR^{C}R^{D}; two or more R^{A} together with the atom(s) to which they are attached form a cyclic group; and/or two R^{A} on the same atom form (═O), (═NR^{B}) or (═S);
R^{B} at each occurrence is independently (i) —H, or (ii) C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆heteroalkyl or C₅₋₁₄heteroaryl-C₂₋₆ heteroalkyl, each of which is unsubstituted or substituted with one or more R^{E};
R^{C} and R^{D} at each occurrence are independently (i) —H, or (ii) C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆heteroalkyl or C₅₋₁₄heteroaryl-C₂₋₆ heteroalkyl, each of which is unsubstituted or substituted with one or more R^{E}, or R^{C} and R^{D} together with the nitrogen atom to which they are attached combine to form a heterocycle that is unsubstituted or substituted with one or more R^{E} groups;
R^{E} at each occurrence is independently —F, —Cl, —Br, —I, —CN, —NO₂, —SO₂, —N₃, —SCN, —NCS, —OR^{F}, —NR^{G}R^{H}, —NR^{F}NR^{G}R^{H}, —SR^{F}, —C(O)R^{F}, —C(O)OR^{F}, —C(O)NR^{G}R^{H}, —C(NR^{F})R^{F}, —C(NR^{F})NR^{G}R^{H}, —S(O)R^{F}, —S(O)₂R^{F}, —S(O)OR^{F}, —S(O)₂OR^{F}, —NR^{F}NR^{G}R^{H}, —NR^{F}C(O)NR^{G}R^{H}, —NR^{F}C(NR^{F})NR^{G}R^{H}, C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆heteroalkyl or C₅₋₁₄heteroaryl-C₂₋₆ heteroalkyl, wherein each C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄ heteroaryl-C₁₋₆alkyl, C₅₋₁₄aryl-C₂₋₆heteroalkyl or C₅₋₁₄heteroaryl-C₂₋₆heteroalkyl is unsubstituted or substituted with one or more —F, —Cl, —Br, —I, —CN, —NO₂, —SO₂, —N₃, —SCN, —NCS, —OR^{F}, —NR^{G}R^{H}, —NR^{F}NR^{G}R^{H}, —SR^{F}, —C(O)R^{F}, —C(O)OR^{F}, —C(O)NR^{G}R^{H}, —C(NR^{F})R^{F}, —C(NR^{F})NR^{G}R^{H}, —NR^{F}C(NR^{F})NR^{G}R^{H}, —S(O)R^{F}, —S(O)₂R^{F}, —S(O)OR^{F}, —S(O)₂OR^{F}, —NR^{F}NR^{G}R^{H} or —NR^{F}C(O)NR^{G}R^{H}; two or more R^{E} together with the atoms to which they are attached form a cyclic group; and/or two R^{E} on the same atom form (═O), (═NR^{F}) or (═S);
R^{F} at each occurrence is independently (i) —H, or (ii) C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₁₋₆ haloalkyl, C₂₋₆ haloalkenyl, C₂₋₆ haloalkynyl, C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₅₋₁₄ aryl, C₅₋₁₄ heteroaryl, haloC₅₋₁₄aryl, haloC₅₋₁₄heteroaryl, C₅₋₁₄aryl-C₁₋₆alkyl, C₅₋₁₄heteroaryl-C₁₋₆alkyl, haloC₅₋₁₄aryl-C₁₋₆alkyl, haloC₅₋₁₄heteroaryl-C₁₋₆alkyl, C₅₋₁₄aryl-C₂₋₆heteroalkyl, C₅₋₁₄ heteroaryl-C₂₋₆heteroalkyl, haloC₅₋₁₄aryl-C₂₋₆heteroalkyl or haloC₅₋₁₄heteroaryl-C₂₋₆ heteroalkyl; and
R^{G} and R^{H} at each occurrence are independently (i) —H, or (ii) C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₁₋₆ haloalkyl, C₂₋₆ haloalkenyl, C₂₋₆ haloalkynyl, C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₅₋₁₄ aryl, C₅₋₁₄ heteroaryl, haloC₅₋₁₄aryl, haloC₅₋₁₄heteroaryl, C₅₋₁₄aryl-C₁₋₆alkyl, C₅₋₁₄heteroaryl-C₁₋₆alkyl, haloC₅₋₁₄aryl-C₁₋₆alkyl, haloC₅₋₁₄heteroaryl-C₁₋₆alkyl, C₅₋₁₄aryl-C₂₋₆heteroalkyl, C₆₋₁₄heteroaryl-C₂₋₆heteroalkyl, haloC₅₋₁₄aryl-C₂₋₆heteroalkyl or haloC₅₋₁₄heteroaryl-C₂₋₆-heteroalkyl, or R^{G} and R^{H} together with the nitrogen atom to which they are attached combine to form a heterocycle,
   or a pharmaceutically acceptable salt, solvate, tautomer, stereoisomer or prodrug thereof, provided that the compound of Formula (I) is not:

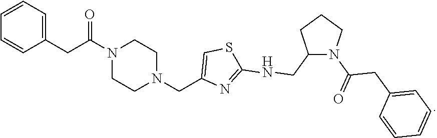 [SMILES: O=C(Cc1ccccc1)N1CCN(Cc2csc(NCC3CCCN3C(=O)Cc3ccccc3)n2)CC1]

In a second aspect, the present disclosure relates to a pharmaceutical composition comprising a compound of the disclosure, or a pharmaceutically acceptable salt, solvate, tautomer, stereoisomer or prodrug thereof. The pharmaceutical composition may also comprise a pharmaceutically acceptable carrier, excipient and/or diluent.

In a third aspect, the present disclosure relates to a method of treating a disease or condition, wherein the method comprises administering a compound of the disclosure, or a pharmaceutically acceptable salt, solvate, tautomer, stereoisomer or prodrug thereof, or a compound which is

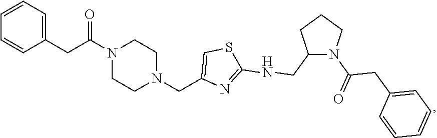 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof, to a subject in need thereof. The disease or condition may be a disease or condition that is amenable to treatment by blocking PD-1, PD-L1 and/or the PD-1/PD-L1 interaction, cancer, sepsis or an autoimmune disease.

In a fourth aspect, the present disclosure relates to the use of a compound of the disclosure, or a pharmaceutically acceptable salt, solvate, tautomer, stereoisomer or prodrug thereof, or a compound which is

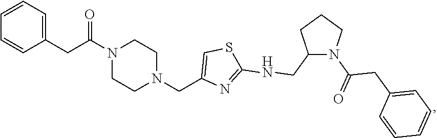 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof, for the treatment of a disease or condition in a subject in need thereof. The disease or condition may be a disease or condition that is amenable to treatment by blocking PD-1, PD-L1 and/or the PD-1/PD-L1 interaction, cancer, sepsis or an autoimmune disease.

In a fifth aspect, the present disclosure relates to the use of a compound of the disclosure, or a pharmaceutically acceptable salt, solvate, tautomer, stereoisomer or prodrug thereof, or a compound which is

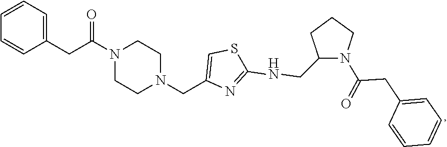 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof, for the manufacture of a medicament for the treatment of a disease or condition. The disease or condition may be a disease or condition that is amenable to treatment by blocking PD-1, PD-L1 and/or the PD-1/PD-L1 interaction, cancer, sepsis or an autoimmune disease.

In a sixth aspect, the present disclosure relates to a kit comprising: (a) a compound of the disclosure, or a pharmaceutically acceptable salt, solvate, tautomer, stereoisomer or prodrug thereof, or a compound which is

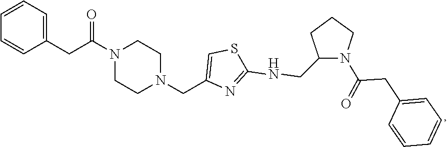 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof; and (b) instructions for using the compound or pharmaceutically acceptable salt, solvate, tautomer, stereoisomer or prodrug thereof to treat a disease or condition. The disease or condition may be a disease or condition that is amenable to treatment by blocking PD-1, PD-L1 and/or the PD-1/PD-L1 interaction, cancer, sepsis or an autoimmune disease.

### BRIEF DESCRIPTION OF THE FIGURES

The figures, which are described below, illustrate embodiments of the disclosure by way of example only.

FIGS. 1(a) and 1(b) each illustrate a profile of the effect of a compound of the disclosure, compound 2678-2, at different concentrations on the PD-1/PD-L1 interaction. The IC₅₀ value of the compound is inset. FIG. 1(a) illustrates the profile obtained using a purified sample of 2678-2. FIG. 1(b) illustrates the profile obtained using a crude sample of 2678-2. The data was obtained using a PD-1/PD-L1 Homogeneous Time-Resolved Fluorescence (HTRF) binding assay. For the purified sample, a 10 mM stock solution of the compound in 100% DMSO was run at 8 doses (5.000 μM, 1.667 μM, 0.556 μM, 0.185 μM, 0.062 μM, 0.021 μM, 0.007 μM and 0.002 μM), which were carried out in duplicate. For the crude sample, a 10 mM stock solution of the compound in 100% DMSO was run at 5 doses (5.000 μM, 0.556 μM, 0.185 μM, 0.021 μM and 0.002 μM), which were carried out in duplicate.

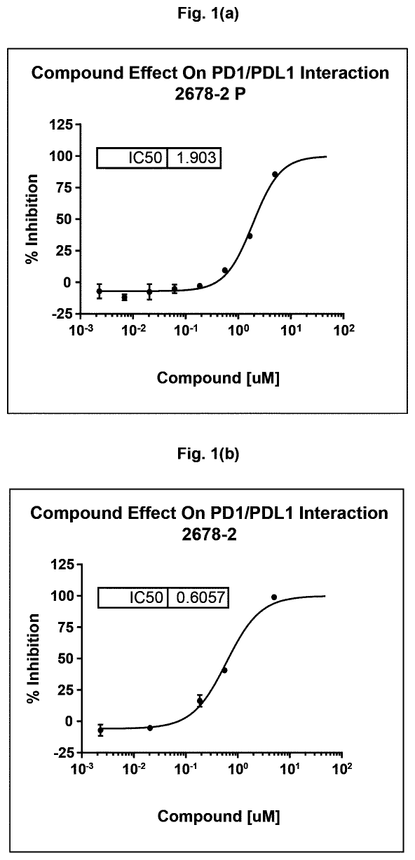

FIGS. 2(a) and 2(b) each illustrate a profile of the effect of a compound of the disclosure, compound 2678-3, at different concentrations on the PD-1/PD-L1 interaction. The IC₅₀ value of the compound is inset. FIG. 2(a) illustrates the profile obtained using a purified sample of 2678-3. FIG. 2(b) illustrates the profile obtained using a crude sample of 2678-3. The data was obtained using a PD-1/PD-L1 HTRF binding assay. For the purified sample, a 10 mM stock solution of the compound in 100% DMSO was run at 8 doses (5.000 μM, 1.667 μM, 0.556 μM, 0.185 μM, 0.062 μM, 0.021 μM, 0.007 μM and 0.002 μM), which were carried out in duplicate. For the crude sample, a 10 mM stock solution of the compound in 100% DMSO was run at 5 doses (5.000 μM, 0.556 μM, 0.185 μM, 0.021 μM and 0.002 μM), which were carried out in duplicate.

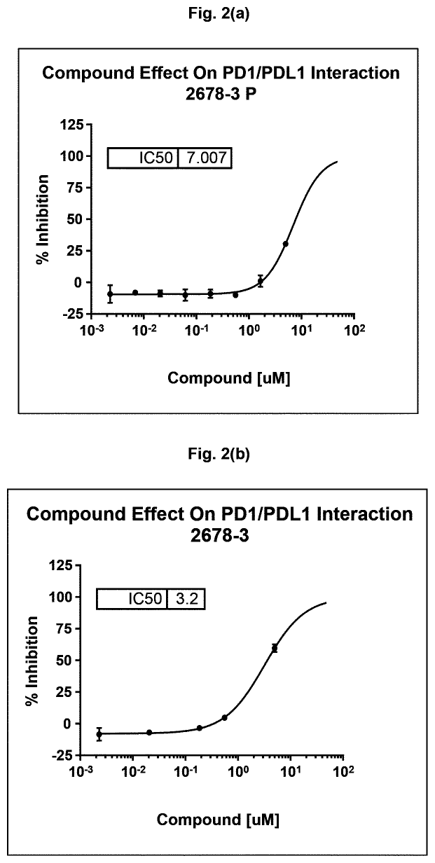

FIGS. 3(a) and 3(b) each illustrate a profile of the effect of a compound of the disclosure, compound 2678-5, at different concentrations on the PD-1/PD-L1 interaction. The IC₅₀ value of the compound is inset. FIG. 3(a) illustrates the profile obtained using a purified sample of 2678-5. FIG. 3(b) illustrates the profile obtained using a crude sample of 2678-5. The data was obtained using a PD-1/PD-L1 HTRF binding assay. For the purified sample, a 10 mM stock solution of the compound in 100% DMSO was run at 8 doses (5.000 μM, 1.667 μM, 0.556 μM, 0.185 μM, 0.062 μM, 0.021 μM, 0.007 μM and 0.002 μM), which were carried out in duplicate. For the crude sample, a 10 mM stock solution of the compound in 100% DMSO was run at 5 doses (5.000 μM, 0.556 μM, 0.185 μM, 0.021 μM and 0.002 μM), which were carried out in duplicate.

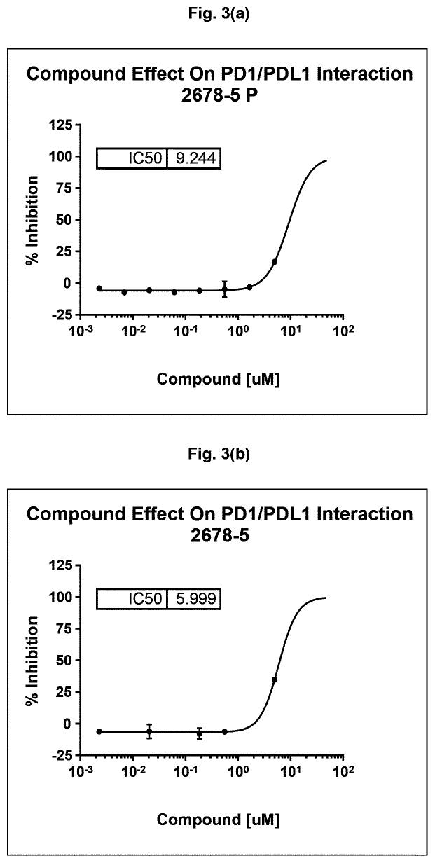

FIGS. 4(a) and 4(b) each illustrate a profile of the effect of a compound of the disclosure, compound 2678-10, at different concentrations on the PD-1/PD-L1 interaction. The IC₅₀ value of the compound is inset. FIG. 4(a) illustrates the profile obtained using a purified sample of 2678-10. FIG. 4(b) illustrates the profile obtained using a crude sample of 2678-10. The data was obtained using a PD-1/PD-L1 HTRF binding assay. For the purified sample, a 10 mM stock solution of the compound in 100% DMSO was run at 8 doses (5.000 μM, 1.667 μM, 0.556 μM, 0.185 μM, 0.062 μM, 0.021 μM, 0.007 μM and 0.002 μM), which were carried out in duplicate. For the crude sample, a 10 mM stock solution of the compound in 100% DMSO was run at 5 doses (5.000 μM, 0.556 μM, 0.185 μM, 0.021 μM and 0.002 μM), which were carried out in duplicate.

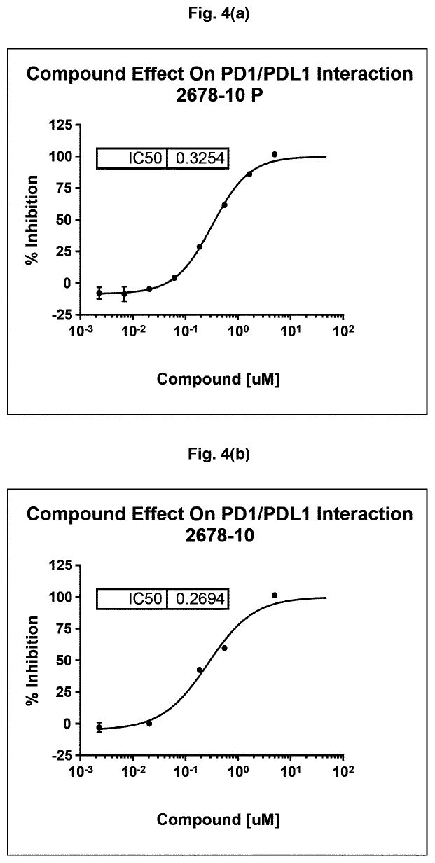

FIGS. 5(a) and 5(b) each illustrate a profile of the effect of a compound of the disclosure, compound 2678-32, at different concentrations on the PD-1/PD-L1 interaction. The IC₅₀ value of the compound is inset. FIG. 5(a) illustrates the profile obtained using a purified sample of 2678-32. FIG. 5(b) illustrates the profile obtained using a crude sample of 2678-32. The data was obtained using a PD-1/PD-L1 HTRF binding assay. For the purified sample, a 10 mM stock solution of the compound in 100% DMSO was run at 8 doses (5.000 μM, 1.667 μM, 0.556 μM, 0.185 μM, 0.062 μM, 0.021 μM, 0.007 μM and 0.002 μM), which were carried out in duplicate. For the crude sample, a 10 mM stock solution of the compound in 100% DMSO was run at 5 doses (5.000 μM, 0.556 μM, 0.185 μM, 0.021 μM and 0.002 μM), which were carried out in duplicate.

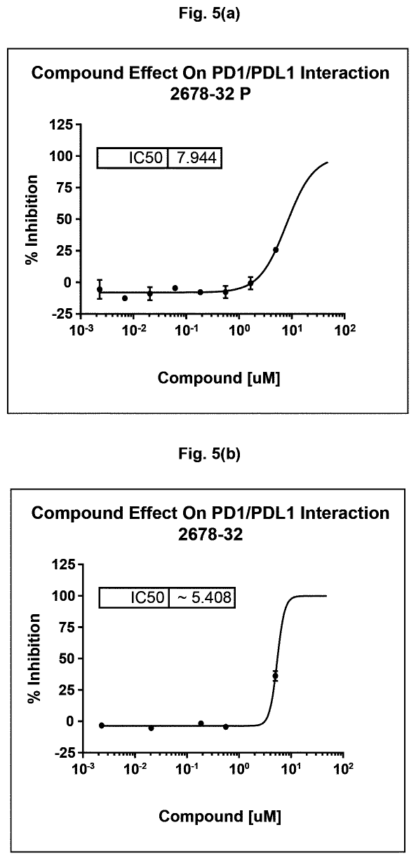

FIGS. 6(a) and 6(b) each illustrate a profile of the effect of a compound of the disclosure, compound 2678-51, at different concentrations on the PD-1/PD-L1 interaction. The IC₅₀ value of the compound is inset. FIG. 6(a) illustrates the profile obtained using a purified sample of 2678-51. FIG. 6(b) illustrates the profile obtained using a crude sample of 2678-51. The data was obtained using a PD-1/PD-L1 HTRF binding assay. For the purified sample, a 10 mM stock solution of the compound in 100% DMSO was run at 8 doses (5.000 μM, 1.667 μM, 0.556 μM, 0.185 μM, 0.062 μM, 0.021 μM, 0.007 μM and 0.002 μM), which were carried out in duplicate. For the crude sample, a 10 mM stock solution of the compound in 100% DMSO was run at 5 doses (5.000 μM, 0.556 μM, 0.185 μM, 0.021 μM and 0.002 μM), which were carried out in duplicate.

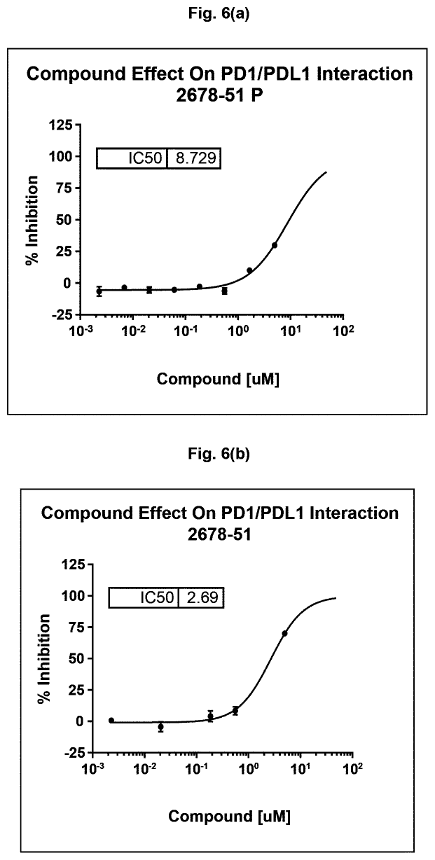

FIGS. 7(a) and 7(b) each illustrate a profile of the effect of a compound of the disclosure, compound 2678-53, at different concentrations on the PD-1/PD-L1 interaction. The IC₅₀ value of the compound is inset. FIG. 7(a) illustrates the profile obtained using a purified sample of 2678-53. FIG. 7(b) illustrates the profile obtained using a crude sample of 2678-53. The data was obtained using a PD-1/PD-L1 HTRF binding assay. For the purified sample, a 10 mM stock solution of the compound in 100% DMSO was run at 8 doses (5.000 μM, 1.667 μM, 0.556 μM, 0.185 μM, 0.062 μM, 0.021 μM, 0.007 μM and 0.002 μM), which were carried out in duplicate. For the crude sample, a 10 mM stock solution of the compound in 100% DMSO was run at 5 doses (5.000 μM, 0.556 μM, 0.185 μM, 0.021 μM and 0.002 μM), which were carried out in duplicate.

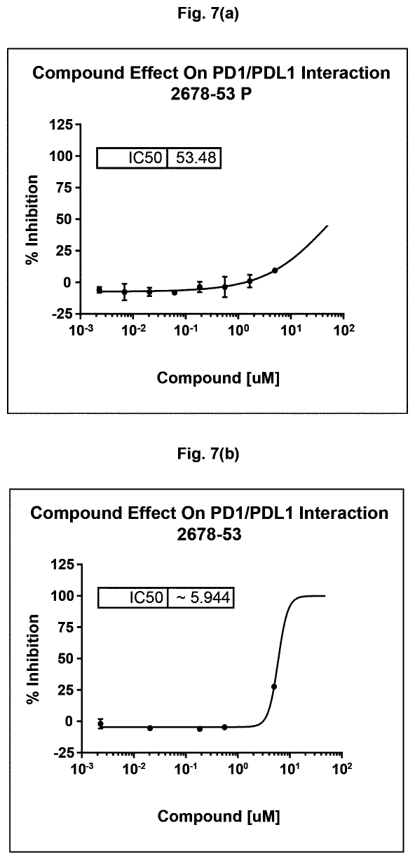

FIGS. 8(a) and 8(b) each illustrate a profile of the effect of a compound of the disclosure, compound 2678-58, at different concentrations on the PD-1/PD-L1 interaction. The IC₅₀ value of the compound is inset. FIG. 8(a) illustrates the profile obtained using a purified sample of 2678-58. FIG. 8(b) illustrates the profile obtained using a crude sample of 2678-58. The data was obtained using a PD-1/PD-L1 HTRF binding assay. For the purified sample, a 10 mM stock solution of the compound in 100% DMSO was run at 8 doses (5.000 μM, 1.667 μM, 0.556 μM, 0.185 μM, 0.062 μM, 0.021 μM, 0.007 μM and 0.002 μM), which were carried out in duplicate. For the crude sample, a 10 mM stock solution of the compound in 100% DMSO was run at 5 doses (5.000 μM, 0.556 μM, 0.185 μM, 0.021 μM and 0.002 μM), which were carried out in duplicate.

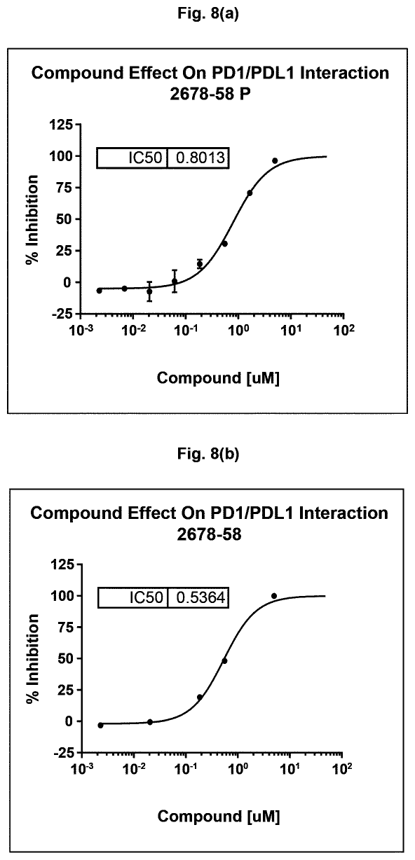

FIGS. 9(a) and 9(b) each illustrate a profile of the effect of a compound of the disclosure, compound 2678-66, at different concentrations on the PD-1/PD-L1 interaction. The IC₅₀ value of the compound is inset. FIG. 9(a) illustrates the profile obtained using a purified sample of 2678-66. FIG. 9(b) illustrates the profile obtained using a crude sample of 2678-66. The data was obtained using a PD-1/PD-L1 HTRF binding assay. For the purified sample, a 10 mM stock solution of the compound in 100% DMSO was run at 8 doses (5.000 μM, 1.667 μM, 0.556 μM, 0.185 μM, 0.062 μM, 0.021 μM, 0.007 μM and 0.002 μM), which were carried out in duplicate. For the crude sample, a 10 mM stock solution of the compound in 100% DMSO was run at 5 doses (5.000 μM, 0.556 μM, 0.185 μM, 0.021 μM and 0.002 μM), which were carried out in duplicate.

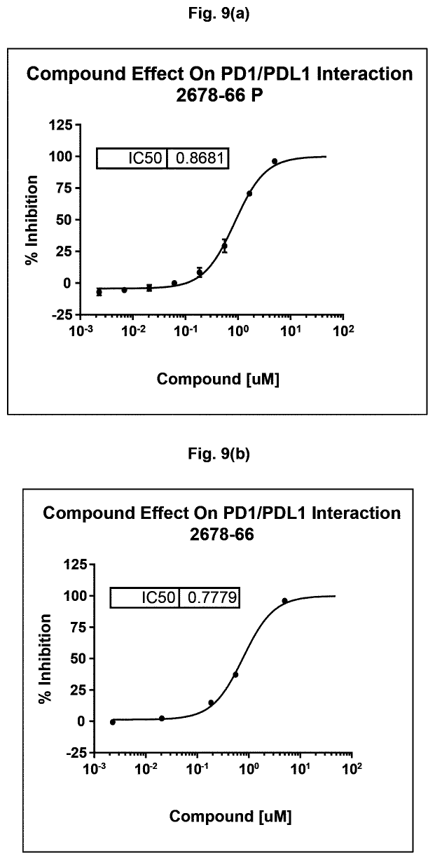

FIG. 10 illustrates a profile of the effect of a compound of the disclosure, compound 2678-72, at different concentrations on the PD-1/PD-L1 interaction. The IC₅₀ value of the compound is inset. The data was obtained using a PD-1/PD-L1 HTRF binding assay. A 10 mM stock solution of the compound in 100% DMSO was run at 8 doses (5.000 μM, 1.667 μM, 0.556 μM, 0.185 μM, 0.062 μM, 0.021 μM, 0.007 μM and 0.002 μM), which were carried out in duplicate.

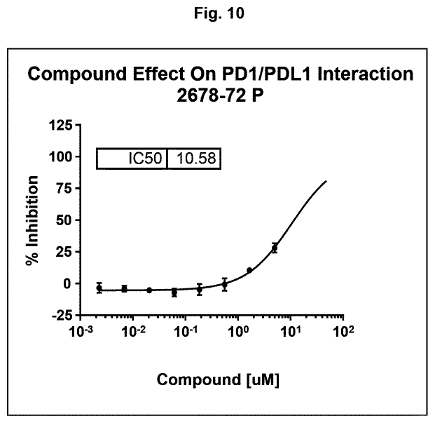

FIGS. 11(a) and 11(b) each illustrate a profile of the effect of a compound of the disclosure, compound 2678-78, at different concentrations on the PD-1/PD-L1 interaction. The IC₅₀ value of the compound is inset. FIG. 11(a) illustrates the profile obtained using a purified sample of 2678-78. FIG. 11(b) illustrates the profile obtained using a crude sample of 2678-78. The data was obtained using a PD-1/PD-L1 HTRF binding assay. For the purified sample, a 10 mM stock solution of the compound in 100% DMSO was run at 8 doses (5.000 μM, 1.667 μM, 0.556 μM, 0.185 μM, 0.062 μM, 0.021 μM, 0.007 μM and 0.002 μM), which were carried out in duplicate. For the crude sample, a 10 mM stock solution of the compound in 100% DMSO was run at 5 doses (5.000 μM, 0.556 μM, 0.185 μM, 0.021 μM and 0.002 μM), which were carried out in duplicate.

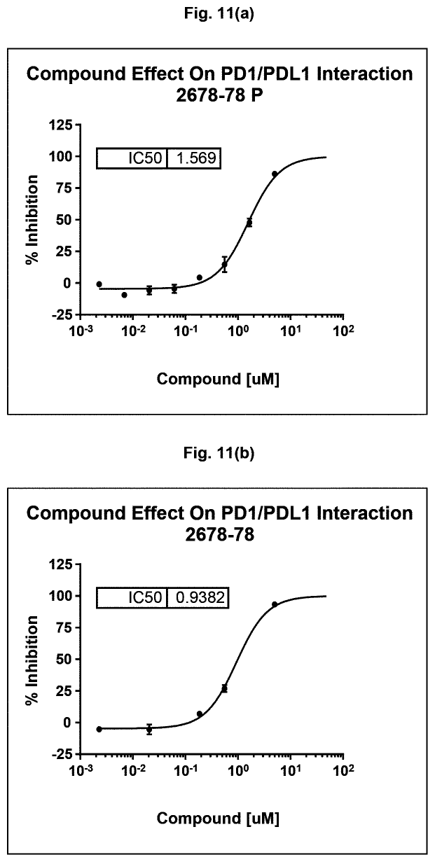

FIGS. 12(a) and 12(b) each illustrate a profile of the effect of a compound of the disclosure, compound 2678-80, at different concentrations on the PD-1/PD-L1 interaction. The IC₅₀ value of the compound is inset. FIG. 12(a) illustrates the profile obtained using a purified sample of 2678-80. FIG. 12(B) illustrates the profile obtained using a crude sample of 2678-80. The data was obtained using a PD-1/PD-L1 HTRF binding assay. For the purified sample, a 10 mM stock solution of the compound in 100% DMSO was run at 8 doses (5.000 μM, 1.667 μM, 0.556 μM, 0.185 μM, 0.062 μM, 0.021 μM, 0.007 μM and 0.002 μM), which were carried out in duplicate. For the crude sample, a 10 mM stock solution of the compound in 100% DMSO was run at 5 doses (5.000 μM, 0.556 μM, 0.185 μM, 0.021 μM and 0.002 μM), which were carried out in duplicate.

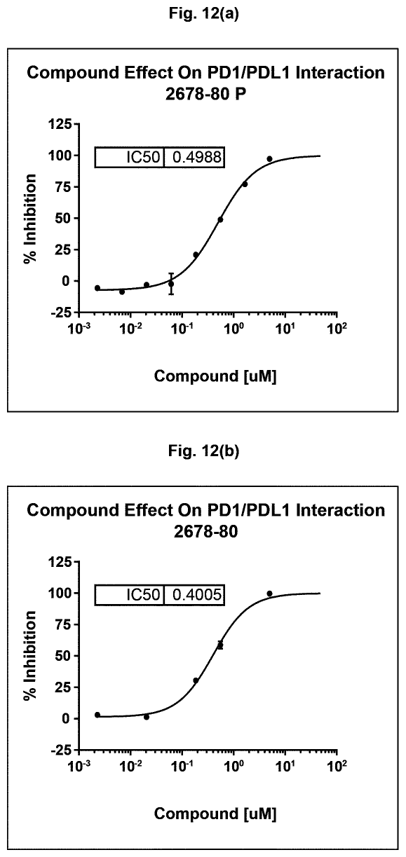

FIG. 13 illustrates a profile of the effect of a compound of the disclosure, compound 2463-262, at different concentrations on the PD-1/PD-L1 interaction. The IC₅₀ value of the compound is inset. The data was obtained using a PD-1/PD-L1 HTRF binding assay. A 10 mM stock solution of the compound in 100% DMSO was run at 5 doses (5.000 μM, 0.556 μM, 0.185 μM, 0.021 μM and 0.002 μM), which were carried out in duplicate.

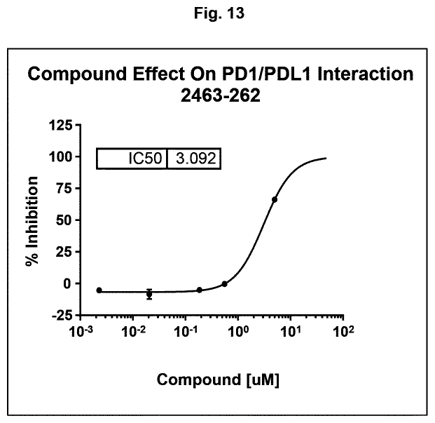

FIG. 14 illustrates a profile of the effect of a comparator compound, compound S7911, on the PD-1/PD-L1 interaction. The IC₅₀ value of the compound is inset. The data was obtained using a PD-1/PD-L1 HTRF binding assay. A 10 mM stock solution of the compound in 100% DMSO was run at 8 doses (5.000 μM, 1.667 μM, 0.556 μM, 0.185 μM, 0.062 μM, 0.021 μM, 0.007 μM and 0.002 μM), which were carried out in duplicate. Compound S7911 has previously been reported to have an IC₅₀ of 0.006 μM in a PD-1 inhibition assay (see, Abdel-Magid, Ahmed F, “Inhibitors of the PD-1/PD-L1 Pathway Can Mobilize the Immune System: An Innovative Potential Therapy for Cancer and Chronic Infections” (14 Apr. 2015) ACS medicinal chemistry letters, 6(5), 489, doi:10.1021/acsmedchemlett.5b00148).

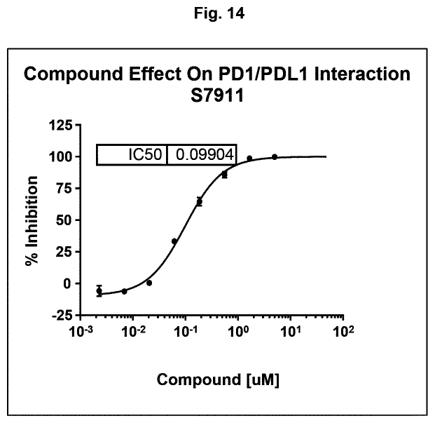

### DETAILED DESCRIPTION

### Definitions

Unless defined otherwise, all technical and scientific terms used herein have the same meaning as commonly understood to one of ordinary skill in the art to which this disclosure pertains.

The abbreviation PD-1 refers to programmed cell death protein 1. PD-1 is also known as cluster of differentiation 279 (CD279). A PD-1 blocker is a compound that interacts with PD-1 to block the PD-1/PD-L1 interaction.

The abbreviation PD-L1 refers to programmed death-ligand 1. PD-L1 is also known as cluster of differentiation 274 (CD274) and B7 homolog 1 (B7-H1). A PD-L1 blocker is a compound that interacts with PD-L1 to block the PD-1/PD-L1 interaction.

The term “substituted”, when used with an atom or group, refers to the designated atom or group where one or more hydrogen atoms on the atom or group is replaced with one or more substituents other than hydrogen, provided that the referred to atom or group's normal valence is not exceeded. The one or more substituents include, but are not limited to: alkyl, alkenyl, alkynyl, alkoxy, acyl, amidino, amido, amino, aryl, azido, carbonyl (oxo), carboxyl, carboxyl ester, cyano, cycloalkyl, cycloalkenyl, guanidino, ureido, halo, haloalkyl, heteroalkyl, heteroaryl, heterocycloalkyl, hydroxy, hydrazino, imino, nitro, alkylsulfinyl, sulfonic acid, alkylsulfonyl, thiocyanate, thiol, alkylthio, thione (thioketone), and combinations thereof. Unless indicated otherwise, the maximum number of serial substitutions in compounds of the disclosure is three. Thus, a structure arrived at by defining substituents with further substituents appended ad infinitum is not covered by the present disclosure.

The terms “aliphatic hydrocarbon” or “aliphatic group” (which may be used interchangeably) refer to a hydrocarbon compound or group containing carbon and hydrogen joined together in straight chains, branched chains or non-aromatic rings.

The term “alkyl”, by itself or as part of another substituent, refers to, unless otherwise stated, a straight or branched chain, substituted or unsubstituted, aliphatic group having any number of carbons, such as for example 1 to 20 carbon atoms, and more particularly having the number of carbon atoms as designated (e.g. C₁₋₆ meaning 1 to 6 carbon atoms). An exemplary “alkyl” group is a methyl group (—CH₃; Me).

The term “alkene” by itself or as part of another substituent, refers to, unless otherwise stated, a straight or branched chain, substituted or unsubstituted, aliphatic group having any number of carbons greater than or equal to 2, such as for example 2 to 20 carbon atoms, and more particularly having the number of carbon atoms as designated (e.g. C₂₋₆ meaning 2 to 6 carbon atoms) and containing at least one carbon-carbon double bond. As a functional group it may be referred to herein also as “alkenyl”. An alkene or alkenyl group may comprise more than one carbon-carbon double bond. The term “dialkenyl”, for example, may be used herein to represent an unsaturated aliphatic hydrocarbon group containing two carbon-carbon double bonds.

The term “alkyne” by itself or as part of another substituent, refers to, unless otherwise stated, a straight or branched chain, substituted or unsubstituted, aliphatic group having any number of carbons greater than or equal to 2, such as for example 2 to 20 carbon atoms, and more particularly having the number of carbon atoms as designated (e.g. C₂₋₆ meaning 2 to 6 carbon atoms) and containing at least one carbon-carbon triple bond. As a functional group it may be referred to herein also as “alkynyl”. An alkyne or alkynyl group may comprise more than one carbon-carbon triple bond. The term “dialkynyl”, for example, is used herein to represent an unsaturated aliphatic hydrocarbon group containing two carbon-carbon triple bonds.

The term “cycloalkyl” refers to a cyclic version of “alkyl” having one of more rings, and includes, for example, fused, bridged, and spiro ring systems. Examples of cycloalkyl include, but are not limited to, cyclopropyl, cyclobutyl, cyclopentyl, cyclohexyl, cycloheptyl, adamantyl, and the like. The term “cycloalkylene” refers to a divalent radical derived from a cycloalkyl.

The term “cycloalkenyl” refers to a cyclic version of “alkenyl” having one of more rings, and includes, for example, fused, bridged, and spiro ring systems (i.e. a non-aromatic carbocyclic group having at least one double bond). Examples of cycloalkenyl include, but are not limited to, cyclopentenyl, cyclohexenyl, cyclopentadienyl, and the like.

The appendation of “hetero” to a defined group, for example alkyl, alkene, alkyne, cycloalkyl, cycloalkenyl and aryl, means that one or more of the carbon atoms (and any associated hydrogen atoms) of the group are each independently replaced with the same or different heteroatom (e.g. nitrogen, oxygen, sulfur, phosphorus, silicon or selenium). Thus, “heteroalkyl”, for example, refers to an alkyl group in which one or more of the carbon atoms are each independently replaced with the same or different heteroatom. For example, the term “C₃heteroalkyl” includes, but is not limited to, —OCH₂CH₃, —CH₂OCH₃, —CH₂SCH₃, and the like; the term “C₅heterocycloalkyl” includes, but is not limited to, C₄H₈N, C₄H₇O, C₃H₆NS, C₃H₆NO, and the like; and the term “C₆heteroaryl” includes, but is not limited to, C₅H₄N, C₄H₃N₂, and the like.

The terms “heterocycloalkyl” or heterocycloalkenyl” may collectively be referred to as “heterocyclic groups” or “heterocycles”. In a heterocycloalkyl or heterocycloalkenyl, a heteroatom can occupy the position at which the heterocycle is attached to the remainder of the molecule. Examples of heterocycloalkyl and heterocycloalkenyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “aromatic group” and “aromatic ring” (which may be used interchangeably) refer to a substituent group that comprises one or more aromatic rings. If the aromatic group comprises more than one aromatic ring, the rings may be attached together in a pendent manner or may be fused. The term “aromatic group” encompasses carbocyclic aromatic groups (containing only carbon atoms in the aromatic ring or rings) and heteroaromatic groups (containing carbon and one or more other atoms in at least one of the aromatic rings).

The term “aryl group” refers a carbocyclic aromatic group having one or more carbon rings wherein such rings may be attached together in a pendent manner or may be fused. An aryl group may contain one, two, three or more rings. Monocyclic embodiments may contain 4 to 10 carbon atoms, more particularly 4 to 7 carbon atoms, and even more particularly 6 carbon atoms in the ring. Bicyclic embodiments may contain 8 to 12 carbon atoms, more particularly 8 to 10, and even more particularly 9 or 10 carbon atoms in the rings. Tricyclic embodiments may contain 12 to 16 carbon atoms, and more particularly 14 carbon atoms in the rings. Examples of aryl groups include, but are not limited to:

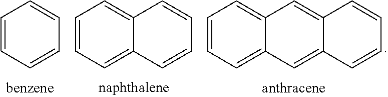

The term “heteroaryl group” refers to a heteroaromatic group having one or more rings wherein such rings may be attached together in a pendent manner or may be fused, wherein the aromatic group has at least one heteroatom such as, for example, nitrogen, oxygen, sulfur, phosphorus, silicon or selenium. A heteroaryl group may contain one, two, three or more rings. Monocyclic embodiments may contain 4 to 10 member atoms, more particularly 4 to 7 member atoms, and even more particularly 5 or 6 member atoms in the ring. Bicyclic embodiments may contain 8 to 12 member atoms, more particularly 8 to 10 member atoms, and even more particularly 9 or 10 member atoms in the rings. Tricyclic embodiments may contain 12 to 16 member atoms, and more particularly 14 member atoms in the rings. Examples of heteroaryl groups include, but are not limited to:

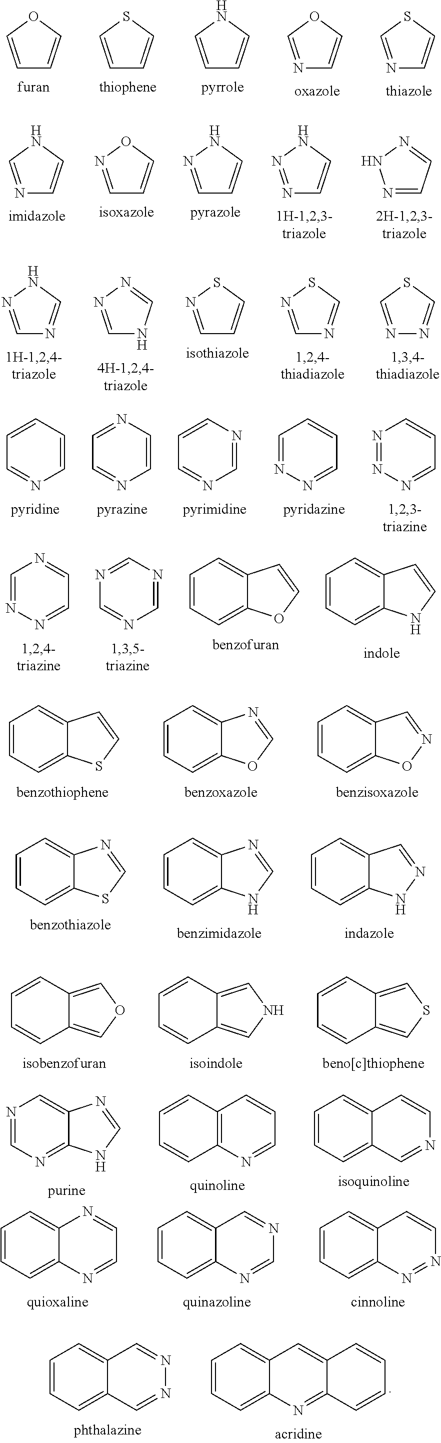

Aryl and heteroaryl groups may be unsubstituted or substituted at one or more positions. Where an aryl or heteroaryl group is substituted at more than one position, multiple substituent groups, together with the atoms to which they are attached may form a further cyclic system. A non-limiting example of an aryl group with two substituent groups forming a further cyclic system is 1,3-benzodioxole.

The terms “arylene” and “heteroarylene”, either alone or as part of another substituent, refer to a divalent radical derived from an aryl and heteroaryl, respectively.

The terms “aryl-alkyl” or “heteroaryl-alkyl” refer to an aryl or heteroaryl group as defined above, respectively, comprising an alkyl group. The alkyl group is the means by which the aryl or heteroaryl group is attached to the remainder of the molecule. An exemplary “aryl-alkyl” group is a benzyl group (—CH₂—C₆H₆; Bzl), where the methylene fragment (—CH₂—) serves as the linking group for the aryl group (—C₆H₆). An exemplary “heteroaryl-alkyl” group is a pyridylmethyl group (—CH₂—C₅H₅N), where the methylene fragment (—CH₂—) serves as the linking group for the heteroaryl group (—C₅H₅N). The terms “aryl-heteroalkyl” or “heteroaryl-heteroalkyl” refer to an “aryl-alkyl” or “heteroaryl-alkyl” group, respectively, where one or more carbon atoms of the alkyl group are replaced by a heteroatom (e.g., phenoxymethyl, 2-pyridyloxymethyl, and the like).

The terms “halo” or “halogen” refer to fluorine, chlorine, bromine and iodine. The appendation of “halo” to a defined group means that one or more of the hydrogen atoms of the group are each independently replaced with the same or different halogen. Thus, the terms “haloalkyl”, “haloalkene” and “halocycloalkyl” refer to an alkyl, alkene and cycloalkyl group, respectively, as defined above, wherein one or more hydrogen atoms are replaced by a halogen. For example, the term “halo(C₁-C₂)alkyl” includes, but is not limited to, fluoromethyl, dichloromethyl, tribromomethyl, 2,2,2-trifluoroethyl, and the like.

The terms “hydroxy” and “hydroxyl” (which may be used interchangeably) refer to a —OH moiety.

The term “alkoxy” refers to a —OR moiety. R may be, for example, alkyl, alkenyl, alkynyl, cycloalkyl, heterocycloalkyl, cycloalkenyl or heterocycloalkenyl; each of which may be substituted or unsubstituted.

The term “aryloxy” refers to a —OR moiety. R may be, for example, aryl or heteroaryl; each of which may be substituted or unsubstituted.

The term “amino” refers to a —NRR moiety. Each R may be, for example, hydrogen, alkyl, alkenyl, alkynyl, cycloalkyl, heterocycloalkyl, cycloalkenyl, heterocycloalkenyl, aryl or heteroaryl; each of which may be substituted or unsubstituted, or both R groups may be joined, together with the N atom to which they are attached, to form a heterocyclic group as described herein, which may be substituted or unsubstituted.

The term “cyano” refers to a —CN moiety.

The term “azido” refers to a —N₃ moiety.

The term “hydrazino” refers to a —NH—NH₂ moiety.

The term “nitro” refers to a —NO₂ moiety.

The term “guanidino” refers to a —NH—C(NH)NH₂ moiety.

The term “ureido” refers to a —NH—C(O)NH₂ moiety.

The term “alkylsulfinyl” refers to a —S(═O)-alkyl moiety.

The term “sulfonic acid” refers to a —S(═O)₂—OH moiety.

The term “alkylsulfonyl” refers to a —S(═O)₂-alkyl moiety.

The term “thiocyanate” refers to a —SCN moiety.

The terms “thiol” and “sulfhydryl” (which may be used interchangeably) refer to a —SH moiety.

The term “thioether” refers to a —SR moiety. R may be, for example, alkyl, alkenyl, alkynyl, cycloalkyl, heterocycloalkyl, cycloalkenyl, heterocycloalkenyl, aryl or heteroaryl; each of which may be substituted or unsubstituted.

The terms “carbonyl” and “oxo” (which may be used interchangeably) refer to a (C═O) moiety. A carbonyl group may also be represented as —C(O)—.

The term “carboxyl” refers to a —C(O)OH moiety.

The terms “ester” and “carboxyl ester” (which may be used interchangeably) refer to a —C(O)OR moiety. R may be, for example, alkyl, alkenyl, alkynyl, cycloalkyl, heterocycloalkyl, cycloalkenyl, heterocycloalkenyl, aryl or heteroaryl; each of which may be substituted or unsubstituted.

The term “acyl” refers to a —C(O)R moiety. R may be, for example, alkyl, alkenyl, alkynyl, cycloalkyl, heterocycloalkyl, cycloalkenyl, heterocycloalkenyl, aryl or heteroaryl; each of which may be substituted or unsubstituted.

The term “amido” refers to a —C(O)NRR moiety. Each R may be, for example, hydrogen, alkyl, alkenyl, alkynyl, cycloalkyl, heterocycloalkyl, cycloalkenyl, heterocycloalkenyl, aryl or heteroaryl; each of which may be substituted or unsubstituted, or both R groups may be joined, together with the N atom to which they are attached, to form a heterocyclic group as described herein, which may be substituted or unsubstituted.

The term “imino” refers to a —C(NR)— moiety. R may be, for example, hydrogen, alkyl, alkenyl, alkynyl, cycloalkyl, heterocycloalkyl, cycloalkenyl, heterocycloalkenyl, aryl or heteroaryl; each of which may be substituted or unsubstituted.

The term “amidino” refers to a —C(NR)NRR moiety. Each R may be, for example, hydrogen, alkyl, alkenyl, alkynyl, cycloalkyl, heterocycloalkyl, cycloalkenyl, heterocycloalkenyl, aryl or heteroaryl; each of which may be substituted or unsubstituted, or both R groups of the “NRR” fragment may be joined to form, together with the N atom to which they are attached, a heterocyclic group as described herein, which may be substituted or unsubstituted.

The term “thione” refers to a (C═S) moiety. A thione group may also be represented as —C(S)—.

A skilled person will recognize that it may be possible to arrive at each of the aforementioned groups via substitution of another group. For example, monofluoro methyl can be described as a haloalkyl group, as well as a substituted alkyl group, and methoxy can be described as a heteroalkyl group or an alkoxy group. Thus, the omission of one of the aforementioned groups does not necessarily preclude the inclusion of a group falling within that definition.

The term “pharmaceutically acceptable” refers to those compounds, materials, compositions, dosage forms, etc. which are, within the scope of sound medical judgment, suitable for use in contact with the tissues of a subject without excessive toxicity, irritation, allergic response, or other problem or complication, commensurate with a reasonable benefit/risk ratio. For example, a “pharmaceutically acceptable salt” of a compound of the disclosure may refer to a salt of a compound of Formula (I) that retains biological activity, and which is not biologically or otherwise undesirable.

The term “isomers” refers to compounds having the same number and kind of atoms but differing in respect to the structural arrangement or configuration of the atoms. Isomers include, for example, structural isomers and stereoisomers (e.g. enantiomers and diastereomers).

The term “tautomer” refers to one of two or more structural isomers of a compound which exist in equilibrium and which are readily converted from one isomeric form to another.

The term “prodrug” refers to compounds that readily undergo chemical changes under physiological conditions, for example by oxidation, reduction, hydrolysis or the like, each of which is carried out enzymatically or without enzyme involvement, to provide a specific compound. For example, a prodrug of a compound of the disclosure is a compound that readily undergoes chemical changes under physiological conditions to form a compound of the disclosure.

Various forms of prodrugs are known in the art (see, for example: A Textbook of Drug Design and Development, P. Krogsgaard-Larson and H. Bundgaard, eds. Ch 5, pgs 113-191 (Harwood Academic Publishers, 1991); and Hydrolysis in Drug and Prodrug Metabolism, Bernard Testa and Joachim M. Mayer, (Wiley-VCH, 2003)). Examples of prodrugs include compounds wherein: an amino group in the compound is acylated, alkylated or phosphorylated; a hydroxyl group in the compound is acylated, alkylated, phosphorylated or converted into the corresponding borate; a carboxyl group in the compound is esterified or amidated; a carboxylate in the compound is converted into an alkyl-, aryl-, choline-, amino, acyloxymethylester, linolenoyl-ester; or a sulfhydryl group in the compound forms a disulfide bridge with a carrier molecule, e.g. a peptide, that delivers the compound selectively to a target and/or to the cytosol of a cell.

The terms “polymorph” and “crystalline form” (which may be used interchangeably) refer to different crystal structures of a crystalline compound. Different polymorphs may result from differences in crystal packing (packing polymorphism) or differences in packing between different conformers of the same molecule (conformational polymorphism).

The term “solvate” refers to a complex formed by combining a compound with a solvent. The term “hydrate” refers to a solvate where the solvent is water.

The terms “treating” or “treatment”, or “preventing” or “prevention”, as used herein, refer to an approach for obtaining beneficial or desired results, including clinical results, in a subject in need thereof. Beneficial or desired results may include, but are not limited to, alleviation or amelioration of one or more symptoms or conditions, diminishment of extent of disease, stabilisation of the state of disease, prevention of development of disease, prevention of spread of disease, delay or slowing of disease progression (e.g. suppression), delay or slowing of disease onset, conferring protective immunity against a disease-causing agent and amelioration or palliation of the disease state. “Treating” or “preventing” can also mean prolonging survival of a patient beyond that expected in the absence of treatment and can also mean inhibiting the progression of disease temporarily or preventing the occurrence of disease, such as by preventing infection in a subject. “Treating” or “preventing” may also refer to a reduction in the size of a tumor mass, reduction in tumor aggressiveness, etc. “Treating” or “preventing” may be measured based on objective or subjective parameters.

The expression “a subject in need thereof”, as used herein, is meant to encompass not only a subject who has a particular disease, disorder or condition, but also a subject who may potentially contract the disease, disorder or condition.

The term “therapeutic agent”, as used herein, is meant to encompass any reagent that is effective in the treatment of a disease or condition. For example, an “anticancer agent” is a therapeutic agent that is effective in treating a malignant, or cancerous disease, i.e. a reagent that may lead to inhibition, partial, or full remission, prolongation of life, improvement in quality of life, or cure.

The term “additional therapeutic agent”, as used herein, refers to a second, third, fourth, etc. therapeutic agent in addition to a compound of the disclosure. An additional therapeutic agent may be another compound of the disclosure or a therapeutic agent that is not a compound of the disclosure.

Reference to singular forms, for example, “a”, “an” and “the”, include plural reference unless the context clearly dictates otherwise. Thus, for example, reference to “a compound of Formula (I)” includes a plurality of such compounds.

The phrase “and/or”, as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The conjunction “or”, as used herein, should be understood to encompass the same meaning as “and/or” as defined above, unless indicated otherwise, or the context clearly dictates otherwise. For example, when separating items in a list, “or” or “and/or” shall be interpreted as being inclusive, i.e., the inclusion of at least one, but also including more than one, of a number or list of elements, and, optionally, additional unlisted items.

The transitional terms “comprising”, “including”, “carrying”, “having”, “containing”, “involving”, and the like, as used herein, are to be understood as being inclusive or open-ended (i.e., to mean including but not limited to), and they do not exclude unrecited elements, materials or method steps. Only the transitional phrases “consisting of” and “consisting essentially of”, respectively, are closed or semi-closed transitional phrases with respect to claims and exemplary embodiment paragraphs herein. The transitional phrase “consisting of” excludes any element, step, or ingredient which is not specifically recited. The transitional phrase “consisting essentially of” limits the scope to the specified elements, materials or steps and to those that do not materially affect the basic characteristic(s) of the invention disclosed and/or claimed herein.

The expressions “one or more” and “at least one” (which may be used interchangeably), unless explicitly stated otherwise herein, refer to the number of different entities (e.g. number of different compounds of Formula (I), etc.), and not to the quantity of any particular entity, in accordance with the ordinary meaning of “at least one” or “one or more”.

The term “about” refers to a variation of plus or minus 10%. For example, when used with a number or range of numbers, the term “about” refers to that number plus or minus 10%, or minus 10% the lower end of the range of numbers to the upper end of the range of numbers plus 10%.

The symbols “ [symbol] ” and “ [symbol] ” (which may be used interchangeably) when placed intersecting with a bond in a structural fragment are used herein to indicate connectivity of the structural fragment to the remainder of a compound as described herein.

Compounds

The present disclosure relates to compounds of Formula (I):

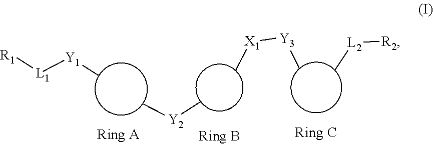

wherein:
R₁ is C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆ alkyl, C₅₋₁₄aryl-C₂₋₆heteroalkyl or C₅₋₁₄heteroaryl-C₂₋₆heteroalkyl, each of which is unsubstituted or substituted with one or more R^{A};
R₂ is C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆ alkyl, C₅₋₁₄aryl-C₂₋₆heteroalkyl or C₅₋₁₄heteroaryl-C₂₋₆heteroalkyl, each of which is unsubstituted or substituted with one or more R^{A};
L₁ is optional and if present is C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl or C₅₋₁₄ heterocycloalkenyl, each of which is unsubstituted or substituted with one or more R^{A};
L₂ is optional and if present is C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl or C₅₋₁₄ heterocycloalkenyl, each of which is unsubstituted or substituted with one or more R^{A};
Y₁, Y₂ and Y₃ are independently —CR₅R₆—, —C(CR₅R₆)—, —C(O)—, —C(NR^{B})—, —C(S)—, —NR^{B}, —O—, —S—, —(O)— or —(O)₂—;
X₁ is —CR^{B}₂—, —C(CR^{B}₂)—, —C(O)—, —C(NR^{B})—, —C(S)—, —(NR^{B})—, —O—, —S—, —(O)— or —S(O)₂—;
Ring A is C₆ cycloalkyl or C₆ heterocycloalkyl, each of which is unsubstituted or substituted with one or more R₃;
Ring B is C₆ aryl or C₅₋₆ heteroaryl, each of which is unsubstituted or substituted with one or more R^{A};
Ring C is C₅₋₆ cycloalkyl or C₅₋₆ heterocycloalkyl, each of which is unsubstituted or substituted with one or more R₄;
R₃ and R₄ at each occurrence are independently C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₁₋₆ haloalkyl, C₂₋₆ haloalkenyl, C₂₋₆ haloalkynyl, C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₃₋₁₄ haloheterocycloalkyl, C₅₋₁₄ haloheterocycloalkenyl, C₅₋₁₄ aryl, C₅₋₁₄ heteroaryl, halo C₅₋₁₄ aryl, halo C₅₋₁₄ heteroaryl, C₅₋₁₄aryl-C₁₋₆alkyl, C₅₋₁₄heteroaryl-C₁₋₆alkyl, haloC₅₋₁₄aryl-C₁₋₆ alkyl, haloC₅₋₁₄heteroaryl-C₁₋₆alkyl, C₅₋₁₄aryl-C₂₋₆heteroalkyl, C₅₋₁₄heteroaryl-C₂₋₆ heteroalkyl, haloC₅₋₁₄aryl-C₂₋₆-heteroalkyl, haloC₅₋₁₄heteroaryl-C₂-heteroalkyl, —F, —Cl, —Br, —I, —CN, —NO₂, —SO₂, —N₃, —SCN, —NCS, —OR^{B}, —NR^{C}R^{D}, —NR^{B}NR^{C}R^{D}, —SR^{B}, —C(O)R^{B}, —C(O)OR^{B}, —C(O)NR^{C}R^{D}, —C(NR^{C})R^{B}, —C(NR^{B})NR^{C}R^{D}, —S(O)R^{B}, —S(O)₂R^{B}, —S(O)OR^{B}, —S(O)₂OR^{B}, —NR^{B}NR^{C}R^{D}, —NR^{B}C(O)NR^{C}R^{D} or —NR^{B}C(NR^{B})NR^{C}R^{D}; two or more R₃ or two or more R₄ together with the atom(s) to which they are attached form a cyclic group; and/or two R₃ or two R₄ on the same atom form (═O), (═NR^{B}) or (═S);
R₅ and R₆ at each occurrence are independently —H, C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₁₋₆ haloalkyl, C₂₋₆ haloalkenyl, C₂₋₆ haloalkynyl, C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₃₋₁₄ haloheterocycloalkyl, C₅₋₁₄ haloheterocycloalkenyl, C₅₋₁₄ aryl, C₅₋₁₄ heteroaryl, halo C₅₋₁₄ aryl, halo C₅₋₁₄ heteroaryl, C₅₋₁₄aryl-C₁₋₆alkyl, C₅₋₁₄heteroaryl-C₁₋₆alkyl, haloC₅₋₁₄aryl-C₁₋₆ alkyl, haloC₅₋₁₄heteroaryl-C₁₋₆alkyl, C₅₋₁₄aryl-C₂₋₆heteroalkyl, C₅₋₁₄heteroaryl-C₂₋₆ heteroalkyl, haloC₅₋₁₄aryl-C₂₋₆heteroalkyl, haloC₅₋₁₄heteroaryl-C₂₋₆heteroalkyl, —F, —Cl, —Br, —I, —CN, —NO₂, —SO₂, —N₃, —SCN, —NCS, —OR^{B}, —NR^{C}R^{D}, —NR^{B}NR^{C}R^{D}—, SR^{B}, —C(O)R^{B}, —C(O)OR^{B}, —C(O)NR^{C}R^{D}, —C(NR^{C})R^{B}, —C(NR^{B})NR^{C}R^{D}, —S(O)R^{B}, —S(O)₂R^{B}, —S(O)OR^{B}, —S(O)₂OR^{B}, —NR^{B}NR^{C}R^{D}, —NR^{B}C(O)NR^{C}R^{D} or —NR^{B}C(NR^{B})NR^{C}R^{D}; or R₅ and R₆ together with the carbon atom to which they are attached form a C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₃₋₁₄ haloheterocycloalkyl, or C₅₋₁₄ haloheterocycloalkenyl;
R^{A} at each occurrence is independently —F, —Cl, —Br, —I, —CN, —NO₂, —SO₂, —N₃, —SCN, —NCS, —OR^{B}, —NR^{C}R^{D}, —NR^{B}NR^{C}R^{D}, —SR^{B}, —C(O)R^{B}, —C(O)OR^{B}, —C(O)NR^{C}R^{D}, —C(NR^{B})R^{B}, —C(NR^{B})NR^{C}R^{D}, —S(O)R^{B}, —S(O)₂R^{B}, —S(O)OR^{B}, —S(O)₂OR^{B}, —NR^{B}NR^{C}R^{D}, —NR^{B}C(O)NR^{C}R^{D}, —NR^{B}C(NR^{B})NR^{C}R^{D}, C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆heteroalkyl or C₅₋₁₄heteroaryl-C₂₋₆ heteroalkyl, wherein each C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆heteroalkyl or C₅₋₁₄heteroaryl-C₂₋₆heteroalkyl is unsubstituted or substituted with one or more —F, —Cl, —Br, —I, —CN, —NO₂, —SO₂, —N₃, —SCN, —NCS, —OR^{B}, —NR^{C}R^{D}, —NR^{B}NR^{C}R^{D}, —SR^{B}, —C(O)R^{B}, —C(O)OR^{B}, —C(O)NR^{C}R^{D}, —C(NR^{B})R^{B}, —C(NR^{B})NR^{C}R^{D}, —NR^{B}C(NR^{B})NR^{C}R^{D}, —S(O)R^{B}, —S(O)₂R^{B}, —S(O)OR^{B}, —S(O)₂OR^{B}, —NR^{B}NR^{C}R^{D} or —NR^{B}C(O)NR^{C}R^{D}; two or more R^{A} together with the atom(s) to which they are attached form a cyclic group; and/or two R^{A} on the same atom form (═O), (═NR^{B}) or (═S);
R^{B} at each occurrence is independently (i) —H, or (ii) C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆heteroalkyl or C₅₋₁₄heteroaryl-C₂₋₆ heteroalkyl, each of which is unsubstituted or substituted with one or more R^{E};
R^{C} and R^{D} at each occurrence are independently (i) —H, or (ii) C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆heteroalkyl or C₅₋₁₄heteroaryl-C₂₋₆ heteroalkyl, each of which is unsubstituted or substituted with one or more R^{E}, or R^{C} and R^{D} together with the nitrogen atom to which they are attached combine to form a heterocycle that is unsubstituted or substituted with one or more R^{E} groups;
R^{E} at each occurrence is independently —F, —Cl, —Br, —I, —CN, —NO₂, —SO₂, —N₃, —SCN, —NCS, —OR^{F}, —NR^{G}R^{H}, —NR^{F}NR^{G}R^{H}, —SR^{F}, —C(O)R^{F}, —C(O)OR^{F}, —C(O)NR^{G}R^{H}, —C(NR^{F})R^{F}, —C(NR^{F})NR^{G}R^{H}, —S(O)R^{F}, —S(O)₂R^{F}, —S(O)OR^{F}, —S(O)₂OR^{F}, —NR^{F}NR^{G}R^{H}, —NR^{F}C(O)NR^{G}R^{H}, —NR^{F}C(NR^{F})NR^{G}R^{H}, C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆heteroalkyl or C₅₋₁₄heteroaryl-C₂₋₆ heteroalkyl, wherein each C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄ heteroaryl-C₁₋₆alkyl, C₅₋₁₄aryl-C₂₋₆heteroalkyl or C₅₋₁₄heteroaryl-C₂₋₆heteroalkyl is unsubstituted or substituted with one or more —F, —Cl, —Br, —I, —CN, —NO₂, —SO₂, —N₃, —SCN, —NCS, —OR^{F}, —NR^{G}R^{H}, —NR^{F}NR^{G}R^{H}, —SR^{F}, —C(O)R^{F}, —C(O)OR^{F}, —C(O)NR^{G}R^{H}, —C(NR^{F})R^{F}, —C(NR^{F})NR^{G}R^{H}, —NR^{F}C(NR^{F})NR^{G}R^{H}, —S(O)R^{F}, —S(O)₂R^{F}, —S(O)OR^{F}, —S(O)₂OR^{F}, —NR^{F}NR^{G}R^{H} or —NR^{F}C(O)NR^{G}R^{H}; two or more R^{E} together with the atoms to which they are attached form a cyclic group; and/or two R^{E} on the same atom form (═O), (═NR^{F}) or (═S);
R^{F} at each occurrence is independently (i) —H, or (ii) C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₁₋₆ haloalkyl, C₂₋₆ haloalkenyl, C₂₋₆ haloalkynyl, C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₅₋₁₄ aryl, C₅₋₁₄ heteroaryl, halo C₅₋₁₄ aryl, halo C₅₋₁₄ heteroaryl, C₅₋₁₄aryl-C₁₋₆alkyl, C₅₋₁₄heteroaryl-C₁₋₆ alkyl, haloC₅₋₁₄aryl-C₁₋₆alkyl, haloC₅₋₁₄heteroaryl-C₁₋₆alkyl, C₅₋₁₄aryl-C₂₋₆heteroalkyl, C₅₋₁₄heteroaryl-C₂₋₆heteroalkyl, haloC₅₋₁₄aryl-C₂₋₆heteroalkyl or haloC₅₋₁₄heteroaryl-C₂₋₆ heteroalkyl; and
R^{G} and R^{H} at each occurrence are independently (i) —H, or (ii) C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₁₋₆ haloalkyl, C₂₋₆ haloalkenyl, C₂₋₆ haloalkynyl, C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₅₋₁₄ aryl, C₅₋₁₄ heteroaryl, halo C₅₋₁₄ aryl, halo C₅₋₁₄ heteroaryl, C₅₋₁₄aryl-C₁₋₆alkyl, C₅₋₁₄heteroaryl-C₁₋₆ alkyl, haloC₅₋₁₄aryl-C₁₋₆alkyl, haloC₅₋₁₄heteroaryl-C₁₋₆alkyl, C₅₋₁₄aryl-C₂₋₆heteroalkyl, C₅₋₁₄ heteroaryl-C₂₋₆heteroalkyl, haloC₅₋₁₄aryl-C₂₋₆heteroalkyl or haloC₅₋₁₄heteroaryl-C₂₋₆ heteroalkyl, or R^{G} and R^{H} together with the nitrogen atom to which they are attached combine to form a heterocycle.

In some embodiments of the compound of Formula (I), the compound is a compound of Formula (II):

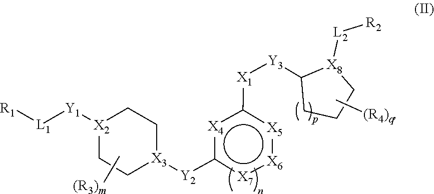

wherein:
X₂, X₃ and X₈ are independently CH, CR^{A} or N;
X₄, X₅ and X₆, and X₇ if present, are independently —CH—, —CR^{A}—, —C(O)—, —C(NR^{B})—, —C(S)—, —N—, —NR^{B}—, —O—, or —S—, provided that the combination of X₄, X₅, X₆ and optionally X₇ together with the atoms to which they are attached forms an aromatic or heteroaromatic group;
m is 0 to 8;
n is 0 or 1;
p is 1 or 2; and
q is 0 to 9.

In some embodiments of the compound of Formula (II), X₂ and X₃ are independently —CH—, —C(C₁₋₆ alkyl)- or —N—.

In some embodiments of the compound of Formula (II), X₂ is —CH— and X₃ is —N—; X₂ is —N— and X₃ is —CH—; X₂ is —CH— and X₃ is —CH—; or X₂ is —N— and X₃ is —N—.

In some embodiments of the compound of Formula (II), m is 0 to 4, and R₃ at each occurrence is independently —F, —C, —Br, —I, —OH, C₁₋₆ alkyl or C₂₋₆ heteroalkyl, wherein each C₁₋₆ alkyl or C₂₋₆ heteroalkyl is unsubstituted; two R₃ together with the atoms to which they are attached form a cyclic group; and/or two R₃ on the same atom form (═O).

In some embodiments of the compound of Formula (II), m is 2, and Ring A is as shown in the following structural fragment:

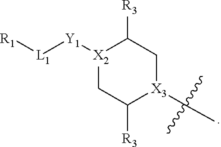

In some embodiments of the compound of Formula (II), m is 2, Ring A is as shown in one of the following structural fragments and the stereochemistry of the positions in Ring A identified by a wedge-shaped bond are as shown:

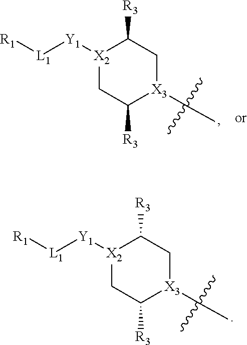

In some embodiments of the compound of Formula (I) or (II), R₃ at each occurrence is independently C₁₋₆ alkyl.

In some embodiments of the compound of Formula (II), m is 0.

In some embodiments of the compound of Formula (II), X₄, X₅ and X₆, and X₇ if present, are independently —CH—, —CR^{A}—, —C(O)—, —N—, —NR^{B}—, —O—, or —S—, provided that the combination of X₄, X₅, X₆ and optionally X₇ together with the atoms to which they are attached forms an aromatic or heteroaromatic group.

In some embodiments of the compound of Formula (II), n is 1, and X₄, X₅, X₆ and X₇ are independently —CH—, —C(C₁₋₆ alkyl)- or —N—, provided that the combination of X₄, X₅, X₆ and X₇ together with the atoms to which they are attached forms an aromatic or heteroaromatic group.

In some embodiments of the compound of Formula (I) or (II), Ring B is defined as follows:

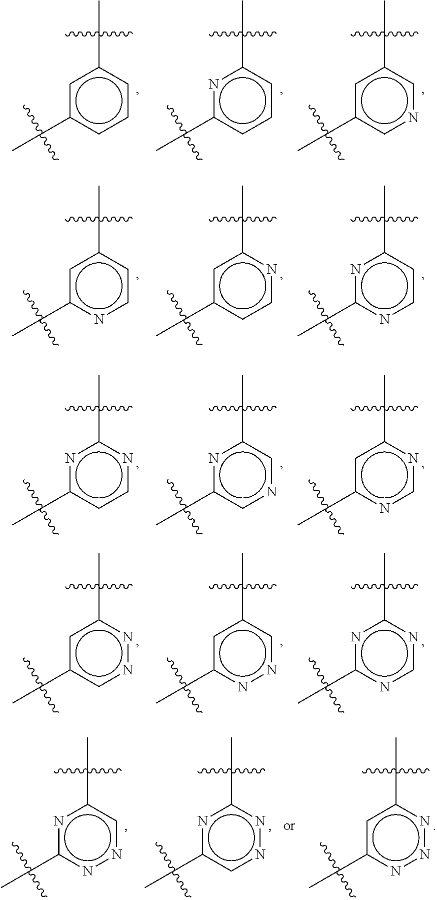

In some embodiments of the compound of Formula (I) or (II), Ring B is defined as follows:

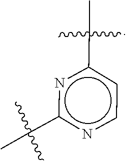

In some embodiments of the compound of Formula (II), n is 0, and X₄, X₅ and X₆ are independently —CH—, —CR^{A}—, —N—, —NR^{B}—, —O—, or —S—, provided that the combination of X₄, X₅ and X₆ together with the atoms to which they are attached forms an aromatic or heteroaromatic group.

In some embodiments of the compound of Formula (II), n is 0, and X₄ is —CH—, —C(C₁₋₆ alkyl)- or —N—; X₅ is —NH—, —N(C₁₋₆ alkyl)-, —O— or —S—; and X₆ is —CH—, —C(C₁₋₆ alkyl)- or —N—.

In some embodiments of the compound of Formula (I) or (II), Ring B is defined as follows:

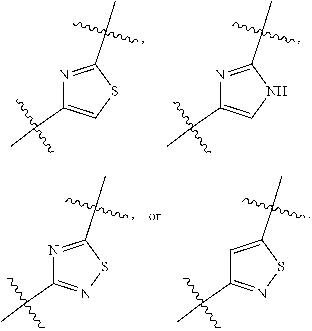 [SMILES: *c1csc(*)n1 ; *c1c[nH]c(*)n1 ; *c1nsc(*)n1 ; *c1cc(*)sn1]

In some embodiments of the compound of Formula (II), q is 0 to 4, and R₄ at each occurrence is independently —F, —Cl, —Br, —I, —OH, C₁₋₆ alkyl, C₂₋₆ heteroalkyl or C₅₋₁₄aryl-C₂₋₆-heteroalkyl, wherein each C₁₋₆ alkyl, C₂₋₆ heteroalkyl or C₅₋₁₄aryl-C₂₋₆heteroalkyl is unsubstituted.

In some embodiments of the compound of Formula (II), the stereochemistry of the position in Ring C identified by a wedge-shaped bond is as shown in one of the following structural fragments:

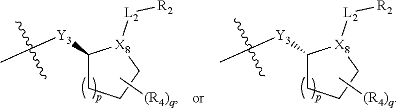

In some embodiments of the compound of Formula (II), q is 0.

In some embodiments of the compound of Formula (II), q is 1, and Ring C is as shown in the following structural fragment:

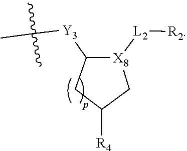

In some embodiments of the compound of Formula (II), q is 1, Ring C is as shown in one of the following structural fragments and the stereochemistry of the positions in Ring C identified by a wedge-shaped bond are as shown in one of the structural fragments:

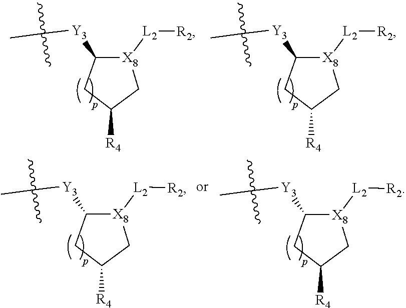

In some embodiments of the compound of Formula (II), p is 1.

In some embodiments of the compound of Formula (II), p is 2.

In some embodiments of the compound of Formula (I) or (II), R₄ at each occurrence is independently —OH, —OC₁₋₆ alkyl, or —O—CH₂-phenyl.

In some embodiments of the compound of Formula (II), X₃ is —CH— or —N—. In some embodiments of the compound of Formula (II), X₃ is —N—.

In some embodiments of the compound of Formula (I) or (II), R₁ is C₁₋₆ alkyl, C₃₋₁₄ cycloalkyl, C₅₋₁₄ heterocycloalkenyl, C₅₋₁₄ aryl or C₅₋₁₄ heteroaryl, each of which is unsubstituted or substituted with one or more R^{A}.

In some embodiments of the compound of Formula (I) or (II), R₂ is C₁₋₆ alkyl, C₂₋₆ heteroalkyl, C₃₋₁₄ cycloalkyl, C₅₋₁₄ aryl or C₅₋₁₄ heteroaryl, each of which is unsubstituted or substituted with one or more R^{A}.

In some embodiments of the compound of Formula (I) or (II), R₅ and R₆ at each occurrence are independently —H, —F, —Cl, —Br, —I, —OH, C₁₋₆ alkyl or —OC₁₋₆ alkyl, wherein each C₁₋₆ alkyl or —OC₁₋₆ alkyl is unsubstituted; or R₅ and R₆ together with the carbon atom to which they are attached form a C₃₋₁₄ cycloalkyl, C₃₋₁₄ heterocycloalkyl, C₃₋₁₄ halocycloalkyl, or C₃₋₁₄ haloheterocycloalkyl.

In some embodiments of the compound of Formula (I) or (II), L₁ is optional and if present is C₁₋₆ alkyl or C₂₋₆ heteroalkyl, each of which is unsubstituted or substituted with one or more R^{A}.

In some embodiments of the compound of Formula (I) or (II), L₂ is optional and if present is C₁₋₆ alkyl or C₂₋₆ heteroalkyl, each of which is unsubstituted or substituted with one or more R^{A}.

In some embodiments of the compound of Formula (I) or (II), Y₁ is —CR₅R₆—, —C(O)—, —NR^{B}—, —O—, —S— or —S(O)₂—.

In some embodiments of the compound of Formula (I) or (II), Y₂ is —CR₅R₆—, —NR^{B}—, —O— or —S—.

In some embodiments of the compound of Formula (I) or (II), Y₃ is —CR₅R₆—, —C(O)—, —NR^{B}—, —O— or —S—.

In some embodiments of the compound of Formula (I) or (II), X₁ is —CR^{B}₂—, —NR^{B}—, —O— or —S—.

In some embodiments of the compound of Formula (I) or (II),
R^{A} at each occurrence is independently —F, —Cl, —Br, —I, —NO₂, —OR^{B}, —SO₂R^{B}, C₁₋₆ alkyl, C₂₋₆ heteroalkyl or C₃₋₁₄ cycloalkyl, wherein each C₁₋₆ alkyl, C₂₋₆ heteroalkyl or C₃₋₁₄ cycloalkyl is unsubstituted or substituted with one or more —F, —Cl, —Br, —I, —CN, —NO₂, —SO₂, —N₃, —SCN, —NCS, —OR^{B}, —NR^{C}R^{D}, —NR^{B}NR^{C}R^{D}, —SR^{B}, —C(O)R^{B}, —C(O)OR^{B}, —C(O)NR^{C}R^{D}, —C(NR^{B})R^{B}, —C(NR^{B})NR^{C}R^{D}, —NR^{B}C(NR^{B})NR^{C}R^{D}, —S(O)R^{B}, —S(O)₂R^{B}, —S(O)OR^{B}, —S(O)₂OR^{B}, —NR^{B}NR^{C}R^{D} or —NR^{B}C(O)NR^{C}R^{D}; two R^{A} together with the atom(s) to which they are attached form a cyclic group; and/or two R^{A} on the same atom form (═O); and/or
R^{B} at each occurrence is independently (i) —H, or (ii) C₁₋₆ alkyl or C₅₋₁₄aryl-C₁₋₆alkyl, each of which is unsubstituted or substituted with one or more R^{E}; and/or
R^{C} and R^{D} at each occurrence are independently (i) —H, or (ii) C₁₋₆ alkyl, which is unsubstituted or substituted with one or more R^{E}, or R^{C} and R^{D} together with the nitrogen atom to which they are attached combine to form a heterocycle that is unsubstituted or substituted with one or more R^{E}; and/or
R^{E} at each occurrence is independently —F, —C, —Br, —I, —CN, —NO₂, —SO₂, —N₃, —SCN, —NCS, —OR^{F}, —NR^{G}R^{H}, —NR^{F}NR^{G}R^{H}, —SR^{F}, —C(O)R^{F}, —C(O)OR^{F}, —C(O)NR^{G}R^{H}, —C(NR^{F})R^{F}, —C(NR^{F})NR^{G}R^{H}, —S(O)R^{F}, —S(O)₂R^{F}, —S(O)OR^{F}, —S(O)₂OR^{F}, —NR^{F}NR^{G}R^{H}, —NR^{F}C(O)NR^{G}R^{H}, —NR^{F}C(NR^{F})NR^{G}R^{H}, C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl or C₅₋₁₄heteroaryl-C₁₋₆alkyl; two or more R^{E} together with the atoms to which they are attached form a cyclic group; and/or two R^{E} on the same atom form (═O); and/or
R^{F} at each occurrence is independently (i) —H, or (ii) C₁₋₆ alkyl; and/or
R^{G} and R^{H} at each occurrence are independently (i) —H, or (ii) C₁₋₆ alkyl, or R^{G} and R^{H} together with the nitrogen atom to which they are attached combine to form a heterocycle.

In some embodiments of the compound of Formula (I) or (II), R^{A} at each occurrence is independently —F, —Cl, —Br, —I, —NO₂, —OH, —SO₂R^{B}, C₁₋₆ alkyl, C₂₋₆ heteroalkyl or C₃₋₆ cycloalkyl, wherein each C₁₋₆ alkyl is unsubstituted or substituted with one or more —F, —Cl, —Br, —I, and each C₂₋₆ heteroalkyl or C₃₋₆ cycloalkyl is unsubstituted; two R^{A} together with the atom(s) to which they are attached form a cyclic group; and/or two R^{A} on the same atom form (═O).

In some embodiments of the compound of Formula (I) or (II), R^{A} at each occurrence is independently —F, —Cl, —Br, —I, —NO₂, —OH, —OC₁₋₆ alkyl, —SO₂C₁₋₆alkyl, unsubstituted C₁₋₆ alkyl, C₁₋₆ alkyl substituted 1 to 3 times with —F, —Cl, —Br, —I, or C₃₋₆ cycloalkyl; two R^{A} together with the atom to which they are attached form a C₃₋₆ cycloalkyl; two R^{A} on adjacent atoms together are —O—(CH₂)_{y}—O—, wherein y is 1 or 2; and/or two R^{A} on the same atom form (═O).

In some embodiments of the compound of Formula (I) or (II), R^{B} at each occurrence is independently (i) —H, or (ii) C₁₋₆ alkyl or C₅₋₁₄aryl-C₁₋₆alkyl. In some embodiments of the compound of Formula (I) or (II), R^{B} at each occurrence is independently (i) —H, or (ii) C₁₋₆ alkyl.

In some embodiments of the compound of Formula (I) or (II), R^{C} and R^{D} at each occurrence are independently (i) —H, or (ii) C₁₋₆ alkyl, which is unsubstituted, or R^{C} and R^{D} together with the nitrogen atom to which they are attached combine to form a heterocycle that is unsubstituted.

In some embodiments of the compound of Formula (I) or (II), R^{C} and R^{D} at each occurrence are independently (i) —H, or (ii) C₁₋₆ alkyl.

In some embodiments of the compound of Formula (I) or (II), R₁ is C₁₋₆ alkyl, C₃₋₁₀ cycloalkyl, C₅₋₁₀ heterocycloalkenyl, C₆₋₁₀ aryl or C₅₋₁₀ heteroaryl, each of which is unsubstituted or substituted one or more times, independently, with —F, —Cl, —Br, —I, —NO₂, —OC₁₋₆ alkyl, unsubstituted C₁₋₆ alkyl, or C₁₋₆ alkyl substituted 1 to 3 times, independently, with —F, —Cl, —Br or —I; or C₆ aryl or C₅₋₆ heteroaryl, wherein two adjacent atoms of the aryl or heteroaryl ring are bonded to the group —O—(CH₂)_{y}—O—, wherein y is 1 or 2.

In some embodiments of the compound of Formula (I) or (II), R₂ is C₁₋₆ alkyl, C₂₋₆ heteroalkyl, C₃₋₆ cycloalkyl, C₆₋₁₀ aryl or C₅₋₁₀ heteroaryl, each of which is unsubstituted or substituted one or more times, independently, with —F, —Cl, —Br, —I, —SO₂C₁₋₆ alkyl, —OC₁₋₆ alkyl, unsubstituted C₁₋₆ alkyl, unsubstituted C₃₋₆ cycloalkyl, or C₁₋₆ alkyl substituted 1 to 3 times, independently, with —F, —Cl, —Br or —I; or C₆ aryl or C₅₋₆ heteroaryl, wherein two adjacent atoms of the aryl or heteroaryl ring are bonded to the group —O—(CH₂)_{y}—O—, wherein y is 1 or 2.

In some embodiments of the compound of Formula (I) or (II), R₁ is:

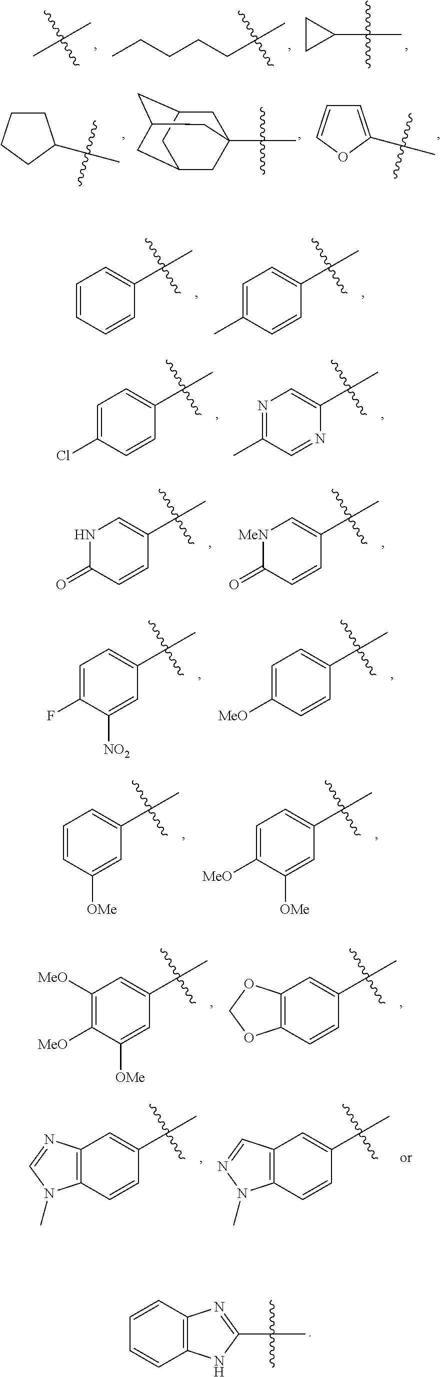

In some embodiments of the compound of Formula (I) or (II), R₂ is:

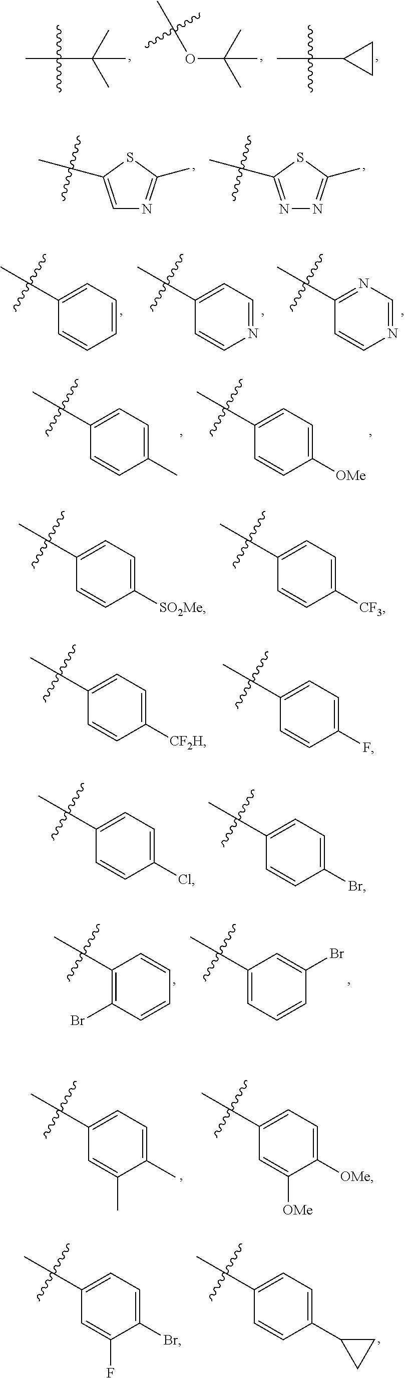

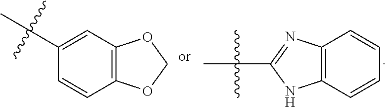 [SMILES: *c1ccc2c(c1)OCO2 ; *c1nc2ccccc2[nH]1]

In some embodiments of the compound of Formula (I) or (II), L₁ is present and is C₁₋₄ alkyl or C₂₋₄ heteroalkyl.

In some embodiments of the compound of Formula (I) or (II), L₁ is absent.

In some embodiments of the compound of Formula (I) or (II), L₂ is present and is: C₁₋₄ alkyl which is unsubstituted or substituted with one C₁₋₆ alkyl group; —C(O)—; or —C(O)O—. In some embodiments of the compound of Formula (I) or (II), L₂ is present and is unsubstituted C₁₋₄ alkyl, —CH(C₁₋₆ alkyl)-, —C(O)— or —C(O)O—.

In some embodiments of the compound of Formula (I) or (II), X₁ is —CH₂—, —NH—, —N(C₁₋₆ alkyl)-, —O— or —S—. In some embodiments of the compound of Formula (I) or (II), X₁ is —NH— or —N(C₁₋₆ alkyl)-. In some embodiments of the compound of Formula (I) or (II), X₁ is —NH—.

In some embodiments of the compound of Formula (I) or (II), R₅ and R₆ at each occurrence are independently —H or unsubstituted C₁₋₆ alkyl; or R₅ and R₆ together with the atom to which they are attached form a C₃₋₆ cycloalkyl group.

In some embodiments of the compound of Formula (I) or (II), Y₁ is —CH₂—, —CH(C₁₋₆ alkyl)-, —(C(C₁₋₆ alkyl)₂)—, —C(O)— or —S(O)₂—. In some embodiments of the compound of Formula (I) or (II), Y₁ is —CH₂—, —C(O)— or —S(O)₂—.

In some embodiments of the compound of Formula (I) or (II), Y₂ is —CH₂—, —CH(C₁₋₆ alkyl)-, —C(C₁₋₆ alkyl)₂-, or is as shown in the following structural fragment:

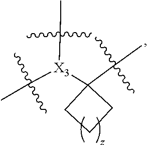

wherein z is 0 to 3.

In some embodiments of the compound of Formula (I) or (II), Y₂ is —CHR₅— and the stereochemistry of Y₂ is as shown in one of the following structural fragments:

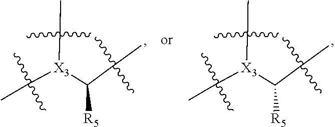

In some embodiments of the compound of Formula (I) or (II), Y₃ is —CH₂—, —CH(C₁₋₆alkyl)-, —C(C₁₋₆ alkyl)₂-, or —C(O)—. In some embodiments of the compound of Formula (I) or (II), Y₃ is —CH₂— or —C(O)—.

In some embodiments of the compound of Formula (I) or (II), L₁ and R₁ together are:

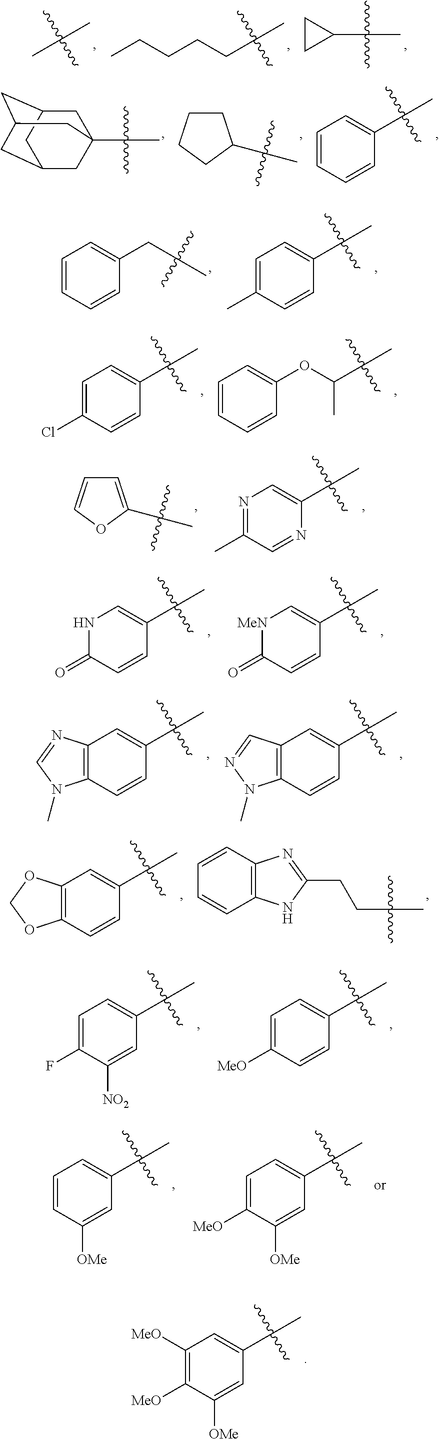

In some embodiments of the compound of Formula (I) or (II), L₂ is —CH(C₁₋₆ alkyl)- and the stereochemistry of L₂ is as shown in one of the following structural fragments:

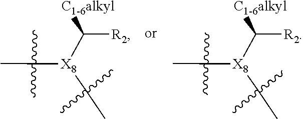

In some embodiments of the compound of Formula (I) or (II), L₂ and R₂ together are:

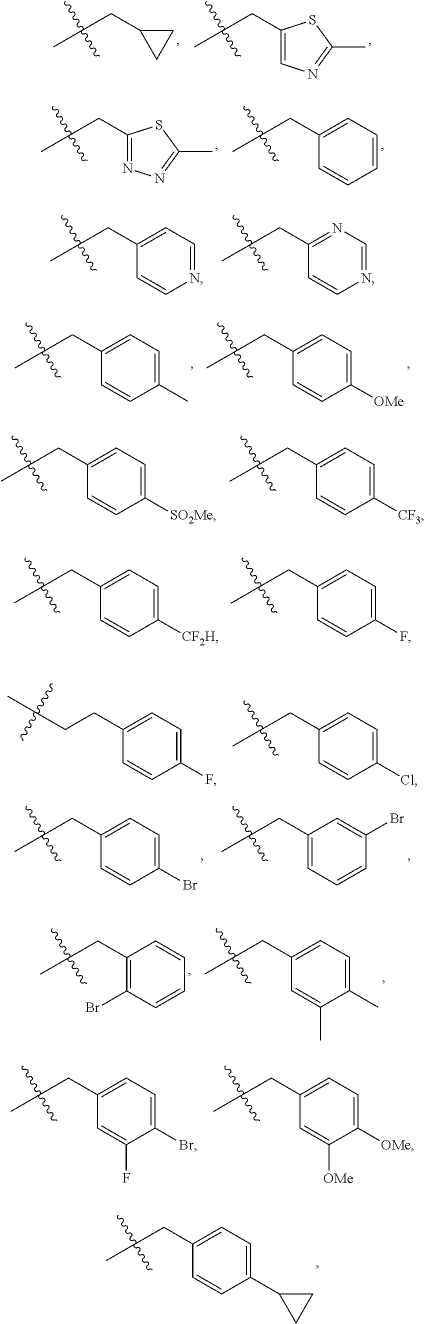

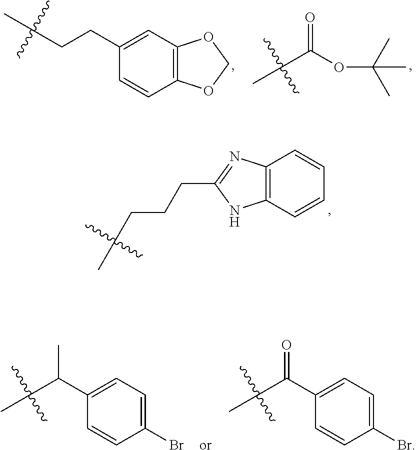

In some embodiments of the compound of Formula (I) or (II), the compound is a compound of Formula (III):

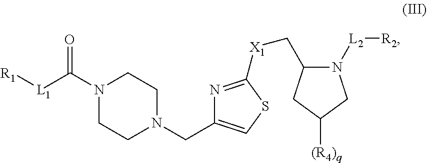

wherein q is 0 or 1.

In some embodiments of the compound of Formula (III), the compound is as shown in Table 2 (below).

TABLE 2

| R₁—L₁ | 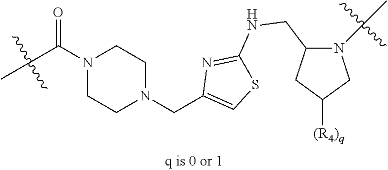 | R₄ | L₂—R₂ |
|---|---|---|---|
| 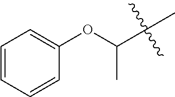 [SMILES: *C(C)Oc1ccccc1] | 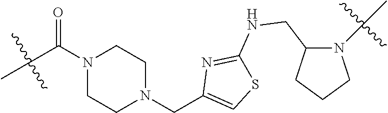 [SMILES: *C(=O)N1CCN(Cc2csc(NCC3CCCN3*)n2)CC1] | absent | 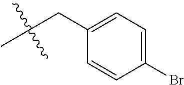 [SMILES: *Cc1ccc(Br)cc1] |
| 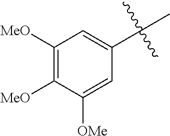 [SMILES: *c1cc(OC)c(OC)c(OC)c1] | 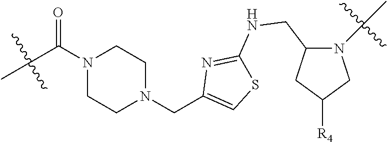 | OBzl | 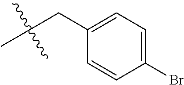 [SMILES: *Cc1ccc(Br)cc1] |
| 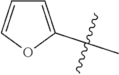 [SMILES: *c1ccco1] | 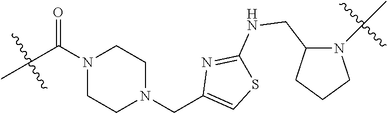 [SMILES: *C(=O)N1CCN(Cc2csc(NCC3CCCN3*)n2)CC1] | absent | 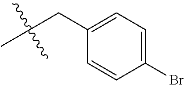 [SMILES: *Cc1ccc(Br)cc1] |
| 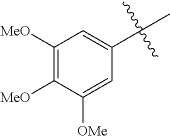 [SMILES: *c1cc(OC)c(OC)c(OC)c1] | 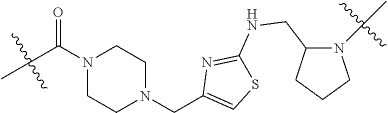 [SMILES: *C(=O)N1CCN(Cc2csc(NCC3CCCN3*)n2)CC1] | absent | 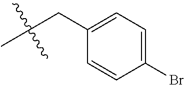 [SMILES: *Cc1ccc(Br)cc1] |
| 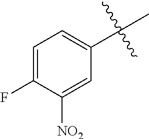 [SMILES: *c1ccc(F)c([N+](=O)[O-])c1] | 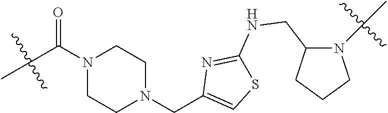 [SMILES: *C(=O)N1CCN(Cc2csc(NCC3CCCN3*)n2)CC1] | absent | 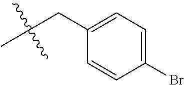 [SMILES: *Cc1ccc(Br)cc1] |
| 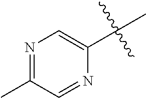 [SMILES: *c1cnc(C)cn1] | 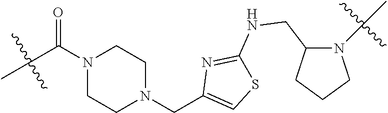 [SMILES: *C(=O)N1CCN(Cc2csc(NCC3CCCN3*)n2)CC1] | absent | 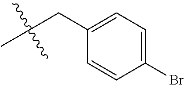 [SMILES: *Cc1ccc(Br)cc1] |
| 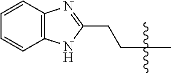 [SMILES: *CCc1nc2ccccc2[nH]1] | 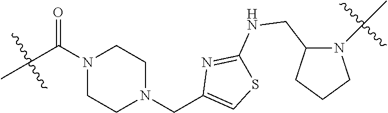 [SMILES: *C(=O)N1CCN(Cc2csc(NCC3CCCN3*)n2)CC1] | absent | 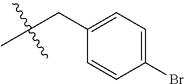 [SMILES: *Cc1ccc(Br)cc1] |
| 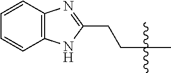 [SMILES: *CCc1nc2ccccc2[nH]1] | 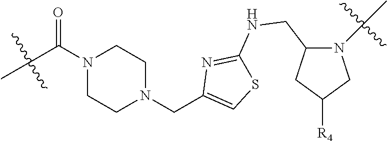 | OBzl | 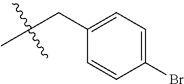 [SMILES: *Cc1ccc(Br)cc1] |
| 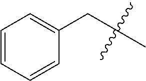 [SMILES: *Cc1ccccc1] | 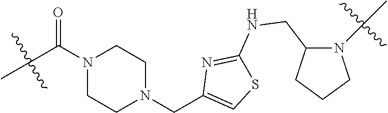 [SMILES: *C(=O)N1CCN(Cc2csc(NCC3CCCN3*)n2)CC1] | absent | 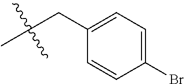 [SMILES: *Cc1ccc(Br)cc1] |
| 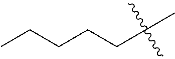 [SMILES: *CCCCC] | 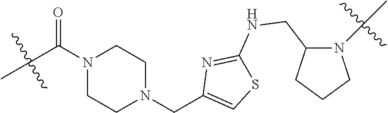 [SMILES: *C(=O)N1CCN(Cc2csc(NCC3CCCN3*)n2)CC1] | absent | 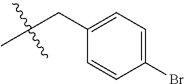 [SMILES: *Cc1ccc(Br)cc1] |
| 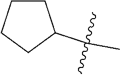 [SMILES: *C1CCCC1] | 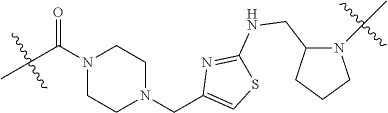 [SMILES: *C(=O)N1CCN(Cc2csc(NCC3CCCN3*)n2)CC1] | absent | 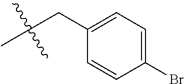 [SMILES: *Cc1ccc(Br)cc1] |
| 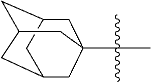 [SMILES: *C12CC3CC(CC(C3)C1)C2] | 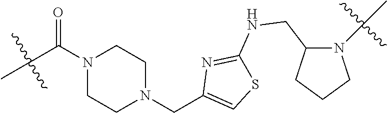 [SMILES: *C(=O)N1CCN(Cc2csc(NCC3CCCN3*)n2)CC1] | absent | 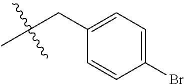 [SMILES: *Cc1ccc(Br)cc1] |
| 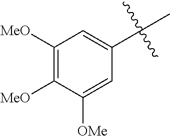 [SMILES: *c1cc(OC)c(OC)c(OC)c1] | 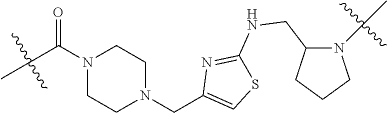 [SMILES: *C(=O)N1CCN(Cc2csc(NCC3CCCN3*)n2)CC1] | absent | 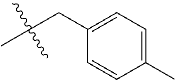 [SMILES: *Cc1ccc(C)cc1] |
| 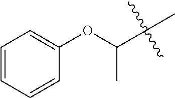 [SMILES: *C(C)Oc1ccccc1] | 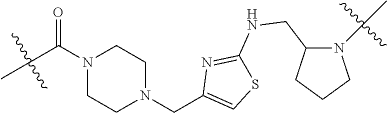 [SMILES: *C(=O)N1CCN(Cc2csc(NCC3CCCN3*)n2)CC1] | absent | 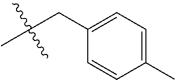 [SMILES: *Cc1ccc(C)cc1] |
| 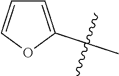 [SMILES: *c1ccco1] | 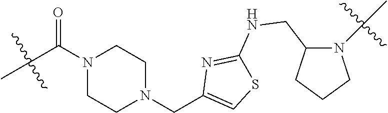 [SMILES: *C(=O)N1CCN(Cc2csc(NCC3CCCN3*)n2)CC1] | absent | 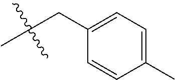 [SMILES: *Cc1ccc(C)cc1] |
| 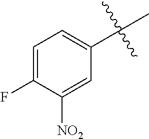 [SMILES: *c1ccc(F)c([N+](=O)[O-])c1] | 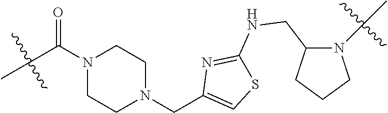 [SMILES: *C(=O)N1CCN(Cc2csc(NCC3CCCN3*)n2)CC1] | absent | 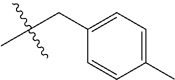 [SMILES: *Cc1ccc(C)cc1] |
| 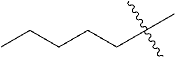 [SMILES: *CCCCC] | 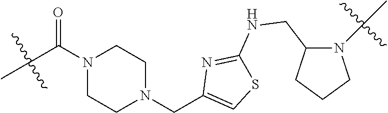 [SMILES: *C(=O)N1CCN(Cc2csc(NCC3CCCN3*)n2)CC1] | absent | 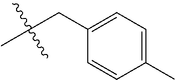 [SMILES: *Cc1ccc(C)cc1] |
| 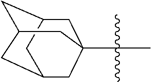 [SMILES: *C12CC3CC(CC(C3)C1)C2] | 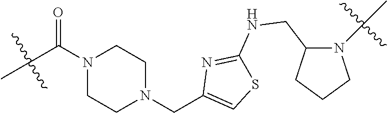 [SMILES: *C(=O)N1CCN(Cc2csc(NCC3CCCN3*)n2)CC1] | absent | 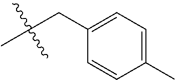 [SMILES: *Cc1ccc(C)cc1] |
| 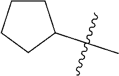 [SMILES: *C1CCCC1] | 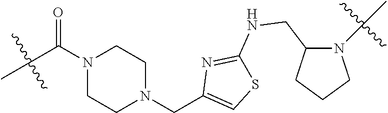 [SMILES: *C(=O)N1CCN(Cc2csc(NCC3CCCN3*)n2)CC1] | absent | 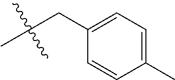 [SMILES: *Cc1ccc(C)cc1] |
| 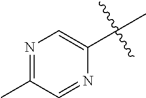 [SMILES: *c1cnc(C)cn1] | 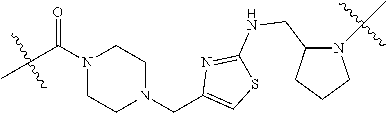 [SMILES: *C(=O)N1CCN(Cc2csc(NCC3CCCN3*)n2)CC1] | absent | 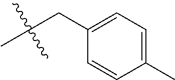 [SMILES: *Cc1ccc(C)cc1] |
| 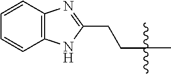 [SMILES: *CCc1nc2ccccc2[nH]1] | 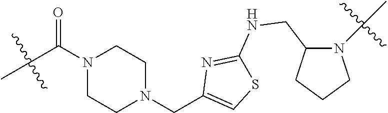 [SMILES: *C(=O)N1CCN(Cc2csc(NCC3CCCN3*)n2)CC1] | absent | 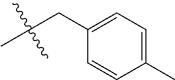 [SMILES: *Cc1ccc(C)cc1] |
| 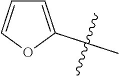 [SMILES: *c1ccco1] | 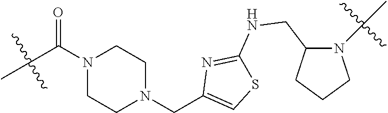 [SMILES: *C(=O)N1CCN(Cc2csc(NCC3CCCN3*)n2)CC1] | absent | 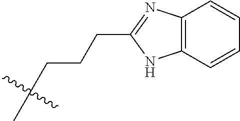 [SMILES: *CCCc1nc2ccccc2[nH]1] |
| 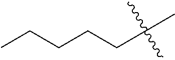 [SMILES: *CCCCC] | 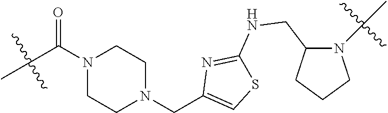 [SMILES: *C(=O)N1CCN(Cc2csc(NCC3CCCN3*)n2)CC1] | absent | 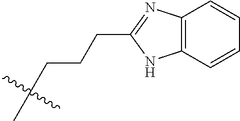 [SMILES: *CCCc1nc2ccccc2[nH]1] |
| 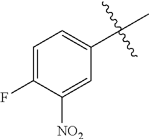 [SMILES: *c1ccc(F)c([N+](=O)[O-])c1] | 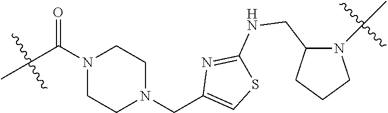 [SMILES: *C(=O)N1CCN(Cc2csc(NCC3CCCN3*)n2)CC1] | absent | 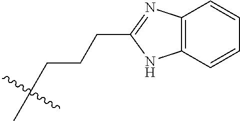 [SMILES: *CCCc1nc2ccccc2[nH]1] |
| 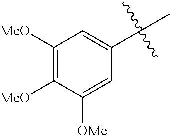 [SMILES: *c1cc(OC)c(OC)c(OC)c1] | 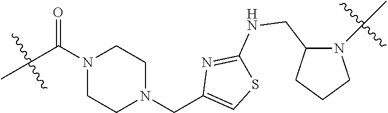 [SMILES: *C(=O)N1CCN(Cc2csc(NCC3CCCN3*)n2)CC1] | absent | 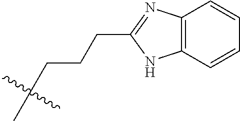 [SMILES: *CCCc1nc2ccccc2[nH]1] |
| 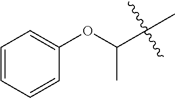 [SMILES: *C(C)Oc1ccccc1] | 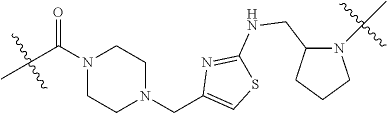 [SMILES: *C(=O)N1CCN(Cc2csc(NCC3CCCN3*)n2)CC1] | absent | 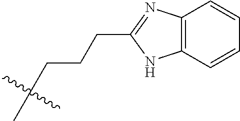 [SMILES: *CCCc1nc2ccccc2[nH]1] |
| 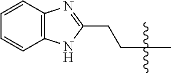 [SMILES: *CCc1nc2ccccc2[nH]1] | 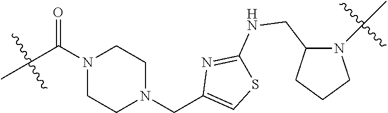 [SMILES: *C(=O)N1CCN(Cc2csc(NCC3CCCN3*)n2)CC1] | absent | 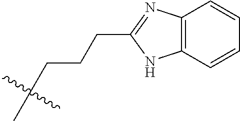 [SMILES: *CCCc1nc2ccccc2[nH]1] |
| 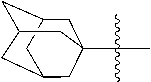 [SMILES: *C12CC3CC(CC(C3)C1)C2] | 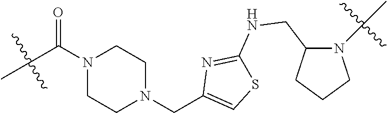 [SMILES: *C(=O)N1CCN(Cc2csc(NCC3CCCN3*)n2)CC1] | absent | 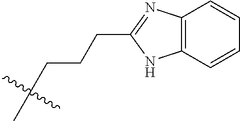 [SMILES: *CCCc1nc2ccccc2[nH]1] |
| 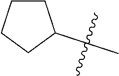 [SMILES: *C1CCCC1] | 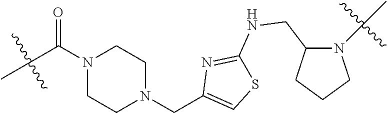 [SMILES: *C(=O)N1CCN(Cc2csc(NCC3CCCN3*)n2)CC1] | absent | 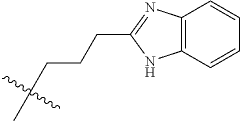 [SMILES: *CCCc1nc2ccccc2[nH]1] |
| 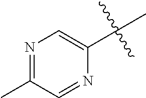 [SMILES: *c1cnc(C)cn1] | 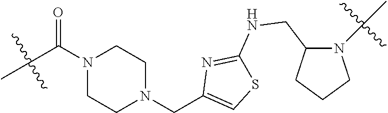 [SMILES: *C(=O)N1CCN(Cc2csc(NCC3CCCN3*)n2)CC1] | absent | 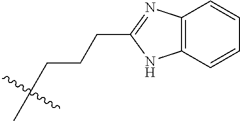 [SMILES: *CCCc1nc2ccccc2[nH]1] |
| 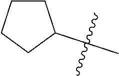 [SMILES: *C1CCCC1] | 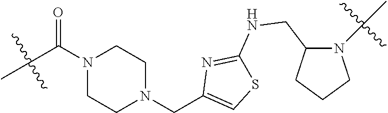 [SMILES: *C(=O)N1CCN(Cc2csc(NCC3CCCN3*)n2)CC1] | absent | 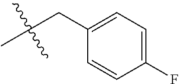 [SMILES: *Cc1ccc(F)cc1] |
| 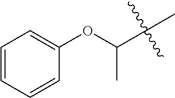 [SMILES: *C(C)Oc1ccccc1] | 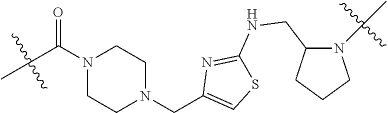 [SMILES: *C(=O)N1CCN(Cc2csc(NCC3CCCN3*)n2)CC1] | absent | 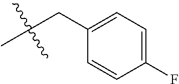 [SMILES: *Cc1ccc(F)cc1] |
| 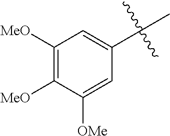 [SMILES: *c1cc(OC)c(OC)c(OC)c1] | 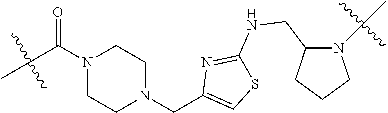 [SMILES: *C(=O)N1CCN(Cc2csc(NCC3CCCN3*)n2)CC1] | absent | 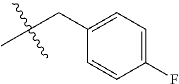 [SMILES: *Cc1ccc(F)cc1] |
| 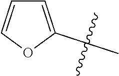 [SMILES: *c1ccco1] | 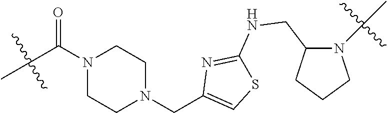 [SMILES: *C(=O)N1CCN(Cc2csc(NCC3CCCN3*)n2)CC1] | absent | 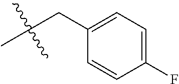 [SMILES: *Cc1ccc(F)cc1] |
| 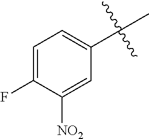 [SMILES: *c1ccc(F)c([N+](=O)[O-])c1] | 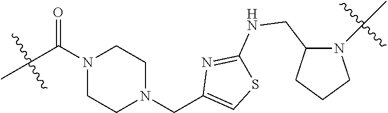 [SMILES: *C(=O)N1CCN(Cc2csc(NCC3CCCN3*)n2)CC1] | absent | 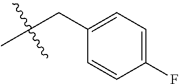 [SMILES: *Cc1ccc(F)cc1] |
| 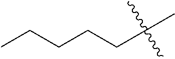 [SMILES: *CCCCC] | 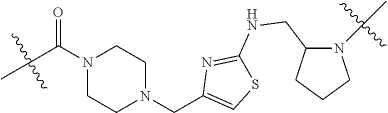 [SMILES: *C(=O)N1CCN(Cc2csc(NCC3CCCN3*)n2)CC1] | absent | 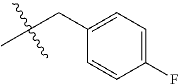 [SMILES: *Cc1ccc(F)cc1] |
| 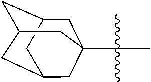 [SMILES: *C12CC3CC(CC(C3)C1)C2] | 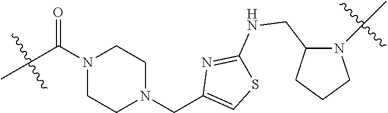 [SMILES: *C(=O)N1CCN(Cc2csc(NCC3CCCN3*)n2)CC1] | absent | 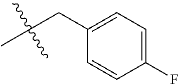 [SMILES: *Cc1ccc(F)cc1] |
| 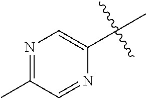 [SMILES: *c1cnc(C)cn1] | 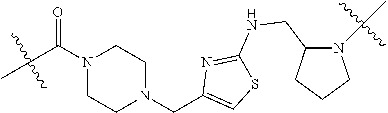 [SMILES: *C(=O)N1CCN(Cc2csc(NCC3CCCN3*)n2)CC1] | absent | 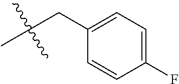 [SMILES: *Cc1ccc(F)cc1] |
| 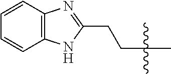 [SMILES: *CCc1nc2ccccc2[nH]1] | 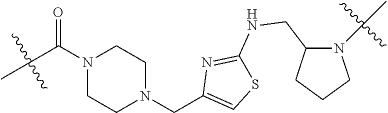 [SMILES: *C(=O)N1CCN(Cc2csc(NCC3CCCN3*)n2)CC1] | absent | 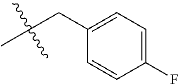 [SMILES: *Cc1ccc(F)cc1] |
| 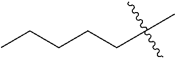 [SMILES: *CCCCC] | 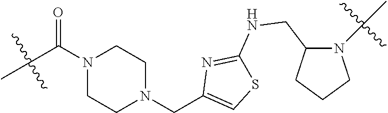 [SMILES: *C(=O)N1CCN(Cc2csc(NCC3CCCN3*)n2)CC1] | absent | 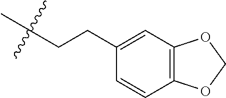 [SMILES: *CCc1ccc2c(c1)OCO2] |
| 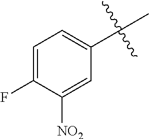 [SMILES: *c1ccc(F)c([N+](=O)[O-])c1] | 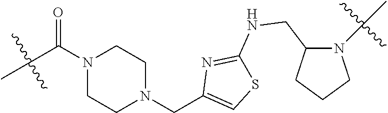 [SMILES: *C(=O)N1CCN(Cc2csc(NCC3CCCN3*)n2)CC1] | absent | 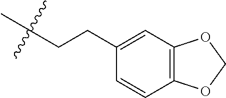 [SMILES: *CCc1ccc2c(c1)OCO2] |
| 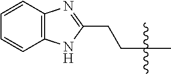 [SMILES: *CCc1nc2ccccc2[nH]1] | 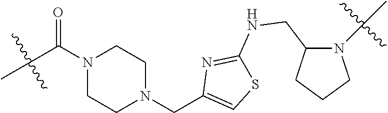 [SMILES: *C(=O)N1CCN(Cc2csc(NCC3CCCN3*)n2)CC1] | absent | 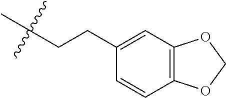 [SMILES: *CCc1ccc2c(c1)OCO2] |
| 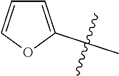 [SMILES: *c1ccco1] | 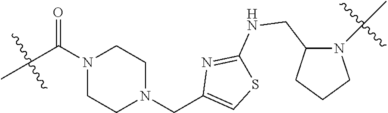 [SMILES: *C(=O)N1CCN(Cc2csc(NCC3CCCN3*)n2)CC1] | absent | 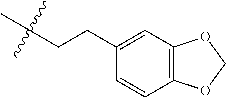 [SMILES: *CCc1ccc2c(c1)OCO2] |
| 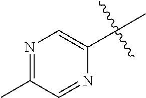 [SMILES: *c1cnc(C)cn1] | 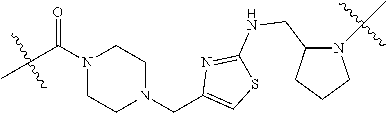 [SMILES: *C(=O)N1CCN(Cc2csc(NCC3CCCN3*)n2)CC1] | absent | 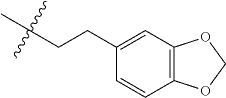 [SMILES: *CCc1ccc2c(c1)OCO2] |
| 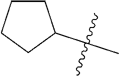 [SMILES: *C1CCCC1] | 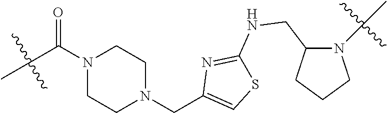 [SMILES: *C(=O)N1CCN(Cc2csc(NCC3CCCN3*)n2)CC1] | absent | 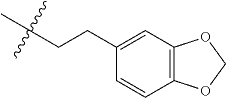 [SMILES: *CCc1ccc2c(c1)OCO2] |
| 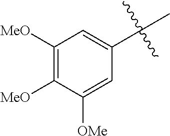 [SMILES: *c1cc(OC)c(OC)c(OC)c1] | 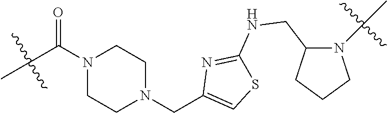 [SMILES: *C(=O)N1CCN(Cc2csc(NCC3CCCN3*)n2)CC1] | absent | 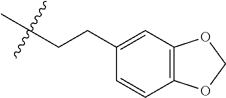 [SMILES: *CCc1ccc2c(c1)OCO2] |
| 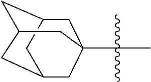 [SMILES: *C12CC3CC(CC(C3)C1)C2] | 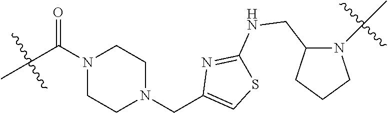 [SMILES: *C(=O)N1CCN(Cc2csc(NCC3CCCN3*)n2)CC1] | absent | 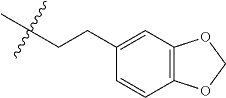 [SMILES: *CCc1ccc2c(c1)OCO2] |
| 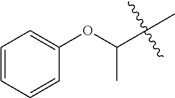 [SMILES: *C(C)Oc1ccccc1] | 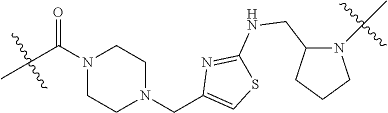 [SMILES: *C(=O)N1CCN(Cc2csc(NCC3CCCN3*)n2)CC1] | absent | 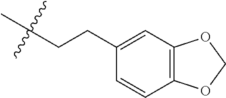 [SMILES: *CCc1ccc2c(c1)OCO2] |
| 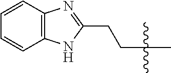 [SMILES: *CCc1nc2ccccc2[nH]1] | 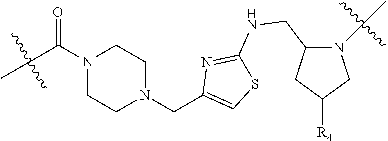 | OBzl | 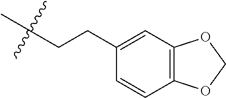 [SMILES: *CCc1ccc2c(c1)OCO2] |
| 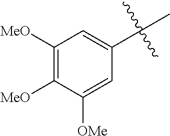 [SMILES: *c1cc(OC)c(OC)c(OC)c1] | 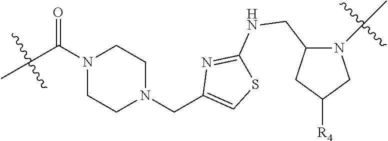 | OBzl | 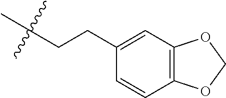 [SMILES: *CCc1ccc2c(c1)OCO2] |
| 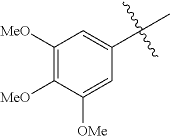 [SMILES: *c1cc(OC)c(OC)c(OC)c1] | 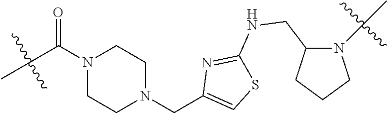 [SMILES: *C(=O)N1CCN(Cc2csc(NCC3CCCN3*)n2)CC1] | absent | 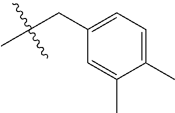 [SMILES: *Cc1ccc(C)c(C)c1] |
| 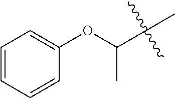 [SMILES: *C(C)Oc1ccccc1] | 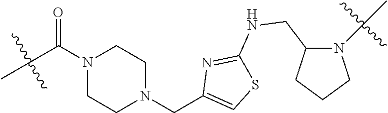 [SMILES: *C(=O)N1CCN(Cc2csc(NCC3CCCN3*)n2)CC1] | absent | 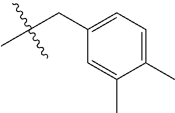 [SMILES: *Cc1ccc(C)c(C)c1] |
| 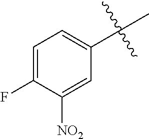 [SMILES: *c1ccc(F)c([N+](=O)[O-])c1] | 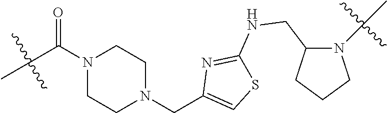 [SMILES: *C(=O)N1CCN(Cc2csc(NCC3CCCN3*)n2)CC1] | absent | 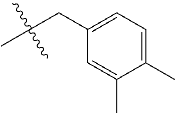 [SMILES: *Cc1ccc(C)c(C)c1] |
| 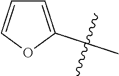 [SMILES: *c1ccco1] | 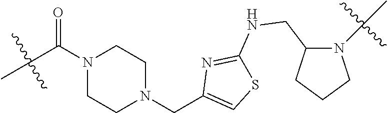 [SMILES: *C(=O)N1CCN(Cc2csc(NCC3CCCN3*)n2)CC1] | absent | 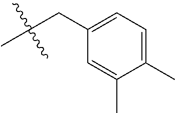 [SMILES: *Cc1ccc(C)c(C)c1] |
| 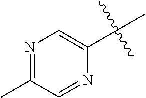 [SMILES: *c1cnc(C)cn1] | 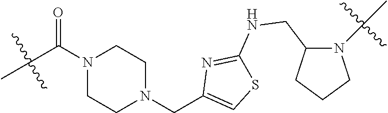 [SMILES: *C(=O)N1CCN(Cc2csc(NCC3CCCN3*)n2)CC1] | absent | 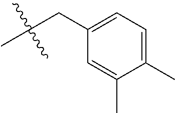 [SMILES: *Cc1ccc(C)c(C)c1] |
| 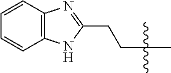 [SMILES: *CCc1nc2ccccc2[nH]1] | 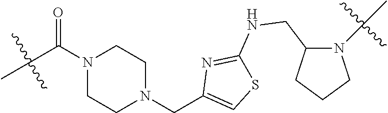 [SMILES: *C(=O)N1CCN(Cc2csc(NCC3CCCN3*)n2)CC1] | absent | 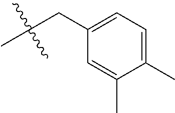 [SMILES: *Cc1ccc(C)c(C)c1] |
| 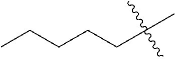 [SMILES: *CCCCC] | 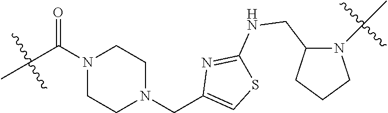 [SMILES: *C(=O)N1CCN(Cc2csc(NCC3CCCN3*)n2)CC1] | absent | 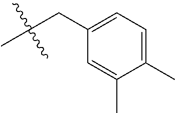 [SMILES: *Cc1ccc(C)c(C)c1] |
| 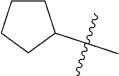 [SMILES: *C1CCCC1] | 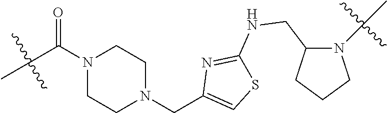 [SMILES: *C(=O)N1CCN(Cc2csc(NCC3CCCN3*)n2)CC1] | absent | 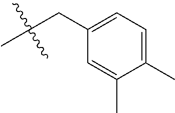 [SMILES: *Cc1ccc(C)c(C)c1] |
| 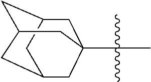 [SMILES: *C12CC3CC(CC(C3)C1)C2] | 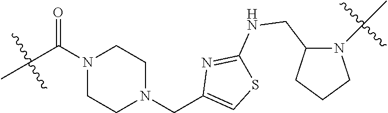 [SMILES: *C(=O)N1CCN(Cc2csc(NCC3CCCN3*)n2)CC1] | absent | 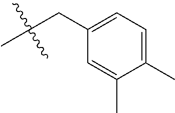 [SMILES: *Cc1ccc(C)c(C)c1] |
| 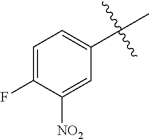 [SMILES: *c1ccc(F)c([N+](=O)[O-])c1] | 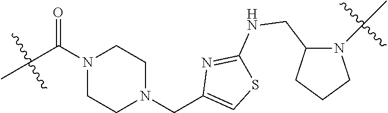 [SMILES: *C(=O)N1CCN(Cc2csc(NCC3CCCN3*)n2)CC1] | absent | 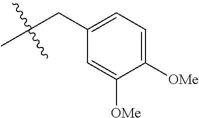 [SMILES: *Cc1ccc(OC)c(OC)c1] |
| 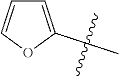 [SMILES: *c1ccco1] | 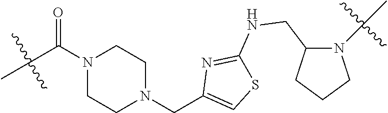 [SMILES: *C(=O)N1CCN(Cc2csc(NCC3CCCN3*)n2)CC1] | absent | 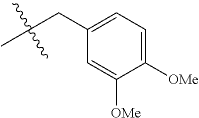 [SMILES: *Cc1ccc(OC)c(OC)c1] |
| 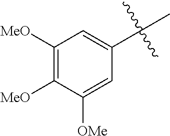 [SMILES: *c1cc(OC)c(OC)c(OC)c1] | 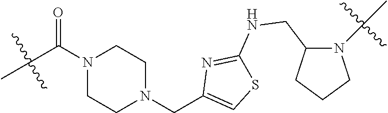 [SMILES: *C(=O)N1CCN(Cc2csc(NCC3CCCN3*)n2)CC1] | absent | 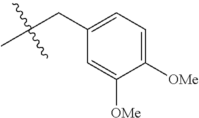 [SMILES: *Cc1ccc(OC)c(OC)c1] |
| 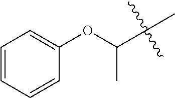 [SMILES: *C(C)Oc1ccccc1] | 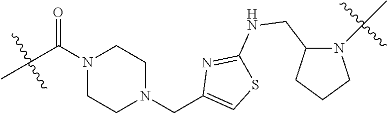 [SMILES: *C(=O)N1CCN(Cc2csc(NCC3CCCN3*)n2)CC1] | absent | 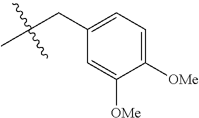 [SMILES: *Cc1ccc(OC)c(OC)c1] |
| 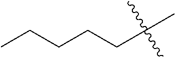 [SMILES: *CCCCC] | 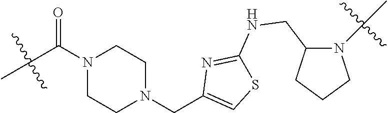 [SMILES: *C(=O)N1CCN(Cc2csc(NCC3CCCN3*)n2)CC1] | absent | 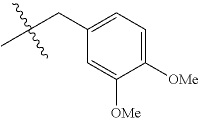 [SMILES: *Cc1ccc(OC)c(OC)c1] |
| 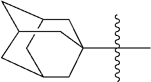 [SMILES: *C12CC3CC(CC(C3)C1)C2] | 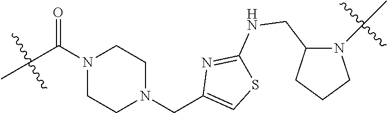 [SMILES: *C(=O)N1CCN(Cc2csc(NCC3CCCN3*)n2)CC1] | absent | 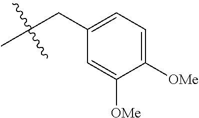 [SMILES: *Cc1ccc(OC)c(OC)c1] |
| 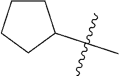 [SMILES: *C1CCCC1] | 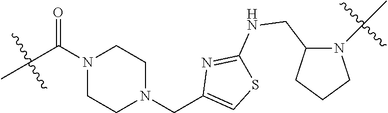 [SMILES: *C(=O)N1CCN(Cc2csc(NCC3CCCN3*)n2)CC1] | absent | 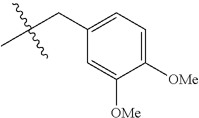 [SMILES: *Cc1ccc(OC)c(OC)c1] |
| 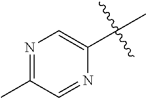 [SMILES: *c1cnc(C)cn1] | 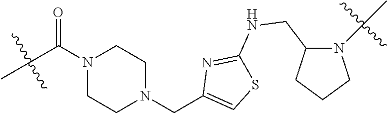 [SMILES: *C(=O)N1CCN(Cc2csc(NCC3CCCN3*)n2)CC1] | absent | 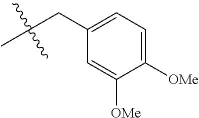 [SMILES: *Cc1ccc(OC)c(OC)c1] |
| 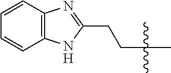 [SMILES: *CCc1nc2ccccc2[nH]1] | 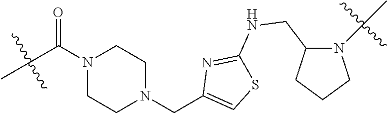 [SMILES: *C(=O)N1CCN(Cc2csc(NCC3CCCN3*)n2)CC1] | absent | 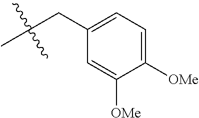 [SMILES: *Cc1ccc(OC)c(OC)c1] |
| 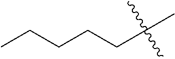 [SMILES: *CCCCC] | 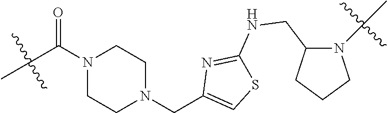 [SMILES: *C(=O)N1CCN(Cc2csc(NCC3CCCN3*)n2)CC1] | absent | 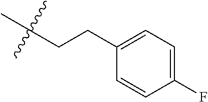 [SMILES: *CCc1ccc(F)cc1] |
| 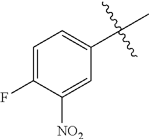 [SMILES: *c1ccc(F)c([N+](=O)[O-])c1] | 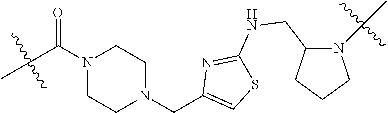 [SMILES: *C(=O)N1CCN(Cc2csc(NCC3CCCN3*)n2)CC1] | absent | 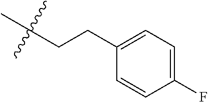 [SMILES: *CCc1ccc(F)cc1] |
| 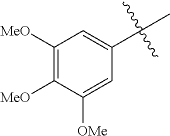 [SMILES: *c1cc(OC)c(OC)c(OC)c1] | 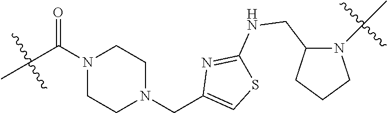 [SMILES: *C(=O)N1CCN(Cc2csc(NCC3CCCN3*)n2)CC1] | absent | 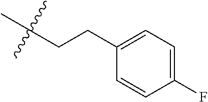 [SMILES: *CCc1ccc(F)cc1] |
| 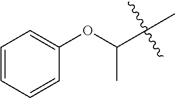 [SMILES: *C(C)Oc1ccccc1] | 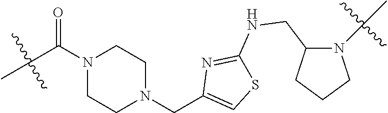 [SMILES: *C(=O)N1CCN(Cc2csc(NCC3CCCN3*)n2)CC1] | absent | 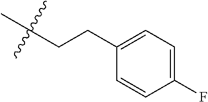 [SMILES: *CCc1ccc(F)cc1] |
| 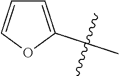 [SMILES: *c1ccco1] | 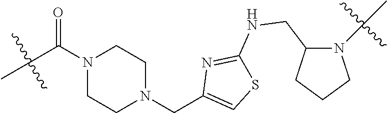 [SMILES: *C(=O)N1CCN(Cc2csc(NCC3CCCN3*)n2)CC1] | absent | 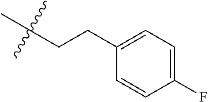 [SMILES: *CCc1ccc(F)cc1] |
| 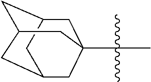 [SMILES: *C12CC3CC(CC(C3)C1)C2] | 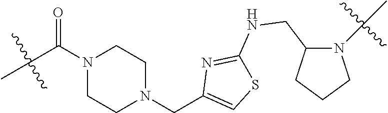 [SMILES: *C(=O)N1CCN(Cc2csc(NCC3CCCN3*)n2)CC1] | absent | 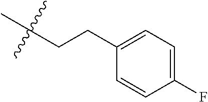 [SMILES: *CCc1ccc(F)cc1] |
| 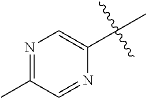 [SMILES: *c1cnc(C)cn1] | 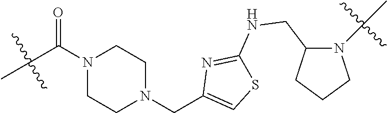 [SMILES: *C(=O)N1CCN(Cc2csc(NCC3CCCN3*)n2)CC1] | absent | 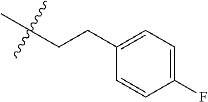 [SMILES: *CCc1ccc(F)cc1] |
| 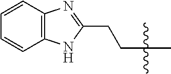 [SMILES: *CCc1nc2ccccc2[nH]1] | 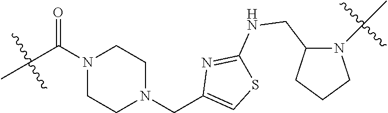 [SMILES: *C(=O)N1CCN(Cc2csc(NCC3CCCN3*)n2)CC1] | absent | 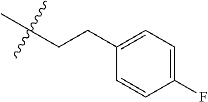 [SMILES: *CCc1ccc(F)cc1] |
| 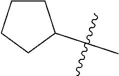 [SMILES: *C1CCCC1] | 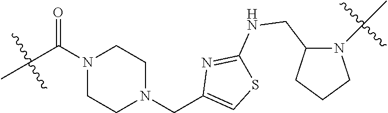 [SMILES: *C(=O)N1CCN(Cc2csc(NCC3CCCN3*)n2)CC1] | absent | 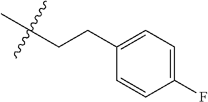 [SMILES: *CCc1ccc(F)cc1] |
| 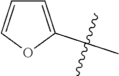 [SMILES: *c1ccco1] | 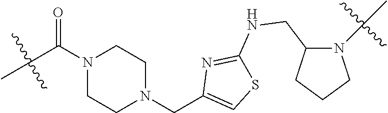 [SMILES: *C(=O)N1CCN(Cc2csc(NCC3CCCN3*)n2)CC1] | absent | 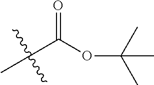 [SMILES: *C(=O)OC(C)(C)C] |
| 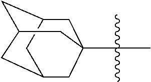 [SMILES: *C12CC3CC(CC(C3)C1)C2] | 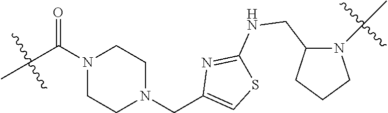 [SMILES: *C(=O)N1CCN(Cc2csc(NCC3CCCN3*)n2)CC1] | absent | 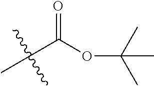 [SMILES: *C(=O)OC(C)(C)C] |
| 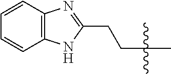 [SMILES: *CCc1nc2ccccc2[nH]1] | 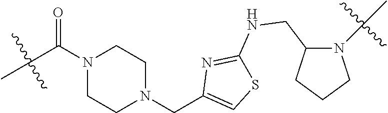 [SMILES: *C(=O)N1CCN(Cc2csc(NCC3CCCN3*)n2)CC1] | absent | 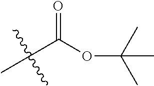 [SMILES: *C(=O)OC(C)(C)C] |
| 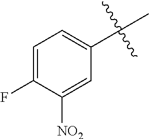 [SMILES: *c1ccc(F)c([N+](=O)[O-])c1] | 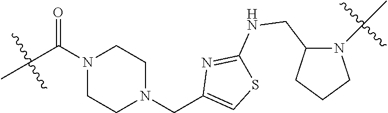 [SMILES: *C(=O)N1CCN(Cc2csc(NCC3CCCN3*)n2)CC1] | absent | 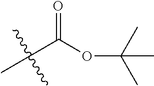 [SMILES: *C(=O)OC(C)(C)C] |
| 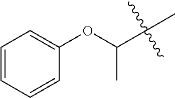 [SMILES: *C(C)Oc1ccccc1] | 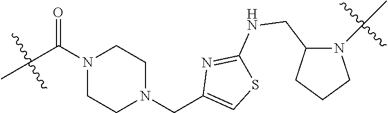 [SMILES: *C(=O)N1CCN(Cc2csc(NCC3CCCN3*)n2)CC1] | absent | 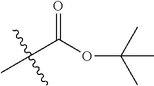 [SMILES: *C(=O)OC(C)(C)C] |
| 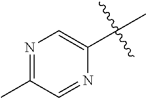 [SMILES: *c1cnc(C)cn1] | 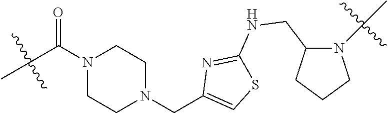 [SMILES: *C(=O)N1CCN(Cc2csc(NCC3CCCN3*)n2)CC1] | absent | 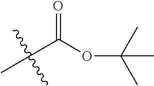 [SMILES: *C(=O)OC(C)(C)C] |
| 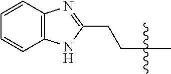 [SMILES: *CCc1nc2ccccc2[nH]1] | 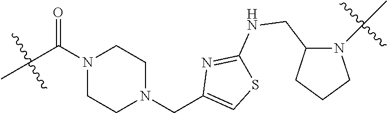 [SMILES: *C(=O)N1CCN(Cc2csc(NCC3CCCN3*)n2)CC1] | absent | 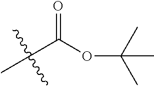 [SMILES: *C(=O)OC(C)(C)C] |
| 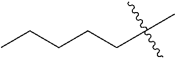 [SMILES: *CCCCC] | 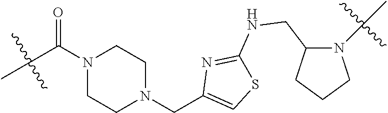 [SMILES: *C(=O)N1CCN(Cc2csc(NCC3CCCN3*)n2)CC1] | absent | 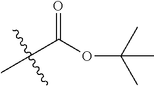 [SMILES: *C(=O)OC(C)(C)C] |
| 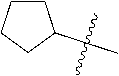 [SMILES: *C1CCCC1] | 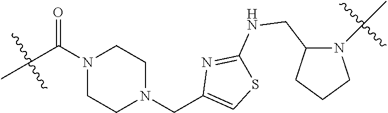 [SMILES: *C(=O)N1CCN(Cc2csc(NCC3CCCN3*)n2)CC1] | absent | 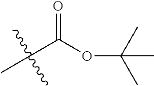 [SMILES: *C(=O)OC(C)(C)C] |
| 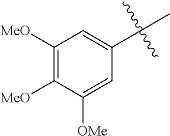 [SMILES: *c1cc(OC)c(OC)c(OC)c1] | 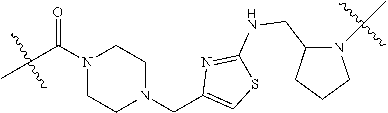 [SMILES: *C(=O)N1CCN(Cc2csc(NCC3CCCN3*)n2)CC1] | absent | 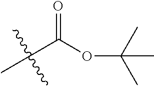 [SMILES: *C(=O)OC(C)(C)C] |

In some embodiments of the compound of Formula (III), the compound is as shown in Table 3 (below).

TABLE 3

| # | R₁-L₁ | 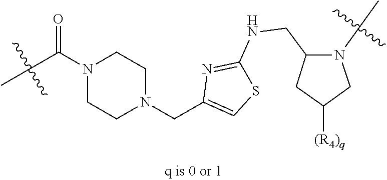 | R₄ | L₂-R₂ |
|---|---|---|---|---|
| 2678-58 | 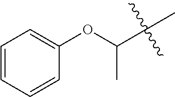 [SMILES: *C(C)Oc1ccccc1] | 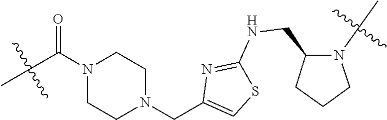 [SMILES: *C(=O)N1CCN(Cc2csc(NC[C@@H]3CCCN3*)n2)CC1] | absent | 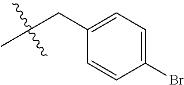 [SMILES: *Cc1ccc(Br)cc1] |
| 2678-80 | 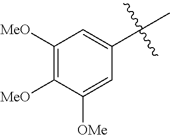 [SMILES: *c1cc(OC)c(OC)c(OC)c1] | 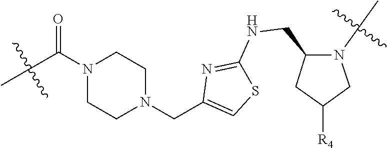 | OBzl | 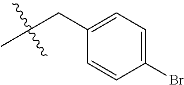 [SMILES: *Cc1ccc(Br)cc1] |
| 2678-66 | 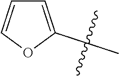 [SMILES: *c1ccco1] | 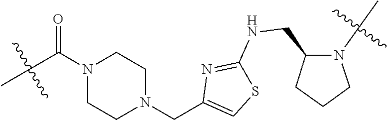 [SMILES: *C(=O)N1CCN(Cc2csc(NC[C@@H]3CCCN3*)n2)CC1] | absent | 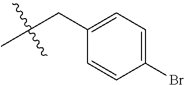 [SMILES: *Cc1ccc(Br)cc1] |
| 2678-10 | 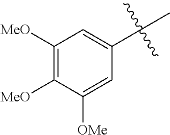 [SMILES: *c1cc(OC)c(OC)c(OC)c1] | 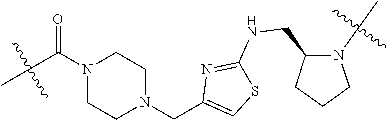 [SMILES: *C(=O)N1CCN(Cc2csc(NC[C@@H]3CCCN3*)n2)CC1] | absent | 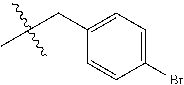 [SMILES: *Cc1ccc(Br)cc1] |
| 2678-18 | 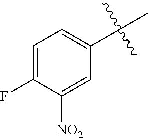 [SMILES: *c1ccc(F)c([N+](=O)[O-])c1] | 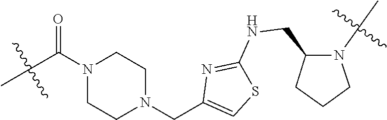 [SMILES: *C(=O)N1CCN(Cc2csc(NC[C@@H]3CCCN3*)n2)CC1] | absent | 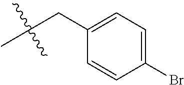 [SMILES: *Cc1ccc(Br)cc1] |
| 2678-50 | 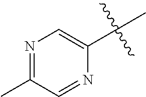 [SMILES: *c1cnc(C)cn1] | 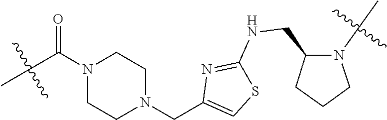 [SMILES: *C(=O)N1CCN(Cc2csc(NC[C@@H]3CCCN3*)n2)CC1] | absent | 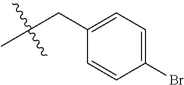 [SMILES: *Cc1ccc(Br)cc1] |
| 2678-2 | 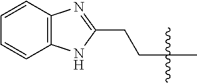 [SMILES: *CCc1nc2ccccc2[nH]1] | 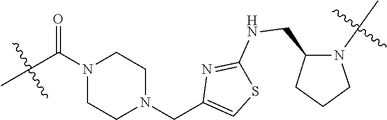 [SMILES: *C(=O)N1CCN(Cc2csc(NC[C@@H]3CCCN3*)n2)CC1] | absent | 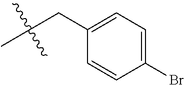 [SMILES: *Cc1ccc(Br)cc1] |
| 2678-78 | 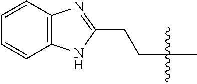 [SMILES: *CCc1nc2ccccc2[nH]1] | 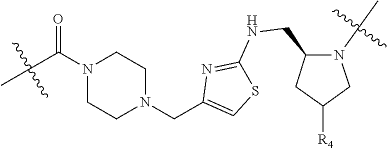 | OBzl | 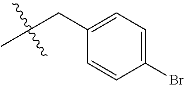 [SMILES: *Cc1ccc(Br)cc1] |
| 2463-262 | 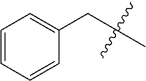 [SMILES: *Cc1ccccc1] | 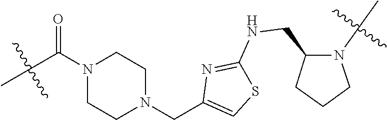 [SMILES: *C(=O)N1CCN(Cc2csc(NC[C@@H]3CCCN3*)n2)CC1] | absent | 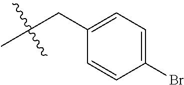 [SMILES: *Cc1ccc(Br)cc1] |
| 2678-26 | 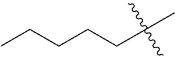 [SMILES: *CCCCC] | 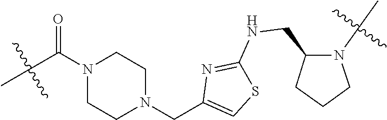 [SMILES: *C(=O)N1CCN(Cc2csc(NC[C@@H]3CCCN3*)n2)CC1] | absent | 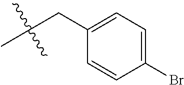 [SMILES: *Cc1ccc(Br)cc1] |
| 2678-42 | 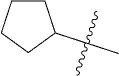 [SMILES: *C1CCCC1] | 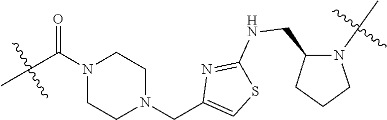 [SMILES: *C(=O)N1CCN(Cc2csc(NC[C@@H]3CCCN3*)n2)CC1] | absent | 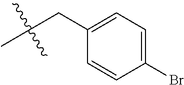 [SMILES: *Cc1ccc(Br)cc1] |
| 2678-34 | 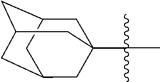 [SMILES: *C12CC3CC(CC(C3)C1)C2] | 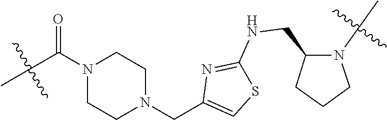 [SMILES: *C(=O)N1CCN(Cc2csc(NC[C@@H]3CCCN3*)n2)CC1] | absent | 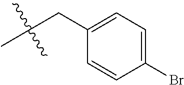 [SMILES: *Cc1ccc(Br)cc1] |
| 2678-74 | 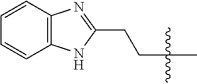 [SMILES: *CCc1nc2ccccc2[nH]1] | 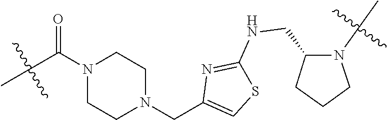 [SMILES: *C(=O)N1CCN(Cc2csc(NC[C@H]3CCCN3*)n2)CC1] | absent | 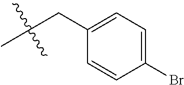 [SMILES: *Cc1ccc(Br)cc1] |
| 2678-76 | 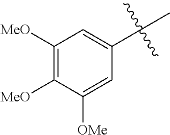 [SMILES: *c1cc(OC)c(OC)c(OC)c1] | 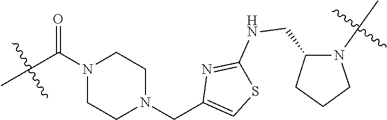 [SMILES: *C(=O)N1CCN(Cc2csc(NC[C@H]3CCCN3*)n2)CC1] | absent | 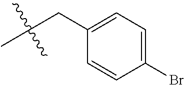 [SMILES: *Cc1ccc(Br)cc1] |
| 2678-12 | 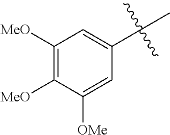 [SMILES: *c1cc(OC)c(OC)c(OC)c1] | 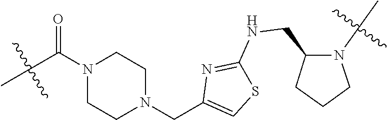 [SMILES: *C(=O)N1CCN(Cc2csc(NC[C@@H]3CCCN3*)n2)CC1] | absent | 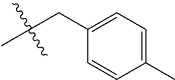 [SMILES: *Cc1ccc(C)cc1] |
| 2678-60 | 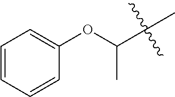 [SMILES: *C(C)Oc1ccccc1] | 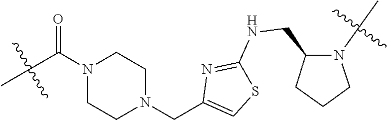 [SMILES: *C(=O)N1CCN(Cc2csc(NC[C@@H]3CCCN3*)n2)CC1] | absent | 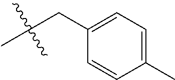 [SMILES: *Cc1ccc(C)cc1] |
| 2678-68 | 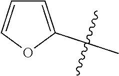 [SMILES: *c1ccco1] | 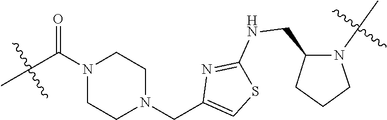 [SMILES: *C(=O)N1CCN(Cc2csc(NC[C@@H]3CCCN3*)n2)CC1] | absent | 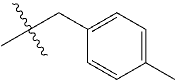 [SMILES: *Cc1ccc(C)cc1] |
| 2678-20 | 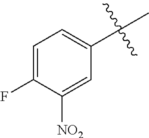 [SMILES: *c1ccc(F)c([N+](=O)[O-])c1] | 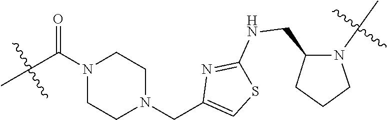 [SMILES: *C(=O)N1CCN(Cc2csc(NC[C@@H]3CCCN3*)n2)CC1] | absent | 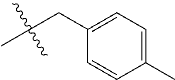 [SMILES: *Cc1ccc(C)cc1] |
| 2678-28 | 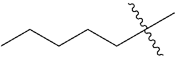 [SMILES: *CCCCC] | 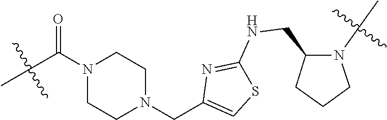 [SMILES: *C(=O)N1CCN(Cc2csc(NC[C@@H]3CCCN3*)n2)CC1] | absent | 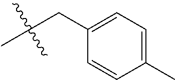 [SMILES: *Cc1ccc(C)cc1] |
| 2678-36 | 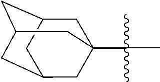 [SMILES: *C12CC3CC(CC(C3)C1)C2] | 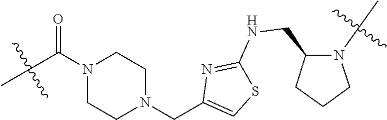 [SMILES: *C(=O)N1CCN(Cc2csc(NC[C@@H]3CCCN3*)n2)CC1] | absent | 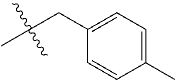 [SMILES: *Cc1ccc(C)cc1] |
| 2678-44 | 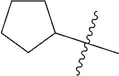 [SMILES: *C1CCCC1] | 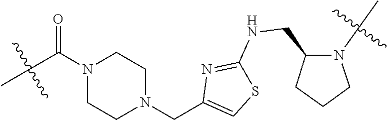 [SMILES: *C(=O)N1CCN(Cc2csc(NC[C@@H]3CCCN3*)n2)CC1] | absent | 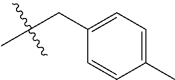 [SMILES: *Cc1ccc(C)cc1] |
| 2678-52 | 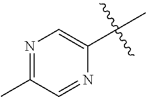 [SMILES: *c1cnc(C)cn1] | 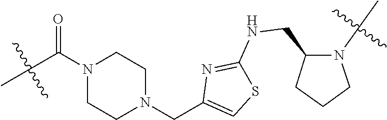 [SMILES: *C(=O)N1CCN(Cc2csc(NC[C@@H]3CCCN3*)n2)CC1] | absent | 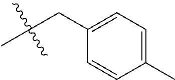 [SMILES: *Cc1ccc(C)cc1] |
| 2678-4 | 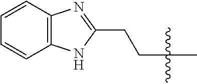 [SMILES: *CCc1nc2ccccc2[nH]1] | 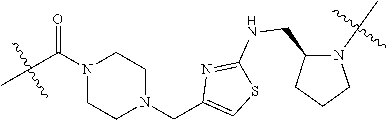 [SMILES: *C(=O)N1CCN(Cc2csc(NC[C@@H]3CCCN3*)n2)CC1] | absent | 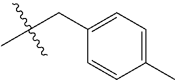 [SMILES: *Cc1ccc(C)cc1] |
| 2678-72 | 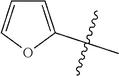 [SMILES: *c1ccco1] | 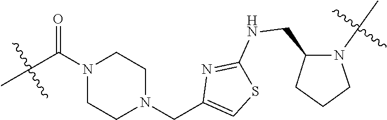 [SMILES: *C(=O)N1CCN(Cc2csc(NC[C@@H]3CCCN3*)n2)CC1] | absent | 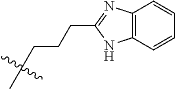 [SMILES: *CCCc1nc2ccccc2[nH]1] |
| 2678-32 | 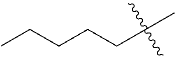 [SMILES: *CCCCC] | 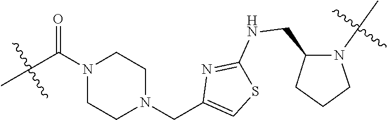 [SMILES: *C(=O)N1CCN(Cc2csc(NC[C@@H]3CCCN3*)n2)CC1] | absent | 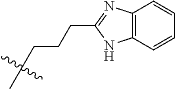 [SMILES: *CCCc1nc2ccccc2[nH]1] |
| 2678-24 | 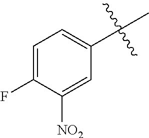 [SMILES: *c1ccc(F)c([N+](=O)[O-])c1] | 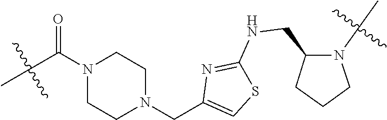 [SMILES: *C(=O)N1CCN(Cc2csc(NC[C@@H]3CCCN3*)n2)CC1] | absent | 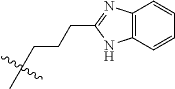 [SMILES: *CCCc1nc2ccccc2[nH]1] |
| 2678-16 | 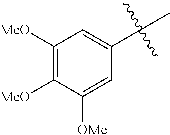 [SMILES: *c1cc(OC)c(OC)c(OC)c1] | 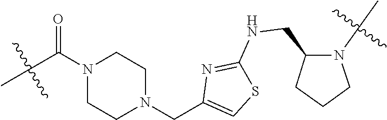 [SMILES: *C(=O)N1CCN(Cc2csc(NC[C@@H]3CCCN3*)n2)CC1] | absent | 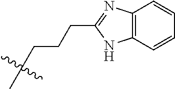 [SMILES: *CCCc1nc2ccccc2[nH]1] |
| 2678-64 | 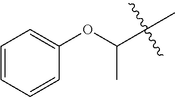 [SMILES: *C(C)Oc1ccccc1] | 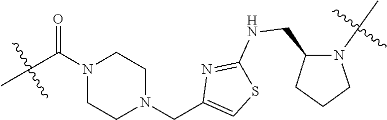 [SMILES: *C(=O)N1CCN(Cc2csc(NC[C@@H]3CCCN3*)n2)CC1] | absent | 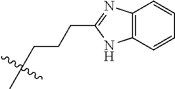 [SMILES: *CCCc1nc2ccccc2[nH]1] |
| 2678-8 | 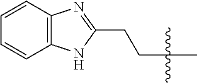 [SMILES: *CCc1nc2ccccc2[nH]1] | 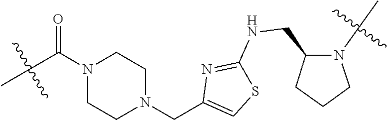 [SMILES: *C(=O)N1CCN(Cc2csc(NC[C@@H]3CCCN3*)n2)CC1] | absent | 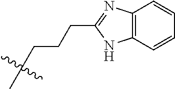 [SMILES: *CCCc1nc2ccccc2[nH]1] |
| 2678-40 | 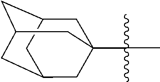 [SMILES: *C12CC3CC(CC(C3)C1)C2] | 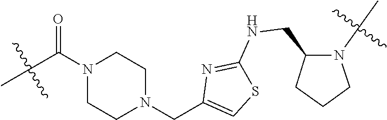 [SMILES: *C(=O)N1CCN(Cc2csc(NC[C@@H]3CCCN3*)n2)CC1] | absent | 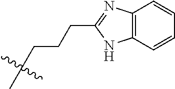 [SMILES: *CCCc1nc2ccccc2[nH]1] |
| 2678-48 | 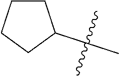 [SMILES: *C1CCCC1] | 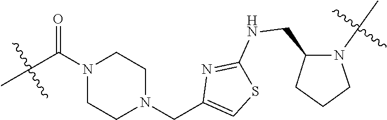 [SMILES: *C(=O)N1CCN(Cc2csc(NC[C@@H]3CCCN3*)n2)CC1] | absent | 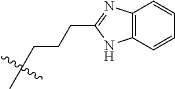 [SMILES: *CCCc1nc2ccccc2[nH]1] |
| 2678-56 | 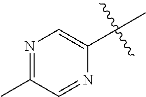 [SMILES: *c1cnc(C)cn1] | 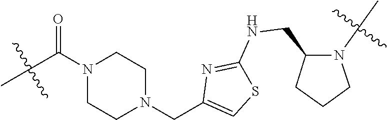 [SMILES: *C(=O)N1CCN(Cc2csc(NC[C@@H]3CCCN3*)n2)CC1] | absent | 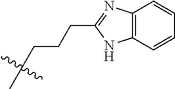 [SMILES: *CCCc1nc2ccccc2[nH]1] |
| 2678-43 | 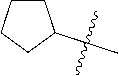 [SMILES: *C1CCCC1] | 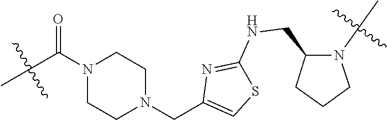 [SMILES: *C(=O)N1CCN(Cc2csc(NC[C@@H]3CCCN3*)n2)CC1] | absent | 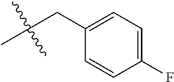 [SMILES: *Cc1ccc(F)cc1] |
| 2678-59 | 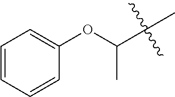 [SMILES: *C(C)Oc1ccccc1] | 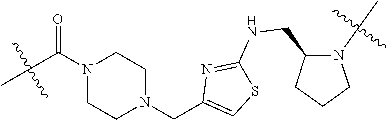 [SMILES: *C(=O)N1CCN(Cc2csc(NC[C@@H]3CCCN3*)n2)CC1] | absent | 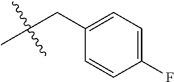 [SMILES: *Cc1ccc(F)cc1] |
| 2678-11 | 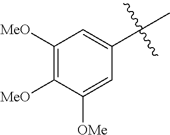 [SMILES: *c1cc(OC)c(OC)c(OC)c1] | 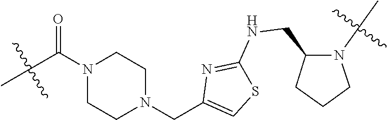 [SMILES: *C(=O)N1CCN(Cc2csc(NC[C@@H]3CCCN3*)n2)CC1] | absent | 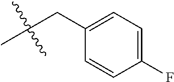 [SMILES: *Cc1ccc(F)cc1] |
| 2678-67 | 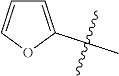 [SMILES: *c1ccco1] | 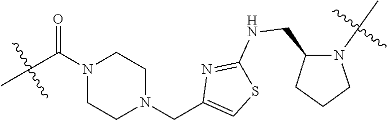 [SMILES: *C(=O)N1CCN(Cc2csc(NC[C@@H]3CCCN3*)n2)CC1] | absent | 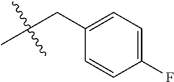 [SMILES: *Cc1ccc(F)cc1] |
| 2678-19 | 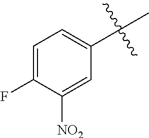 [SMILES: *c1ccc(F)c([N+](=O)[O-])c1] | 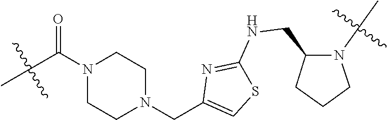 [SMILES: *C(=O)N1CCN(Cc2csc(NC[C@@H]3CCCN3*)n2)CC1] | absent | 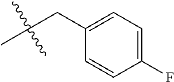 [SMILES: *Cc1ccc(F)cc1] |
| 2678-27 | 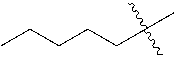 [SMILES: *CCCCC] | 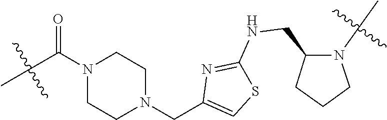 [SMILES: *C(=O)N1CCN(Cc2csc(NC[C@@H]3CCCN3*)n2)CC1] | absent | 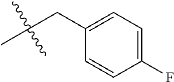 [SMILES: *Cc1ccc(F)cc1] |
| 2678-35 | 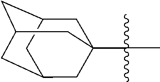 [SMILES: *C12CC3CC(CC(C3)C1)C2] | 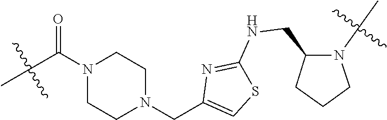 [SMILES: *C(=O)N1CCN(Cc2csc(NC[C@@H]3CCCN3*)n2)CC1] | absent | 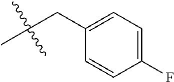 [SMILES: *Cc1ccc(F)cc1] |
| 2678-51 | 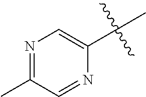 [SMILES: *c1cnc(C)cn1] | 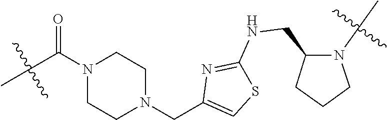 [SMILES: *C(=O)N1CCN(Cc2csc(NC[C@@H]3CCCN3*)n2)CC1] | absent | 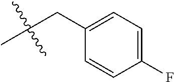 [SMILES: *Cc1ccc(F)cc1] |
| 2678-3 | 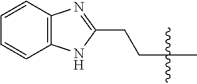 [SMILES: *CCc1nc2ccccc2[nH]1] | 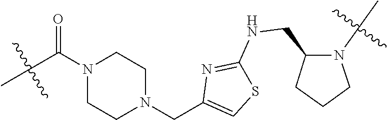 [SMILES: *C(=O)N1CCN(Cc2csc(NC[C@@H]3CCCN3*)n2)CC1] | absent | 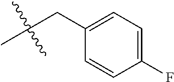 [SMILES: *Cc1ccc(F)cc1] |
| 2678-73 | 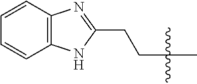 [SMILES: *CCc1nc2ccccc2[nH]1] | 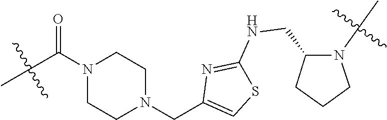 [SMILES: *C(=O)N1CCN(Cc2csc(NC[C@H]3CCCN3*)n2)CC1] | absent | 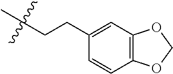 [SMILES: *CCc1ccc2c(c1)OCO2] |
| 2678-25 | 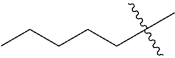 [SMILES: *CCCCC] | 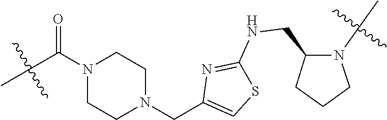 [SMILES: *C(=O)N1CCN(Cc2csc(NC[C@@H]3CCCN3*)n2)CC1] | absent | 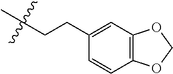 [SMILES: *CCc1ccc2c(c1)OCO2] |
| 2678-17 | 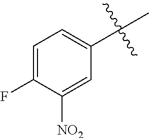 [SMILES: *c1ccc(F)c([N+](=O)[O-])c1] | 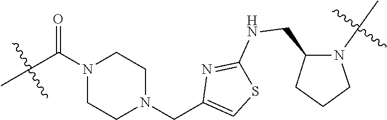 [SMILES: *C(=O)N1CCN(Cc2csc(NC[C@@H]3CCCN3*)n2)CC1] | absent | 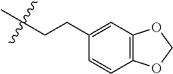 [SMILES: *CCc1ccc2c(c1)OCO2] |
| 2678-1 | 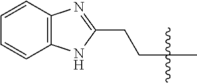 [SMILES: *CCc1nc2ccccc2[nH]1] | 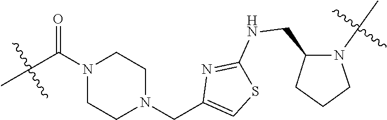 [SMILES: *C(=O)N1CCN(Cc2csc(NC[C@@H]3CCCN3*)n2)CC1] | absent | 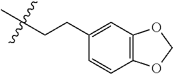 [SMILES: *CCc1ccc2c(c1)OCO2] |
| 2678-17 | 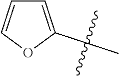 [SMILES: *c1ccco1] | 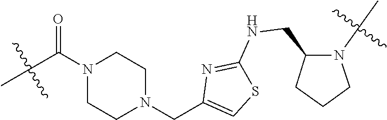 [SMILES: *C(=O)N1CCN(Cc2csc(NC[C@@H]3CCCN3*)n2)CC1] | absent | 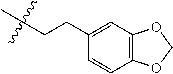 [SMILES: *CCc1ccc2c(c1)OCO2] |
| 2678-49 | 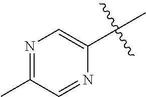 [SMILES: *c1cnc(C)cn1] | 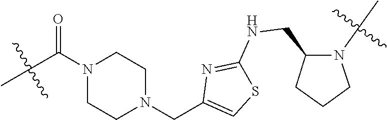 [SMILES: *C(=O)N1CCN(Cc2csc(NC[C@@H]3CCCN3*)n2)CC1] | absent | 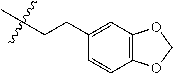 [SMILES: *CCc1ccc2c(c1)OCO2] |
| 2678-75 | 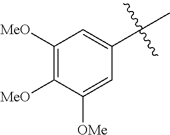 [SMILES: *c1cc(OC)c(OC)c(OC)c1] | 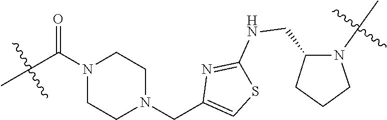 [SMILES: *C(=O)N1CCN(Cc2csc(NC[C@H]3CCCN3*)n2)CC1] | absent | 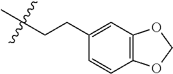 [SMILES: *CCc1ccc2c(c1)OCO2] |
| 2678-41 | 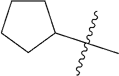 [SMILES: *C1CCCC1] | 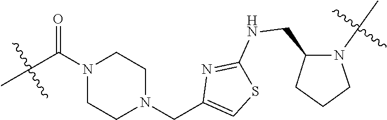 [SMILES: *C(=O)N1CCN(Cc2csc(NC[C@@H]3CCCN3*)n2)CC1] | absent | 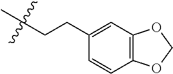 [SMILES: *CCc1ccc2c(c1)OCO2] |
| 2678-9 | 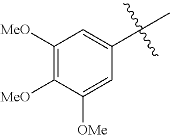 [SMILES: *c1cc(OC)c(OC)c(OC)c1] | 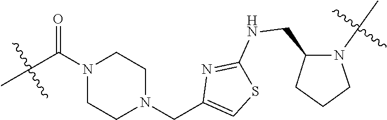 [SMILES: *C(=O)N1CCN(Cc2csc(NC[C@@H]3CCCN3*)n2)CC1] | absent | 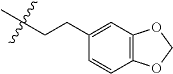 [SMILES: *CCc1ccc2c(c1)OCO2] |
| 2678-33 | 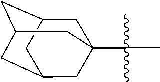 [SMILES: *C12CC3CC(CC(C3)C1)C2] | 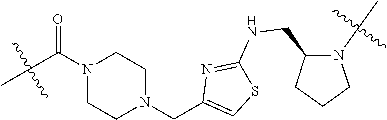 [SMILES: *C(=O)N1CCN(Cc2csc(NC[C@@H]3CCCN3*)n2)CC1] | absent | 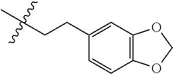 [SMILES: *CCc1ccc2c(c1)OCO2] |
| 2678-57 | 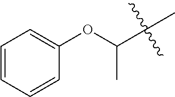 [SMILES: *C(C)Oc1ccccc1] | 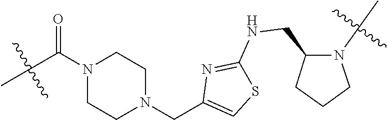 [SMILES: *C(=O)N1CCN(Cc2csc(NC[C@@H]3CCCN3*)n2)CC1] | absent | 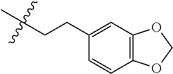 [SMILES: *CCc1ccc2c(c1)OCO2] |
| 2678-77 | 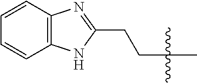 [SMILES: *CCc1nc2ccccc2[nH]1] | 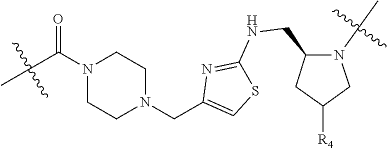 | OBzl | 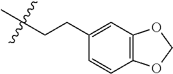 [SMILES: *CCc1ccc2c(c1)OCO2] |
| 2678-79 | 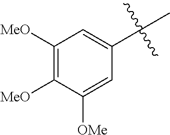 [SMILES: *c1cc(OC)c(OC)c(OC)c1] | 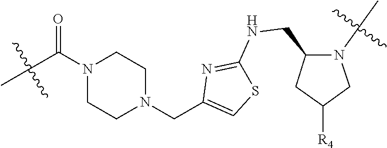 | OBzl | 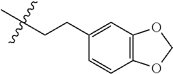 [SMILES: *CCc1ccc2c(c1)OCO2] |
| 2678-15 | 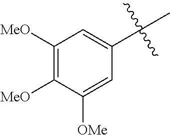 [SMILES: *c1cc(OC)c(OC)c(OC)c1] | 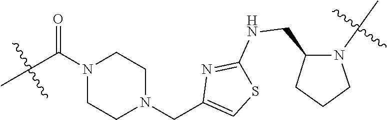 [SMILES: *C(=O)N1CCN(Cc2csc(NC[C@@H]3CCCN3*)n2)CC1] | absent | 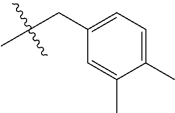 [SMILES: *Cc1ccc(C)c(C)c1] |
| 2678-63 | 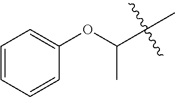 [SMILES: *C(C)Oc1ccccc1] | 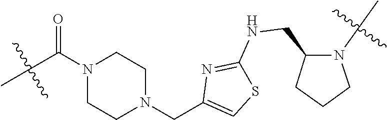 [SMILES: *C(=O)N1CCN(Cc2csc(NC[C@@H]3CCCN3*)n2)CC1] | absent | 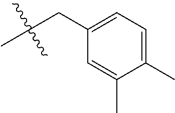 [SMILES: *Cc1ccc(C)c(C)c1] |
| 2678-23 | 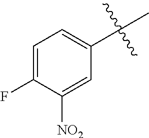 [SMILES: *c1ccc(F)c([N+](=O)[O-])c1] | 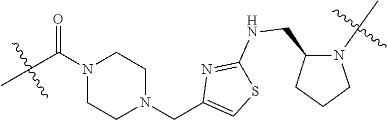 [SMILES: *C(=O)N1CCN(Cc2csc(NC[C@@H]3CCCN3*)n2)CC1] | absent | 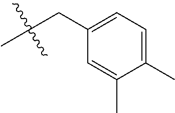 [SMILES: *Cc1ccc(C)c(C)c1] |
| 2678-71 | 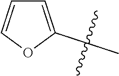 [SMILES: *c1ccco1] | 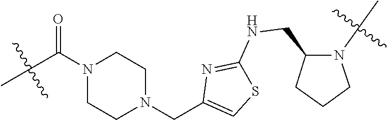 [SMILES: *C(=O)N1CCN(Cc2csc(NC[C@@H]3CCCN3*)n2)CC1] | absent | 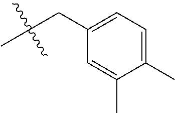 [SMILES: *Cc1ccc(C)c(C)c1] |
| 2678-55 | 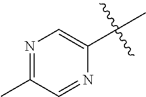 [SMILES: *c1cnc(C)cn1] | 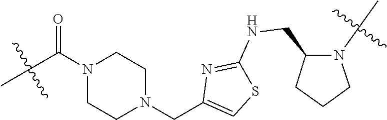 [SMILES: *C(=O)N1CCN(Cc2csc(NC[C@@H]3CCCN3*)n2)CC1] | absent | 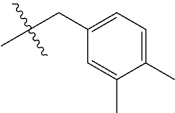 [SMILES: *Cc1ccc(C)c(C)c1] |
| 2678-7 | 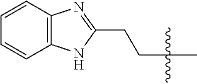 [SMILES: *CCc1nc2ccccc2[nH]1] | 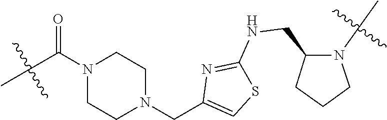 [SMILES: *C(=O)N1CCN(Cc2csc(NC[C@@H]3CCCN3*)n2)CC1] | absent | 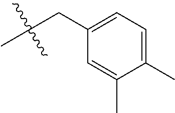 [SMILES: *Cc1ccc(C)c(C)c1] |
| 2678-31 | 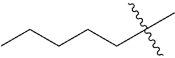 [SMILES: *CCCCC] | 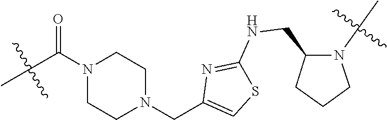 [SMILES: *C(=O)N1CCN(Cc2csc(NC[C@@H]3CCCN3*)n2)CC1] | absent | 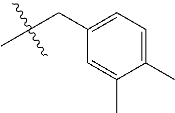 [SMILES: *Cc1ccc(C)c(C)c1] |
| 2678-47 | 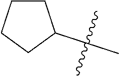 [SMILES: *C1CCCC1] | 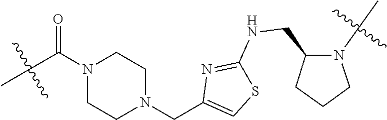 [SMILES: *C(=O)N1CCN(Cc2csc(NC[C@@H]3CCCN3*)n2)CC1] | absent | 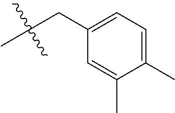 [SMILES: *Cc1ccc(C)c(C)c1] |
| 2678-39 | 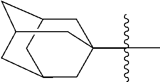 [SMILES: *C12CC3CC(CC(C3)C1)C2] | 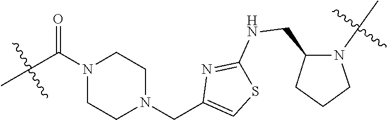 [SMILES: *C(=O)N1CCN(Cc2csc(NC[C@@H]3CCCN3*)n2)CC1] | absent | 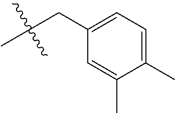 [SMILES: *Cc1ccc(C)c(C)c1] |
| 2678-21 | 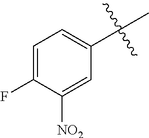 [SMILES: *c1ccc(F)c([N+](=O)[O-])c1] | 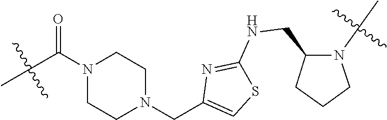 [SMILES: *C(=O)N1CCN(Cc2csc(NC[C@@H]3CCCN3*)n2)CC1] | absent | 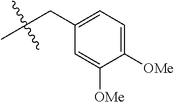 [SMILES: *Cc1ccc(OC)c(OC)c1] |
| 2678-69 | 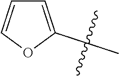 [SMILES: *c1ccco1] | 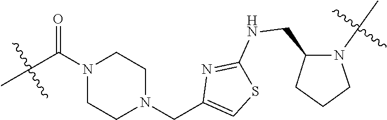 [SMILES: *C(=O)N1CCN(Cc2csc(NC[C@@H]3CCCN3*)n2)CC1] | absent | 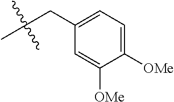 [SMILES: *Cc1ccc(OC)c(OC)c1] |
| 2678-13 | 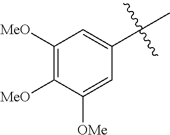 [SMILES: *c1cc(OC)c(OC)c(OC)c1] | 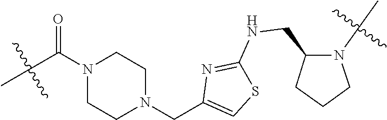 [SMILES: *C(=O)N1CCN(Cc2csc(NC[C@@H]3CCCN3*)n2)CC1] | absent | 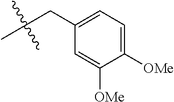 [SMILES: *Cc1ccc(OC)c(OC)c1] |
| 2678-61 | 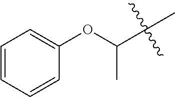 [SMILES: *C(C)Oc1ccccc1] | 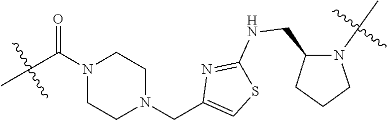 [SMILES: *C(=O)N1CCN(Cc2csc(NC[C@@H]3CCCN3*)n2)CC1] | absent | 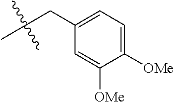 [SMILES: *Cc1ccc(OC)c(OC)c1] |
| 2678-29 | 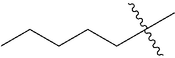 [SMILES: *CCCCC] | 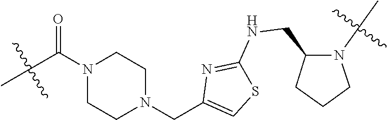 [SMILES: *C(=O)N1CCN(Cc2csc(NC[C@@H]3CCCN3*)n2)CC1] | absent | 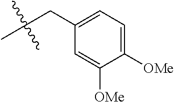 [SMILES: *Cc1ccc(OC)c(OC)c1] |
| 2678-37 | 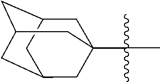 [SMILES: *C12CC3CC(CC(C3)C1)C2] | 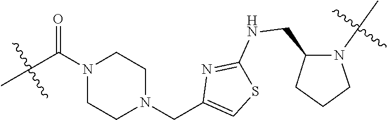 [SMILES: *C(=O)N1CCN(Cc2csc(NC[C@@H]3CCCN3*)n2)CC1] | absent | 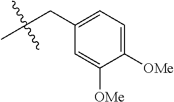 [SMILES: *Cc1ccc(OC)c(OC)c1] |
| 2678-45 | 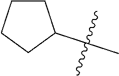 [SMILES: *C1CCCC1] | 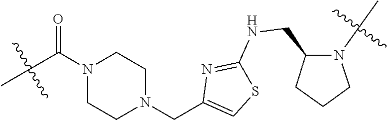 [SMILES: *C(=O)N1CCN(Cc2csc(NC[C@@H]3CCCN3*)n2)CC1] | absent | 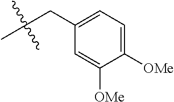 [SMILES: *Cc1ccc(OC)c(OC)c1] |
| 2678-53 | 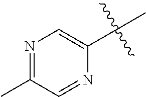 [SMILES: *c1cnc(C)cn1] | 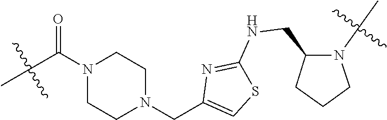 [SMILES: *C(=O)N1CCN(Cc2csc(NC[C@@H]3CCCN3*)n2)CC1] | absent | 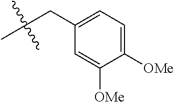 [SMILES: *Cc1ccc(OC)c(OC)c1] |
| 2678-5 | 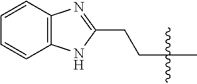 [SMILES: *CCc1nc2ccccc2[nH]1] | 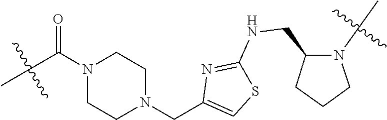 [SMILES: *C(=O)N1CCN(Cc2csc(NC[C@@H]3CCCN3*)n2)CC1] | absent | 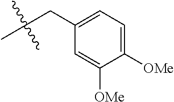 [SMILES: *Cc1ccc(OC)c(OC)c1] |
| 2678-30 | 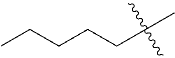 [SMILES: *CCCCC] | 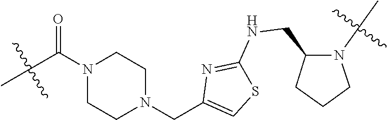 [SMILES: *C(=O)N1CCN(Cc2csc(NC[C@@H]3CCCN3*)n2)CC1] | absent | 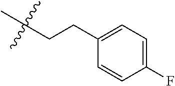 [SMILES: *CCc1ccc(F)cc1] |
| 2678-22 | 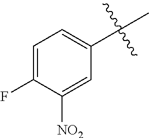 [SMILES: *c1ccc(F)c([N+](=O)[O-])c1] | 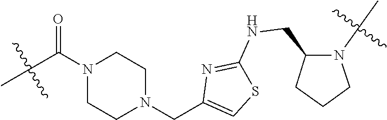 [SMILES: *C(=O)N1CCN(Cc2csc(NC[C@@H]3CCCN3*)n2)CC1] | absent | 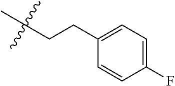 [SMILES: *CCc1ccc(F)cc1] |
| 2678-14 | 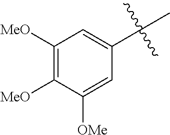 [SMILES: *c1cc(OC)c(OC)c(OC)c1] | 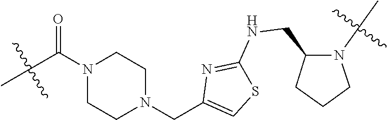 [SMILES: *C(=O)N1CCN(Cc2csc(NC[C@@H]3CCCN3*)n2)CC1] | absent | 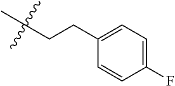 [SMILES: *CCc1ccc(F)cc1] |
| 2678-62 | 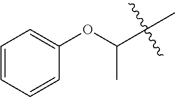 [SMILES: *C(C)Oc1ccccc1] | 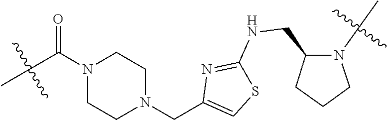 [SMILES: *C(=O)N1CCN(Cc2csc(NC[C@@H]3CCCN3*)n2)CC1] | absent | 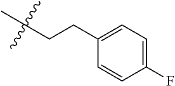 [SMILES: *CCc1ccc(F)cc1] |
| 2678-70 | 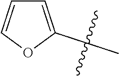 [SMILES: *c1ccco1] | 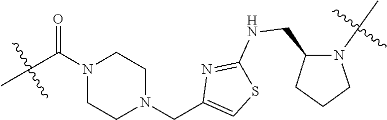 [SMILES: *C(=O)N1CCN(Cc2csc(NC[C@@H]3CCCN3*)n2)CC1] | absent | 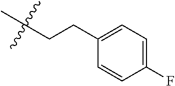 [SMILES: *CCc1ccc(F)cc1] |
| 2678-38 | 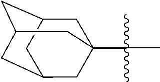 [SMILES: *C12CC3CC(CC(C3)C1)C2] | 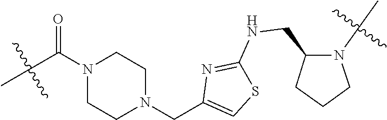 [SMILES: *C(=O)N1CCN(Cc2csc(NC[C@@H]3CCCN3*)n2)CC1] | absent | 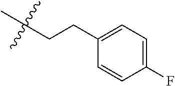 [SMILES: *CCc1ccc(F)cc1] |
| 2678-54 | 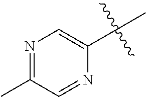 [SMILES: *c1cnc(C)cn1] | 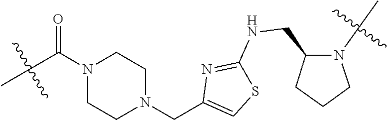 [SMILES: *C(=O)N1CCN(Cc2csc(NC[C@@H]3CCCN3*)n2)CC1] | absent | 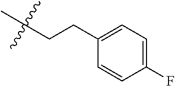 [SMILES: *CCc1ccc(F)cc1] |
| 2678-6 | 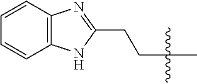 [SMILES: *CCc1nc2ccccc2[nH]1] | 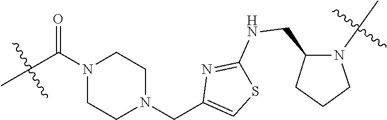 [SMILES: *C(=O)N1CCN(Cc2csc(NC[C@@H]3CCCN3*)n2)CC1] | absent | 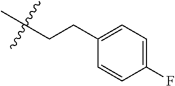 [SMILES: *CCc1ccc(F)cc1] |
| 2678-89 | 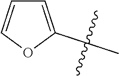 [SMILES: *c1ccco1] | 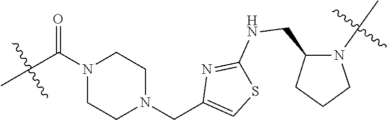 [SMILES: *C(=O)N1CCN(Cc2csc(NC[C@@H]3CCCN3*)n2)CC1] | absent | 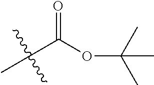 [SMILES: *C(=O)OC(C)(C)C] |
| 2678-85 | 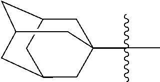 [SMILES: *C12CC3CC(CC(C3)C1)C2] | 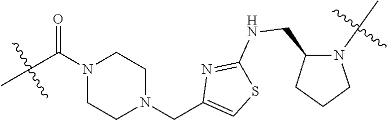 [SMILES: *C(=O)N1CCN(Cc2csc(NC[C@@H]3CCCN3*)n2)CC1] | absent | 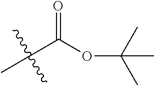 [SMILES: *C(=O)OC(C)(C)C] |
| 2678-81 | 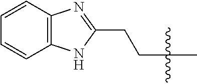 [SMILES: *CCc1nc2ccccc2[nH]1] | 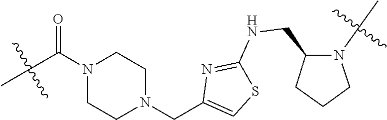 [SMILES: *C(=O)N1CCN(Cc2csc(NC[C@@H]3CCCN3*)n2)CC1] | absent | 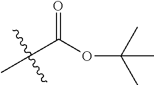 [SMILES: *C(=O)OC(C)(C)C] |
| 2678-83 | 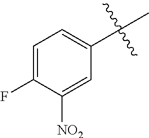 [SMILES: *c1ccc(F)c([N+](=O)[O-])c1] | 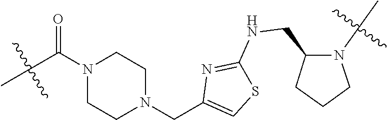 [SMILES: *C(=O)N1CCN(Cc2csc(NC[C@@H]3CCCN3*)n2)CC1] | absent | 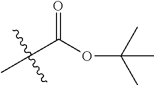 [SMILES: *C(=O)OC(C)(C)C] |
| 2678-88 | 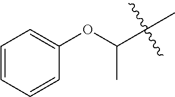 [SMILES: *C(C)Oc1ccccc1] | 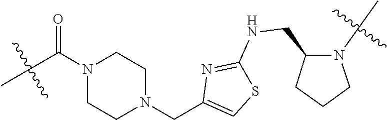 [SMILES: *C(=O)N1CCN(Cc2csc(NC[C@@H]3CCCN3*)n2)CC1] | absent | 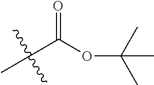 [SMILES: *C(=O)OC(C)(C)C] |
| 2678-87 | 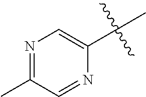 [SMILES: *c1cnc(C)cn1] | 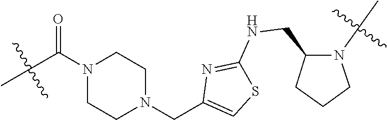 [SMILES: *C(=O)N1CCN(Cc2csc(NC[C@@H]3CCCN3*)n2)CC1] | absent | 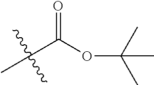 [SMILES: *C(=O)OC(C)(C)C] |
| 2678-90 | 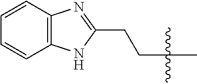 [SMILES: *CCc1nc2ccccc2[nH]1] | 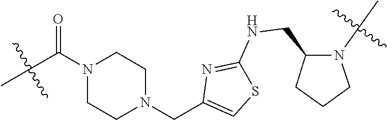 [SMILES: *C(=O)N1CCN(Cc2csc(NC[C@@H]3CCCN3*)n2)CC1] | absent | 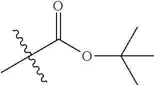 [SMILES: *C(=O)OC(C)(C)C] |
| 2678-84 | 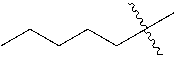 [SMILES: *CCCCC] | 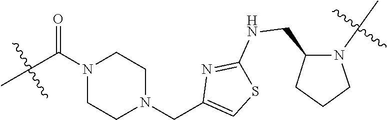 [SMILES: *C(=O)N1CCN(Cc2csc(NC[C@@H]3CCCN3*)n2)CC1] | absent | 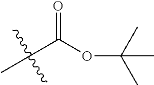 [SMILES: *C(=O)OC(C)(C)C] |
| 2678-86 | 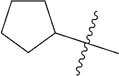 [SMILES: *C1CCCC1] | 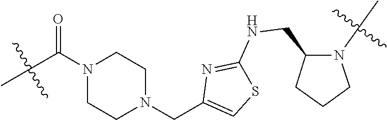 [SMILES: *C(=O)N1CCN(Cc2csc(NC[C@@H]3CCCN3*)n2)CC1] | absent | 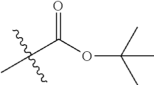 [SMILES: *C(=O)OC(C)(C)C] |
| 2678-82 | 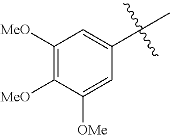 [SMILES: *c1cc(OC)c(OC)c(OC)c1] | 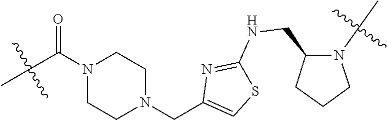 [SMILES: *C(=O)N1CCN(Cc2csc(NC[C@@H]3CCCN3*)n2)CC1] | absent | 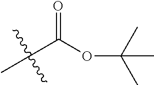 [SMILES: *C(=O)OC(C)(C)C] |

In some embodiments of the compound of Formula (I) or (II), the compound is a compound of Formula (IV):

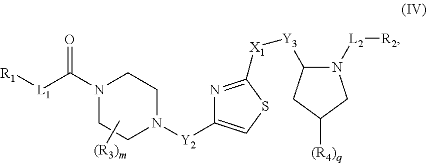

wherein m is 0 or 2, q is 0 or 1, and X₂ is —N— or —OH—.

In some embodiments of the compound of Formula (IV), the compound is as shown in Table 4 (below).

TABLE 4

| R₁-L₁ | 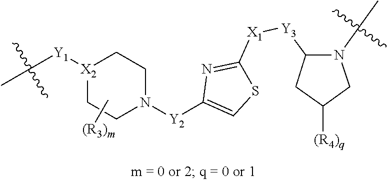 | R₄ | L₂-R₂ |
|---|---|---|---|
| 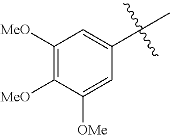 [SMILES: *c1cc(OC)c(OC)c(OC)c1] | 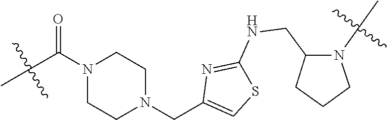 [SMILES: *C(=O)N1CCN(Cc2csc(NCC3CCCN3*)n2)CC1] | absent | 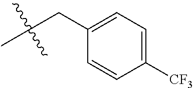 [SMILES: *Cc1ccc(C(F)(F)F)cc1] |
| 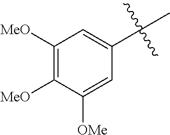 [SMILES: *c1cc(OC)c(OC)c(OC)c1] | 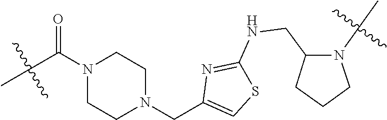 [SMILES: *C(=O)N1CCN(Cc2csc(NCC3CCCN3*)n2)CC1] | absent | 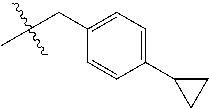 [SMILES: *Cc1ccc(C2CC2)cc1] |
| 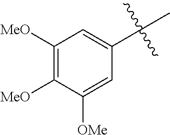 [SMILES: *c1cc(OC)c(OC)c(OC)c1] | 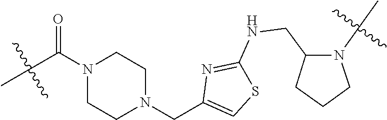 [SMILES: *C(=O)N1CCN(Cc2csc(NCC3CCCN3*)n2)CC1] | absent | 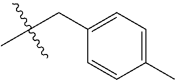 [SMILES: *Cc1ccc(C)cc1] |
| 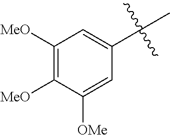 [SMILES: *c1cc(OC)c(OC)c(OC)c1] | 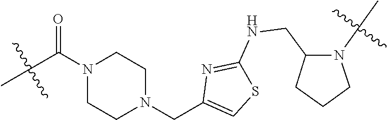 [SMILES: *C(=O)N1CCN(Cc2csc(NCC3CCCN3*)n2)CC1] | absent | 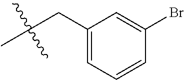 [SMILES: *Cc1cccc(Br)c1] |
| 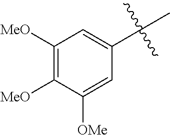 [SMILES: *c1cc(OC)c(OC)c(OC)c1] | 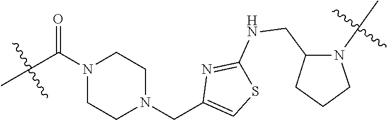 [SMILES: *C(=O)N1CCN(Cc2csc(NCC3CCCN3*)n2)CC1] | absent | 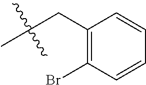 [SMILES: *Cc1ccccc1Br] |
| 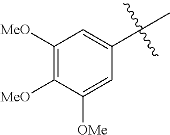 [SMILES: *c1cc(OC)c(OC)c(OC)c1] | 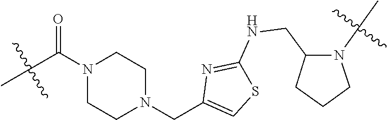 [SMILES: *C(=O)N1CCN(Cc2csc(NCC3CCCN3*)n2)CC1] | absent | 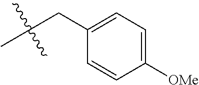 [SMILES: *Cc1ccc(OC)cc1] |
| 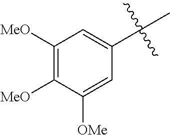 [SMILES: *c1cc(OC)c(OC)c(OC)c1] | 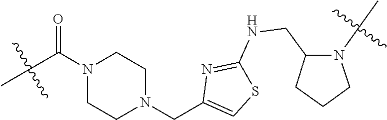 [SMILES: *C(=O)N1CCN(Cc2csc(NCC3CCCN3*)n2)CC1] | absent | 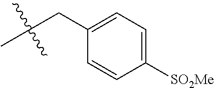 [SMILES: *Cc1ccc(S(C)(=O)=O)cc1] |
| 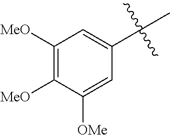 [SMILES: *c1cc(OC)c(OC)c(OC)c1] | 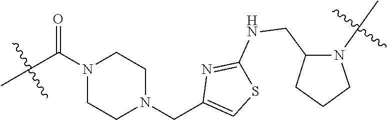 [SMILES: *C(=O)N1CCN(Cc2csc(NCC3CCCN3*)n2)CC1] | absent | 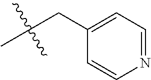 [SMILES: *Cc1ccncc1] |
| 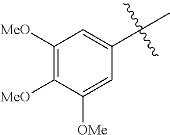 [SMILES: *c1cc(OC)c(OC)c(OC)c1] | 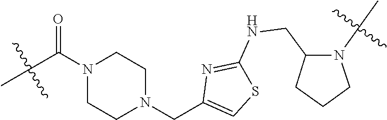 [SMILES: *C(=O)N1CCN(Cc2csc(NCC3CCCN3*)n2)CC1] | absent | 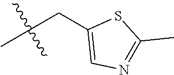 [SMILES: *Cc1cnc(C)s1] |
| 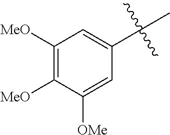 [SMILES: *c1cc(OC)c(OC)c(OC)c1] | 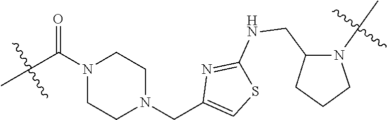 [SMILES: *C(=O)N1CCN(Cc2csc(NCC3CCCN3*)n2)CC1] | absent | 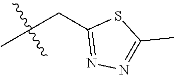 [SMILES: *Cc1nnc(C)s1] |
| 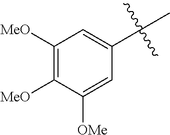 [SMILES: *c1cc(OC)c(OC)c(OC)c1] | 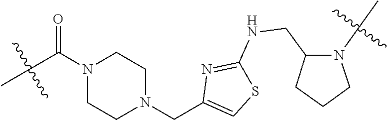 [SMILES: *C(=O)N1CCN(Cc2csc(NCC3CCCN3*)n2)CC1] | absent | 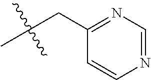 [SMILES: *Cc1ccncn1] |
| 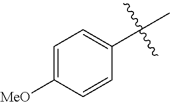 [SMILES: *c1ccc(OC)cc1] | 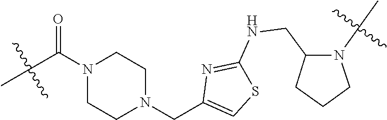 [SMILES: *C(=O)N1CCN(Cc2csc(NCC3CCCN3*)n2)CC1] | absent | 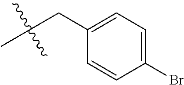 [SMILES: *Cc1ccc(Br)cc1] |
| 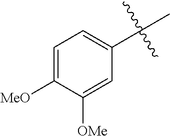 [SMILES: *c1ccc(OC)c(OC)c1] | 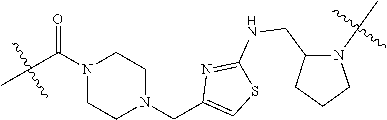 [SMILES: *C(=O)N1CCN(Cc2csc(NCC3CCCN3*)n2)CC1] | absent | 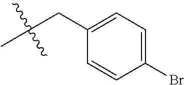 [SMILES: *Cc1ccc(Br)cc1] |
| 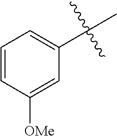 [SMILES: *c1cccc(OC)c1] | 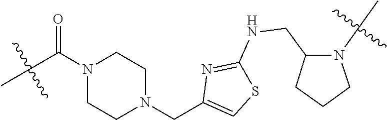 [SMILES: *C(=O)N1CCN(Cc2csc(NCC3CCCN3*)n2)CC1] | absent | 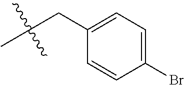 [SMILES: *Cc1ccc(Br)cc1] |
| 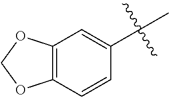 [SMILES: *c1ccc2c(c1)OCO2] | 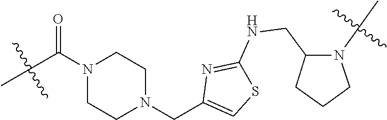 [SMILES: *C(=O)N1CCN(Cc2csc(NCC3CCCN3*)n2)CC1] | absent | 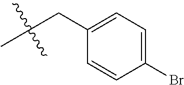 [SMILES: *Cc1ccc(Br)cc1] |
| 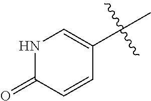 [SMILES: *c1ccc(=O)[nH]c1] | 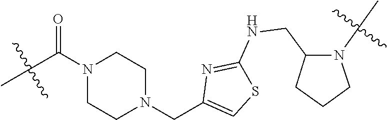 [SMILES: *C(=O)N1CCN(Cc2csc(NCC3CCCN3*)n2)CC1] | absent | 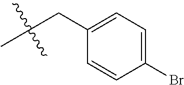 [SMILES: *Cc1ccc(Br)cc1] |
| 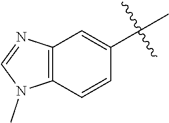 [SMILES: *c1ccc2c(c1)ncn2C] | 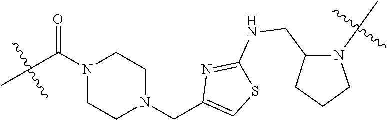 [SMILES: *C(=O)N1CCN(Cc2csc(NCC3CCCN3*)n2)CC1] | absent | 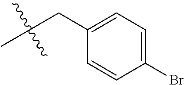 [SMILES: *Cc1ccc(Br)cc1] |
| 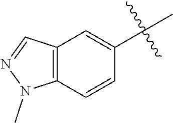 [SMILES: *c1ccc2c(cnn2C)c1] | 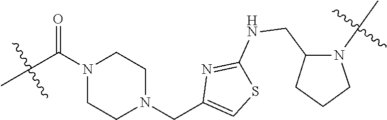 [SMILES: *C(=O)N1CCN(Cc2csc(NCC3CCCN3*)n2)CC1] | absent | 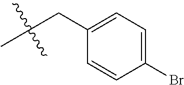 [SMILES: *Cc1ccc(Br)cc1] |
| 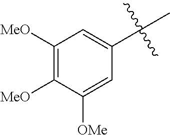 [SMILES: *c1cc(OC)c(OC)c(OC)c1] | 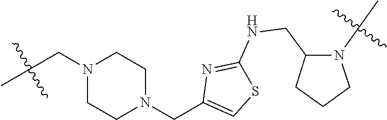 [SMILES: *CN1CCN(Cc2csc(NCC3CCCN3*)n2)CC1] | absent | 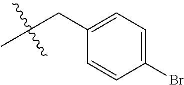 [SMILES: *Cc1ccc(Br)cc1] |
| 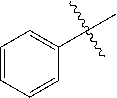 [SMILES: *c1ccccc1] | 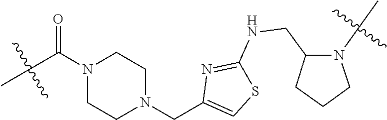 [SMILES: *C(=O)N1CCN(Cc2csc(NCC3CCCN3*)n2)CC1] | absent | 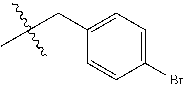 [SMILES: *Cc1ccc(Br)cc1] |
| 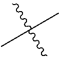 | 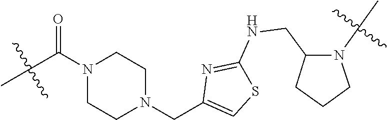 [SMILES: *C(=O)N1CCN(Cc2csc(NCC3CCCN3*)n2)CC1] | absent | 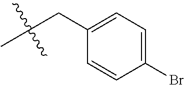 [SMILES: *Cc1ccc(Br)cc1] |
| 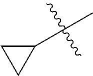 [SMILES: *C1CC1] | 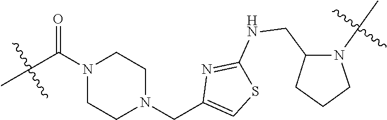 [SMILES: *C(=O)N1CCN(Cc2csc(NCC3CCCN3*)n2)CC1] | absent | 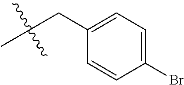 [SMILES: *Cc1ccc(Br)cc1] |
| 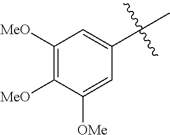 [SMILES: *c1cc(OC)c(OC)c(OC)c1] | 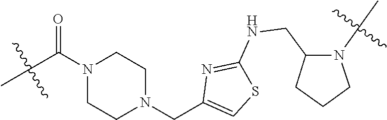 [SMILES: *C(=O)N1CCN(Cc2csc(NCC3CCCN3*)n2)CC1] | absent | 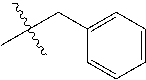 [SMILES: *Cc1ccccc1] |
| 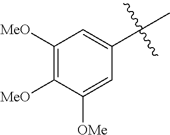 [SMILES: *c1cc(OC)c(OC)c(OC)c1] | 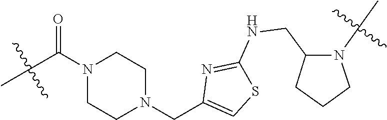 [SMILES: *C(=O)N1CCN(Cc2csc(NCC3CCCN3*)n2)CC1] | absent | 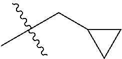 [SMILES: *CC1CC1] |
| 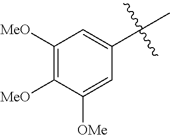 [SMILES: *c1cc(OC)c(OC)c(OC)c1] | 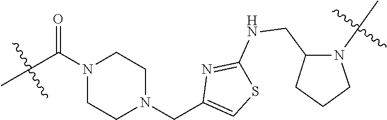 [SMILES: *C(=O)N1CCN(Cc2csc(NCC3CCCN3*)n2)CC1] | absent | 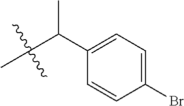 [SMILES: *C(C)c1ccc(Br)cc1] |
| 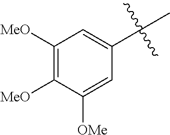 [SMILES: *c1cc(OC)c(OC)c(OC)c1] | 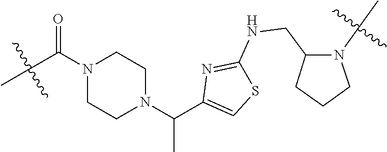 [SMILES: *C(=O)N1CCN(C(C)c2csc(NCC3CCCN3*)n2)CC1] | absent | 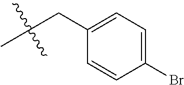 [SMILES: *Cc1ccc(Br)cc1] |
| 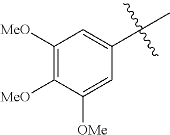 [SMILES: *c1cc(OC)c(OC)c(OC)c1] | 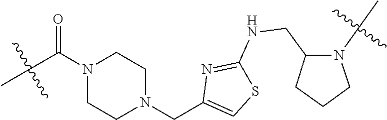 [SMILES: *C(=O)N1CCN(Cc2csc(NCC3CCCN3*)n2)CC1] | absent | 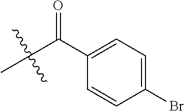 [SMILES: *C(=O)c1ccc(Br)cc1] |
| 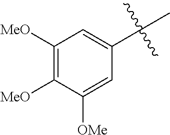 [SMILES: *c1cc(OC)c(OC)c(OC)c1] | 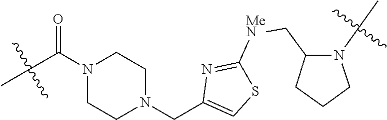 [SMILES: *C(=O)N1CCN(Cc2csc(N(C)CC3CCCN3*)n2)CC1] | absent | 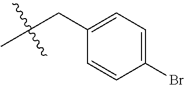 [SMILES: *Cc1ccc(Br)cc1] |
| 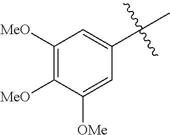 [SMILES: *c1cc(OC)c(OC)c(OC)c1] | 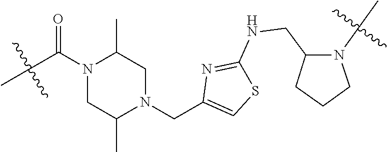 [SMILES: *C(=O)N1CC(C)N(Cc2csc(NCC3CCCN3*)n2)CC1C] | absent | 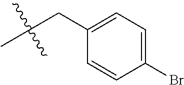 [SMILES: *Cc1ccc(Br)cc1] |
| 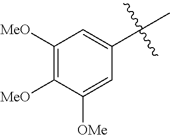 [SMILES: *c1cc(OC)c(OC)c(OC)c1] | 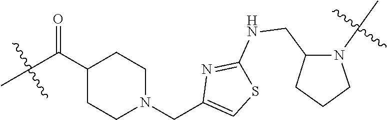 [SMILES: *C(=O)C1CCN(Cc2csc(NCC3CCCN3*)n2)CC1] | absent | 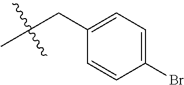 [SMILES: *Cc1ccc(Br)cc1] |
| 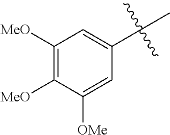 [SMILES: *c1cc(OC)c(OC)c(OC)c1] | 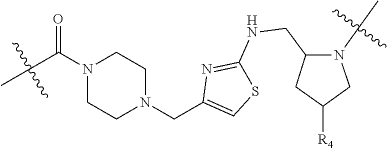 | OH | 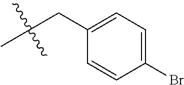 [SMILES: *Cc1ccc(Br)cc1] |
| 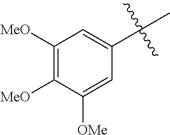 [SMILES: *c1cc(OC)c(OC)c(OC)c1] | 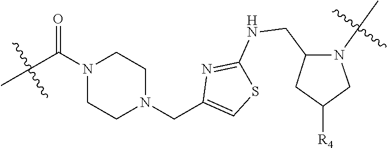 | OMe | 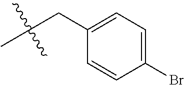 [SMILES: *Cc1ccc(Br)cc1] |
| 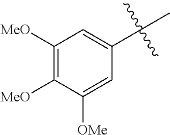 [SMILES: *c1cc(OC)c(OC)c(OC)c1] | 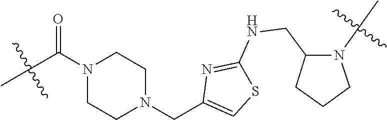 [SMILES: *C(=O)N1CCN(Cc2csc(NCC3CCCN3*)n2)CC1] | absent | 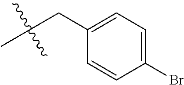 [SMILES: *Cc1ccc(Br)cc1] |
| 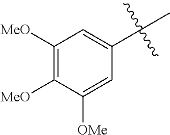 [SMILES: *c1cc(OC)c(OC)c(OC)c1] | 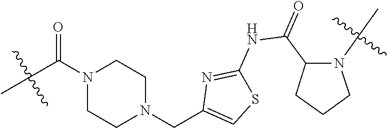 [SMILES: *C(=O)N1CCN(Cc2csc(NC(=O)C3CCCN3*)n2)CC1] | absent | 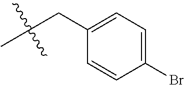 [SMILES: *Cc1ccc(Br)cc1] |
| 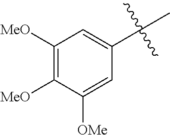 [SMILES: *c1cc(OC)c(OC)c(OC)c1] | 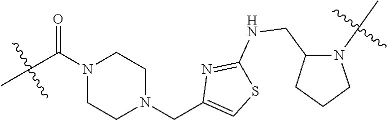 [SMILES: *C(=O)N1CCN(Cc2csc(NCC3CCCN3*)n2)CC1] | absent | 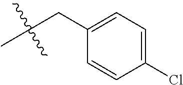 [SMILES: *Cc1ccc(Cl)cc1] |
| 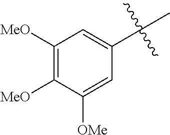 [SMILES: *c1cc(OC)c(OC)c(OC)c1] | 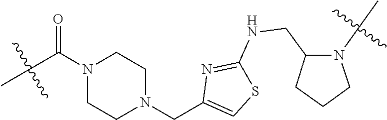 [SMILES: *C(=O)N1CCN(Cc2csc(NCC3CCCN3*)n2)CC1] | absent | 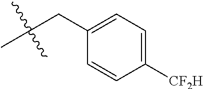 [SMILES: *Cc1ccc(C(F)F)cc1] |
| 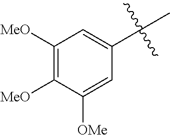 [SMILES: *c1cc(OC)c(OC)c(OC)c1] | 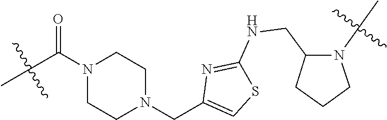 [SMILES: *C(=O)N1CCN(Cc2csc(NCC3CCCN3*)n2)CC1] | absent | 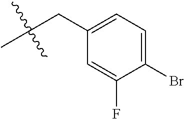 [SMILES: *Cc1ccc(Br)c(F)c1] |
| 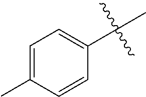 [SMILES: *c1ccc(C)cc1] | 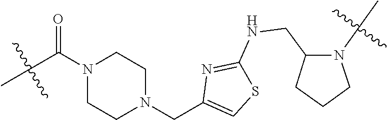 [SMILES: *C(=O)N1CCN(Cc2csc(NCC3CCCN3*)n2)CC1] | absent | 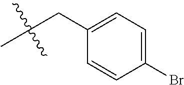 [SMILES: *Cc1ccc(Br)cc1] |
| 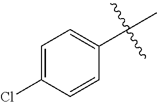 [SMILES: *c1ccc(Cl)cc1] | 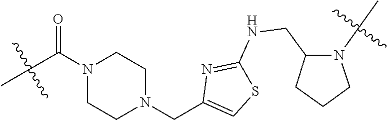 [SMILES: *C(=O)N1CCN(Cc2csc(NCC3CCCN3*)n2)CC1] | absent | 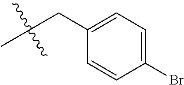 [SMILES: *Cc1ccc(Br)cc1] |
| 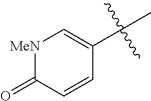 [SMILES: *c1ccc(=O)n(C)c1] | 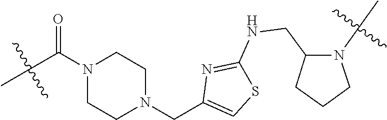 [SMILES: *C(=O)N1CCN(Cc2csc(NCC3CCCN3*)n2)CC1] | absent | 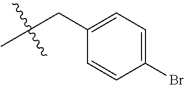 [SMILES: *Cc1ccc(Br)cc1] |

In some embodiments of the compound of Formula (IV), the compound is as shown in Table 5 (below).

TABLE 5

| # | R₁-L₁ | 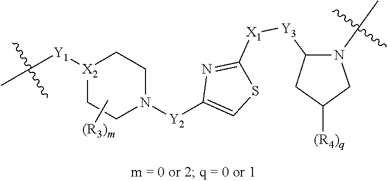 | R₄ | L₂-R₂ |
|---|---|---|---|---|
| A302 | 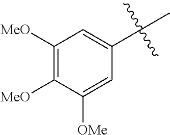 [SMILES: *c1cc(OC)c(OC)c(OC)c1] | 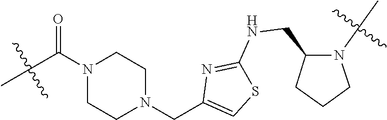 [SMILES: *C(=O)N1CCN(Cc2csc(NC[C@@H]3CCCN3*)n2)CC1] | absent | 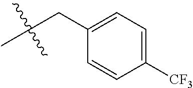 [SMILES: *Cc1ccc(C(F)(F)F)cc1] |
| A303 | 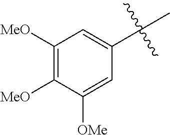 [SMILES: *c1cc(OC)c(OC)c(OC)c1] | 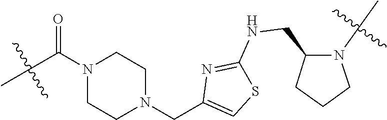 [SMILES: *C(=O)N1CCN(Cc2csc(NC[C@@H]3CCCN3*)n2)CC1] | absent | 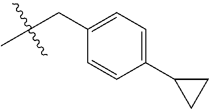 [SMILES: *Cc1ccc(C2CC2)cc1] |
| A304 | 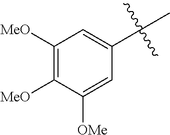 [SMILES: *c1cc(OC)c(OC)c(OC)c1] | 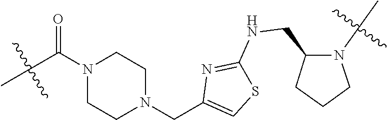 [SMILES: *C(=O)N1CCN(Cc2csc(NC[C@@H]3CCCN3*)n2)CC1] | absent | 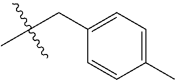 [SMILES: *Cc1ccc(C)cc1] |
| A305 | 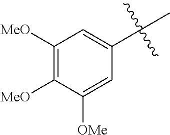 [SMILES: *c1cc(OC)c(OC)c(OC)c1] | 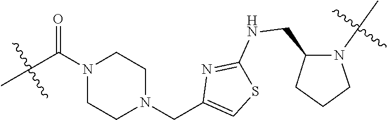 [SMILES: *C(=O)N1CCN(Cc2csc(NC[C@@H]3CCCN3*)n2)CC1] | absent | 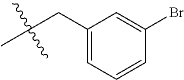 [SMILES: *Cc1cccc(Br)c1] |
| A306 | 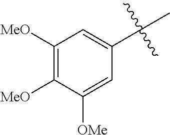 [SMILES: *c1cc(OC)c(OC)c(OC)c1] | 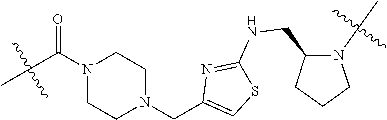 [SMILES: *C(=O)N1CCN(Cc2csc(NC[C@@H]3CCCN3*)n2)CC1] | absent | 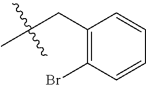 [SMILES: *Cc1ccccc1Br] |
| A307 | 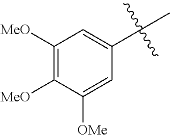 [SMILES: *c1cc(OC)c(OC)c(OC)c1] | 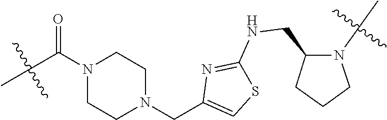 [SMILES: *C(=O)N1CCN(Cc2csc(NC[C@@H]3CCCN3*)n2)CC1] | absent | 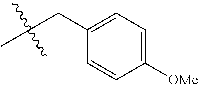 [SMILES: *Cc1ccc(OC)cc1] |
| A308 | 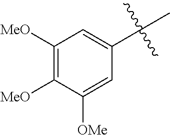 [SMILES: *c1cc(OC)c(OC)c(OC)c1] | 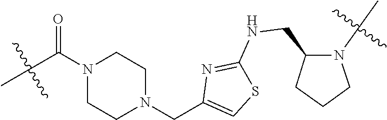 [SMILES: *C(=O)N1CCN(Cc2csc(NC[C@@H]3CCCN3*)n2)CC1] | absent | 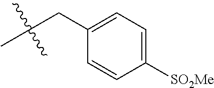 [SMILES: *Cc1ccc(S(C)(=O)=O)cc1] |
| A309 | 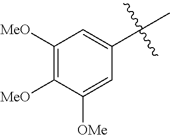 [SMILES: *c1cc(OC)c(OC)c(OC)c1] | 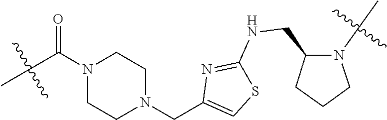 [SMILES: *C(=O)N1CCN(Cc2csc(NC[C@@H]3CCCN3*)n2)CC1] | absent | 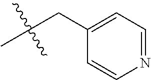 [SMILES: *Cc1ccncc1] |
| A310 | 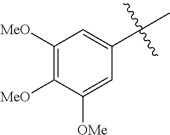 [SMILES: *c1cc(OC)c(OC)c(OC)c1] | 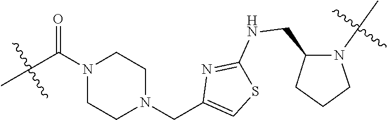 [SMILES: *C(=O)N1CCN(Cc2csc(NC[C@@H]3CCCN3*)n2)CC1] | absent | 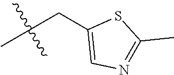 [SMILES: *Cc1cnc(C)s1] |
| A311 | 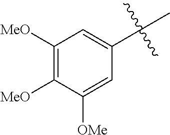 [SMILES: *c1cc(OC)c(OC)c(OC)c1] | 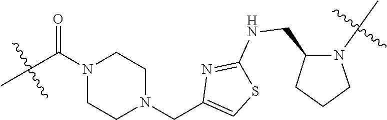 [SMILES: *C(=O)N1CCN(Cc2csc(NC[C@@H]3CCCN3*)n2)CC1] | absent | 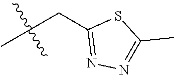 [SMILES: *Cc1nnc(C)s1] |
| A312 | 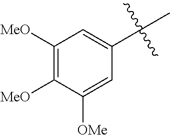 [SMILES: *c1cc(OC)c(OC)c(OC)c1] | 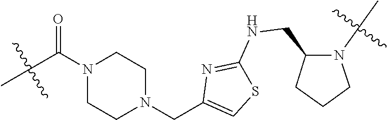 [SMILES: *C(=O)N1CCN(Cc2csc(NC[C@@H]3CCCN3*)n2)CC1] | absent | 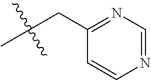 [SMILES: *Cc1ccncn1] |
| A401 | 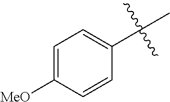 [SMILES: *c1ccc(OC)cc1] | 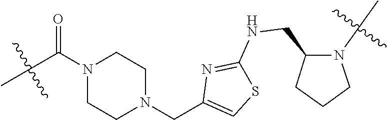 [SMILES: *C(=O)N1CCN(Cc2csc(NC[C@@H]3CCCN3*)n2)CC1] | absent | 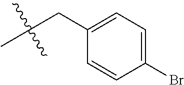 [SMILES: *Cc1ccc(Br)cc1] |
| A402 | 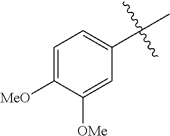 [SMILES: *c1ccc(OC)c(OC)c1] | 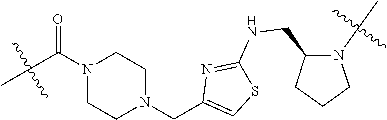 [SMILES: *C(=O)N1CCN(Cc2csc(NC[C@@H]3CCCN3*)n2)CC1] | absent | 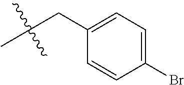 [SMILES: *Cc1ccc(Br)cc1] |
| A403 | 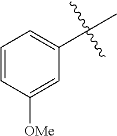 [SMILES: *c1cccc(OC)c1] | 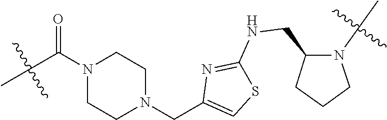 [SMILES: *C(=O)N1CCN(Cc2csc(NC[C@@H]3CCCN3*)n2)CC1] | absent | 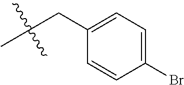 [SMILES: *Cc1ccc(Br)cc1] |
| A404 | 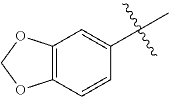 [SMILES: *c1ccc2c(c1)OCO2] | 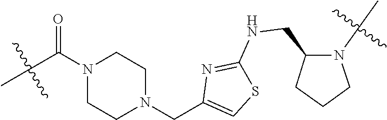 [SMILES: *C(=O)N1CCN(Cc2csc(NC[C@@H]3CCCN3*)n2)CC1] | absent | 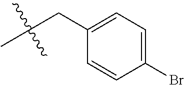 [SMILES: *Cc1ccc(Br)cc1] |
| A405 | 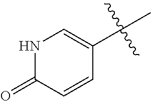 [SMILES: *c1ccc(=O)[nH]c1] | 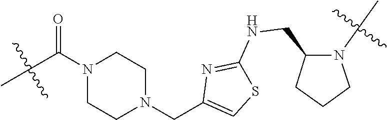 [SMILES: *C(=O)N1CCN(Cc2csc(NC[C@@H]3CCCN3*)n2)CC1] | absent | 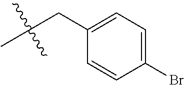 [SMILES: *Cc1ccc(Br)cc1] |
| A406 | 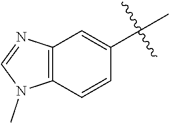 [SMILES: *c1ccc2c(c1)ncn2C] | 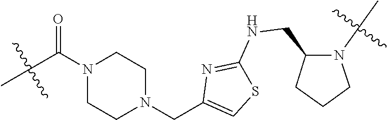 [SMILES: *C(=O)N1CCN(Cc2csc(NC[C@@H]3CCCN3*)n2)CC1] | absent | 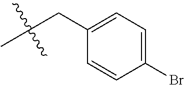 [SMILES: *Cc1ccc(Br)cc1] |
| A407 | 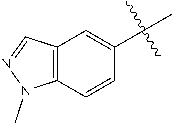 [SMILES: *c1ccc2c(cnn2C)c1] | 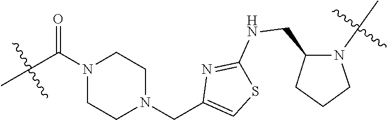 [SMILES: *C(=O)N1CCN(Cc2csc(NC[C@@H]3CCCN3*)n2)CC1] | absent | 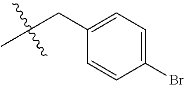 [SMILES: *Cc1ccc(Br)cc1] |
| A501 | 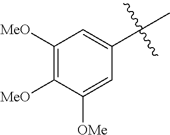 [SMILES: *c1cc(OC)c(OC)c(OC)c1] | 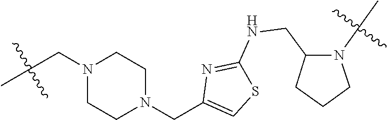 [SMILES: *CN1CCN(Cc2csc(NCC3CCCN3*)n2)CC1] | absent | 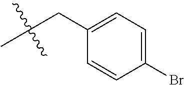 [SMILES: *Cc1ccc(Br)cc1] |
| A502 | 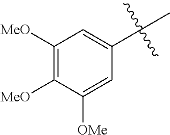 [SMILES: *c1cc(OC)c(OC)c(OC)c1] | 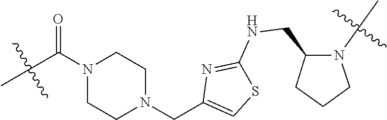 [SMILES: *C(=O)N1CCN(Cc2csc(NC[C@@H]3CCCN3*)n2)CC1] | absent | 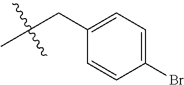 [SMILES: *Cc1ccc(Br)cc1] |
| A503 | 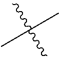 | 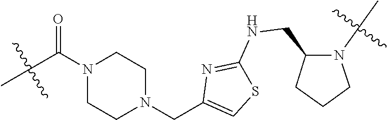 [SMILES: *C(=O)N1CCN(Cc2csc(NC[C@@H]3CCCN3*)n2)CC1] | absent | 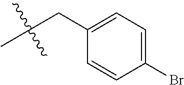 [SMILES: *Cc1ccc(Br)cc1] |
| A504 | 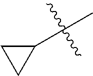 [SMILES: *C1CC1] | 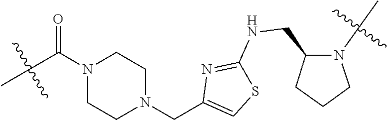 [SMILES: *C(=O)N1CCN(Cc2csc(NC[C@@H]3CCCN3*)n2)CC1] | absent | 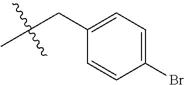 [SMILES: *Cc1ccc(Br)cc1] |
| A505 | 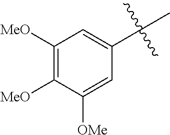 [SMILES: *c1cc(OC)c(OC)c(OC)c1] | 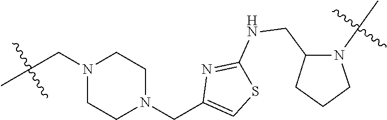 [SMILES: *CN1CCN(Cc2csc(NCC3CCCN3*)n2)CC1] | absent | 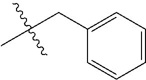 [SMILES: *Cc1ccccc1] |
| A506 | 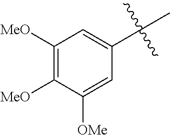 [SMILES: *c1cc(OC)c(OC)c(OC)c1] | 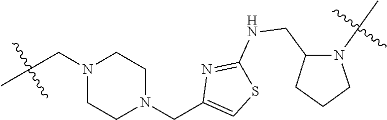 [SMILES: *CN1CCN(Cc2csc(NCC3CCCN3*)n2)CC1] | absent | 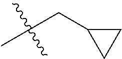 [SMILES: *CC1CC1] |
| A601 | 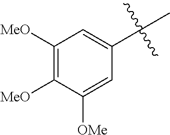 [SMILES: *c1cc(OC)c(OC)c(OC)c1] | 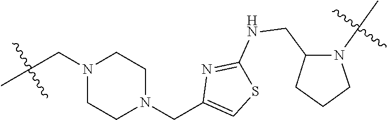 [SMILES: *CN1CCN(Cc2csc(NCC3CCCN3*)n2)CC1] | absent | 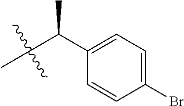 [SMILES: *[C@@H](C)c1ccc(Br)cc1] |
| A602 | 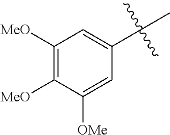 [SMILES: *c1cc(OC)c(OC)c(OC)c1] | 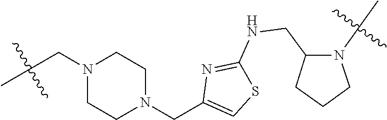 [SMILES: *CN1CCN(Cc2csc(NCC3CCCN3*)n2)CC1] | absent | 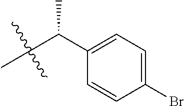 [SMILES: *[C@H](C)c1ccc(Br)cc1] |
| A603 | 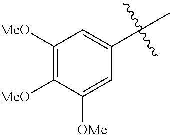 [SMILES: *c1cc(OC)c(OC)c(OC)c1] | 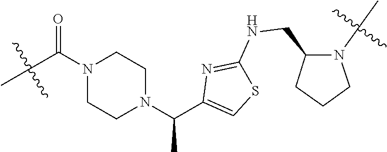 [SMILES: *C(=O)N1CCN([C@H](C)c2csc(NC[C@@H]3CCCN3*)n2)CC1] | absent | 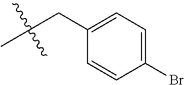 [SMILES: *Cc1ccc(Br)cc1] |
| A604 | 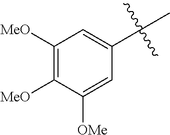 [SMILES: *c1cc(OC)c(OC)c(OC)c1] | 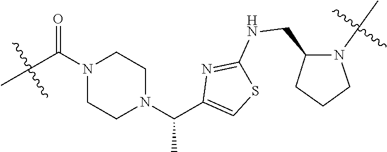 [SMILES: *C(=O)N1CCN([C@@H](C)c2csc(NC[C@@H]3CCCN3*)n2)CC1] | absent | 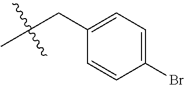 [SMILES: *Cc1ccc(Br)cc1] |
| A605 | 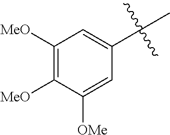 [SMILES: *c1cc(OC)c(OC)c(OC)c1] | 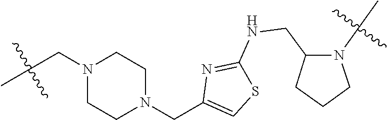 [SMILES: *CN1CCN(Cc2csc(NCC3CCCN3*)n2)CC1] | absent | 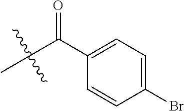 [SMILES: *C(=O)c1ccc(Br)cc1] |
| A606 | 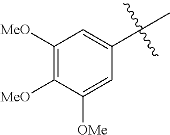 [SMILES: *c1cc(OC)c(OC)c(OC)c1] | 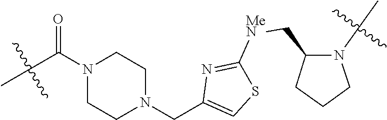 [SMILES: *C(=O)N1CCN(Cc2csc(N(C)C[C@@H]3CCCN3*)n2)CC1] | absent | 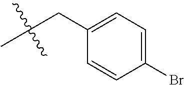 [SMILES: *Cc1ccc(Br)cc1] |
| A607 | 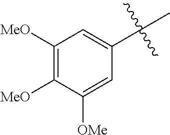 [SMILES: *c1cc(OC)c(OC)c(OC)c1] | 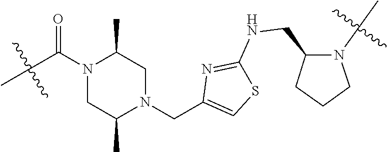 [SMILES: *C(=O)N1C[C@H](C)N(Cc2csc(NC[C@@H]3CCCN3*)n2)C[C@@H]1C] | absent | 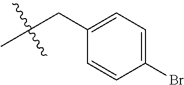 [SMILES: *Cc1ccc(Br)cc1] |
| A608 | 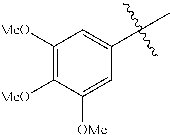 [SMILES: *c1cc(OC)c(OC)c(OC)c1] | 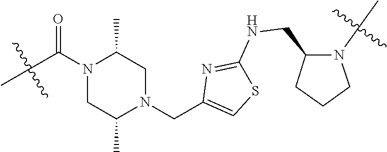 [SMILES: *C(=O)N1C[C@@H](C)N(Cc2csc(NC[C@@H]3CCCN3*)n2)C[C@H]1C] | absent | 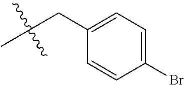 [SMILES: *Cc1ccc(Br)cc1] |
| A703 | 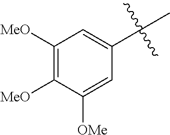 [SMILES: *c1cc(OC)c(OC)c(OC)c1] | 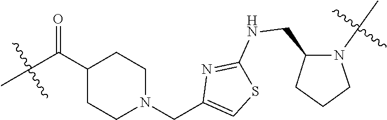 [SMILES: *C(=O)C1CCN(Cc2csc(NC[C@@H]3CCCN3*)n2)CC1] | absent | 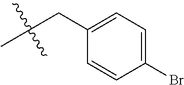 [SMILES: *Cc1ccc(Br)cc1] |
| A704 | 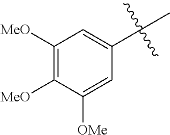 [SMILES: *c1cc(OC)c(OC)c(OC)c1] | 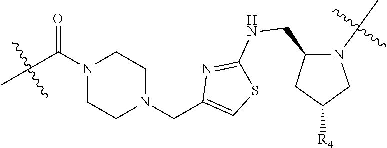 | OH | 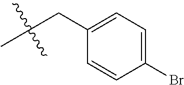 [SMILES: *Cc1ccc(Br)cc1] |
| A705 | 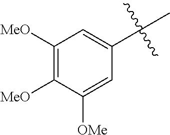 [SMILES: *c1cc(OC)c(OC)c(OC)c1] | 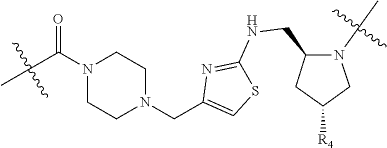 | OMe | 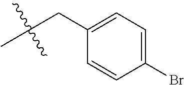 [SMILES: *Cc1ccc(Br)cc1] |
| A706 | 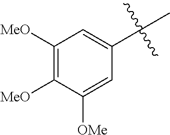 [SMILES: *c1cc(OC)c(OC)c(OC)c1] | 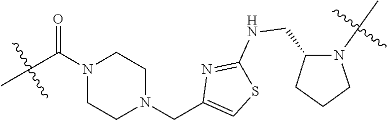 [SMILES: *C(=O)N1CCN(Cc2csc(NC[C@H]3CCCN3*)n2)CC1] | absent | 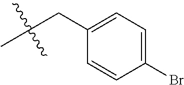 [SMILES: *Cc1ccc(Br)cc1] |
| A707 | 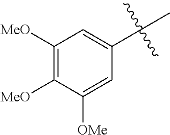 [SMILES: *c1cc(OC)c(OC)c(OC)c1] | 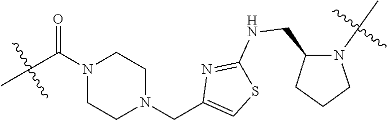 [SMILES: *C(=O)N1CCN(Cc2csc(NC[C@@H]3CCCN3*)n2)CC1] | absent | 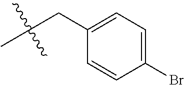 [SMILES: *Cc1ccc(Br)cc1] |
| B313 | 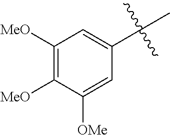 [SMILES: *c1cc(OC)c(OC)c(OC)c1] | 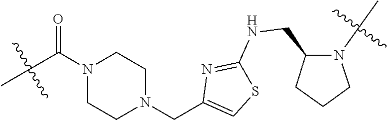 [SMILES: *C(=O)N1CCN(Cc2csc(NC[C@@H]3CCCN3*)n2)CC1] | absent | 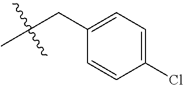 [SMILES: *Cc1ccc(Cl)cc1] |
| B315 | 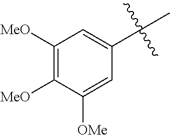 [SMILES: *c1cc(OC)c(OC)c(OC)c1] | 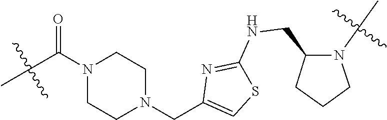 [SMILES: *C(=O)N1CCN(Cc2csc(NC[C@@H]3CCCN3*)n2)CC1] | absent | 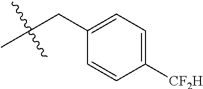 [SMILES: *Cc1ccc(C(F)F)cc1] |
| B318 | 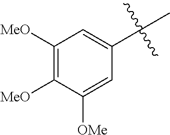 [SMILES: *c1cc(OC)c(OC)c(OC)c1] | 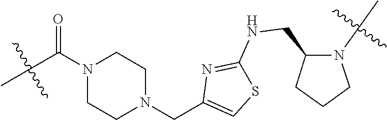 [SMILES: *C(=O)N1CCN(Cc2csc(NC[C@@H]3CCCN3*)n2)CC1] | absent | 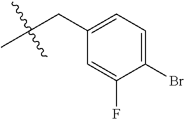 [SMILES: *Cc1ccc(Br)c(F)c1] |
| B408 | 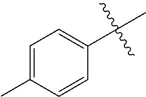 [SMILES: *c1ccc(C)cc1] | 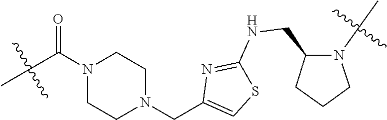 [SMILES: *C(=O)N1CCN(Cc2csc(NC[C@@H]3CCCN3*)n2)CC1] | absent | 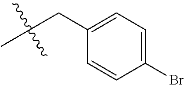 [SMILES: *Cc1ccc(Br)cc1] |
| B410 | 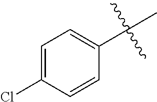 [SMILES: *c1ccc(Cl)cc1] | 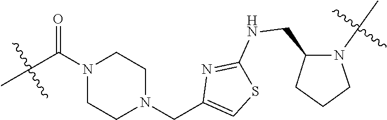 [SMILES: *C(=O)N1CCN(Cc2csc(NC[C@@H]3CCCN3*)n2)CC1] | absent | 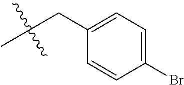 [SMILES: *Cc1ccc(Br)cc1] |
| B414 | 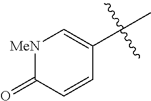 [SMILES: *c1ccc(=O)n(C)c1] | 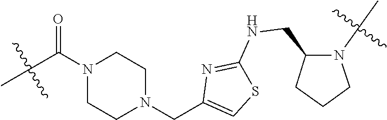 [SMILES: *C(=O)N1CCN(Cc2csc(NC[C@@H]3CCCN3*)n2)CC1] | absent | 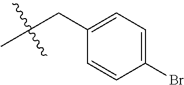 [SMILES: *Cc1ccc(Br)cc1] |

In some embodiments of the compound of Formula (I) or (II), the compound is a compound of Formula (V):

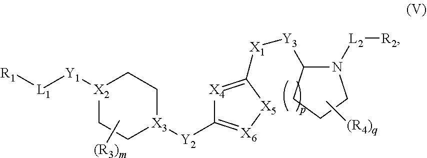

wherein m is 0 or 2; q is 0 or 1; X₄ and X₆ independent are —N— or —CH—; and X₅ is —NH— or —S—.

In some embodiments of the compound of Formula (V), the compound is defined as shown in Table 6 (below).

TABLE 6

| R₁-L₁ | 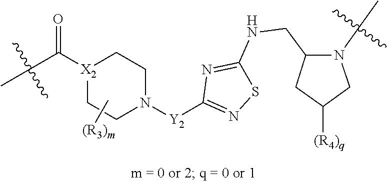 | L₂-R₂ |
|---|---|---|
| 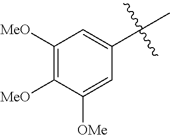 [SMILES: *c1cc(OC)c(OC)c(OC)c1] | 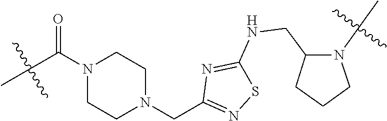 [SMILES: *C(=O)N1CCN(Cc2nsc(NCC3CCCN3*)n2)CC1] | 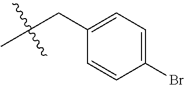 [SMILES: *Cc1ccc(Br)cc1] |
| 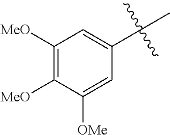 [SMILES: *c1cc(OC)c(OC)c(OC)c1] | 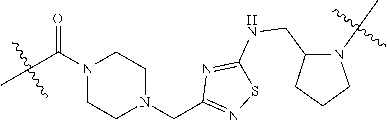 [SMILES: *C(=O)N1CCN(Cc2nsc(NCC3CCCN3*)n2)CC1] | 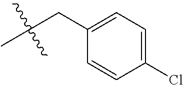 [SMILES: *Cc1ccc(Cl)cc1] |
| 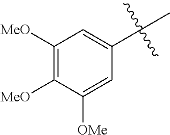 [SMILES: *c1cc(OC)c(OC)c(OC)c1] | 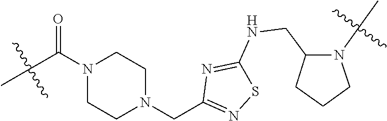 [SMILES: *C(=O)N1CCN(Cc2nsc(NCC3CCCN3*)n2)CC1] | 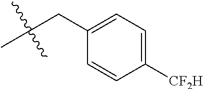 [SMILES: *Cc1ccc(C(F)F)cc1] |
| 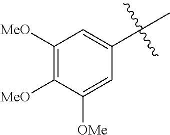 [SMILES: *c1cc(OC)c(OC)c(OC)c1] | 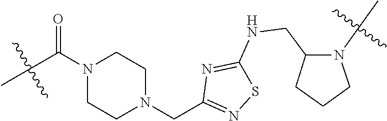 [SMILES: *C(=O)N1CCN(Cc2nsc(NCC3CCCN3*)n2)CC1] | 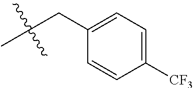 [SMILES: *Cc1ccc(C(F)(F)F)cc1] |
| 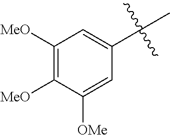 [SMILES: *c1cc(OC)c(OC)c(OC)c1] | 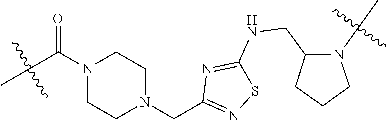 [SMILES: *C(=O)N1CCN(Cc2nsc(NCC3CCCN3*)n2)CC1] | 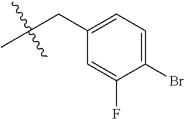 [SMILES: *Cc1ccc(Br)c(F)c1] |
| 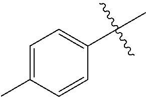 [SMILES: *c1ccc(C)cc1] | 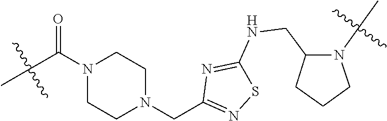 [SMILES: *C(=O)N1CCN(Cc2nsc(NCC3CCCN3*)n2)CC1] | 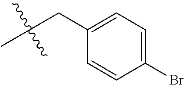 [SMILES: *Cc1ccc(Br)cc1] |
| 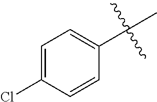 [SMILES: *c1ccc(Cl)cc1] | 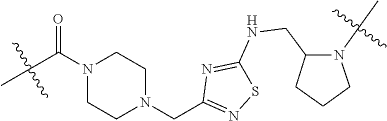 [SMILES: *C(=O)N1CCN(Cc2nsc(NCC3CCCN3*)n2)CC1] | 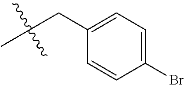 [SMILES: *Cc1ccc(Br)cc1] |
| 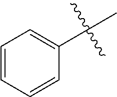 [SMILES: *c1ccccc1] | 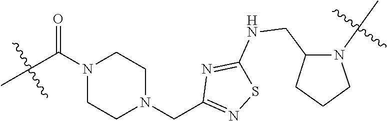 [SMILES: *C(=O)N1CCN(Cc2nsc(NCC3CCCN3*)n2)CC1] | 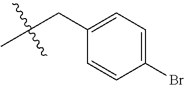 [SMILES: *Cc1ccc(Br)cc1] |
| 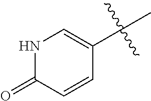 [SMILES: *c1ccc(=O)[nH]c1] | 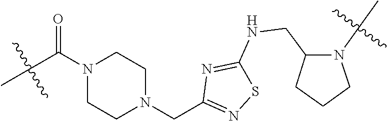 [SMILES: *C(=O)N1CCN(Cc2nsc(NCC3CCCN3*)n2)CC1] | 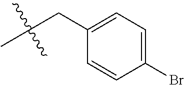 [SMILES: *Cc1ccc(Br)cc1] |
| 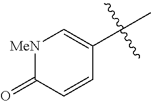 [SMILES: *c1ccc(=O)n(C)c1] | 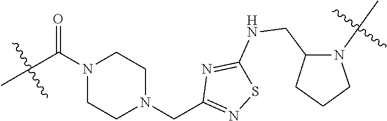 [SMILES: *C(=O)N1CCN(Cc2nsc(NCC3CCCN3*)n2)CC1] | 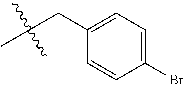 [SMILES: *Cc1ccc(Br)cc1] |
| 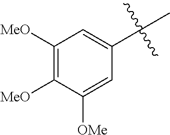 [SMILES: *c1cc(OC)c(OC)c(OC)c1] | 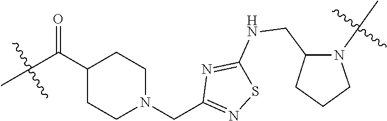 [SMILES: *C(=O)C1CCN(Cc2nsc(NCC3CCCN3*)n2)CC1] | 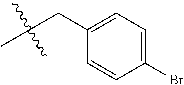 [SMILES: *Cc1ccc(Br)cc1] |
| 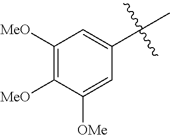 [SMILES: *c1cc(OC)c(OC)c(OC)c1] | 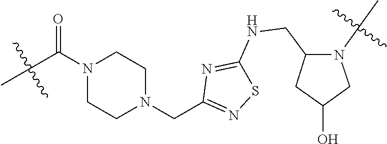 [SMILES: *C(=O)N1CCN(Cc2nsc(NCC3CC(O)CN3*)n2)CC1] | 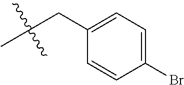 [SMILES: *Cc1ccc(Br)cc1] |
| 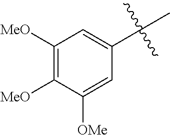 [SMILES: *c1cc(OC)c(OC)c(OC)c1] | 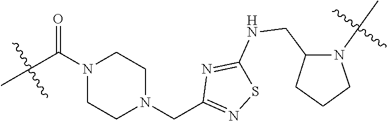 [SMILES: *C(=O)N1CCN(Cc2nsc(NCC3CCCN3*)n2)CC1] | 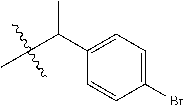 [SMILES: *C(C)c1ccc(Br)cc1] |
| 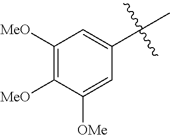 [SMILES: *c1cc(OC)c(OC)c(OC)c1] | 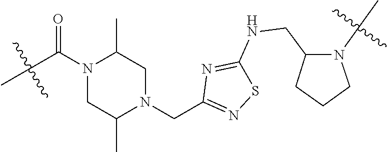 [SMILES: *C(=O)N1CC(C)N(Cc2nsc(NCC3CCCN3*)n2)CC1C] | 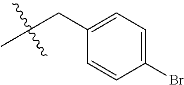 [SMILES: *Cc1ccc(Br)cc1] |
| 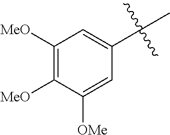 [SMILES: *c1cc(OC)c(OC)c(OC)c1] | 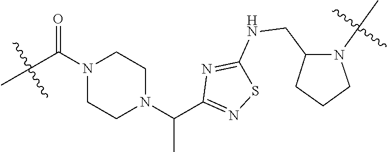 [SMILES: *C(=O)N1CCN(C(C)c2nsc(NCC3CCCN3*)n2)CC1] | 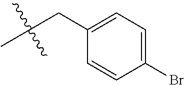 [SMILES: *Cc1ccc(Br)cc1] |
| 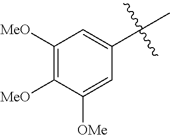 [SMILES: *c1cc(OC)c(OC)c(OC)c1] | 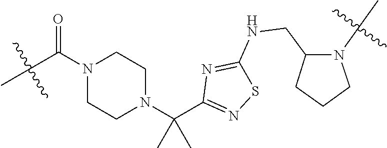 [SMILES: *C(=O)N1CCN(C(C)(C)c2nsc(NCC3CCCN3*)n2)CC1] | 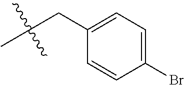 [SMILES: *Cc1ccc(Br)cc1] |
| 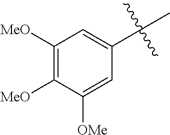 [SMILES: *c1cc(OC)c(OC)c(OC)c1] | 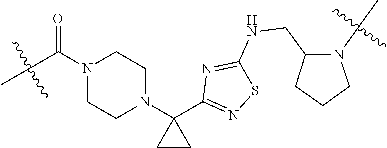 [SMILES: *C(=O)N1CCN(C2(c3nsc(NCC4CCCN4*)n3)CC2)CC1] | 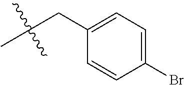 [SMILES: *Cc1ccc(Br)cc1] |
| 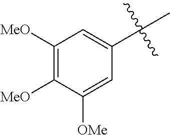 [SMILES: *c1cc(OC)c(OC)c(OC)c1] | 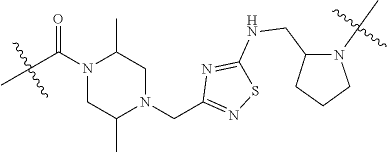 [SMILES: *C(=O)N1CC(C)N(Cc2nsc(NCC3CCCN3*)n2)CC1C] | 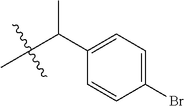 [SMILES: *C(C)c1ccc(Br)cc1] |
| 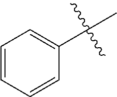 [SMILES: *c1ccccc1] | 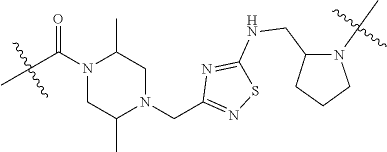 [SMILES: *C(=O)N1CC(C)N(Cc2nsc(NCC3CCCN3*)n2)CC1C] | 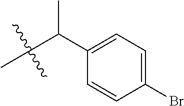 [SMILES: *C(C)c1ccc(Br)cc1] |
| 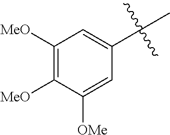 [SMILES: *c1cc(OC)c(OC)c(OC)c1] | 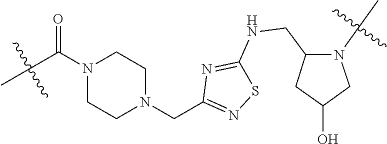 [SMILES: *C(=O)N1CCN(Cc2nsc(NCC3CC(O)CN3*)n2)CC1] | 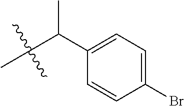 [SMILES: *C(C)c1ccc(Br)cc1] |
| 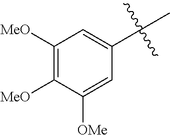 [SMILES: *c1cc(OC)c(OC)c(OC)c1] | 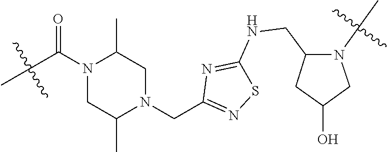 [SMILES: *C(=O)N1CC(C)N(Cc2nsc(NCC3CC(O)CN3*)n2)CC1C] | 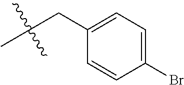 [SMILES: *Cc1ccc(Br)cc1] |

In some embodiments of the compound of Formula (V), the compound is defined as shown in Table 7 (below).

TABLE 7

| # | R₁—L₁ | 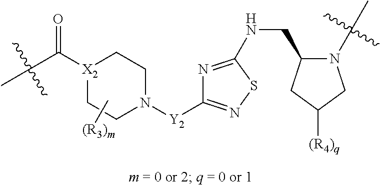 | L₂—R₂ |
|---|---|---|---|
| A702 | 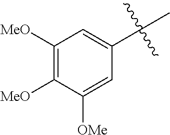 [SMILES: *c1cc(OC)c(OC)c(OC)c1] | 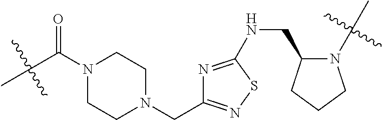 [SMILES: *C(=O)N1CCN(Cc2nsc(NC[C@@H]3CCCN3*)n2)CC1] | 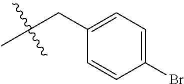 [SMILES: *Cc1ccc(Br)cc1] |
| B314 | 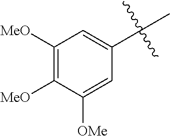 [SMILES: *c1cc(OC)c(OC)c(OC)c1] | 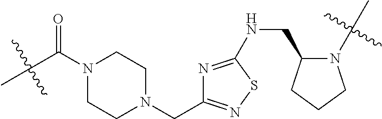 [SMILES: *C(=O)N1CCN(Cc2nsc(NC[C@@H]3CCCN3*)n2)CC1] | 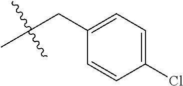 [SMILES: *Cc1ccc(Cl)cc1] |
| B316 | 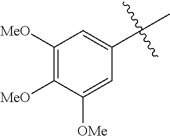 [SMILES: *c1cc(OC)c(OC)c(OC)c1] | 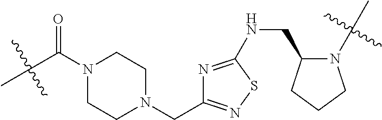 [SMILES: *C(=O)N1CCN(Cc2nsc(NC[C@@H]3CCCN3*)n2)CC1] | 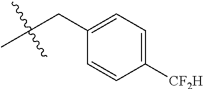 [SMILES: *Cc1ccc(C(F)F)cc1] |
| B317 | 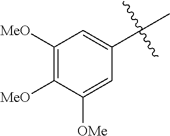 [SMILES: *c1cc(OC)c(OC)c(OC)c1] | 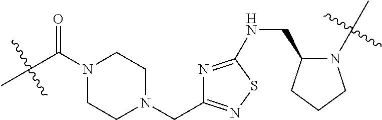 [SMILES: *C(=O)N1CCN(Cc2nsc(NC[C@@H]3CCCN3*)n2)CC1] | 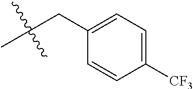 [SMILES: *Cc1ccc(C(F)(F)F)cc1] |
| B319 | 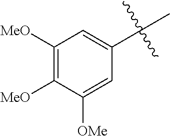 [SMILES: *c1cc(OC)c(OC)c(OC)c1] | 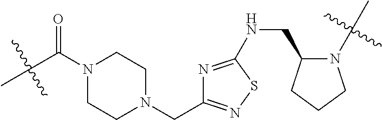 [SMILES: *C(=O)N1CCN(Cc2nsc(NC[C@@H]3CCCN3*)n2)CC1] | 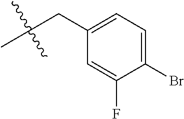 [SMILES: *Cc1ccc(Br)c(F)c1] |
| B409 | 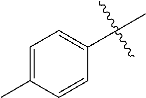 [SMILES: *c1ccc(C)cc1] | 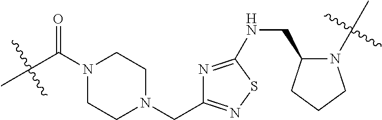 [SMILES: *C(=O)N1CCN(Cc2nsc(NC[C@@H]3CCCN3*)n2)CC1] | 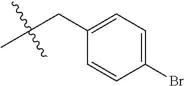 [SMILES: *Cc1ccc(Br)cc1] |
| B411 | 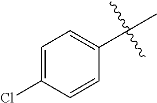 [SMILES: *c1ccc(Cl)cc1] | 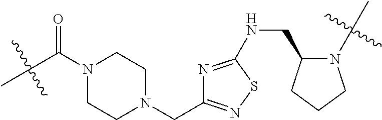 [SMILES: *C(=O)N1CCN(Cc2nsc(NC[C@@H]3CCCN3*)n2)CC1] | 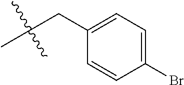 [SMILES: *Cc1ccc(Br)cc1] |
| B412 | 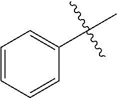 [SMILES: *c1ccccc1] | 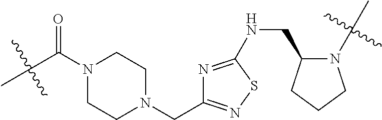 [SMILES: *C(=O)N1CCN(Cc2nsc(NC[C@@H]3CCCN3*)n2)CC1] | 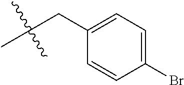 [SMILES: *Cc1ccc(Br)cc1] |
| B413 | 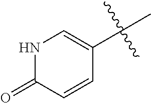 [SMILES: *c1ccc(=O)[nH]c1] | 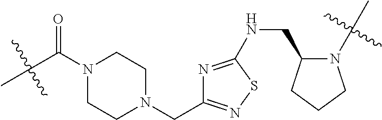 [SMILES: *C(=O)N1CCN(Cc2nsc(NC[C@@H]3CCCN3*)n2)CC1] | 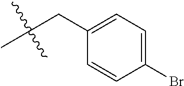 [SMILES: *Cc1ccc(Br)cc1] |
| B415 | 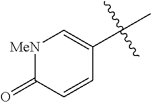 [SMILES: *c1ccc(=O)n(C)c1] | 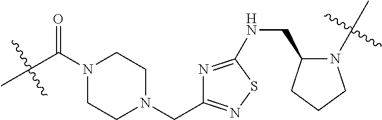 [SMILES: *C(=O)N1CCN(Cc2nsc(NC[C@@H]3CCCN3*)n2)CC1] | 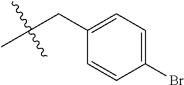 [SMILES: *Cc1ccc(Br)cc1] |
| B801 | 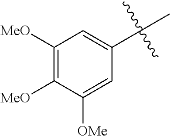 [SMILES: *c1cc(OC)c(OC)c(OC)c1] | 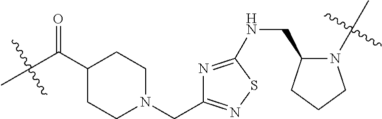 [SMILES: *C(=O)C1CCN(Cc2nsc(NC[C@@H]3CCCN3*)n2)CC1] | 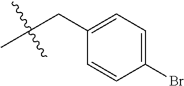 [SMILES: *Cc1ccc(Br)cc1] |
| B802 | 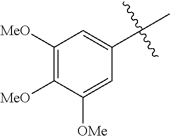 [SMILES: *c1cc(OC)c(OC)c(OC)c1] | 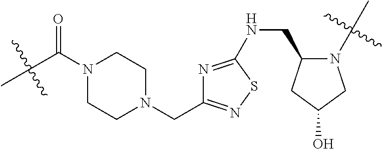 [SMILES: *C(=O)N1CCN(Cc2nsc(NC[C@@H]3C[C@@H](O)CN3*)n2)CC1] | 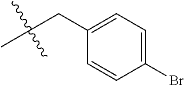 [SMILES: *Cc1ccc(Br)cc1] |
| B803 | 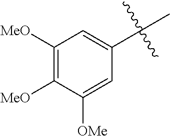 [SMILES: *c1cc(OC)c(OC)c(OC)c1] | 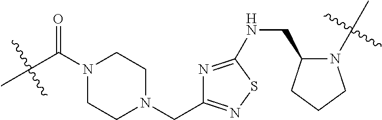 [SMILES: *C(=O)N1CCN(Cc2nsc(NC[C@@H]3CCCN3*)n2)CC1] | 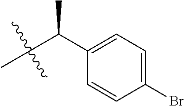 [SMILES: *[C@@H](C)c1ccc(Br)cc1] |
| B804 | 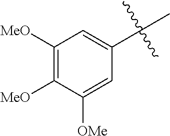 [SMILES: *c1cc(OC)c(OC)c(OC)c1] | 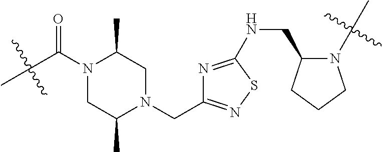 [SMILES: *C(=O)N1C[C@H](C)N(Cc2nsc(NC[C@@H]3CCCN3*)n2)C[C@@H]1C] | 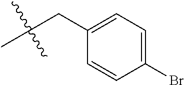 [SMILES: *Cc1ccc(Br)cc1] |
| B805 | 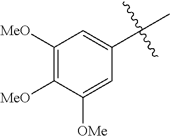 [SMILES: *c1cc(OC)c(OC)c(OC)c1] | 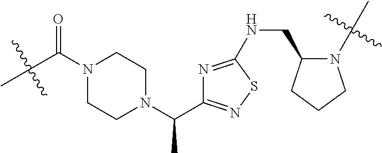 [SMILES: *C(=O)N1CCN([C@H](C)c2nsc(NC[C@@H]3CCCN3*)n2)CC1] | 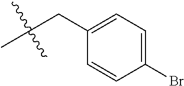 [SMILES: *Cc1ccc(Br)cc1] |
| B806 | 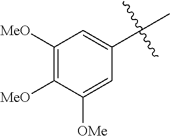 [SMILES: *c1cc(OC)c(OC)c(OC)c1] | 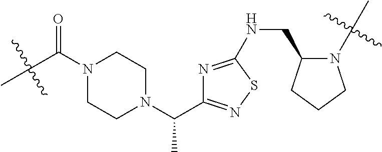 [SMILES: *C(=O)N1CCN([C@@H](C)c2nsc(NC[C@@H]3CCCN3*)n2)CC1] | 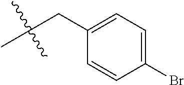 [SMILES: *Cc1ccc(Br)cc1] |
| B807 | 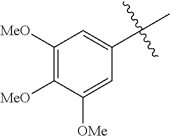 [SMILES: *c1cc(OC)c(OC)c(OC)c1] | 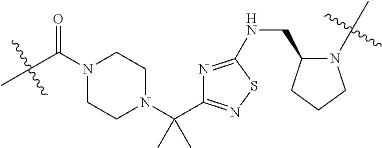 [SMILES: *C(=O)N1CCN(C(C)(C)c2nsc(NC[C@@H]3CCCN3*)n2)CC1] | 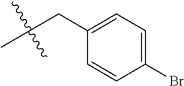 [SMILES: *Cc1ccc(Br)cc1] |
| B808 | 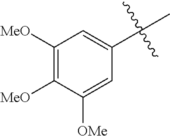 [SMILES: *c1cc(OC)c(OC)c(OC)c1] | 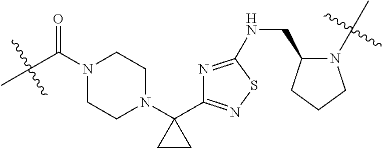 [SMILES: *C(=O)N1CCN(C2(c3nsc(NC[C@@H]4CCCN4*)n3)CC2)CC1] | 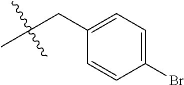 [SMILES: *Cc1ccc(Br)cc1] |
| B908 | 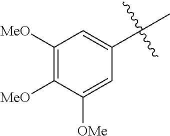 [SMILES: *c1cc(OC)c(OC)c(OC)c1] | 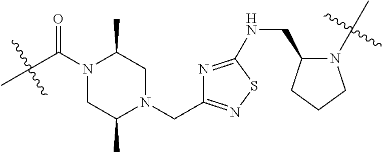 [SMILES: *C(=O)N1C[C@H](C)N(Cc2nsc(NC[C@@H]3CCCN3*)n2)C[C@@H]1C] | 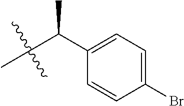 [SMILES: *[C@@H](C)c1ccc(Br)cc1] |
| B909 | 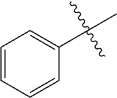 [SMILES: *c1ccccc1] | 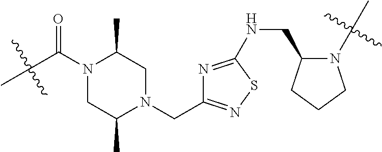 [SMILES: *C(=O)N1C[C@H](C)N(Cc2nsc(NC[C@@H]3CCCN3*)n2)C[C@@H]1C] | 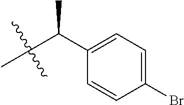 [SMILES: *[C@@H](C)c1ccc(Br)cc1] |
| B910 | 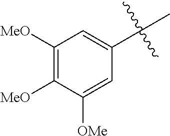 [SMILES: *c1cc(OC)c(OC)c(OC)c1] | 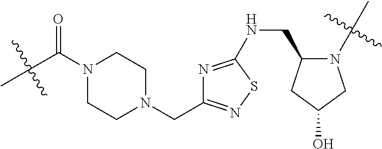 [SMILES: *C(=O)N1CCN(Cc2nsc(NC[C@@H]3C[C@@H](O)CN3*)n2)CC1] | 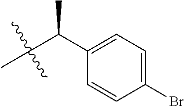 [SMILES: *[C@@H](C)c1ccc(Br)cc1] |
| B911 | 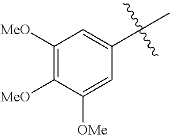 [SMILES: *c1cc(OC)c(OC)c(OC)c1] | 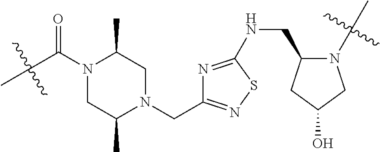 [SMILES: *C(=O)N1C[C@H](C)N(Cc2nsc(NC[C@@H]3C[C@@H](O)CN3*)n2)C[C@@H]1C] | 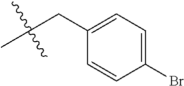 [SMILES: *Cc1ccc(Br)cc1] |

In one embodiment of the compound of Formula (I), the compound is:

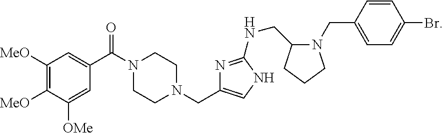 [SMILES: COc1cc(C(=O)N2CCN(Cc3c[nH]c(NCC4CCCN4Cc4ccc(Br)cc4)n3)CC2)cc(OC)c1OC]

In one embodiment of the compound of Formula (I), the compound is:

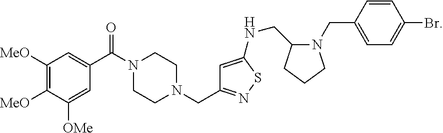 [SMILES: COc1cc(C(=O)N2CCN(Cc3cc(NCC4CCCN4Cc4ccc(Br)cc4)sn3)CC2)cc(OC)c1OC]

In one embodiment of the compound of Formula (I), the compound is:

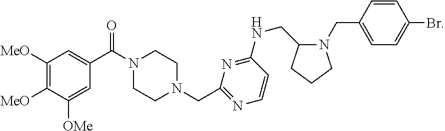 [SMILES: COc1cc(C(=O)N2CCN(Cc3nccc(NCC4CCCN4Cc4ccc(Br)cc4)n3)CC2)cc(OC)c1OC]

In one embodiment of the compound of Formula (I), the compound is:

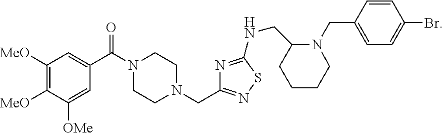 [SMILES: COc1cc(C(=O)N2CCN(Cc3nsc(NCC4CCCCN4Cc4ccc(Br)cc4)n3)CC2)cc(OC)c1OC]

In one embodiment of the compound of Formula (I), the compound is:

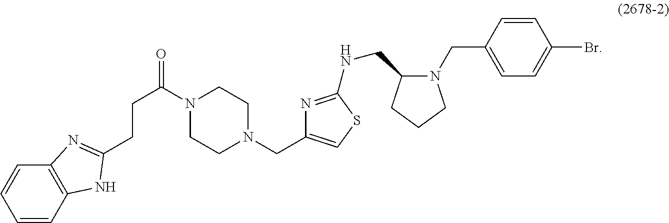 [SMILES: O=C(CCc1nc2ccccc2[nH]1)N1CCN(Cc2csc(NC[C@@H]3CCCN3Cc3ccc(Br)cc3)n2)CC1]

In one embodiment of the compound of Formula (I), the compound is:

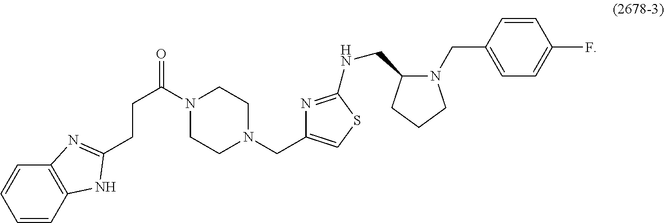 [SMILES: O=C(CCc1nc2ccccc2[nH]1)N1CCN(Cc2csc(NC[C@@H]3CCCN3Cc3ccc(F)cc3)n2)CC1]

In one embodiment of the compound of Formula (I), the compound is:

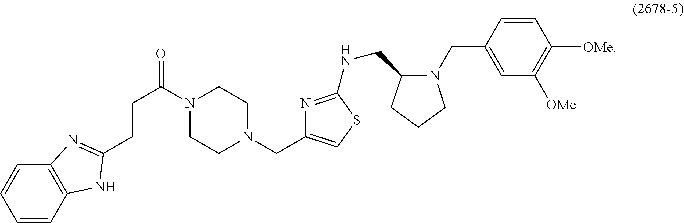 [SMILES: COc1ccc(CN2CCC[C@H]2CNc2nc(CN3CCN(C(=O)CCc4nc5ccccc5[nH]4)CC3)cs2)cc1OC]

In one embodiment of the compound of Formula (I), the compound is:

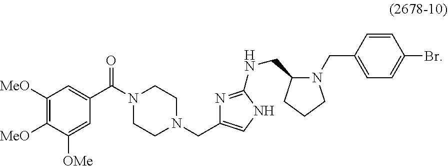 [SMILES: COc1cc(C(=O)N2CCN(Cc3c[nH]c(NC[C@@H]4CCCN4Cc4ccc(Br)cc4)n3)CC2)cc(OC)c1OC]

In one embodiment of the compound of Formula (I), the compound is:

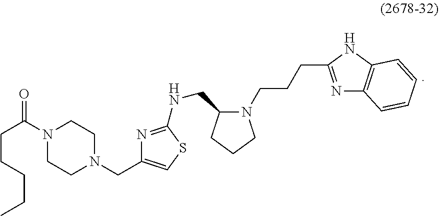 [SMILES: CCCCCC(=O)N1CCN(Cc2csc(NC[C@@H]3CCCN3CCCc3nc4ccccc4[nH]3)n2)CC1]

In one embodiment of the compound of Formula (I), the compound is:

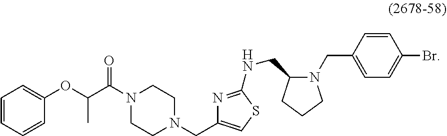 [SMILES: CC(Oc1ccccc1)C(=O)N1CCN(Cc2csc(NC[C@@H]3CCCN3Cc3ccc(Br)cc3)n2)CC1]

In one embodiment of the compound of Formula (I), the compound is:

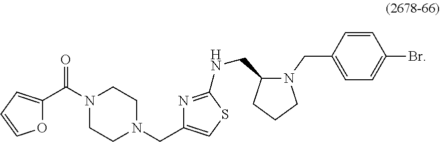 [SMILES: O=C(c1ccco1)N1CCN(Cc2csc(NC[C@@H]3CCCN3Cc3ccc(Br)cc3)n2)CC1]

In one embodiment of the compound of Formula (I), the compound is:

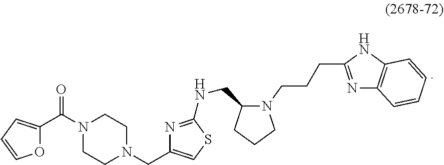 [SMILES: O=C(c1ccco1)N1CCN(Cc2csc(NC[C@@H]3CCCN3CCCc3nc4ccccc4[nH]3)n2)CC1]

In one embodiment of the compound of Formula (I), the compound is:

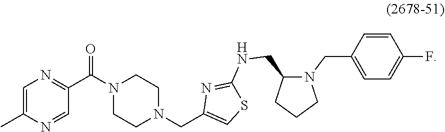 [SMILES: Cc1cnc(C(=O)N2CCN(Cc3csc(NC[C@@H]4CCCN4Cc4ccc(F)cc4)n3)CC2)cn1]

In one embodiment of the compound of Formula (I), the compound is:

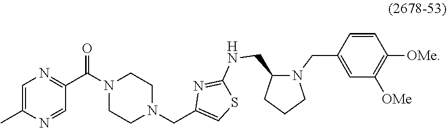 [SMILES: COc1ccc(CN2CCC[C@H]2CNc2nc(CN3CCN(C(=O)c4cnc(C)cn4)CC3)cs2)cc1OC]

In one embodiment of the compound of Formula (I), the compound is:

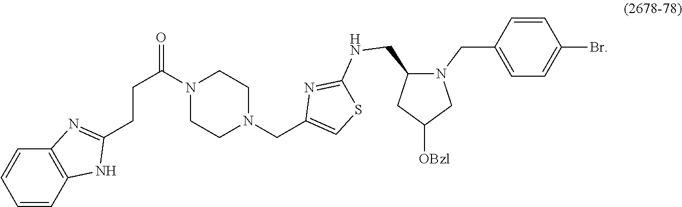

In one embodiment of the compound of Formula (I), the compound is:

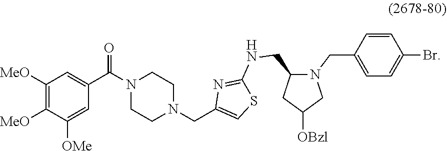

In one embodiment of the compound of Formula (I), the compound is:

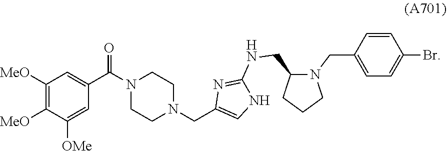 [SMILES: COc1cc(C(=O)N2CCN(Cc3c[nH]c(NC[C@@H]4CCCN4Cc4ccc(Br)cc4)n3)CC2)cc(OC)c1OC]

In one embodiment of the compound of Formula (I), the compound is:

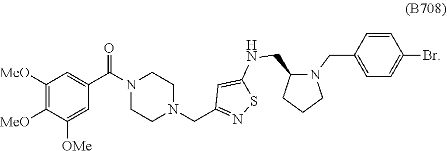 [SMILES: COc1cc(C(=O)N2CCN(Cc3cc(NC[C@@H]4CCCN4Cc4ccc(Br)cc4)sn3)CC2)cc(OC)c1OC]

In one embodiment of the compound of Formula (I), the compound is:

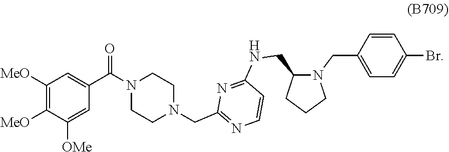 [SMILES: COc1cc(C(=O)N2CCN(Cc3nccc(NC[C@@H]4CCCN4Cc4ccc(Br)cc4)n3)CC2)cc(OC)c1OC]

In one embodiment of the compound of Formula (I), the compound is:

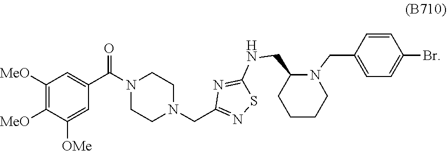 [SMILES: COc1cc(C(=O)N2CCN(Cc3nsc(NC[C@@H]4CCCCN4Cc4ccc(Br)cc4)n3)CC2)cc(OC)c1OC]

In one embodiment of the compound of Formula (I), the compound is:

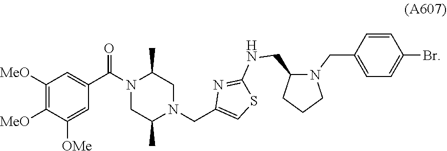 [SMILES: COc1cc(C(=O)N2C[C@H](C)N(Cc3csc(NC[C@@H]4CCCN4Cc4ccc(Br)cc4)n3)C[C@@H]2C)cc(OC)c1OC]

In one embodiment of the compound of Formula (I), the compound is:

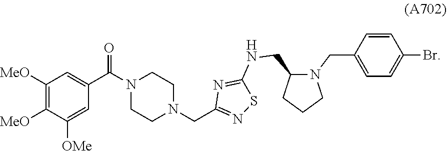 [SMILES: COc1cc(C(=O)N2CCN(Cc3nsc(NC[C@@H]4CCCN4Cc4ccc(Br)cc4)n3)CC2)cc(OC)c1OC]

In one embodiment of the compound of Formula (I), the compound is:

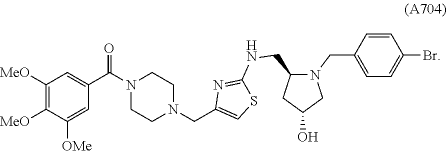 [SMILES: COc1cc(C(=O)N2CCN(Cc3csc(NC[C@@H]4C[C@@H](O)CN4Cc4ccc(Br)cc4)n3)CC2)cc(OC)c1OC]

In one embodiment of the compound of Formula (I), the compound is:

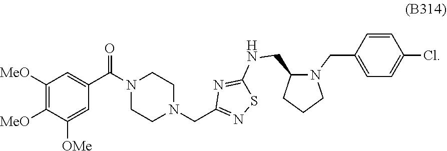 [SMILES: COc1cc(C(=O)N2CCN(Cc3nsc(NC[C@@H]4CCCN4Cc4ccc(Cl)cc4)n3)CC2)cc(OC)c1OC]

In one embodiment of the compound of Formula (I), the compound is:

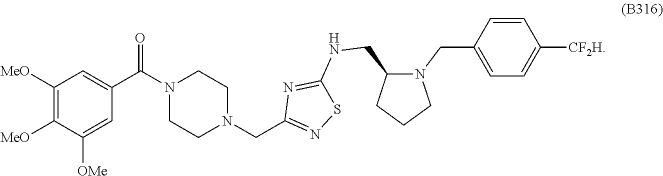 [SMILES: COc1cc(C(=O)N2CCN(Cc3nsc(NC[C@@H]4CCCN4Cc4ccc(C(F)F)cc4)n3)CC2)cc(OC)c1OC]

In one embodiment of the compound of Formula (I), the compound is:

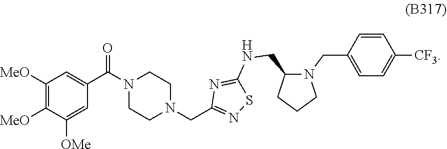 [SMILES: COc1cc(C(=O)N2CCN(Cc3nsc(NC[C@@H]4CCCN4Cc4ccc(C(F)(F)F)cc4)n3)CC2)cc(OC)c1OC]

In one embodiment of the compound of Formula (I), the compound is:

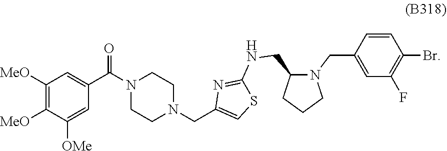 [SMILES: COc1cc(C(=O)N2CCN(Cc3csc(NC[C@@H]4CCCN4Cc4ccc(Br)c(F)c4)n3)CC2)cc(OC)c1OC]

In one embodiment of the compound of Formula (I), the compound is:

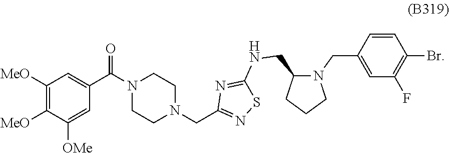 [SMILES: COc1cc(C(=O)N2CCN(Cc3nsc(NC[C@@H]4CCCN4Cc4ccc(Br)c(F)c4)n3)CC2)cc(OC)c1OC]

In one embodiment of the compound of Formula (I), the compound is:

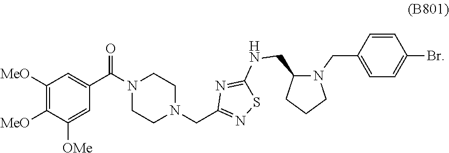 [SMILES: COc1cc(C(=O)N2CCN(Cc3nsc(NC[C@@H]4CCCN4Cc4ccc(Br)cc4)n3)CC2)cc(OC)c1OC]

In one embodiment of the compound of Formula (I), the compound is:

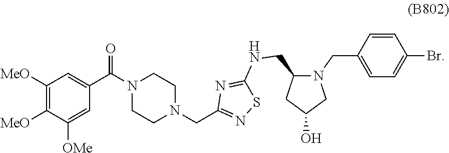 [SMILES: COc1cc(C(=O)N2CCN(Cc3nsc(NC[C@@H]4C[C@@H](O)CN4Cc4ccc(Br)cc4)n3)CC2)cc(OC)c1OC]

In one embodiment of the compound of Formula (I), the compound is:

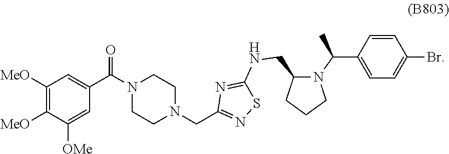 [SMILES: COc1cc(C(=O)N2CCN(Cc3nsc(NC[C@@H]4CCCN4[C@@H](C)c4ccc(Br)cc4)n3)CC2)cc(OC)c1OC]

In one embodiment of the compound of Formula (I), the compound is:

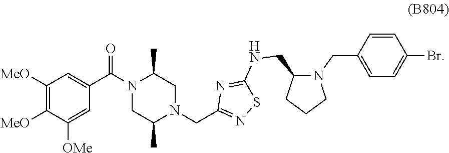 [SMILES: COc1cc(C(=O)N2C[C@H](C)N(Cc3nsc(NC[C@@H]4CCCN4Cc4ccc(Br)cc4)n3)C[C@@H]2C)cc(OC)c1OC]

In one embodiment of the compound of Formula (I), the compound is:

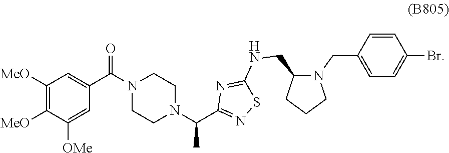 [SMILES: COc1cc(C(=O)N2CCN([C@H](C)c3nsc(NC[C@@H]4CCCN4Cc4ccc(Br)cc4)n3)CC2)cc(OC)c1OC]

In one embodiment of the compound of Formula (I), the compound is:

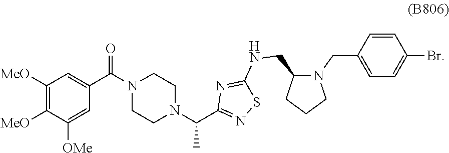 [SMILES: COc1cc(C(=O)N2CCN([C@@H](C)c3nsc(NC[C@@H]4CCCN4Cc4ccc(Br)cc4)n3)CC2)cc(OC)c1OC]

In one embodiment of the compound of Formula (I), the compound is:

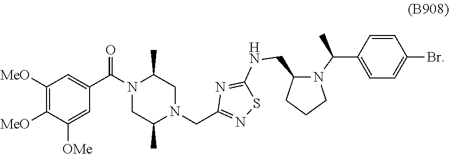 [SMILES: COc1cc(C(=O)N2C[C@H](C)N(Cc3nsc(NC[C@@H]4CCCN4[C@@H](C)c4ccc(Br)cc4)n3)C[C@@H]2C)cc(OC)c1OC]

In one embodiment of the compound of Formula (I), the compound is:

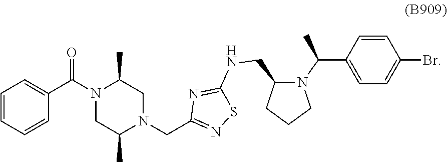 [SMILES: C[C@H]1CN(C(=O)c2ccccc2)[C@@H](C)CN1Cc1nsc(NC[C@@H]2CCCN2[C@@H](C)c2ccc(Br)cc2)n1]

In one embodiment of the compound of Formula (I), the compound is:

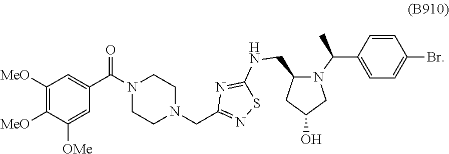 [SMILES: COc1cc(C(=O)N2CCN(Cc3nsc(NC[C@@H]4C[C@@H](O)CN4[C@@H](C)c4ccc(Br)cc4)n3)CC2)cc(OC)c1OC]

In one embodiment of the compound of Formula (I), the compound is:

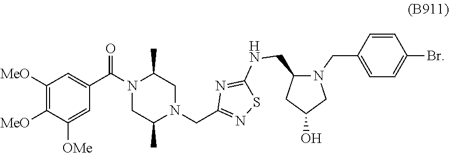 [SMILES: COc1cc(C(=O)N2C[C@H](C)N(Cc3nsc(NC[C@@H]4C[C@@H](O)CN4Cc4ccc(Br)cc4)n3)C[C@@H]2C)cc(OC)c1OC]

Pharmaceutically Acceptable Salts, Isomers, Tautomers, Solvates, Crystalline Forms, Prodrugs and/or Isotopically Enriched/Labelled Compounds

Certain compounds of the disclosure are capable of forming acid and/or base salts by virtue of the presence of acidic or basic functionalities, such as amino and/or carboxyl groups or groups similar thereto. Accordingly, the present disclosure also relates to pharmaceutically acceptable salts of the compounds of the disclosure.

Pharmaceutically acceptable base addition salts can be prepared from inorganic and organic bases. Salts derived from inorganic bases include, but are not limited to, sodium, potassium, lithium, calcium and magnesium salts. Salts derived from organic bases include, but are not limited to, salts of primary, secondary and tertiary amines.

Pharmaceutically acceptable acid addition salts may be prepared from inorganic and organic acids. Salts derived from inorganic acids include, but are not limited to, hydrochloric acid, hydrobromic acid, sulfuric acid, nitric acid and phosphoric acid salts, and the like. Salts derived from organic acids include, but are not limited to, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene sulfonic acid and salicylic acid salts, and the like.

Certain a compounds of the disclosure possess asymmetric carbon atoms (optical or chiral centers) and/or double bonds. Accordingly, the present disclosure also relates to individual isomers of the compounds of the disclosure.

Unless otherwise indicated herein, structures depicted herein are meant to include all stereochemical forms of the structure, for example the R and S configurations for each asymmetric center (D and L configurations for amino acids). Optically active (R)- and (S)- (or (D)- and (L)-isomers) may be prepared using chiral synthons or chiral reagents, or resolved using conventional techniques. When the compounds of the disclosure contain olefinic bonds or other centers of geometric asymmetry, and unless indicated otherwise, it is intended that the compounds include both E and Z geometric isomers.

Certain compounds of the disclosure may exist in tautomeric forms. Accordingly, the present disclosure also relates to tautomers of the compounds of the disclosure.

Certain compounds of the disclosure may exist in unsolvated and solvated forms, including hydrated forms, and/or in crystalline or amorphous forms. In general, all physical and solvated forms are equivalent for the uses contemplated by the present disclosure. Accordingly, the present disclosure also relates to solvated and crystalline forms of the compounds of the disclosure.

Certain compounds of the disclosure may be generated from prodrugs. Accordingly, the present disclosure also relates to prodrugs of the compounds of the disclosure. Relevant prodrugs may be produced from compounds of the disclosure according to well-known methods.

Certain compounds of the disclosure may contain unnatural proportions of atomic isotopes at one or more of the atoms of said compounds. For example, the compounds of the disclosure may comprise deuterium (²H) at at least one position in the compound rather than hydrogen (¹H). The compounds of the disclosure may also or alternatively be radiolabeled with radioactive isotopes, such as for example tritium (³H), iodine-125 (¹²⁵I), and/or carbon-14 (¹⁴C). Such compounds can generally be prepared by conventional techniques known to those skilled in the art or by processes analogous to those described herein, using an appropriate isotopically-enriched/labeled reagent in place of the non-labeled reagent otherwise employed. Accordingly, the present disclosure also relates to isotopic variations of the compounds of the disclosure.

Unless indicated otherwise, for the remainder of the description, reference to a compound of the disclosure is intended to cover the compound of the disclosure, and the pharmaceutically acceptable salts, solvates, tautomers, stereoisomers, and prodrugs thereof, in any physical form (i.e. crystal or amorphous form), as well as isotopic variations thereof.

Pharmaceutical Compositions

The compounds of the disclosure may be formulated in a pharmaceutical composition, optionally together with a pharmaceutically acceptable carrier, excipient and/or diluent.

In one embodiment, the present disclosure relates to a pharmaceutical composition comprising a compound of the disclosure, together with a pharmaceutically acceptable carrier, excipient and/or diluent.

As used herein, the term “pharmaceutically acceptable carrier” refers to a carrier that is ‘acceptable’ in the sense of being compatible with the other ingredients of a composition and not deleterious (e.g. toxic) to the recipient thereof. Typically, the pharmaceutically acceptable carrier is a medium that does not interfere with the activity of the compound of the disclosure.

Some examples of pharmaceutically acceptable carriers include, but are not limited to, e.g., water, phosphate buffered saline, glycerol, ethanol, Ringer's solution, dextrose solution, serum-containing solutions, Hank's solution, other aqueous physiologically balanced solutions, oil-in-water emulsions, oils, water-in-oil emulsions, esters, poly(ethylene-vinyl acetate), copolymers of lactic acid and glycolic acid, poly(lactic acid), gelatin, collagen matrices, polysaccharides, poly(D,L-lactide), poly(malic acid), poly(caprolactone), celluloses, albumin, starch, casein, dextran, polyesters, methacrylate, polyurethane, polyethylene, vinyl polymers, glycols, thyroglobulin, albumins such as human serum albumin, tetanus toxoid, polyamino acids such as poly L-lysine, poly L-glutamic acid, influenza, hepatitis B virus core protein, mixtures thereof and the like (see, for example, Remington: The Science and Practice of Pharmacy, 2000, Gennaro, A R ed., Eaton, Pa.: Mack Publishing Co.).

The pharmaceutical compositions disclosed herein may also comprise excipients. Examples of pharmaceutically acceptable excipients include, but are not limited to, e.g., lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose.

The pharmaceutical compositions disclosed herein may also comprise auxiliary agents or diluents which are known in the art, such as and without limitation: salts; lubricating agents, such as talc, magnesium stearate, and mineral oil; buffering agents; wetting agents; emulsifying agents; suspending agents; and preservatives, such as such as methyl- and propylhydroxybenzoates (see, for example, Porter et al., eds., The Merck Manual, 19th edition, Merck and Co., Rahway, N.J., 2011). When used in pharmaceutical compositions, the salts should typically be pharmaceutically acceptable salts as described herein, but non-pharmaceutically acceptable salts may conveniently be used to prepare pharmaceutically acceptable salts thereof and are not excluded from the scope of the disclosure.

The pharmaceutical compositions disclosed herein may also comprise at least one agent selected from sweetening agents, flavoring agents, coloring agents, demulcents and antioxidants.

Additionally or alternatively, the pharmaceutical compositions may include one or more other compounds as additional therapeutic agents, such as one or more additional compound of the disclosure, or additional therapeutic agent, such as another PD-1/PD-L1 blocker. The additional therapeutic agents may be useful for treating a disease or condition that is amenable to treatment by blocking PD-1, PD-L1 and/or the PD-1/PD-L1 interaction, a cancer, an allergic disorder, sepsis, an autoimmune disease, an inflammatory disease, and/or an acute inflammatory reaction incident to or co-presenting with a cancer.

In one embodiment, the present disclosure relates to a pharmaceutical composition comprising a compound of the disclosure, together with an additional therapeutic agent. The additional therapeutic agent may be selected based on the disease or condition to be treated.

In one embodiment, the present disclosure relates to a pharmaceutical composition comprising a compound of the disclosure, together with an additional anticancer agent.

Many anticancer agents are known in the art, and include, without limitation:

Alkylating Agents such as altretamine, bendamustine, busulfan, carnnustine, chlorambucil, chlormethine, cyclophosphamide, dacarbazine, ifosfamide, improsulfan, tosilate, lomustine, melphalan, mitobronitol, mitolactol, nimustine, ranimustine, temozolomide, thiotepa, treosulfan, mechloretamine, carboquone, apaziquone, fotemustine, glufosfamide, palifosfamide, pipobroman, trofosfamide, uramustine, TH-302 and VAL-083;

Platinum Compounds such as carboplatin, cisplatin, eptaplatin, miriplatine hydrate, oxaliplatin, lobaplatin, nedaplatin, picoplatin, satraplatin, lobaplatin, nedaplatin, picoplatin and satraplatin;

DNA Altering Agents such as amrubicin, bisantrene, decitabine, mitoxantrone, procarbazine, trabectedin, clofarabine, amsacrine, brostallicin, pixantrone and laromustine;

Topoisomerase Inhibitors such as etoposide, irinotecan, razoxane, sobuzoxane, teniposide, topotecan, amonafide, belotecan, elliptinium acetate and voreloxin;

Microtubule Modifiers such as cabazitaxel, docetaxel, eribulin, ixabepilone, paclitaxel, vinblastine, vincristine, vinorelbine, vindesine, vinflunine, fosbretabulin and tesetaxel;

Antimetabolites such as asparaginase, azacitidine, calcium levofolinate, capecitabine, cladribine, cytarabine, enocitabine, floxuridine, fludarabine, fluorouracil, gemcitabine, mercaptopurine, methotrexate, nelarabine, pemetrexed, pralatrexate, azathioprine, thioguanine, carmofur, doxifluridine, elacytarabine, raltitrexed, sapacitabine, tegafur and trimetrexate;

Anticancer Antibiotics such as bleomycin, dactinomycin, doxorubicin, epirubicin, idarubicin, levamisole, miltefosine, mitomycin C, romidepsin, streptozocin, valrubicin, zinostatin, zorubicin, daunurobicin, plicamycin, aclarubicin, peplomycin and pirarubicin;

Hormones/Antagonists such as abarelix, abiraterone, bicalutamide, buserelin, calusterone, chlorotrianisene, degarelix, dexamethasone, estradiol, fluocortolone fluoxymesterone, flutamide, fulvestrant, goserelin, histrelin, leuprorelin, megestrol, mitotane, nafarelin, nandrolone, nilutamide, octreotide, prednisolone, raloxifene, tamoxifen, thyrotropin alfa, toremifene, trilostane, triptorelin, diethylstilbestrol, acolbifene, danazol, deslorelin, epitiostanol, orteronel and enzalutamide;

Aromatase Inhibitors such as aminoglutethimide, anastrozole, exemestane, fadrozole, letrozole, testolactone and formestane;

Small Molecule Kinase Inhibitors such as crizotinib, dasatinib, erlotinib, imatinib, lapatinib, nilotinib, pazopanib, regorafenib, ruxolitinib, sorafenib, sunitinib, vandetanib, vemurafenib, bosutinib, gefitinib, axitinib, afatinib, alisertib, dabrafenib, dacomitinib, dinaciclib, dovitinib, enzastaurin, nintedanib, lenvatinib, linifanib, linsitinib, masitinib, midostaurin, motesanib, neratinib, orantinib, perifosine, ponatinib, radotinib, rigosertib, tipifarnib, tivantinib, tivozanib, trametinib, pimasertib, brivanib alaninate, cediranib, apatinib 4, cabozantinib S-malate, ibrutinib, icotinib, buparlisib, cipatinib, cobimetinib, idelalisib, fedratinib and XL-647;

Photosensitizers such as methoxsalen, porfimer sodium, talaporfin and temoporfin;

Antibodies such as alemtuzumab, besilesomab, brentuximab vedotin, cetuximab, denosumab, ipilimumab, ofatumumab, panitumumab, rituximab, tositumomab, trastuzumab, bevacizumab, pertuzumab, catumaxomab, elotuzumab, epratuzumab, farletuzumab, mogamulizumab, necitumumab, nimotuzumab, obinutuzumab, ocaratuzumab, oregovomab, ramucirumab, rilotumumab, siltuximab, tocilizumab, zalutumumab, zanolimumab, matuzumab, dalotuzumab, onartuzumab, racotumomab, tabalumab, EMD-525797 and nivolumab;

Cytokines such as aldesleukin, interferon alfa2, interferon alfa2a3, interferon alfa2b, celmoleukin, tasonermin, teceleukin, oprelvekin and recombinant interferon beta-1a;

Drug Conjugates such as denileukin diftitox, ibritumomab tiuxetan, iobenguane 1123, prednimustine, trastuzumab emtansine, estramustine, gemtuzumab, ozogamicin, aflibercept, cintredekin besudotox, edotreotide, inotuzumab ozogamicin, naptumomab estafenatox, oportuzumab monatox, technetium (^{99m}Tc) arcitumomab and vintafolide;

Vaccines such as sipuleucel, vitespen, emepepimut-S, oncoVAX, rindopepimut3, troVax, MGN-1601 and MGN-1703; and

Miscellaneous: alitretinoin, bexarotene, bortezonnib, everolimus, ibandronic acid, imiquimod, lenalidomide, lentinan, metirosine, mifamurtide, pamidronic acid, pegaspargase, pentostatin, sipuleucel, sizofiran, tamibarotene, temsirolimus, thalidomide, tretinoin, vismodegib, zoledronic acid, vorinostat, celecoxib, cilengitide, entinostat, etanidazole, ganetespib, idronoxil, iniparib, ixazomib, lonidamine, nimorazole, panobinostat, peretinoin, plitidepsin, pomalidomide, procodazol, ridaforolimus, tasquinimod, telotristat, thymalfasin, tirapazamine, tosedostat, trabedersen, ubenimex, valspodar, gendicine, picibanil, reolysin, retaspimycin hydrochloride, trebananib, virulizin, carfilzomib, endostatin, immucothel, belinostat and MGN-1703.

In one embodiment, the present disclosure relates to a pharmaceutical composition comprising a compound of the disclosure, together with a Nivolumab (Opdivo), Pembrolizumab (Keytruda), Atezolizumab (Tecentriq), Avelumab (Bavencio), Durvalumab (Imfinzi), or Cemiplimab (Libtayo).

In some embodiments, the pharmaceutical compositions further comprise at least one viral chemotherapeutic compound, such as for example, and without limitation, one selected from gamma globulin, amantadine, guanidine, hydroxybenzimidazole, interferon-α, interferon-β, interferon-γ, thiosemicarbarzones, methisazone, rifampin, ribvirin, a pyrimidine analog, a purine analog, foscarnet, phosphonoacetic acid, acyclovir, dideoxynucleosides, or ganciclovir (see, e.g., Katzung, ed., Basic and Clinical Pharmacology, Fifth Edition, Appleton and Lange, Norwalk, Conn., (1992)).

In one embodiment, the present disclosure relates to a pharmaceutical composition comprising a compound of the disclosure, together with an additional therapeutic agent that is used in the treatment of autoimmune diseases.

The additional therapeutic agent used in the treatment of autoimmune diseases may be selected based on the autoimmune disease to be treated. Many therapeutic agents for treating autoimmune diseases are known in the art, and include, without limitation: insulin preparations, such as human insulin, insulin glargine, insulin lispro, insulin detemir and insulin aspart; sulfonylurea agents, such as glibenclamide, gliclazide and glimepiride; quick-acting insulin secretion promoters, such as nateglinide; biguanide preparations, such as metformin; insulin sensitizers, such as pioglitazone; α-glucosidase inhibitors, such as acarbose and voglibose; diabetic neuropathy therapeutic agents, such as epalrestat, mexiletine and imidapril; GLP-1 analog preparations, such as liraglutide, exenatide and lixisenatide; DPP-4 inhibitors, such as sitagliptin, vildagliptin and alogliptin; steroid agents, such as cortisone acetate, hydrocortisone, hydrocortisone sodium phosphate, hydrocortisone sodium succinate, fludrocortisone acetate, prednisolone, prednisolone acetate, prednisolone sodium succinate, prednisolone butylacetate, prednisolone sodium phosphate, halopredone acetate, methylprednisolone, methylprednisolone acetate, methylprednisolone sodium succinate, triamcinolone, triamcinolone acetate, triamcinolone acetonide, dexamethasone, dexamethasone acetate, dexamethasone sodium phosphate, dexamethasone palmitate, paramethasone acetate and betamethasone; interferon β-1a; interferon β-1b; glatiramer acetate; mitoxantrone; azathioprine; cyclophosphamide; cladribine; adrenocorticotropic hormone (ACTH); corticotropin; alemtuzumab; immunosuppressive agents, such as cyclosporin, tacrolimus and fingolimod; anti-rheumatic agents, such as methotrexate, sulfasalazine, bucillamine, leflunomide, mizoribine and tacrolimus; and anti-cytokine agents, such as infliximab, adalimumab, tocilizumab, etanercept and abatacept.

In some embodiments, the pharmaceutical compositions contain, as an active ingredient, a compound of the disclosure in a therapeutically effective amount. As used in these embodiments, a “therapeutically effective amount” refers to an amount of the compound of the disclosure effective to treat a condition associated with PD-1, PD-L1 and/or the PD-1/PD-L1 interaction, such as cancer, sepsis or an autoimmune disease. In these embodiments, the compound of the disclosure may block PD-1 and PD-L1 from interacting, either by blocking PD-1 or PD-L1, or by an alternative mechanism.

Generally, methods of preparing pharmaceutical compositions are well known in the art, and any of these methods may be employed in order to prepare the compositions described herein.

Methods and Uses

The compounds and pharmaceutical compositions of the disclosure may function as PD-1 blockers, PD-L1 blockers and/or block the PD-1/PD-L1 interaction by an alternative mechanism.

In one embodiment, the present disclosure relates to a method of treating a disease or condition that is amenable to treatment by blocking PD-1, PD-L1 and/or the PD-1/PD-L1 interaction, wherein the method comprises administering a compound or a pharmaceutical composition of the disclosure to a subject in need thereof.

In another embodiment, the present disclosure relates to the use of a compound or a pharmaceutical composition of the disclosure for the treatment of a disease or condition that is amenable to treatment by blocking PD-1, PD-L1 and/or the PD-1/PD-L1 interaction in a subject in need thereof.

In still another embodiment, the present disclosure relates to the use of a compound or a pharmaceutical composition of the disclosure for the manufacture of a medicament for the treatment of a disease or condition that is amenable to treatment by blocking PD-1, PD-L1 and/or the PD-1/PD-L1 interaction.

A compound or a pharmaceutical composition of the disclosure may be suitable for use in the treatment and/or prevention of cancer in a subject in need thereof. The subject may have cancer or may be at risk of developing cancer. The compound or pharmaceutical composition may, for example, reduce the severity of cancer (e.g. size of the tumor, aggressiveness and/or invasiveness, malignancy, etc.) or prevent cancer recurrences.

In one embodiment, the present disclosure relates to a method of treating cancer, wherein the method comprises administering a compound or a pharmaceutical composition of the disclosure to a subject in need thereof.

In another embodiment, the present disclosure relates to the use of a compound or a pharmaceutical composition of the disclosure for the treatment of cancer in a subject in need thereof.

In still another embodiment, the present disclosure relates to the use of a compound or a pharmaceutical composition of the disclosure for the manufacture of a medicament for the treatment of cancer.

As used herein, the terms “cancer”, “cancer cells”, “tumor” and “tumor cells” (which may be used interchangeably) refer to cells that exhibit abnormal growth, characterized by a significant loss of control of cell proliferation or cells that have been immortalized. The term “cancer” or “tumor” includes metastatic as well as non-metastatic cancer or tumors. A cancer may be diagnosed using criteria generally accepted in the art, including the presence of a malignant tumor.

Without limitation, cancers that may be capable of being treated and/or prevented by the use or administration of a compound or pharmaceutical composition of the disclosure include bone cancer, cancer of the head or neck, pancreatic cancer, skin cancer, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumours of childhood, lymphocytic lymphoma, cancer of the bladder, cancer of the kidney or ureter, carcinoma of the renal pelvis, neoplasm of the central nervous system (CNS), primary CNS lymphoma, tumour angiogenesis, spinal axis tumour, brain stem glioma, pituitary adenoma, Kaposi's sarcoma, epidermoid cancer, squamous cell cancer, T-cell lymphoma, environmentally induced cancers including those induced by asbestos, and combinations of said cancers.

As reagents targeting the PD-1/PD-L1 axis may be effective in the treatment of sepsis—it has been postulated that anti-PD-1 and anti-PD-L1 therapy could reduce sepsis-induced immune dysfunction which drives ongoing infectious complications-compounds that interact with PD-1, PD-L1 and/or PD-L2 have been explored as potential treatments for sepsis (Zhang, Bei. “New paradigm of immune checkpoint immunotherapy in sepsis” (15 Jun. 2019) International Journal of Clinical and Experimental Medicine, 12(6), 7692). In this regard, anti-PD-1 and anti-PD-L1 therapies have demonstrated promising results in human trials involving sepsis (Shindo, Yuichiro, et al. “Anti-PD-L1 peptide improves survival in sepsis” (8 Sep. 2016) Journal of Surgical Research, 208, 33).

The compounds and pharmaceutical compositions of the disclosure may be effective in the treatment of sepsis.

In one embodiment, the present disclosure relates to a method of treating sepsis, wherein the method comprises administering a compound or a pharmaceutical composition of the disclosure to a subject in need thereof.

In another embodiment, the present disclosure relates to the use of a compound or a pharmaceutical composition of the disclosure for the treatment of sepsis in a subject in need thereof.

In still another embodiment, the present disclosure relates to the use of a compound or a pharmaceutical composition of the disclosure for the manufacture of a medicament for the treatment of sepsis.

The PD-1 signalling pathway is also known to be involved in the inhibition of self-reactive T cells, and to play a role in the suppression of autoimmune diseases. As such, compounds that interact with PD-1, PD-L1 and/or PD-L2 have been explored as potential treatments for autoimmune diseases (see, for example: Guo, Yanxia et al. “Immune checkpoint inhibitor PD-1 pathway is down-regulated in synovium at various stages of rheumatoid arthritis disease progression” (28 Feb. 2018) PLOS ONE 13(2), e0192704, https://doi.org/10.1371/journal.pone.0192704; Qin, Weiting et al. “The Diverse Function of PD-1/PD-L Pathway Beyond Cancer” (4 Oct. 2019) Frontiers in Immunology, 10, 2298; U.S. Pat. Nos. 10,493,148; and 9,701,749).

The compounds and pharmaceutical compositions of the disclosure may be effective in the treatment of an autoimmune disease.

In one embodiment, the present disclosure relates to a method of treating an autoimmune disease, wherein the method comprises administering a compound or a pharmaceutical composition of the disclosure to a subject in need thereof.

In another embodiment, the present disclosure relates to the use of a compound or a pharmaceutical composition of the disclosure for the treatment of an autoimmune disease in a subject in need thereof.

In still another embodiment, the present disclosure relates to the use of a compound or a pharmaceutical composition of the disclosure for the manufacture of a medicament for the treatment of an autoimmune disease.

Without limitation, autoimmune diseases that may be capable of being treated and/or prevented by the use or administration of a compound or pharmaceutical composition of the disclosure include Behcet disease, systemic lupus erythematosus, multiple sclerosis (systemic scleroderma and progressive systemic scleroderma), scleroderma, polymyositis, dermatomyositis, periarteritis nodosa (polyarteritis nodosa and microscopic polyangiitis), aortitis syndrome (Takayasu arteritis), malignant rheumatoid arthritis, rheumatoid arthritis, Wegner's granulomatosis, mixed connective tissue disease, Sjogren syndrome, adult-onset Still's disease, allergic granulomatous angiitis, hypersensitivity angiitis, Cogan's syndrome, RS3PE, temporal arteritis, polymyalgia rheumatica, fibromyalgia syndrome, antiphospholipid antibody syndrome, eosinophilic fasciitis, IgG4-related diseases (e.g., primary sclerosing cholangitis and autoimmune pancreatitis), Guillain-Barre syndrome, myasthenia gravis, chronic atrophic gastritis, autoimmune hepatitis, primary biliary cirrhosis, aortitis syndrome, Goodpasture's syndrome, rapidly progressive glomerulonephritis, megaloblastic anemia, autoimmune hemolytic anemia, autoimmune neutropenia, idiopathic thrombocytopenic purpura, Graves' disease (hyperthyroidism), Hashimoto's thyroiditis, autoimmune adrenal insufficiency, primary hypothyroidism, idiopathic Addison's disease (chronic adrenal insufficiency), type I diabetes mellitus, chronic discoid lupus erythematosus, localized scleroderma, psoriasis, psoriatic arthritis, pemphigus, pemphigoid, herpes gestationis, linear IgA bullous skin disease, epidermolysis bullosa acquisita, alopecia areata, vitiligo, Harada disease, autoimmune optic neuropathy, idiopathic azoospermia, recurrent fetal loss, inflammatory bowel diseases (ulcerative colitis and Crohn's disease), and graft-versus-host disease (GVHD).

Compounds and pharmaceutical compositions of the disclosure may be used together with additional therapeutic agents. The additional therapeutic agents may be useful for treating a disease or condition that is amenable to treatment by blocking PD-1, PD-L1 and/or the PD-1/PD-L1 interaction, a cancer, an allergic disorder, sepsis, an autoimmune disease, an inflammatory disease, and/or an acute inflammatory reaction incident to or co-presenting with a cancer.

The compound or pharmaceutical composition of the disclosure and additional therapeutic agent may be administered as a combination preparation in which both components are contained in a single formulation (a combined dosage unit), or administered as separate formulations. Where the compound or pharmaceutical composition of the disclosure and additional therapeutic agent are for administration by separate formulations, administration may be simultaneous or with some time intervals. In the case of the administration with some time intervals, the compound or pharmaceutical composition of the disclosure can be administered first, followed the additional therapeutic agent or the additional therapeutic agent can be administered first, followed by the compound or pharmaceutical composition. The administration method of the compound or pharmaceutical composition of the disclosure and additional therapeutic agent may be same or different. The intervals will depend on the pharmacokinetic and/or pharmacodynamics properties of the compound or pharmaceutical composition of the disclosure and the additional therapeutic agent.

Anti-inflammatory agents that may be used together with a compound or pharmaceutical composition of the disclosure include, but are not limited to, NSAIDs, non-specific and COX-2 specific cyclooxgenase enzyme inhibitors, gold compounds, corticosteroids, metabolic inhibitors, dihydrofolate reductase inhibitors, dihydroorotate dehydrogenase inhibitors, tumor necrosis factor receptor (TNF) receptors antagonists, immunosuppressants, and methotrexate.

In one embodiment, the present disclosure relates to a method of treating a disease or condition that is amenable to treatment by blocking PD-1, PD-L1 and/or the PD-1/PD-L1 interaction, wherein the method comprises administering a compound or a pharmaceutical composition of the disclosure in combination with an additional therapeutic agent to a subject in need thereof, wherein the compound or pharmaceutical composition is administered with, before or after the additional therapeutic agent.

In another embodiment, the present disclosure relates to the use of a compound or a pharmaceutical composition of the disclosure and an additional therapeutic agent for the treatment of a disease or condition that is amenable to treatment by blocking PD-1, PD-L1 and/or the PD-1/PD-L1 interaction in a subject in need thereof, wherein the compound or pharmaceutical composition is for administration with, before or after the additional therapeutic agent.

In yet another embodiment, the present disclosure relates to a method of treating cancer, wherein the method comprises administering a compound or a pharmaceutical composition of the disclosure in combination with an additional anticancer agent to a subject in need thereof, wherein the compound or pharmaceutical composition is administered with, before or after the additional anticancer agent.

In still another embodiment, the present disclosure relates to the use of a compound or a pharmaceutical composition of the disclosure and an additional anticancer agent for the treatment of cancer in a subject in need thereof, wherein the compound or pharmaceutical composition is for administration before, after or simultaneously with the additional anticancer agent.

The compounds and pharmaceutical compositions of the disclosure may be used together with one or more standard therapies including, but not limited to, chemotherapy, radiotherapy, immunotherapy, surgery, or combination thereof.

In one embodiment, the present disclosure relates to a method of treating cancer, wherein the method comprises administering a compound or a pharmaceutical composition of the disclosure before, after or during administration of a chemotherapy, radiotherapy, immunotherapy, surgery or a combination thereof.

In another embodiment, the present disclosure relates to the use of a compound or a pharmaceutical composition of the disclosure together with a chemotherapy, radiotherapy, immunotherapy, surgery or combination thereof for the treatment of cancer in a subject in need thereof, wherein the compound or pharmaceutical composition is for administration before, after, or during the administration of a chemotherapy, radiotherapy, immunotherapy, surgery or combination thereof.

In one embodiment, the present disclosure relates to a method of treating sepsis, wherein the method comprises administering a compound or a pharmaceutical composition of the disclosure in combination with an additional therapeutic agent to a subject in need thereof, wherein the compound or pharmaceutical composition is administered with, before or after the additional therapeutic agent.

In another embodiment, the present disclosure relates to the use of a compound or a pharmaceutical composition of the disclosure and an additional therapeutic agent for the treatment of sepsis in a subject in need thereof, wherein the compound or pharmaceutical composition is for administration with, before or after the additional therapeutic agent.

In yet another embodiment, the present disclosure relates to a method of treating an autoimmune disease, wherein the method comprises administering a compound or a pharmaceutical composition of the disclosure in combination with an additional therapeutic agent to a subject in need thereof, wherein the compound or pharmaceutical composition is administered with, before or after the additional therapeutic agent.

In still another embodiment, the present disclosure relates to the use of a compound or a pharmaceutical composition of the disclosure and an additional therapeutic agent for the treatment of an autoimmune disease in a subject in need thereof, wherein the compound or pharmaceutical composition is for administration with, before or after the additional therapeutic agent.

In some embodiments, the compounds and pharmaceutical compositions of the disclosure may be effective when administered in a single application.

The subject to be treated with a compound or pharmaceutical composition of the disclosure may be any vertebrate, more particularly a mammal. In some embodiments, the subject is a human.

The optimal amount of the compound or pharmaceutical composition of the disclosure may depend on a number of factors including, without limitation, the compound or composition, the disease, and/or the subject, and may be readily ascertained by the skilled person using standard studies including, for example, observations of antibody titers, antigen-specific IFN-gamma responses, measurements of tumor volume or other characteristics, and other immunogenic responses in the host.

A skilled person can determine suitable treatment regimes, routes of administration, dosages, etc., for any particular application. Factors that may be taken into account include, e.g., the disease state to be prevented or treated; the age, physical condition, body weight, sex and diet of the subject; and other clinical factors.

Modes of Administration

The compounds and pharmaceutical compositions of the disclosure may be administered by any means known in the art. For example, and without limitation, the compounds and pharmaceutical compositions of the disclosure may be formulated in a form that is suitable for oral, rectal, ocular, pulmonary or parenteral administration, and if parenteral, either locally or systemically. Parenteral administration includes, without limitation, intravenous, intraperitoneal, intradermal, subcutaneous, intramuscular, intranasal, transdermal, transepithelial, intrapulmonary, intrathecal, and topical or buccal modes of administration. Parenteral administration can be by bolus injection or by gradual perfusion over time.

In some embodiments, a compound or pharmaceutical composition of the disclosure may be suitable for oral administration. A compound may be suitable for oral administration if the compound has appropriate MW, Log P, TPSA, Fsp3 and LE parameters.

MW, as used herein, refers to the molecular weight of a compound. For a compound to be orally bioavailable it is desirable for the compound to have a MW of <450 Daltons, it is acceptable for the compound to have a MW of 450-600 Daltons, and it is undesirable for the compound to have a MW of >600 Daltons.

Log P, as used herein, refers to a measure of the lipophilicity of a compound (the logarithm of the octanol-water partition coefficient). For a compound to be orally bioavailable, it is desirable for the compound to have a Log P of <3, it is acceptable for the compound to have a Log P of 3-5, and it is undesirable for the compound to have a Log P of >5 or <0.

TPSA, as used herein, refers to a measure of the transport properties of a compound, for example, the ability of the compound to permeate cells. For a compound to be orally bioavailable, it is desirable for the compound to have a TPSA of <100 Å², it is acceptable for the compound to have a TPSA of 100-150 Å², and it is undesirable for the compound to have a TPSA of >150 Å².

Fsp³, as used herein, refers to a measure of the fraction of sp³ hybridized centres in a compound (Fsp³=number of sp³-hybridized carbons/total carbon count). For a compound to be orally bioavailable, it is desirable for the compound to have a Fsp³ of >0.5, it is acceptable for the compound to have a Fsp³ of 0.3-0.5, and it is undesirable for the compound to have a Fsp³ of <0.3.

LE, as used herein, refers to the ligand efficiency of a compound. The LE value expresses the binding energy of a compound normalized by the compound's size. For a compound to be orally bioavailable, it is desirable for the compound to have a LE of >0.5, it is acceptable for the compound to have a LE of 0.3-0.5, and it is undesirable for the compound to have a LE of <0.3.

A skilled person would understand that the above ranges are only a guide, and that a compound may still be orally bioavailable if each of the parameters does not fall in the desirable or acceptable ranges noted above.

A compound of the disclosure, where suitable for oral administration, may be formulated in any acceptable and suitable oral preparation, including, but are not limited to, tablets, troches, lozenges, aqueous and oily suspensions, dispersible powders or granules, emulsions, hard and soft capsules, liquid capsules, syrups, and elixirs. The oral preparations can be prepared according to any methods known in the art.

In some embodiments, a compound or pharmaceutical composition of the disclosure may be suitable for intravenous, subcutaneous, and/or intramuscular delivery via any pharmaceutically acceptable and suitable injectable form, including, but not limited to, sterile aqueous solutions comprising acceptable vehicles and solvents, such as, for example, water, Ringer's solution, and isotonic sodium chloride solution; sterile oil-in-water microemulsions; and aqueous or oleaginous suspensions.

Kits

The compounds and pharmaceutical compositions of the disclosure are optionally provided to a user as a kit. For example, a kit may comprise one or more components of the pharmaceutical compositions of the disclosure, one or more additional therapeutic agent, packaging material, containers for holding the components of the kit, and/or instructions or a user manual detailing preferred methods of using the kit components.

In one embodiment, the present disclosure relates to a kit comprising (a) compound of the disclosure, or a pharmaceutical composition of the disclosure; and (b) an additional therapeutic agent.

In another embodiment, the present disclosure relates to a kit comprising (a) compound of the disclosure, or a pharmaceutical composition of the disclosure; and (b) instructions for using the compound or pharmaceutical composition to treat a disease or condition. The disease or condition may be a disease or condition that is amenable to treatment by blocking PD-1, PD-L1 and/or the PD-1/PD-L1 interaction, cancer, sepsis or an autoimmune disease.

### Embodiments of the Invention

Particular embodiments of the invention include, without limitation, the following:

Embodiment 1. A compound of Formula (I):

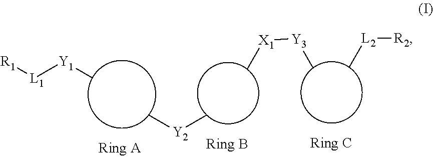

wherein:
R₁ is C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆heteroalkyl or C₅₋₁₄heteroaryl-C₂₋₆heteroalkyl, each of which is unsubstituted or substituted with one or more R^{A};
R₂ is C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆heteroalkyl or C₅₋₁₄heteroaryl-C₂₋₆heteroalkyl, each of which is unsubstituted or substituted with one or more R^{A};
L₁ is optional and if present is C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl or C₅₋₁₄ heterocycloalkenyl, each of which is unsubstituted or substituted with one or more R^{A};
L₂ is optional and if present is C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl or C₅₋₁₄ heterocycloalkenyl, each of which is unsubstituted or substituted with one or more R^{A};
Y₁, Y₂ and Y₃ are independently —CR₅R₆—, —C(CR₅R₆)—, —C(O)—, —C(NR^{B})—, —C(S)—, —NR^{B}—, —O—, —S—, —(O)— or —S(O)₂—; X₁ is —CR^{B}₂—, —C(CR^{B}₂)—, —C(O)—, —C(NR^{B})—, —C(S)—, —NR^{B}—, —O—, —S—, —(O)— or —S(O)₂—;
Ring A is a C₆ cycloalkyl or C₆ heterocycloalkyl, each of which is unsubstituted or substituted with one or more R₃;
Ring B is a C₆ aryl or C₅₋₆ heteroaryl, each of which is unsubstituted or substituted with one or more R^{A};
Ring C is a C₅₋₆ cycloalkyl or C₅₋₆ heterocycloalkyl, each of which is unsubstituted or substituted with one or more R₄;
R₃ and R₄ at each occurrence are independently C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₁₋₆ haloalkyl, C₂₋₆ haloalkenyl, C₂₋₆ haloalkynyl, C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₃₋₁₄ haloheterocycloalkyl, C₅₋₁₄ haloheterocycloalkenyl, C₅₋₁₄ aryl, C₅₋₁₄ heteroaryl, haloC₅₋₁₄ aryl, haloC₅₋₁₄heteroaryl, C₅₋₁₄aryl-C₁₋₆alkyl, C₅₋₁₄heteroaryl-C₁₋₆alkyl, haloC₅₋₁₄aryl-C₁₋₆ alkyl, haloC₅₋₁₄heteroaryl-C₁₋₆alkyl, C₅₋₁₄aryl-C₂₋₆heteroalkyl, C₅₋₁₄heteroaryl-C₂₋₆ heteroalkyl, haloC₅₋₁₄aryl-C₂₋₆heteroalkyl, haloC₅₋₁₄heteroaryl-C₂₋₆heteroalkyl, —F, —Cl, —Br, —I, —CN, —NO₂, —SO₂, —N₃, —SCN, —NCS, —OR^{B}, —NR^{C}R^{D}, —NR^{B}NR^{C}R^{D}—, SR^{B}, —C(O)R^{B}, —C(O)OR^{B}, —C(O)NR^{C}R^{D}, —C(NR^{C})R^{B}, —C(NR^{B})NR^{C}R^{D}, —S(O)R^{B}, —S(O)₂R^{B}, —S(O)OR^{B}, —S(O)₂OR^{B}, —NR^{B}NR^{C}R^{D}, —NR^{B}C(O)NR^{C}R^{D} or —NR^{B}C(NR^{B})NR^{C}R^{D}; two or more R₃ or two or more R₄ together with the atom(s) to which they are attached form a cyclic group; and/or two R₃ or two R₄ on the same atom form (═O), (═NR^{B}) or (═S);
R₅ and R₆ at each occurrence are independently —H, C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₁₋₆ haloalkyl, C₂₋₆ haloalkenyl, C₂₋₆ haloalkynyl, C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₃₋₁₄ haloheterocycloalkyl, C₅₋₁₄ haloheterocycloalkenyl, C₅₋₁₄ aryl, C₅₋₁₄ heteroaryl, haloC₅₋₁₄ aryl, haloC₅₋₁₄heteroaryl, C₅₋₁₄aryl-C₁₋₆alkyl, C₅₋₁₄heteroaryl-C₁₋₆alkyl, haloC₅₋₁₄aryl-C₁₋₆ alkyl, haloC₅₋₁₄heteroaryl-C₁₋₆alkyl, C₅₋₁₄aryl-C₂₋₆heteroalkyl, C₅₋₁₄heteroaryl-C₂₋₆ heteroalkyl, haloC₅₋₁₄aryl-C₂₋₆heteroalkyl, haloC₅₋₁₄heteroaryl-C₂₋₆heteroalkyl, —F, —Cl, —Br, —I, —CN, —NO₂, —SO₂, —N₃, —SCN, —NCS, —OR^{B}, —NR^{C}R^{D}, —NR^{B}NR^{C}R^{D}, —SR^{B}, —C(O)R^{B}, —C(O)OR^{B}, —C(O)NR^{C}R^{D}, —C(NR^{C})R^{B}, —C(NR^{B})NR^{C}R^{D}, —S(O)R^{B}, —S(O)₂R^{B}, —S(O)OR^{B}, —S(O)₂OR^{B}, —NR^{B}NR^{C}R^{D}, —NR^{B}C(O)NR^{C}R^{D} or —NR^{B}C(NR^{B})NR^{C}R^{D}; or R₅ and R₆ together with the carbon atom to which they are attached form a C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₃₋₁₄ haloheterocycloalkyl, or C₅₋₁₄ haloheterocycloalkenyl;
R^{A} at each occurrence is independently —F, —Cl, —Br, —I, —CN, —NO₂, —SO₂, —N₃, —SCN, —NCS, —OR^{B}, —NR^{C}R^{D}, —NR^{B}NR^{C}R^{D}, —SR^{B}, —C(O)R^{B}, —C(O)OR^{B}, —C(O)NR^{C}R^{D}, —C(NR^{B})R^{B}, —C(NR^{B})NR^{C}R^{D}, —S(O)R^{B}, —S(O)₂R^{B}, —S(O)OR^{B}, —S(O)₂OR^{B}, —NR^{B}NR^{C}R^{D}, —NR^{B}C(O)NR^{C}R^{D}, —NR^{B}C(NR^{B})NR^{C}R^{D}, C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆heteroalkyl or C₅₋₁₄heteroaryl-C₂₋₆ heteroalkyl, wherein each C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄ heteroaryl-C₁₋₆alkyl, C₅₋₁₄aryl-C₂₋₆heteroalkyl or C₅₋₁₄heteroaryl-C₂₋₆heteroalkyl is unsubstituted or substituted with one or more —F, —Cl, —Br, —I, —CN, —NO₂, —SO₂, —N₃, —SCN, —NCS, —OR^{B}, —NR^{C}R^{D}, —NR^{B}NR^{C}R^{D}, —SR^{B}, —C(O)R^{B}, —C(O)OR^{B}, —C(O)NR^{C}R^{D}, —C(NR^{B})R^{B}, —C(NR^{B})NR^{C}R^{D}, —NR^{B}C(NR^{B})NR^{C}R^{D}, —S(O)R^{B}, —S(O)₂R^{B}, —S(O)OR^{B}, —S(O)₂OR^{B}, —NR^{B}NR^{C}R^{D} or —NR^{B}C(O)NR^{C}R^{D}; two or more R^{A} together with the atom(s) to which they are attached form a cyclic group; and/or two R^{A} on the same atom form (═O), (═NR^{B}) or (═S);
R^{B} at each occurrence is independently (i) —H, or (ii) C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆heteroalkyl or C₅₋₁₄heteroaryl-C₂₋₆ heteroalkyl, each of which is unsubstituted or substituted with one or more R^{E}R^{C} and R^{D} at each occurrence are independently (i) —H, or (ii) C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆heteroalkyl or C₅₋₁₄heteroaryl-C₂₋₆ heteroalkyl, each of which is unsubstituted or substituted with one or more R^{E}, or R^{C} and R^{D} together with the nitrogen atom to which they are attached combine to form a heterocycle that is unsubstituted or substituted with one or more R^{E} groups;
R^{E} at each occurrence is independently —F, —Cl, —Br, —I, —CN, —NO₂, —SO₂, —N₃, —SCN, —NCS, —OR^{F}, —NR^{G}R^{H}, —NR^{F}NR^{G}R^{H}, —SR^{F}, —C(O)R^{F}, —C(O)OR^{F}, —C(O)NR^{G}R^{H}, —C(NR^{F})R^{F}, —C(NR^{F})NR^{G}R^{H}, —S(O)R^{F}, —S(O)₂R^{F}, —S(O)OR^{F}, —S(O)₂OR^{F}, —NR^{F}NR^{G}R^{H}, —NR^{F}C(O)NR^{G}R^{H}, —NR^{F}C(NR^{F})NR^{G}R^{H}, C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆heteroalkyl or C₅₋₁₄heteroaryl-C₂₋₆ heteroalkyl, wherein each C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₆₋₁₄heteroaryl-C₁₋₆alkyl, C₅₋₁₄aryl-C₂₋₆heteroalkyl or C₅₋₁₄heteroaryl-C₂₋₆heteroalkyl is unsubstituted or substituted with one or more —F, —Cl, —Br, —I, —CN, —NO₂, —SO₂, —N₃, —SCN, —NCS, —OR^{F}, —NR^{G}R^{H}, —NR^{F}NR^{G}R^{H}, —SR^{F}, —C(O)R^{F}, —C(O)OR^{F}, —C(O)NR^{G}R^{H}, —C(NR^{F})R^{F}, —C(NR^{F})NR^{G}R^{H}, —NR^{F}C(NR^{F})NR^{G}R^{H}, —S(O)R^{F}, —S(O)₂R^{F}, —S(O)OR^{F}, —S(O)₂OR^{F}, —NR^{F}NR^{G}R^{H} or —NR^{F}C(O)NR^{G}R^{H}; two or more R^{E} together with the atoms to which they are attached form a cyclic group; and/or two R^{E} on the same atom form (═O), (═NR^{F}) or (═S);
R^{F} at each occurrence is independently (i) —H, or (ii) C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₁₋₆ haloalkyl, C₂₋₆ haloalkenyl, C₂₋₆ haloalkynyl, C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₅₋₁₄ aryl, C₅₋₁₄ heteroaryl, haloC₅₋₁₄aryl, haloC₅₋₁₄heteroaryl, C₅₋₁₄aryl-C₁₋₆alkyl, C₅₋₁₄heteroaryl-C₁₋₆alkyl, haloC₅₋₁₄aryl-C₁₋₆alkyl, haloC₅₋₁₄heteroaryl-C₁₋₆alkyl, C₅₋₁₄aryl-C₂₋₆heteroalkyl, C₅₋₁₄ heteroaryl-C₂₋₆heteroalkyl, haloC₅₋₁₄aryl-C₂₋₆heteroalkyl or haloC₅₋₁₄heteroaryl-C₂₋₆ heteroalkyl; and
R^{G} and R^{H} at each occurrence are independently (i) —H, or (ii) C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₁₋₆ haloalkyl, C₂₋₆ haloalkenyl, C₂₋₆ haloalkynyl, C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₅₋₁₄ aryl, C₅₋₁₄ heteroaryl, haloC₅₋₁₄aryl, haloC₅₋₁₄heteroaryl, C₅₋₁₄aryl-C₁₋₆alkyl, C₅₋₁₄heteroaryl-C₁₋₆alkyl, haloC₅₋₁₄aryl-C₁₋₆alkyl, haloC₅₋₁₄heteroaryl-C₁₋₆alkyl, C₅₋₁₄aryl-C₂₋₆heteroalkyl, C₅₋₁₄heteroaryl-C₂₋₆heteroalkyl, haloC₅₋₁₄aryl-C₂₋₆heteroalkyl or haloC₅₋₁₄heteroaryl-C₂₋₆ heteroalkyl, or R^{G} and R^{H} together with the nitrogen atom to which they are attached combine to form a heterocycle,
   or a pharmaceutically acceptable salt, solvate, tautomer, stereoisomer or prodrug thereof, provided that the compound of Formula (I) is not:

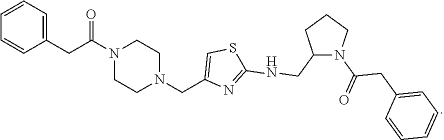 [SMILES: O=C(Cc1ccccc1)N1CCN(Cc2csc(NCC3CCCN3C(=O)Cc3ccccc3)n2)CC1]

Embodiment 2. The compound according to Embodiment 1, or a pharmaceutically acceptable salt, solvate, tautomer, stereoisomer or prodrug thereof, wherein the compound is a compound of Formula (II):

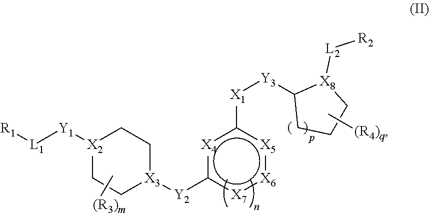

wherein:
X₂, X₃ and X₈ are independently —CH—, —CR^{A}— or —N—;
X₄, X₅ and X₆, and X₇ if present, are independently —CH—, —CR^{A}—, —C(O)—, —C(NR^{B})—, —C(S)—, —N—, —NR^{B}—, —O— or —S—, provided that the combination of X₄, X₅, X₆ and optionally X₇ together with the atoms to which they are attached forms an aromatic or heteroaromatic group;
m is 0 to 8;
n is 0 or 1;
p is 1 or 2; and
q is 0 to 9.

Embodiment 3. The compound according to Embodiment 2, or a pharmaceutically acceptable salt, solvate, tautomer, stereoisomer or prodrug thereof, wherein X₂, X₃ and X₃ are independently —CH—, —C(C₁₋₆ alkyl)- or —N—.

Embodiment 4. The compound according to Embodiment 2 or 3, or a pharmaceutically acceptable salt, solvate, tautomer, stereoisomer or prodrug thereof, wherein: X₂ is —CH— and X₃ is —N—; X₂ is —N— and X₃ is —CH—; X₂ is —CH— and X₃ is —CH—; or X₂ is —N— and X₃ is —N—.

Embodiment 5. The compound according to any one of Embodiments 2 to 4, or a pharmaceutically acceptable salt, solvate, tautomer, stereoisomer or prodrug thereof, wherein m is 0 to 4, and R₃ at each occurrence is independently —F, —C, —Br, —I, —OH, C₁₋₆ alkyl or C₂₋₆ heteroalkyl, wherein each C₁₋₆ alkyl or C₂₋₆ heteroalkyl is unsubstituted; two R₃ together with the atoms to which they are attached form a cyclic group; and/or two R₃ on the same atom form (═O).

Embodiment 6. The compound according to any one of Embodiments 2 to 5, or a pharmaceutically acceptable salt, solvate, tautomer, stereoisomer or prodrug thereof, wherein m is 2 and Ring A is as shown in the following structural fragment:

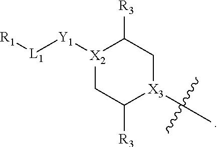

Embodiment 7. The compound according to Embodiment 6, or a pharmaceutically acceptable salt, solvate, tautomer, stereoisomer or prodrug thereof, wherein the stereochemistry of the positions in Ring A identified by a wedge-shaped bond in the following structural fragment are as shown:

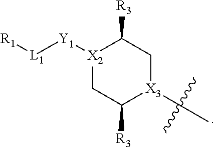

Embodiment 8. The compound according to Embodiment 6, or a pharmaceutically acceptable salt, solvate, tautomer, stereoisomer or prodrug thereof, wherein the stereochemistry of the positions in Ring A identified by a wedge-shaped bond in the following structural fragment are as shown:

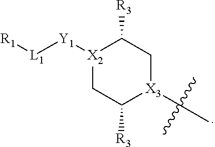

Embodiment 9. The compound according to any one of Embodiments 2 to 8, or a pharmaceutically acceptable salt, solvate, tautomer, stereoisomer or prodrug thereof, wherein R₃ at each occurrence is independently C₁₋₆ alkyl.

Embodiment 10. The compound according to any one of Embodiments 2 to 5, or a pharmaceutically acceptable salt, solvate, tautomer, stereoisomer or prodrug thereof, wherein m is 0.

Embodiment 11. The compound according to any one of Embodiments 2 to 10, or a pharmaceutically acceptable salt, solvate, tautomer, stereoisomer or prodrug thereof, wherein X₄, X₅ and X₆, and X₇ if present, are independently —CH—, —CR^{A}—, —C(O)—, —N—, —NR^{B}—, —O— or —S—, provided that the combination of X₄, X₅, X₆ and optionally X, together with the atoms to which they are attached forms an aromatic or heteroaromatic group.

Embodiment 12. The compound according to any one of Embodiments 2 to 11, or a pharmaceutically acceptable salt, solvate, tautomer, stereoisomer or prodrug thereof, wherein n is 1, and X₄, X₅, X₆ and X₇ are independently —CH—, —C(C₁₋₆ alkyl)- or —N—.

Embodiment 13. The compound according to Embodiment 12, or a pharmaceutically acceptable salt, solvate, tautomer, stereoisomer or prodrug thereof, wherein Ring B is defined as follows:

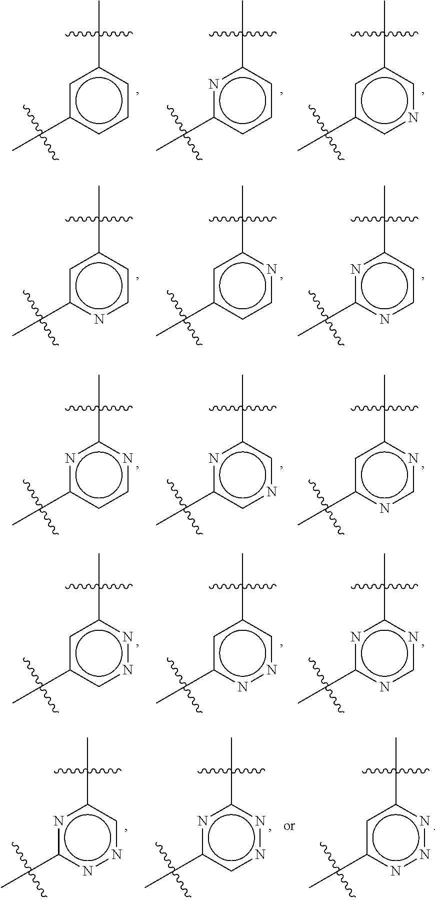

Embodiment 14. The compound according to Embodiment 13, or a pharmaceutically acceptable salt, solvate, tautomer, stereoisomer or prodrug thereof, wherein Ring B is defined as follows:

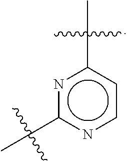

Embodiment 15. The compound according to any one of Embodiments 2 to 11, or a pharmaceutically acceptable salt, solvate, tautomer, stereoisomer or prodrug thereof, wherein n is 0, and X₄, X₅ and X₆ are independently —CH—, —CR^{A}—, —N—, —NR^{B}—, —O— or —S—, provided that the combination of X₄, X₅ and X₆ together with the atoms to which they are attached forms a heteroaromatic group.

Embodiment 16. The compound according to Embodiment 15, or a pharmaceutically acceptable salt, solvate, tautomer, stereoisomer or prodrug thereof, wherein: X₄ is —CH—, —C(C₁₋₆ alkyl)- or —N—; X₅ is —NH—, —N(C₁₋₆ alkyl)-, —O— or —S—; and X₆ is —CH—, —C(C₁₋₆ alkyl)- or —N—.

Embodiment 17. The compound according to Embodiment 16, or a pharmaceutically acceptable salt, solvate, tautomer, stereoisomer or prodrug thereof, wherein Ring B is defined as follows:

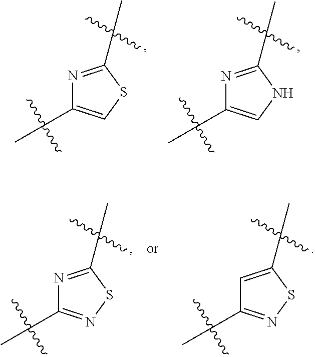 [SMILES: *c1csc(*)n1 ; *c1c[nH]c(*)n1 ; *c1nsc(*)n1 ; *c1cc(*)sn1]

Embodiment 18. The compound according to any one of Embodiments 2 to 17, or a pharmaceutically acceptable salt, solvate, tautomer, stereoisomer or prodrug thereof, wherein q is 0 to 4, and R₄, if present, at each occurrence is independently —F, —Cl, —Br, —I, —OH, C₁₋₆ alkyl, C₂₋₆ heteroalkyl or C₅₋₁₄aryl-C₂₋₆heteroalkyl, wherein each C₁₋₆ alkyl, C₂₋₆ heteroalkyl or C₅₋₁₄aryl-C₂₋₆heteroalkyl is unsubstituted.

Embodiment 19. The compound according to any one of Embodiments 2 to 18, or a pharmaceutically acceptable salt, solvate, tautomer, stereoisomer or prodrug thereof, wherein the stereochemistry of the position in Ring C identified by a wedge-shaped bond in the following structural fragment is as shown:

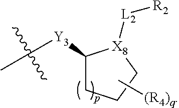

Embodiment 20. The compound according to any one of Embodiments 2 to 18, or a pharmaceutically acceptable salt, solvate, tautomer, stereoisomer or prodrug thereof, wherein the stereochemistry of the position in Ring C identified by a wedge-shaped bond in the following structural fragment is as shown:

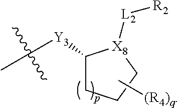

Embodiment 21. The compound according to any one of Embodiments 2 to 18, or a pharmaceutically acceptable salt, solvate, tautomer, stereoisomer or prodrug thereof, wherein q is 1, and Ring C is as shown in the following structural fragment:

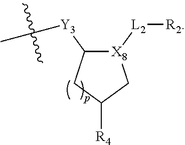

Embodiment 22. The compound according to Embodiment 21, or a pharmaceutically acceptable salt, solvate, tautomer, stereoisomer or prodrug thereof, wherein the stereochemistry of the positions in Ring C identified by a wedge-shaped bond in the following structural fragment are as shown:

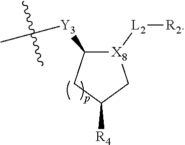

Embodiment 23. The compound according to Embodiment 21, or a pharmaceutically acceptable salt, solvate, tautomer, stereoisomer or prodrug thereof, wherein the stereochemistry of the positions in Ring C identified by a wedge-shaped bond in the following structural fragment are as shown:

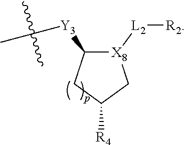

Embodiment 24. The compound according to Embodiment 21, or a pharmaceutically acceptable salt, solvate, tautomer, stereoisomer or prodrug thereof, wherein the stereochemistry of the positions in Ring C identified by a wedge-shaped bond in the following structural fragment are as shown:

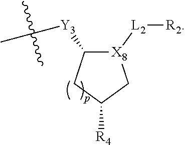

Embodiment 25. The compound according to Embodiment 21, or a pharmaceutically acceptable salt, solvate, tautomer, stereoisomer or prodrug thereof, wherein the stereochemistry of the positions in Ring C identified by a wedge-shaped bond in the following structural fragment are as shown:

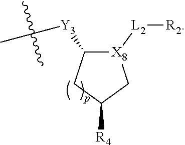

Embodiment 26. The compound according to any one of Embodiments 2 to 25, or a pharmaceutically acceptable salt, solvate, tautomer, stereoisomer or prodrug thereof, wherein p is 1.

Embodiment 27. The compound according to any one of Embodiments 2 to 25, or a pharmaceutically acceptable salt, solvate, tautomer, stereoisomer or prodrug thereof, wherein p is 2.

Embodiment 28. The compound according to any one of Embodiments 1 to 27, or a pharmaceutically acceptable salt, solvate, tautomer, stereoisomer or prodrug thereof, wherein R₄ at each occurrence is independently —OH, —OC₁₋₆ alkyl, or —O—CH₂-phenyl.

Embodiment 29. The compound according to any one of Embodiments 2 to 20, or a pharmaceutically acceptable salt, solvate, tautomer, stereoisomer or prodrug thereof, wherein q is 0.

Embodiment 30. The compound according to any one of Embodiments 2 to 29, or a pharmaceutically acceptable salt, solvate, tautomer, stereoisomer or prodrug thereof, wherein X₈ is —CH— or —N—.

Embodiment 31. The compound according to Embodiment 30, or a pharmaceutically acceptable salt, solvate, tautomer, stereoisomer or prodrug thereof, wherein X₃ is —N—.

Embodiment 32. The compound according to any one of Embodiments 1 to 31, or a pharmaceutically acceptable salt, solvate, tautomer, stereoisomer or prodrug thereof, wherein R₁ is C₁₋₆ alkyl, C₃₋₁₄ cycloalkyl, C₅₋₁₄ heterocycloalkenyl, C₅₋₁₄ aryl or C₅₋₁₄ heteroaryl, each of which is unsubstituted or substituted with one or more R^{A}.

Embodiment 33. The compound according to any one of Embodiments 1 to 32, or a pharmaceutically acceptable salt, solvate, tautomer, stereoisomer or prodrug thereof, wherein R₂ is C₁₋₆ alkyl, C₂₋₆ heteroalkyl, C₃₋₁₄ cycloalkyl, C₅₋₁₄ aryl or C₅₋₁₄ heteroaryl, each of which is unsubstituted or substituted with one or more R^{A}.

Embodiment 34. The compound according to any one of Embodiments 1 to 33, or a pharmaceutically acceptable salt, solvate, tautomer, stereoisomer or prodrug thereof, wherein R₅ and R₆ at each occurrence are independently —H, —F, —Cl, —Br, —I, —OH, C₁₋₆ alkyl or —OC₁₋₆ alkyl, wherein each C₁₋₆ alkyl or —OC₁₋₆ alkyl is unsubstituted; or R₅ and R₆ together with the carbon atom to which they are attached form a C₃₋₁₄ cycloalkyl, C₃₋₁₄ heterocycloalkyl, C₃₋₁₄ halocycloalkyl, or C₃₋₁₄ haloheterocycloalkyl.

Embodiment 35. The compound according to any one of Embodiments 1 to 34, or a pharmaceutically acceptable salt, solvate, tautomer, stereoisomer or prodrug thereof, wherein L₁ is optional and if present is C₁₋₆ alkyl or C₂₋₆ heteroalkyl, each of which is unsubstituted or substituted with one or more R^{A}.

Embodiment 36. The compound according to any one of Embodiments 1 to 35, or a pharmaceutically acceptable salt, solvate, tautomer, stereoisomer or prodrug thereof, wherein L₂ is optional and if present is C₁₋₆ alkyl or C₂₋₆ heteroalkyl, each of which is unsubstituted or substituted with one or more R^{A}.

Embodiment 37. The compound according to any one of Embodiments 1 to 36, or a pharmaceutically acceptable salt, solvate, tautomer, stereoisomer or prodrug thereof, wherein Y₁ is —CR₅R₆—, —C(O)—, —NR^{B}—, —O—, —S— or —S(O)₂—.

Embodiment 38. The compound according to any one of Embodiments 1 to 37, or a pharmaceutically acceptable salt, solvate, tautomer, stereoisomer or prodrug thereof, wherein Y₂ is —CR₅R₆—, —NR^{B}—, —O— or —S—.

Embodiment 39. The compound according to any one of Embodiments 1 to 38, or a pharmaceutically acceptable salt, solvate, tautomer, stereoisomer or prodrug thereof, wherein Y₃ is —CR₅R₆—, —C(O)—, —NR^{B}—, —O— or —S—.

Embodiment 40. The compound according to any one of Embodiments 1 to 39, or a pharmaceutically acceptable salt, solvate, tautomer, stereoisomer or prodrug thereof, wherein X₁ is —CR^{B}₂—, —NR^{B}—, —O— or —S—.

Embodiment 41. The compound according to any one of Embodiments 1 to 40, or a pharmaceutically acceptable salt, solvate, tautomer, stereoisomer or prodrug thereof, wherein R^{A} at each occurrence is independently —F, —Cl, —Br, —I, —NO₂, —OR^{B}, —SO₂R^{B}, C₁₋₆ alkyl, C₂₋₆ heteroalkyl, or C₃₋₁₄ cycloalkyl, wherein each C₁₋₆ alkyl, C₂₋₆ heteroalkyl, or C₃₋₁₄ cycloalkyl is unsubstituted or substituted with one or more —F, —Cl, —Br, —I, —CN, —NO₂, —SO₂, —N₃, —SCN, —NCS, —OR^{B}, —NR^{C}R^{D}, —NR^{B}NR^{C}R^{D}, —SR^{B}, —C(O)R^{B}, —C(O)OR^{B}, —C(O)NR^{C}R^{D}, —C(NR^{B})R^{B}, —C(NR^{B})NR^{C}R^{D}, —NR^{B}C(NR^{B})NR^{C}R^{D}, —S(O)R^{B}, —S(O)₂R^{B}, —S(O)OR^{B}, —S(O)₂OR^{B}, —NR^{B}NR^{C}R^{D} or —NR^{B}C(O)NR^{C}R^{D}; two R^{A} together with the atoms to which they are attached form a cyclic group; and/or two R^{A} on the same atom form (═O).

Embodiment 42. The compound according to any one of Embodiments 1 to 41, or a pharmaceutically acceptable salt, solvate, tautomer, stereoisomer or prodrug thereof, wherein R^{B} at each occurrence is independently (i) —H, or (ii) C₁₋₆ alkyl or C₅₋₁₄aryl-C₁₋₆alkyl, each of which is unsubstituted or substituted with one or more R^{E}.

Embodiment 43. The compound according to any one of Embodiments 1 to 42, or a pharmaceutically acceptable salt, solvate, tautomer, stereoisomer or prodrug thereof, wherein R^{C} and R^{D} at each occurrence are independently (i) —H, or (ii) C₁₋₆ alkyl, which is unsubstituted or substituted with one or more R^{E}, or R^{C} and R^{D} together with the nitrogen atom to which they are attached combine to form a heterocycle that is unsubstituted or substituted with one or more R^{E}.

Embodiment 44. The compound according to any one of Embodiments 1 to 43, or a pharmaceutically acceptable salt, solvate, tautomer, stereoisomer or prodrug thereof, wherein R^{E} at each occurrence is independently —F, —Cl, —Br, —I, —CN, —NO₂, —SO₂, —N₃, —SCN, —NCS, —OR^{F}, —NR^{G}R^{H}, —NR^{F}NR^{G}R^{H}, —SR^{F}, —C(O)R^{F}, —C(O)OR^{F}, —C(O)NR^{G}R^{H}, —C(NR^{F})R^{F}, —C(NR^{F})NR^{G}R^{H}, —S(O)R^{F}, —S(O)₂R^{F}, —S(O)OR^{F}, —S(O)₂OR^{F}, —NR^{F}NR^{G}R^{H}, —NR^{F}C(O)NR^{G}R^{H}, —NR^{F}C(NR^{F})NR^{G}R^{H}, C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl or C₅₋₁₄heteroaryl-C₁₋₆alkyl; two or more R^{E} together with the atoms to which they are attached form a cyclic group; and/or two R^{E} on the same atom form (═O).

Embodiment 45. The compound according to any one of Embodiments 1 to 44, or a pharmaceutically acceptable salt, solvate, tautomer, stereoisomer or prodrug thereof, wherein R^{F} at each occurrence is independently (i) —H, or (ii) C₁₋₆ alkyl.

Embodiment 46. The compound according to any one of Embodiments 1 to 45, or a pharmaceutically acceptable salt, solvate, tautomer, stereoisomer or prodrug thereof, wherein R^{G} and R^{H} at each occurrence are independently (i) —H, or (ii) C₁₋₆ alkyl, or R^{G} and R^{H} together with the nitrogen atom to which they are attached combine to form a heterocycle.

Embodiment 47. The compound according to any one of Embodiments 1 to 46, or a pharmaceutically acceptable salt, solvate, tautomer, stereoisomer or prodrug thereof, wherein R^{A} at each occurrence is independently —F, —Cl, —Br, —I, —NO₂, —OH, —SO₂R^{B}, C₁₋₆ alkyl, C₂₋₆ heteroalkyl or C₃₋₆ cycloalkyl, wherein each C₁₋₆ alkyl is unsubstituted or substituted with one or more —F, —Cl, —Br, —I, and each C₂₋₆ heteroalkyl or C₃₋₆ cycloalkyl is unsubstituted; two R^{A} together with the atom(s) to which they are attached form a cyclic group; and/or two R^{A} on the same atom form (═O).

Embodiment 48. The compound according to Embodiment 47, or a pharmaceutically acceptable salt, solvate, tautomer, stereoisomer or prodrug thereof, wherein R^{A} at each occurrence is independently —F, —Cl, —Br, —I, —NO₂, —OH, —OC₁₋₆ alkyl, —SO₂C₁₋₆alkyl, unsubstituted C₁₋₆ alkyl, C₁₋₆ alkyl substituted 1 to 3 times with —F, —Cl, —Br, —I, or C₃₋₆ cycloalkyl; two R^{A} together with the atom to which they are attached form a C₃₋₆ cycloalkyl; two R^{A} on adjacent atoms together are —O—(CH₂)_{y}—O—, wherein y is 1 or 2; and/or two R^{A} on the same atom form (═O).

Embodiment 49. The compound according to any one of Embodiments 1 to 48, or a pharmaceutically acceptable salt, solvate, tautomer, stereoisomer or prodrug thereof, wherein R^{B} at each occurrence is independently (i) —H, or (ii) C₁₋₆ alkyl or C₅₋₁₄aryl-C₁₋₆alkyl.

Embodiment 50. The compound according to Embodiment 49, or a pharmaceutically acceptable salt, solvate, tautomer, stereoisomer or prodrug thereof, wherein R^{B} at each occurrence is independently (i) —H, or (ii) C₁₋₆ alkyl.

Embodiment 51. The compound according to any one of Embodiments 1 to 50, or a pharmaceutically acceptable salt, solvate, tautomer, stereoisomer or prodrug thereof, wherein R^{C} and R^{D} at each occurrence are independently (i) —H, or (ii) C₁₋₆ alkyl, which is unsubstituted, or R^{C} and R^{D} together with the nitrogen atom to which they are attached combine to form a heterocycle that is unsubstituted.

Embodiment 52. The compound according to Embodiment 51, or a pharmaceutically acceptable salt, solvate, tautomer, stereoisomer or prodrug thereof, wherein R^{C} and R^{D} at each occurrence are independently (i) —H, or (ii) C₁₋₆ alkyl.

Embodiment 53. The compound according to any one of Embodiments 1 to 52, or a pharmaceutically acceptable salt, solvate, tautomer, stereoisomer or prodrug thereof, wherein L₁ is absent, or L₁ is present and is C₁₋₄ alkyl or C₂₋₄ heteroalkyl.

Embodiment 54. The compound according to any one of Embodiments 1 to 53, or a pharmaceutically acceptable salt, solvate, tautomer, stereoisomer or prodrug thereof, wherein L₂ is present and is C₁₋₄ alkyl which is unsubstituted or substituted with one C₁₋₆ alkyl group, —C(O)— or —C(O)O—.

Embodiment 55. The compound according to any one of Embodiments 1 to 53, or a pharmaceutically acceptable salt, solvate, tautomer, stereoisomer or prodrug thereof, wherein L₂ is present and is unsubstituted C₁₋₄ alkyl, —(CH(C₁₋₆ alkyl))-, —C(O)— or —C(O)O—.

Embodiment 56. The compound according to any one of Embodiments 1 to 55, or a pharmaceutically acceptable salt, solvate, tautomer, stereoisomer or prodrug thereof, wherein X₁ is —CH₂—, —NH—, —N(C₁₋₆ alkyl)-, —O— or —S—.

Embodiment 57. The compound according to Embodiment 56, or a pharmaceutically acceptable salt, solvate, tautomer, stereoisomer or prodrug thereof, wherein X₁ is —NH— or —N(C₁₋₆ alkyl)-.

Embodiment 58. The compound according to Embodiment 57, or a pharmaceutically acceptable salt, solvate, tautomer, stereoisomer or prodrug thereof, wherein X₁ is —NH—.

Embodiment 59. The compound according to any one of Embodiments 1 to 58, or a pharmaceutically acceptable salt, solvate, tautomer, stereoisomer or prodrug thereof, wherein R₁ is C₁₋₆ alkyl, C₃₋₁₀ cycloalkyl, C₅₋₁₀ heterocycloalkenyl, C₆₋₁₀ aryl or C₅₋₁₀ heteroaryl, each of which is unsubstituted or substituted one or more times, independently, with —F, —Cl, —Br, —I, —NO₂, —OC₁₋₆ alkyl, unsubstituted C₁₋₆ alkyl, or C₁₋₆ alkyl substituted 1 to 3 times, independently, with —F, —Cl, —Br or —I; or C₆ aryl or C₅₋₆ heteroaryl, wherein two adjacent atoms of the aryl or heteroaryl ring are bonded to the group —O—(CH₂)_{y}—O—, wherein y is 1 or 2.

Embodiment 60. The compound according to any one of Embodiments 1 to 59, or a pharmaceutically acceptable salt, solvate, tautomer, stereoisomer or prodrug thereof, wherein R₂ is C₁₋₆ alkyl, C₂₋₆ heteroalkyl, C₃₋₆ cycloalkyl, C₆₋₁₀ aryl or C₅₋₁₀ heteroaryl, each of which is unsubstituted or substituted one or more times, independently, with —F, —Cl, —Br, —I, —SO₂C₁₋₆ alkyl, —OC₁₋₆ alkyl, unsubstituted C₁₋₆ alkyl, unsubstituted C₃₋₆ cycloalkyl, or C₁₋₆ alkyl substituted 1 to 3 times, independently, with —F, —Cl, —Br or —I; or C₆ aryl or C₅₋₆ heteroaryl, wherein two adjacent atoms of the aryl or heteroaryl ring are bonded to the group —O—(CH₂)_{y}—O—, wherein y is 1 or 2.

Embodiment 61. The compound according to any one of Embodiments 1 to 60, or a pharmaceutically acceptable salt, solvate, tautomer, stereoisomer or prodrug thereof, wherein R₁ is:

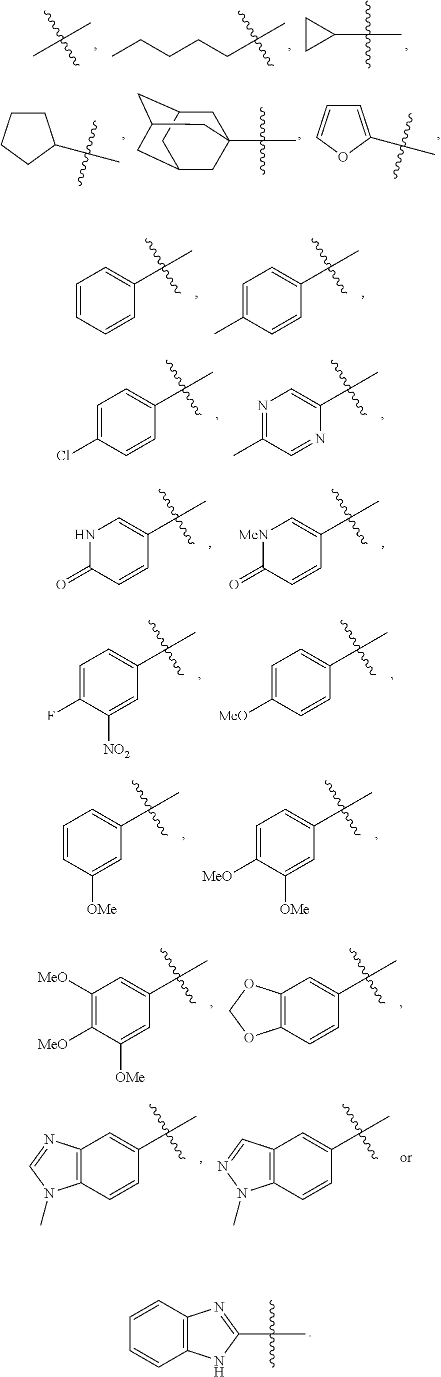

Embodiment 62. The compound according to any one of Embodiments 1 to 61, or a pharmaceutically acceptable salt, solvate, tautomer, stereoisomer or prodrug thereof, wherein R₂ is:

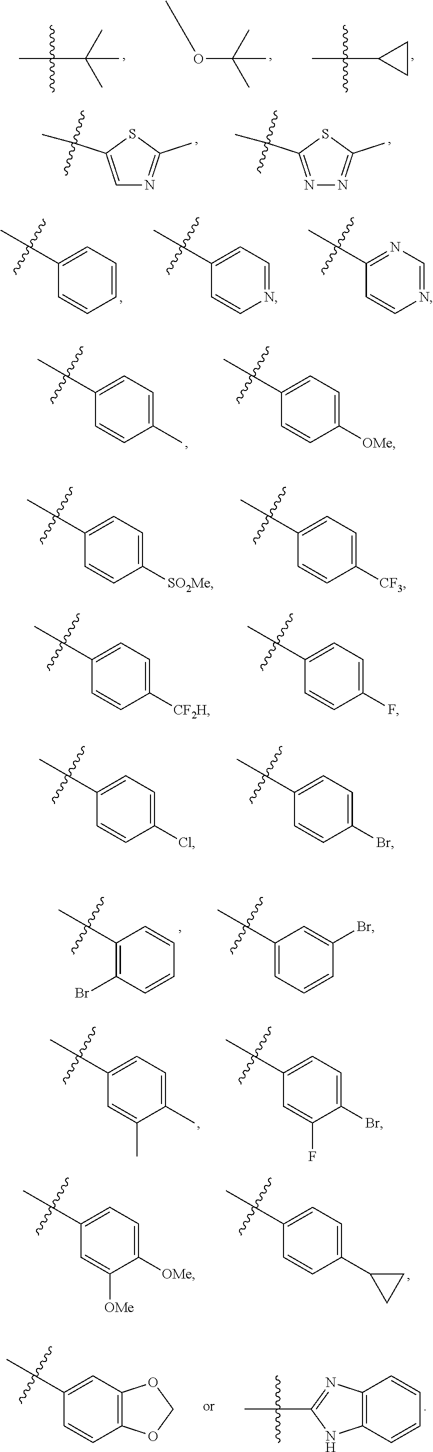

Embodiment 63. The compound according to any one of Embodiments 1 to 62, or a pharmaceutically acceptable salt, solvate, tautomer, stereoisomer or prodrug thereof, wherein R₅ and R₆ at each occurrence are independently —H or unsubstituted C₁₋₆ alkyl; or R₅ and R₆ together with the atom to which they are attached form a C₃₋₆ cycloalkyl.

Embodiment 64. The compound according to any one of Embodiments 1 to 63, or a pharmaceutically acceptable salt, solvate, tautomer, stereoisomer or prodrug thereof, wherein Y₁ is —CH₂—, —CH(C₁₋₆ alkyl)-, —C(C₁₋₆ alkyl)₂-, —C(O)— or —S(O)₂—.

Embodiment 65. The compound according to Embodiment 64, or a pharmaceutically acceptable salt, solvate, tautomer, stereoisomer or prodrug thereof, wherein Y₁ is —CH₂—, —C(O)— or —S(O)₂—.

Embodiment 66. The compound according to any one of Embodiments 1 to 65, or a pharmaceutically acceptable salt, solvate, tautomer, stereoisomer or prodrug thereof, wherein Y₂ is —CH₂—, —CH(C₁₋₆ alkyl)-, —C(C₁₋₆ alkyl)₂-, or is as shown in the following structural fragment:

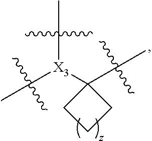

wherein z is 0 to 3.

Embodiment 67. The compound according to any one of Embodiments 1 to 65, or a pharmaceutically acceptable salt, solvate, tautomer, stereoisomer or prodrug thereof, wherein Y₂ is —CHR₅— and the stereochemistry of Y₂ is as shown in the following structural fragment:

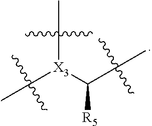

Embodiment 68. The compound according to any one of Embodiments 1 to 65, or a pharmaceutically acceptable salt, solvate, tautomer, stereoisomer or prodrug thereof, wherein Y₂ is —CHR₅— and the stereochemistry of Y₂ is as shown in the following structural fragment:

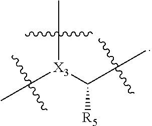

Embodiment 69. The compound according to any one of Embodiments 1 to 68, or a pharmaceutically acceptable salt, solvate, tautomer, stereoisomer or prodrug thereof, wherein Y₃ is —CH₂—, —CH(C₁₋₆ alkyl)-, —C(C₁₋₆ alkyl)₂- or —C(O)—.

Embodiment 70. The compound according to Embodiment 69, or a pharmaceutically acceptable salt, solvate, tautomer, stereoisomer or prodrug thereof, wherein Y₃ is —CH₂— or —C(O)—.

Embodiment 71. The compound according to any one of Embodiments 1 to 70, or a pharmaceutically acceptable salt, solvate, tautomer, stereoisomer or prodrug thereof, wherein L₁ and R₁ together are:

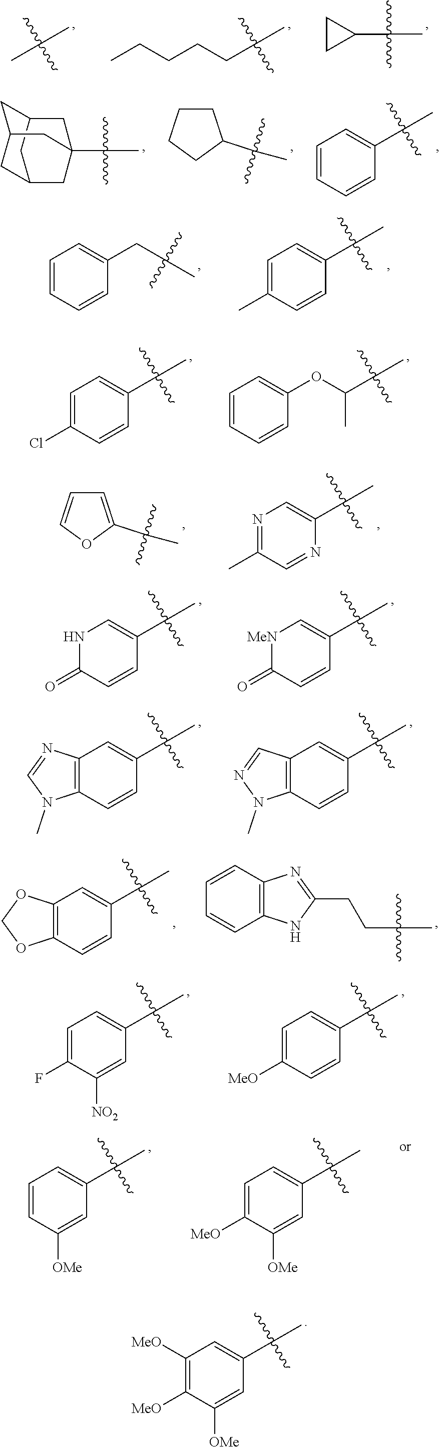

Embodiment 72. The compound according to any one of Embodiments 1 to 71, or a pharmaceutically acceptable salt, solvate, tautomer, stereoisomer or prodrug thereof, wherein L₂ is —CH(C₁₋₆ alkyl)- and the stereochemistry of L₂ is as shown in the following structural fragment:

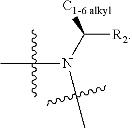

Embodiment 73. The compound according to any one of Embodiments 1 to 71, or a pharmaceutically acceptable salt, solvate, tautomer, stereoisomer or prodrug thereof, wherein L₂ is —CH(C₁₋₆ alkyl)- and the stereochemistry of L₂ is as shown in the following structural fragment:

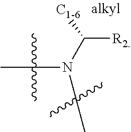

Embodiment 74. The compound according to any one of Embodiments 1 to 71, or a pharmaceutically acceptable salt, solvate, tautomer, stereoisomer or prodrug thereof, wherein L₂ and R₂ together are:

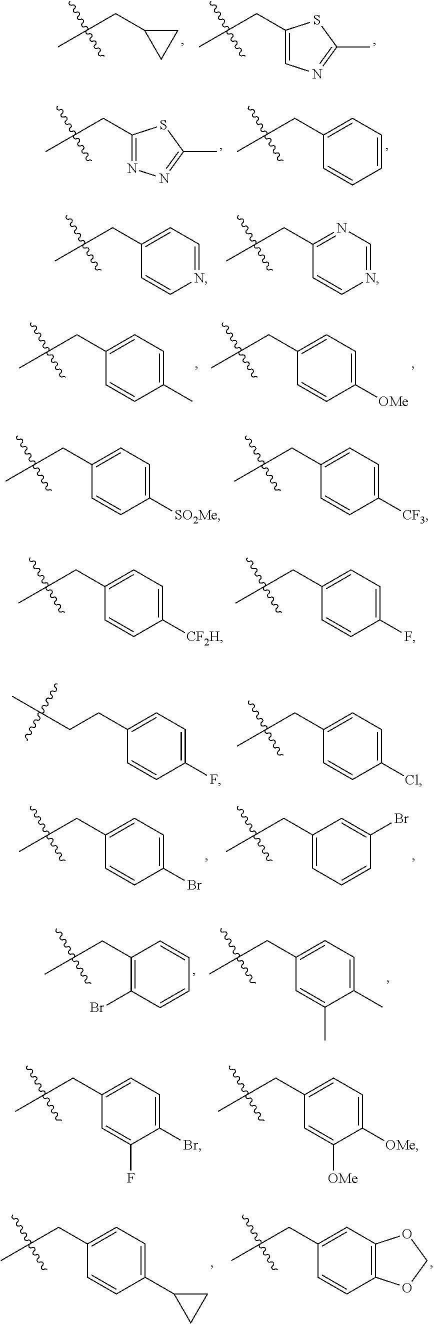

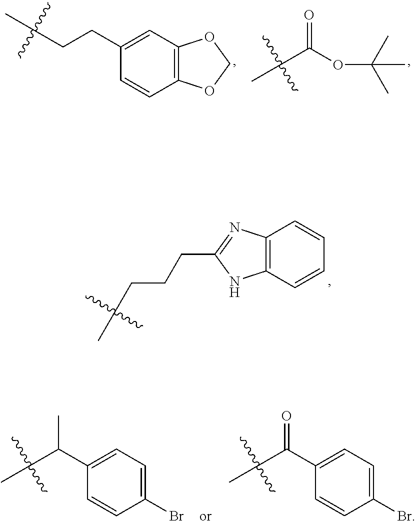

Embodiment 75. The compound according to Embodiment 1, wherein the compound is as defined in Table 2, or a pharmaceutically acceptable salt, solvate, tautomer, stereoisomer or prodrug thereof.

Embodiment 76. The compound according to Embodiment 1, wherein the compound is as defined in Table 3, or a pharmaceutically acceptable salt, solvate, tautomer, stereoisomer or prodrug thereof.

Embodiment 77. The compound according to Embodiment 1, wherein the compound is as defined in Table 4, or a pharmaceutically acceptable salt, solvate, tautomer, stereoisomer or prodrug thereof.

Embodiment 78. The compound according to Embodiment 1, wherein the compound is as defined in Table 5, or a pharmaceutically acceptable salt, solvate, tautomer, stereoisomer or prodrug thereof.

Embodiment 79. The compound according to Embodiment 1, wherein the compound is as defined in Table 6, or a pharmaceutically acceptable salt, solvate, tautomer, stereoisomer or prodrug thereof.

Embodiment 80. The compound according to Embodiment 1, wherein the compound is as defined in Table 7, or a pharmaceutically acceptable salt, solvate, tautomer, stereoisomer or prodrug thereof.

Embodiment 81. The compound according to Embodiment 1, wherein the compound is:

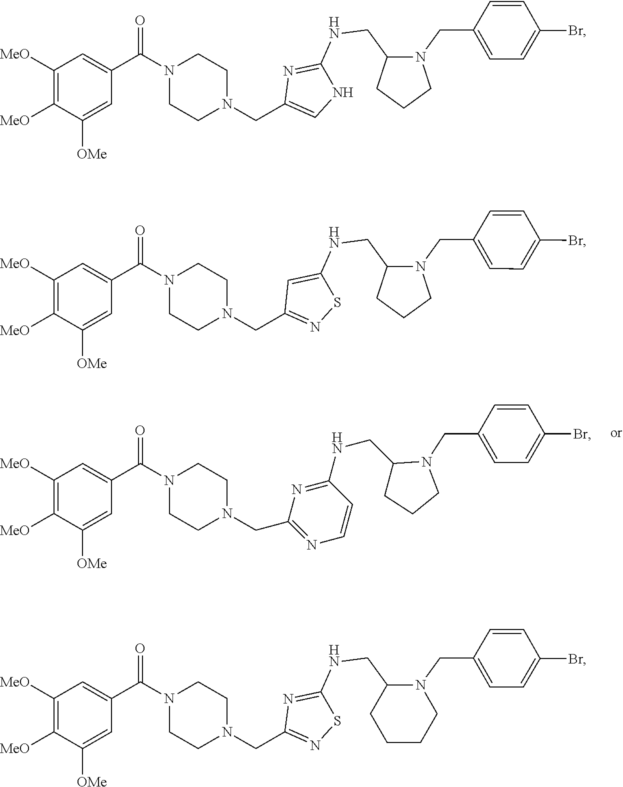 [SMILES: COc1cc(C(=O)N2CCN(Cc3c[nH]c(NCC4CCCN4Cc4ccc(Br)cc4)n3)CC2)cc(OC)c1OC ; COc1cc(C(=O)N2CCN(Cc3cc(NCC4CCCN4Cc4ccc(Br)cc4)sn3)CC2)cc(OC)c1OC ; COc1cc(C(=O)N2CCN(Cc3nccc(NCC4CCCN4Cc4ccc(Br)cc4)n3)CC2)cc(OC)c1OC ; "or" ; COc1cc(C(=O)N2CCN(Cc3nsc(NCC4CCCCN4Cc4ccc(Br)cc4)n3)CC2)cc(OC)c1OC]

or a pharmaceutically acceptable salt, solvate, tautomer, stereoisomer or prodrug thereof.

Embodiment 82. The compound according to Embodiment 1, wherein the compound is compound: 2678-2, 2678-3, 2678-5, 2678-10,2678-32, 2678-58, 2678-66, 2678-72, 2678-51, 2678-3, 2678-78, or 2678-80, or a pharmaceutically acceptable salt, solvate, tautomer, stereoisomer or prodrug thereof.

Embodiment 83. The compound according to Embodiment 1, wherein the compound is compound: A701, B708, B709, or B710, or a pharmaceutically acceptable salt, solvate, tautomer, stereoisomer or prodrug thereof.

Embodiment 84. The compound according to Embodiment 1, wherein the compound is compound: A607, A702, A704, B314, B316, B317, B318, B319, B708, B710, B801, B802, B803, B804, B805, B806, B908, B909, B910, or B911, or a pharmaceutically acceptable salt, solvate, tautomer, stereoisomer or prodrug thereof.

Embodiment 85. The compound according to Embodiment 1, wherein the compound is B319, or a pharmaceutically acceptable salt, solvate, tautomer, stereoisomer or prodrug thereof.

Embodiment 86. A pharmaceutical composition comprising the compound according to any one of Embodiments 1 to 85, or a pharmaceutically acceptable salt, solvate, tautomer, stereoisomer or prodrug thereof, and a pharmaceutically acceptable carrier, excipient and/or diluent.

Embodiment 87. A pharmaceutical composition comprising the compound according to any one of Embodiments 1 to 85, or a pharmaceutically acceptable salt, solvate, tautomer, stereoisomer or prodrug thereof, and an additional therapeutic agent.

Embodiment 88. A method of treating a disease or condition that is amenable to treatment by blocking PD-1, PD-L1 and/or the PD-1/PD-L1 interaction, wherein the method comprises administering a compound according to any one of Embodiments 1 to 85, or a pharmaceutically acceptable salt, solvate, tautomer, stereoisomer or prodrug thereof, or a compound which is

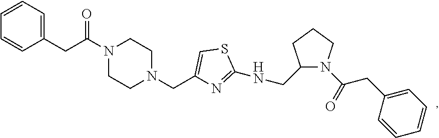 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof, to a subject in need thereof.

Embodiment 89. The method of Embodiment 88, further comprising administering an additional therapeutic agent to the subject.

Embodiment 90. The method of Embodiment 89, wherein the compound or pharmaceutically acceptable salt, solvate, tautomer, stereoisomer or prodrug thereof, is administered before, after or simultaneously with the additional therapeutic agent.

Embodiment 91. Use of a compound according to any one of Embodiments 1 to 85, or a pharmaceutically acceptable salt, solvate, tautomer, stereoisomer or prodrug thereof, or a compound which is

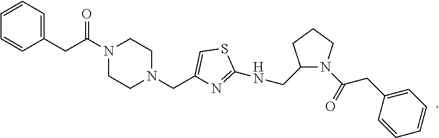 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof, for the treatment of a disease or condition that is amenable to treatment by blocking PD-1, PD-L1 and/or the PD-1/PD-L1 interaction in a subject in need thereof.

Embodiment 92. The use of Embodiment 91, further comprising the use of an additional therapeutic agent.

Embodiment 93. The use of Embodiment 92, wherein the compound or pharmaceutically acceptable salt, solvate, tautomer, stereoisomer or prodrug thereof, is for administration before, after or simultaneously with the additional therapeutic agent.

Embodiment 94. Use of a compound according to any one of Embodiments 1 to 85, or a pharmaceutically acceptable salt, solvate, tautomer, stereoisomer or prodrug thereof, or a compound which is

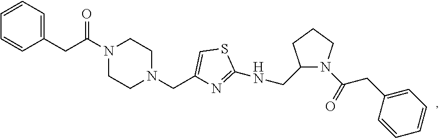 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof, for the manufacture of a medicament for the treatment of a disease or condition that is amenable to treatment by blocking PD-1, PD-L1 and/or the PD-1/PD-L1 interaction.

Embodiment 95. A method of treating cancer, wherein the method comprises administering a compound according to any one of Embodiments 1 to 85, or a pharmaceutically acceptable salt, solvate, tautomer, stereoisomer or prodrug thereof, or a compound which is

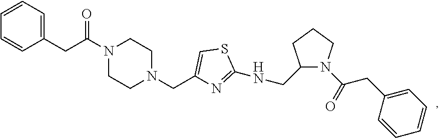 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof, to a subject in need thereof.

Embodiment 96. The method of Embodiment 95, further comprising administering an additional therapeutic agent to the subject.

Embodiment 97. The method of Embodiment 96, wherein the compound or pharmaceutically acceptable salt, solvate, tautomer, stereoisomer or prodrug thereof, is administered before, after or simultaneously with the additional therapeutic agent.

Embodiment 98. Use of a compound according to any one of Embodiments 1 to 85, or a pharmaceutically acceptable salt, solvate, tautomer, stereoisomer or prodrug thereof, or a compound which is

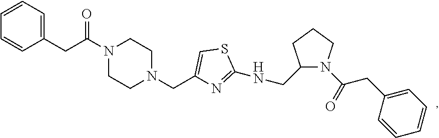 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof, for the treatment of cancer in a subject in need thereof.

Embodiment 99. The use of Embodiment 98, further comprising the use of an additional therapeutic agent.

Embodiment 100. The use of Embodiment 99, wherein the compound or pharmaceutically acceptable salt, solvate, tautomer, stereoisomer or prodrug thereof, is for administration before, after or simultaneously with the additional therapeutic agent.

Embodiment 101. Use of a compound according to any one of Embodiments 1 to 85, or a pharmaceutically acceptable salt, solvate, tautomer, stereoisomer or prodrug thereof, or a compound which is

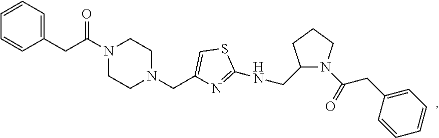 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof, for the manufacture of a medicament for the treatment of cancer.

Embodiment 102. A method of treating sepsis, wherein the method comprises administering a compound according to any one of Embodiments 1 to 85, or a pharmaceutically acceptable salt, solvate, tautomer, stereoisomer or prodrug thereof, or a compound which is

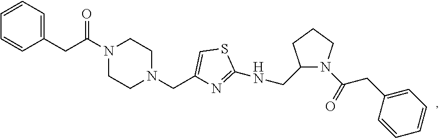 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof, to a subject in need thereof.

Embodiment 103. The method of Embodiment 102, further comprising administering an additional therapeutic agent to the subject.

Embodiment 104. The method of Embodiment 103, wherein the compound or pharmaceutically acceptable salt, solvate, tautomer, stereoisomer or prodrug thereof, is administered before, after or simultaneously with the additional therapeutic agent.

Embodiment 105. Use of a compound according to any one of Embodiments 1 to 85, or a pharmaceutically acceptable salt, solvate, tautomer, stereoisomer or prodrug thereof, or a compound which is

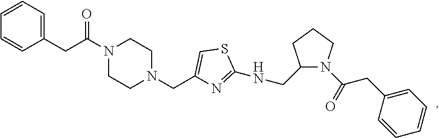 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof, for the treatment of sepsis in a subject in need thereof.

Embodiment 106. The use of Embodiment 105, further comprising the use of an additional therapeutic agent.

Embodiment 107. The use of Embodiment 106, wherein the compound or pharmaceutically acceptable salt, solvate, tautomer, stereoisomer or prodrug thereof, is for administration before, after or simultaneously with the additional therapeutic agent.

Embodiment 108. Use of a compound according to any one of Embodiments 1 to 85, or a pharmaceutically acceptable salt, solvate, tautomer, stereoisomer or prodrug thereof, or a compound which is

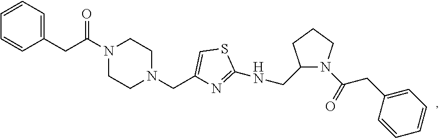 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof, for the manufacture of a medicament for the treatment of sepsis.

Embodiment 109. A method of treating an autoimmune disease, wherein the method comprises administering a compound according to any one of Embodiments 1 to 85, or a pharmaceutically acceptable salt, solvate, tautomer, stereoisomer or prodrug thereof, or a compound which is

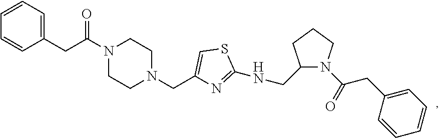 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof, to a subject in need thereof.

Embodiment 110. The method of Embodiment 109, further comprising administering an additional therapeutic agent to the subject.

Embodiment 111. The method of Embodiment 110, wherein the compound or pharmaceutically acceptable salt, solvate, tautomer, stereoisomer or prodrug thereof, is administered before, after or simultaneously with the additional therapeutic agent.

Embodiment 112. Use of a compound according to any one of Embodiments 1 to 85, or a pharmaceutically acceptable salt, solvate, tautomer, stereoisomer or prodrug thereof, or a compound which is

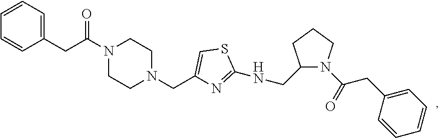 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof, for the treatment of an autoimmune disease in a subject in need thereof.

Embodiment 113. The use of Embodiment 112, further comprising the use of an additional therapeutic agent.

Embodiment 114. The use of Embodiment 113, wherein the compound or pharmaceutically acceptable salt, solvate, tautomer, stereoisomer or prodrug thereof, is for administration before, after or simultaneously with the additional therapeutic agent.

Embodiment 115. Use of a compound according to any one of Embodiments 1 to 85, or a pharmaceutically acceptable salt, solvate, tautomer, stereoisomer or prodrug thereof, or a compound which is

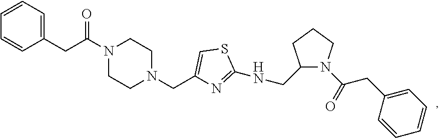 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof, for the manufacture of a medicament for the treatment of an autoimmune disease.

Embodiment 116. A compound according to any one of Embodiments 1 to 85, or a pharmaceutically acceptable salt, solvate, tautomer, stereoisomer or prodrug thereof, for use in a method of therapy.

Embodiment 117. A kit comprising: (a) a compound according to any one of Embodiments 1 to 85, or a pharmaceutically acceptable salt, solvate, tautomer, stereoisomer or prodrug thereof; and (b) an additional therapeutic agent.

Embodiment 118. A kit comprising: (a) a compound according to any one of Embodiments 1 to 85, or a pharmaceutically acceptable salt, solvate, tautomer, stereoisomer or prodrug thereof, or a compound which is

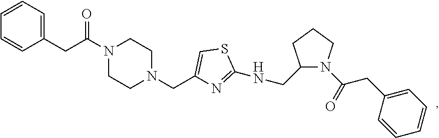 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof; and (b) instructions for using the compound or pharmaceutically acceptable salt, solvate, tautomer, stereoisomer or prodrug thereof to treat a disease or condition that is amenable to treatment by blocking PD-1, PD-L1 and/or the PD-1/PD-L1 interaction.

Embodiment 119. A kit comprising: (a) a compound according to any one of Embodiments 1 to 85, or a pharmaceutically acceptable salt, solvate, tautomer, stereoisomer or prodrug thereof, or a compound which is

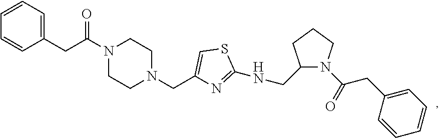 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof; and (b) instructions for using the compound or pharmaceutically acceptable salt, solvate, tautomer, stereoisomer or prodrug thereof to treat cancer.

Embodiment 120. A kit comprising: (a) a compound according to any one of Embodiments 1 to 85, or a pharmaceutically acceptable salt, solvate, tautomer, stereoisomer or prodrug thereof, or a compound which is

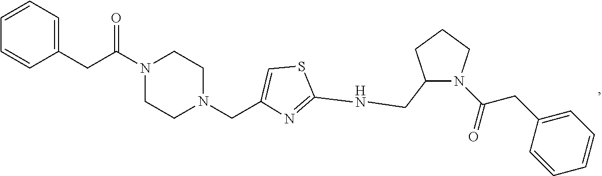 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof; and (b) instructions for using the compound or pharmaceutically acceptable salt, solvate, tautomer, stereoisomer or prodrug thereof to treat sepsis.

Embodiment 121. A kit comprising: (a) a compound according to any one of Embodiments 1 to 85, or a pharmaceutically acceptable salt, solvate, tautomer, stereoisomer or prodrug thereof, or a compound which is

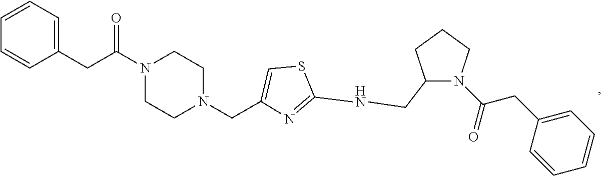 [SMILES: O=C(Cc1ccccc1)N1CCN(Cc2csc(NCC3CCCN3C(=O)Cc3ccccc3)n2)CC1]

or a pharmaceutically acceptable salt, solvate, tautomer, stereoisomer or prodrug thereof; and (b) instructions for using the compound or pharmaceutically acceptable salt, solvate, tautomer, stereoisomer or prodrug thereof to treat an autoimmune disease.

The present disclosure is further illustrated by the following non-limiting examples.

### EXAMPLES

Compound Design

The compounds of the disclosure were designed through the use of a high-throughput screening method using mixture-based libraries combined with positional scanning deconvolution.

The method used, which is related to that described in Pinilla C, et al. “Selective agonists and antagonists of formylpeptide receptors: duplex flow cytometry and mixture-based positional scanning libraries” (2013) Mol Pharmacol., 84(3), 314-324, doi:10.1124/mol.113.086595, allows for the identification of specific functionalities at each variable position of a chemical scaffold that may be responsible for driving the activity of the scaffold. The general method is also described in Pinilla C et al. “Rapid identification of high affinity peptide ligands using positional scanning synthetic peptide combinatorial libraries” (1992) Biotechniques, 13(6), 901; U.S. Pat. No. 5,556,762; and EP 0558674.

### Exemplary Preparations of Compounds of the Disclosure

Compounds of the disclosure can be prepared using known organic synthesis techniques and can be synthesized according to any of numerous possible synthetic routes, such as those shown in Schemes 1 to 3 (shown and described below).

Unless otherwise stated, the starting materials (reactants), solvents, and reagents may be purchased from commercial sources or synthesised by procedures known in the art.

The reactions for preparing compounds of the disclosure can be carried out in suitable solvents which can be readily selected by a person skilled in the art of organic synthesis. Suitable solvents can be substantially non-reactive with the starting materials, the reagents, the intermediates or products at the temperatures at which the reactions are carried out. A given reaction can be carried out in one solvent or a mixture of more than one solvent. Depending on the particular reaction step, suitable solvents for a particular reaction step, as well as the quantity of the solvents required, can be selected by the skilled person.

Reactions can be monitored according to any suitable method known in the art. For example, product formation can be monitored by spectroscopic means, such as nuclear magnetic resonance spectroscopy (e.g., ¹H or ¹³C NMR), infrared spectroscopy, spectrophotometry (e.g., UV-visible), mass spectrometry or by chromatographic methods such as high performance liquid chromatography (HPLC) or thin layer chromatography (TLC).

Schemes 1, 2 and 3 (below), which describe syntheses of compounds 2678-2, 2678-10 and 2678-53, provide general guidance in connection with preparing compounds of the disclosure. A person of ordinary skill in the art would understand that the preparations shown in the Schemes can be modified or optimized using general knowledge of organic chemistry to prepare various compounds of the disclosure.

Preparation of Compound 2678-2

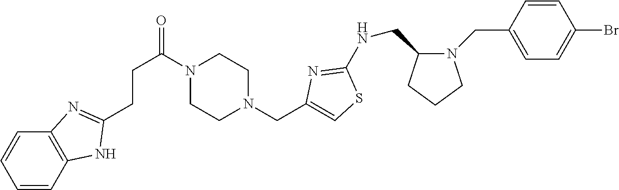 [SMILES: O=C(CCc1nc2ccccc2[nH]1)N1CCN(Cc2csc(NC[C@@H]3CCCN3Cc3ccc(Br)cc3)n2)CC1]

Compound 2678-2 may be synthesised as shown in Scheme 1.

The synthetic schemes and preparations disclosed herein are provided merely for the purpose of illustration and are non-limiting. For example, while steps 1 to 4 of Scheme 1 have been described in a specific order, this is not an indication that the steps necessarily must be carried out in the recited order. For example, step 3 may be carried out prior to step 1.

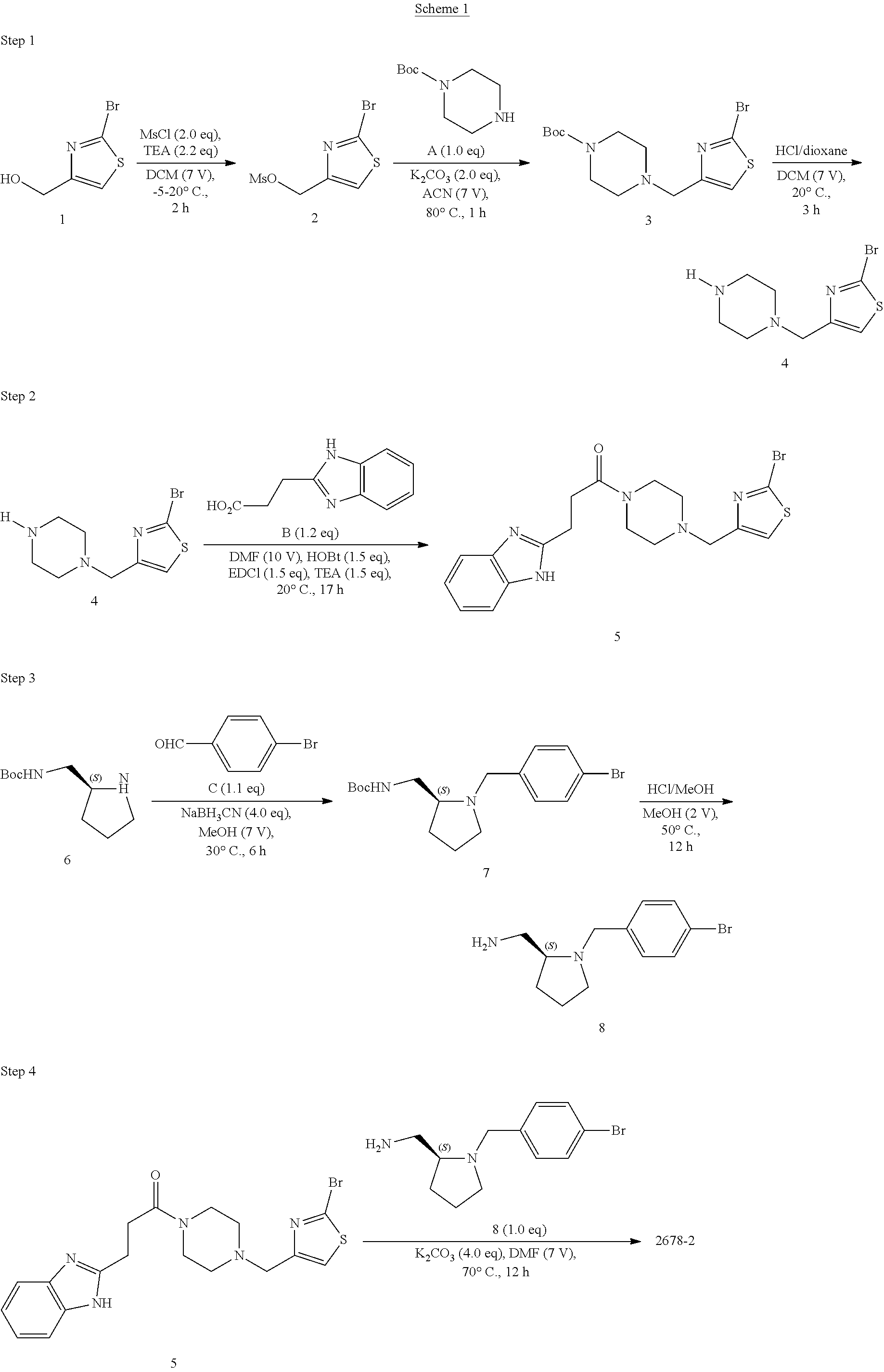

In step 1 of Scheme 1, 2-bromo-4-hydroxymethylthiazole (1) is reacted with 2.0 equivalents of methanesulfonyl chloride (MsCl) in the presence of 2.2 equivalents of triethylamine (TEA) in dichloromethane (DCM) to generate intermediate 2. Intermediate 2 is then reacted with 1.0 equivalent of 1-Boc-piperazine (A) in the presence of 2.0 equivalents of potassium carbonate (K₂CO₃) in acetonitrile (ACN) at elevated temperature (80° C.) to generate intermediate 3, which is subsequently deprotected using hydrochloric acid in dioxane (HCl/dioxane) in DCM to generate intermediate 4.

In step 2 of Scheme 1, intermediate 5 is obtained via the reaction of intermediate 4 with 1.2 equivalents of 2-benzimidazolepropionic acid (B) in the presence of 1.5 equivalents of hydroxybenzotriazole (HOBt), 1.5 equivalents of 1-ethyl-3-(3-dimethylaminopropyl)carbodiimide (EDCI) and 1.5 equivalents of triethylamine (TEA) in dimethylformamide (DMF).

Steps 1 and 2 of Scheme 1 may also be carried out using modified conditions. For example, in step 1, deprotection of intermediate 3 may be carried out in ethyl acetate (2 V) at 20° C. (reaction time: 0.5 h) using hydrochloric acid in ethyl acetate (HCl/EtOAc) to generate intermediate 4, as well as compound 4′ which corresponds in structure to intermediate 4 but contains a chlorine rather than bromine atom on the 5-membered ring. In step 2, intermediate 5 may obtained via the reaction of intermediate 4 with 1.2 equivalents of 2-benzimidazolepropionic acid (B) in the presence of 1.5 equivalents of HOBt, 1.5 equivalents of EDCI and 4 equivalents of TEA in DMF (5 V) (reaction time: 15 h; temperature: 20° C.).

In step 3 of Scheme 1, (S)-2-N-Boc-aminomethylpyrrolidine (6) is reacted with 1.1 equivalents of 4-bromobenzaldehyde (C) in the presence of 4.0 equivalents of sodium cyanoborohydride (NaBH₃CN) in methanol to generate intermediate 7, which is deprotected with hydrochloric acid in methanol (HCl/MeOH) at elevated temperature (50° C.) using methanol (MeOH) as a solvent.

In step 4 of Scheme 1, compound 2678-2 is obtained via the reaction of intermediate 5 with 1.0 equivalent of intermediate 8 in the presence of 4.0 equivalents of potassium carbonate (K₂CO₃) in dimethylformamide at elevated temperature (70° C.).

Alternatively, in step 4, compound 2678-2 may obtained via the reaction of intermediate 5 with 1.0 equivalent of intermediate 8 in the presence of 2.0 equivalents of cesium carbonate (Cs₂CO₃), 0.1 equivalents of tris(dibenzylideneacetone) dipalladium(0) and 0.2 equivalents BrettPhos in DMF (10 V) at elevated temperature (temperature: 110° C.; reaction time: 12 h).

Preparation of Compound 2678-10

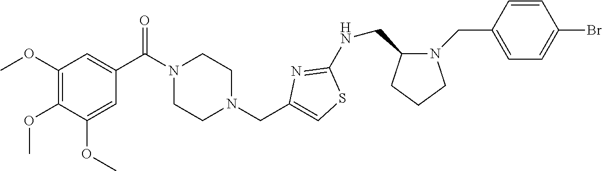 [SMILES: COc1cc(C(=O)N2CCN(Cc3csc(NC[C@@H]4CCCN4Cc4ccc(Br)cc4)n3)CC2)cc(OC)c1OC]

Compound 2678-10 may be synthesised as shown in Scheme 2.

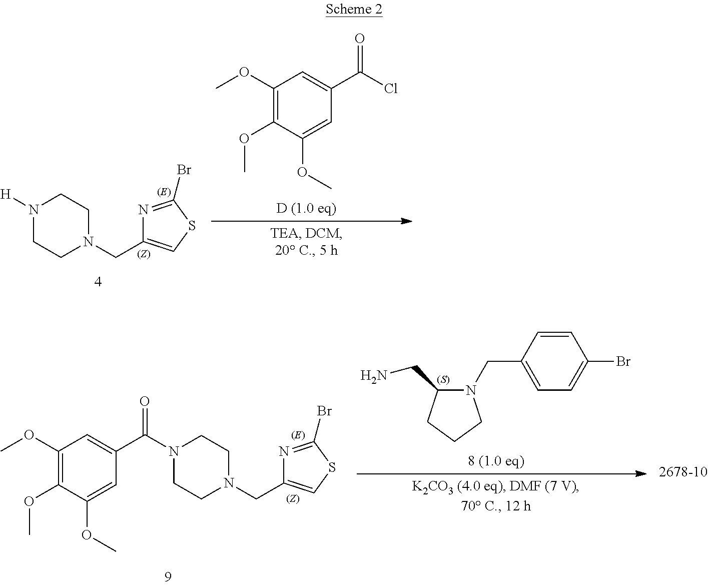

Intermediates 4 and 8 may be synthesised as shown in Scheme 1, steps 1 and 3, respectively.

In Scheme 2, intermediate 9 is synthesised from intermediate 4 and 1.0 equivalent of 3,4,5-trimethoxybenzoyl chloride (D) in the presence of triethylamine (TEA) in dichloromethane (DCM). Intermediate 9 is then reacted with 1.0 equivalent of intermediate 8 in the presence of 4.0 equivalents of potassium carbonate (K₂CO₃) in dimethylformamide (DMF) at elevated temperature (70° C.) to generate compound 2678-10.

Alternatively, intermediate 9 may be synthesised from intermediate 4 and 1.0 equivalent of 3,4,5-trimethoxybenzoyl chloride (D) in the presence of 3 equivalents of TEA in DCM (6 V) in a reaction carried out for 3 h at 20° C., and then reacted with 1.0 equivalent of intermediate 8 in the presence of 2.0 equivalents of cesium carbonate (Cs₂CO₃), 0.1 equivalents of tris(dibenzylideneacetone) dipalladium(0) and 0.2 equivalents BrettPhos in DMF (10 V) at elevated temperature (temperature: 110° C.; reaction time: 12 h) to generate compound 2678-10.

Preparation of Compound 2678-53

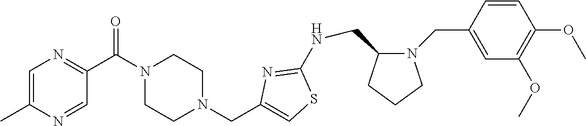

Compound 2678-53 may be synthesised as shown in Scheme 3. While steps 1 to 3 of Scheme 3 are described in a specific order, this is not an indication that the steps necessarily must be carried out in the recited order. For example, step 2 may be carried out prior to step 1.

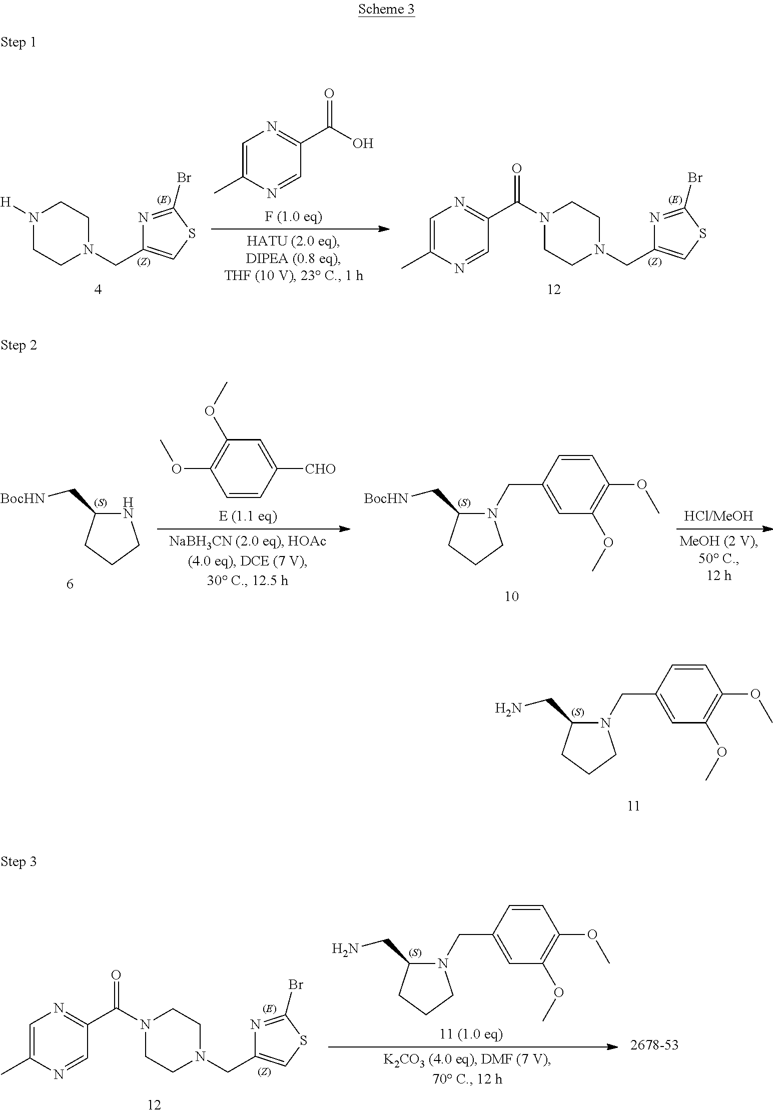

Intermediate 4 may be synthesised as shown in step 1 of Scheme 1. Intermediates 12 and 11 may be synthesised as shown in steps 1 and 2, respectively, of Scheme 3.

In step 1 of Scheme 3, intermediate 4 is reacted with 1.0 equivalent of 5-methyl-2-pyrazinecarboxylic acid (F) in the presence 2.0 equivalents of hexafluorophosphate azabenzotriazole tetramethyl uronium (HATU) and 0.8 equivalents of diisopropylethylamine in tetrahydrofuran (THF) to generate intermediate 12.

In step 2 of Scheme 3, (S)-2-N-Boc-aminomethylpyrrolidine is reacted with 1.1 equivalents of veratraldehyde (E) in the presence of 2.0 equivalents of sodium cyanoborohydride (NaBH₃CN) and 4.0 equivalents of acetic acid (HOAc) in dichloroethane (DCE) to generate intermediate 10. To generate intermediate 11, intermediate 10 is deprotected with hydrochloric acid in methanol (HCl/MeOH) at elevated temperature (50° C.) using methanol (MeOH) as a solvent.

In step 3 of Scheme 3, intermediate 12 is reacted with 1.0 equivalent of intermediate 11 in the presence of 4.0 equivalents of potassium carbonate (K₂CO₃) in dimethylformamide (DMF) at elevated temperature (70° C.) to generate compound 2678-53.

Biological Activity

The ability of compounds of the disclosure to inhibit the PD-1/PD-L1 interaction was investigated using a Cisbio PD-1/PD-L1 Homogeneous Time-Resolved Fluorescence (HTRF) binding assay (https://ca.cisbio.net/human-pd1-pd-l1-biochemical-binding-assay-44666).

With the assay, the interaction between PD-L1 and PD-1 is detected by using anti-Tag1 labeled with Europium (HTRF donor) and anti-Tag2 labeled with XL665 (HTRF acceptor). When the donor and acceptor antibodies are brought into close proximity due to PD-L1 and PD-1 binding, excitation of the donor antibody triggers fluorescence resonance energy transfer (FRET) towards the acceptor antibody, which in turn emits specifically at 665 nm. This specific signal is directly proportional to the extent of PD-1/PD-L1 interaction. Thus, a compound blocking the PD-1/PD-L1 interaction causes a reduction in the HTRF signal.

The assay was carried out in accordance with the product insert.

IC₅₀ Values for Selected Compounds of the Disclosure

The determined IC₅₀ values for selected compounds of the disclosure measured in the PD-1/PD-L1 HTRF binding assay are provided in Table 8 to 10. Also listed in Table 8 are the determined IC₅₀ values for different control compounds measured in the PD-1/PD-L1 HTRF binding assay.

TABLE 8
PD-1/PD-L1 Inhibitory Activity

| Compound ID | Run | Reported IC₅₀ (μM) |
|---|---|---|
| 2678-2 | Pure | 1.9 |
| 2678-3 | Pure | 7.0 |
| 2678-5 | Pure | 9.2 |
| 2678-10 | Pure | 0.33 |
| 2678-32 | Pure (70% Pure) | 7.9 |
| 2678-51 | Pure | 8.7 |
| 2678-53 | Pure | 53 |
| 2678-58 | Pure | 0.80 |
| 2678-66 | Pure | 0.87 |
| 2678-72 | Pure (70% Pure) | 11 |
| 2678-78 | Pure | 1.6 |
| 2678-80 | Pure | 0.50 |
| 2463-262 | Crude | 3.1 |
| S7911 | Pure | 0.099 |

TABLE 9
PD-1/PD-L1 Inhibitory Activity

| | Reported IC₅₀ | |
|---|---|---|
| Compound ID | in nM (in μM) | |
| 2678-10 (A301) | 51 | (0.051) |
| A302 | 140 | (0.14) |
| A303 | 9100 | (9.1) |
| A304 | 3500 | (3.5) |
| A305 | 36,000 | (36) |
| A306 | 40,000 | (40) |
| A307 | 9200 | (9.2) |
| A308 | 690 | (0.69) |
| A309 | 10,400 | (10) |
| A310 | 26,000 | (26) |
| A311 | >50,000 | (>50) |
| A312 | >50,000 | (>50) |
| A401 | 1200 | (1.2) |
| A402 | 620 | (0.62) |
| A403 | 530 | (0.53) |
| A404 | 880 | (0.88) |
| A405 | 247 | (0.25) |
| A406 | 980 | (0.98) |
| A407 | 490 | (0.49) |
| A501 | 740 | (0.74) |
| A502 | 590 | (0.59) |
| A503 | 2600 | (0.26) |
| A504 | 2000 | (2.0) |
| A505 | >50,000 | (>50) |
| A506 | >50,000 | (>50) |
| A601 | 159 | (0.16) |
| A602 | 324 | (0.32) |
| A603 | 110 | (0.11) |
| A604 | 215 | (0.22) |
| A605 | >50,000 | (>50) |
| A606 | >50,000 | (>50) |
| A607 | 96 | (0.096) |
| A608 | 1600 | (1.6) |
| A703 | 109 | (0.11) |
| A704 | 89 | (0.089) |
| A705 | 171 | (0.17) |
| A706 | 13,000 | (13) |
| A707 | >50,000 | (>50) |
| B313 | 205 | (0.21) |
| B315 | 353 | (0.35) |
| B318 | 92 | (0.092) |
| B408 | >50,000 | (>50) |
| B410 | >5000 | (>5) |
| B414 | >5000 | (>5) |

TABLE 10
PD-1/PD-L1 Inhibitory Activity

| | Reported IC₅₀ | |
|---|---|---|
| Compound ID | in nM (in μM) | |
| A702 | *15 | (0.015) |
| B314 | 18 | (0.018) |
| B316 | 25 | (0.025) |
| B317 | 18 | (0.018) |
| B319 | *15 | (0.015) |
| B409 | 650 | (0.65) |
| B411 | 1020 | (1.0) |
| B412 | 4400 | (4.4) |
| B413 | 440 | (0.44) |
| B415 | 216 | (0.22) |
| B708 | 28 | (0.028) |
| B709 | 8000 | (8.0) |
| B710 | 96 | (0.096) |
| B801 | 18 | (0.018) |
| B802 | 12 | (0.012) |
| B803 | P1: 21 | (0.021) |
| | P2: 27 | (0.027) |
| B804 | 21 | (0.021) |
| B805 | P1: 14 | (0.014) |
| B806 | P2: 12 | (0.012) |
| B908 | P1: 24 | (0.024) |
| | P2: 79 | (0.079) |
| B909 | P1: 544 | (0.54) |
| | P2: 44 | (0.044) |
| B910 | 44 | (0.044) |
| B911 | 33 | (0.033) |

*n = 7, median
“P1” and “P2”, where listed, refer to different isomers.

The HTRF assay results demonstrate that compounds of the disclosure possess activity as inhibitors of the PD-1/PD-L1 interaction. Accordingly, compounds of the disclosure may be used in the treatment of diseases or deficiencies associated with the PD-1/PD-L1 interaction, such as cancer.

Oral Bioavailability

A comparison of several properties of known small molecule PD-1/PD-L1 blockers with a compound of the disclosure is shown in Table 11.

TABLE 11
Comparison of Physicochemical Parameters
that Impact Oral Bioavailability

| | K_{D}/IC₅₀ | MW | | TPSA | | |
|---|---|---|---|---|---|---|
| Compound | (nM) | (Da) | Log P | (Å²) | Fsp³ | LE |
| INB086550 | <10 | 773 | 6.8 | 165 | 0.35 | 0.20 |
| BMS-1166 | 1.4 | 641 | 4.2 | 121 | 0.32 | 0.27 |
| GS-4224, | 0.09 | 866 | 6.4 | 132 | 0.26 | 0.25 |
| Example A | | | | | | |
| GS-4224, | 92 | 460 | 2.1 | 110 | 0.24 | 0.29 |
| Example B | | | | | | |
| CA-170 | NA | 360 | −5.4 | 227 | 0.58 | [0.44] |
| | [EC₅₀ = 17] | | | | | |
| Compound | 330 | 645 | 3.6 | 79 | 0.48 | 0.22 |
| 2678-10 | | | | | | |

Table 11 demonstrates that compound 2678-10 compares favorably with other small molecule agents that have been developed.

Although the foregoing invention has been described in some detail by way of illustration and example for purposes of clarity of understanding, the description is not intended as a limitation on the scope of the present disclosure but is instead provided as a description of exemplary embodiments. It will be readily apparent to those of ordinary skill in the art in light of the teachings of this disclosure that certain changes and modifications may be made thereto without departing from the scope of the appended claims.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present disclosure is not entitled to antedate such publication by virtue of prior invention.

1. A compound of Formula (I): 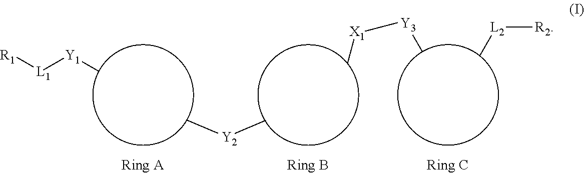
wherein:
R₁ is C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆-heteroalkyl or C₅₋₁₄heteroaryl-C₂-heteroalkyl, each of which is unsubstituted or substituted with one or more R^{A};
R₂ is C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆-heteroalkyl or C₅₋₁₄heteroaryl-C₂-heteroalkyl, each of which is unsubstituted or substituted with one or more R^{A};
L₁ is optional and if present is C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl or C₅₋₁₄ heterocycloalkenyl, each of which is unsubstituted or substituted with one or more R^{A};
L₂ is optional and if present is C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl or C₅₋₁₄ heterocycloalkenyl, each of which is unsubstituted or substituted with one or more R^{A};
Y₁, Y₂ and Y₃ are independently —CR₅R₆—, —C(CR₅R₆)—, —C(O)—, —C(NR^{B})—, —C(S)—, —NR^{B}—, —O—, —S—, —(O)— or —S(O)₂—;
X₁ is —CR^{B}₂—, —C(CR^{B}₂)—, —C(O)—, —C(NR^{B})—, —C(S)—, —NR^{B}—, —O—, —S—, —(O)— or —S(O)₂—;
Ring A is a C cycloalkyl or C heterocycloalkyl, each of which is unsubstituted or substituted with one or more R₃;
Ring B is a C aryl or C₅₋₆ heteroaryl, each of which is unsubstituted or substituted with one or more R^{A};
Ring C is a C₅₋₆ cycloalkyl or C₅₋₆ heterocycloalkyl, each of which is unsubstituted or substituted with one or more R₄;
R₃ and R₄ at each occurrence are independently C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₁₋₆ haloalkyl, C₂₋₆ haloalkenyl, C₂₋₆ haloalkynyl, C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₃₋₁₄ haloheterocycloalkyl, C₅₋₁₄ haloheterocycloalkenyl, C₅₋₁₄ aryl, C₅₋₁₄ heteroaryl, haloC₅₋₁₄ aryl, haloC₅₋₁₄heteroaryl, C₅₋₁₄aryl-C₁₋₆alkyl, C₅₋₁₄heteroaryl-C₁₋₆alkyl, haloC₅₋₁₄aryl-C₁₋₆ alkyl, haloC₅₋₁₄heteroaryl-C₁₋₆alkyl, C₅₋₁₄aryl-C₂₋₆heteroalkyl, C₅₋₁₄heteroaryl-C₂₋₆ heteroalkyl, haloC₅₋₁₄aryl-C₂₋₆heteroalkyl, haloC₅₋₁₄heteroaryl-C₂₋₆heteroalkyl, —F, —Cl, —Br, —I, —CN, —NO₂, —SO₂, —N₃, —SCN, —NCS, —OR^{B}, —NR^{C}R^{D}, —NR^{B}NR^{C}R^{D}, —SR^{B}, —C(O)R^{B}, —C(O)OR^{B}, —C(O)NR^{C}R^{D}, —C(NR^{C})R^{B}, —C(NR^{B})NR^{C}R^{D}, —S(O)R^{B}, —S(O)₂R^{B}, —S(O)OR^{B}, —S(O)₂OR^{B}, —NR^{B}NR^{C}R^{D}, —NR^{B}C(O)NR^{C}R^{D} or —NR^{B}C(NR^{B})NR^{C}R^{D}; two or more R₃ or two or more R₄ together with the atom(s) to which they are attached form a cyclic group; and/or two R₃ or two R₄ on the same atom form (═O), (═NR^{B}) or (═S);
R₅ and R₆ at each occurrence are independently —H, C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₁₋₆ haloalkyl, C₂₋₆ haloalkenyl, C₂₋₆ haloalkynyl, C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₃₋₁₄ haloheterocycloalkyl, C₅₋₁₄ haloheterocycloalkenyl, C₅₋₁₄ aryl, C₅₋₁₄ heteroaryl, haloC₅₋₁₄ aryl, haloC₅₋₁₄heteroaryl, C₅₋₁₄aryl-C₁₋₆alkyl, C₅₋₁₄heteroaryl-C₁₋₆alkyl, haloC₅₋₁₄aryl-C₁₋₆ alkyl, haloC₅₋₁₄heteroaryl-C₁₋₆alkyl, C₅₋₁₄aryl-C₂₋₆heteroalkyl, C₅₋₁₄heteroaryl-C₂₋₆ heteroalkyl, haloC₅₋₁₄aryl-C₂₋₆heteroalkyl, haloC₅₋₁₄heteroaryl-C₂₋₆heteroalkyl, —F, —Cl, —Br, —I, —CN, —NO₂, —SO₂, —N₃, —SCN, —NCS, —OR^{B}, —NR^{C}R^{D}, —NR^{B}NR^{C}R^{D}, —SR^{B}, —C(O)R^{B}, —C(O)OR^{B}, —C(O)NR^{C}R^{D}, —C(NR^{C})R^{B}, —C(NR^{B})NR^{C}R^{D}, —S(O)R^{B}, —S(O)₂R^{B}, —S(O)OR^{B}, —S(O)₂OR^{B}, —NR^{B}NR^{C}R^{D}, —NR^{B}C(O)NR^{C}R^{D} or —NR^{B}C(NR^{B})NR^{C}R^{D}; or R₅ and R₆ together with the carbon atom to which they are attached form a C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₃₋₁₄ haloheterocycloalkyl, or C₅₋₁₄ haloheterocycloalkenyl;
R^{A} at each occurrence is independently —F, —Cl, —Br, —I, —CN, —NO₂, —SO₂, —N₃, —SCN, —NCS, —OR^{B}, —NR^{C}R^{D}, —NR^{B}NR^{C}R^{D}, —SR^{B}, —C(O)R^{B}, —C(O)OR^{B}, —C(O)NR^{C}R^{D}, —C(NR^{B})R^{B}, —C(NR^{B})NR^{C}R^{D}, —S(O)R^{B}, —S(O)₂R^{B}, —S(O)OR^{B}, —S(O)₂OR^{B}, —NR^{B}NR^{C}R^{D}, —NR^{B}C(O)NR^{C}R^{D}, —NR^{B}C(NR^{B})NR^{C}R^{D}, C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂-heteroalkyl or C₅₋₁₄heteroaryl-C₂₋₆ heteroalkyl, wherein each C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₆₋₁₄heteroaryl-C₁₋₆alkyl, C₅₋₁₄aryl-C₂-heteroalkyl or C₅₋₁₄heteroaryl-C₂₋₆-heteroalkyl is unsubstituted or substituted with one or more —F, —Cl, —Br, —I, —CN, —NO₂, —SO₂, —N₃, —SCN, —NCS, —OR^{B}, —NR^{C}R^{D}, —NR^{B}NR^{C}R^{D}, —SR^{B}, —C(O)R^{B}, —C(O)OR^{B}, —C(O)NR^{C}R^{D}, —C(NR^{B})R^{B}, —C(NR^{B})NR^{C}R^{D}, —NR^{B}C(NR^{B})NR^{C}R^{D}, —S(O)R^{B}, —S(O)₂R^{B}, —S(O)OR^{B}, —S(O)₂OR^{B}, —NR^{B}NR^{C}R^{D} or —NR^{B}C(O)NR^{C}R^{D}; two or more R^{A} together with the atom(s) to which they are attached form a cyclic group; and/or two R^{A} on the same atom form (═O), (═NR^{B}) or (═S);
R^{B} at each occurrence is independently (i) —H, or (ii) C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂₋₆-heteroalkyl or C₅₋₁₄heteroaryl-C₂₋₆ heteroalkyl, each of which is unsubstituted or substituted with one or more R^{E};
R^{C} and R^{D} at each occurrence are independently (i) —H, or (ii) C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-CO₂-heteroalkyl or C₅₋₁₄heteroaryl-C₂₋₆-heteroalkyl, each of which is unsubstituted or substituted with one or more R^{E}, or R^{C} and R^{D} together with the nitrogen atom to which they are attached combine to form a heterocycle that is unsubstituted or substituted with one or more R^{E} groups;
R^{E} at each occurrence is independently —F, —Cl, —Br, —I, —CN, —NO₂, —SO₂, —N₃, —SCN, —NCS, —OR^{F}, —NR^{G}R^{H}, —NR^{F}NR^{G}R^{H}, —SR^{F}, —C(O)R^{F}, —C(O)OR^{F}, —C(O)NR^{G}R^{H}, —C(NR^{F})R^{F}, —C(NR^{F})NR^{G}R^{H}, —S(O)R^{F}, —S(O)₂R^{F}, —S(O)OR^{F}, —S(O)₂OR^{F}, —NR^{F}NR^{G}R^{H}, —NR^{F}C(O)NR^{G}R^{H}, —NR^{F}C(NR^{F})NR^{G}R^{H}, C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₅₋₁₄heteroaryl-C₁₋₆alkyl, C₅₋₁₄aryl-C₂-heteroalkyl or C₅₋₁₄heteroaryl-C₂₋₆ heteroalkyl, wherein each C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₃₋₁₄ cycloalkyl, C₃₋₁₄ heterocycloalkyl, C₅₋₁₄ cycloalkenyl, C₅₋₁₄ heterocycloalkenyl, C₅₋₁₄ aryl, C₅₋₁₄ heteroaryl, C₅₋₁₄aryl-C₁₋₆alkyl, C₆₋₁₄heteroaryl-C₁₋₆alkyl, C₅₋₁₄aryl-CO₂-heteroalkyl or C₅₋₁₄heteroaryl-C₂-heteroalkyl is unsubstituted or substituted with one or more —F, —Cl, —Br, —I, —CN, —NO₂, —SO₂, —N₃, —SCN, —NCS, —OR^{F}, —NR^{G}R^{H}, —NR^{F}NR^{G}R^{H}, —SR^{F}, —C(O)R^{F}, —C(O)OR^{F}, —C(O)NR^{G}R^{H}, —C(NR^{F})R^{F}, —C(NR^{F})NR^{G}R^{H}, —NR^{F}C(NR^{F})NR^{G}R^{H}, —S(O)R^{F}, —S(O)₂R^{F}, —S(O)OR^{F}, —S(O)₂OR^{F}, —NR^{F}NR^{G}R^{H} or —NR^{F}C(O)NR^{G}R^{H}; two or more R^{E} together with the atoms to which they are attached form a cyclic group; and/or two R^{E} on the same atom form (═O), (═NR^{F}) or (═S);
R^{F} at each occurrence is independently (i) —H, or (ii) C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₁₋₆ haloalkyl, C₂₋₆ haloalkenyl, C₂₋₆ haloalkynyl, C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₅₋₁₄ aryl, C₅₋₁₄ heteroaryl, haloC₅₋₁₄aryl, haloC₅₋₁₄heteroaryl, C₅₋₁₄aryl-C₁₋₆alkyl, C₅₋₁₄heteroaryl-C₁₋₆alkyl, haloC₅₋₁₄aryl-C₁₋₆alkyl, haloC₅₋₁₄heteroaryl-C₁₋₆alkyl, C₅₋₁₄aryl-C₂₋₆-heteroalkyl, C₅₋₁₄ heteroaryl-C₂₋₆heteroalkyl, haloC₅₋₁₄aryl-C₂₋₆-heteroalkyl or haloC₅₋₁₄heteroaryl-C₂₋₆ heteroalkyl; and
R^{G} and R^{H} at each occurrence are independently (i) —H, or (ii) C₁₋₆ alkyl, C₂₋₆ alkenyl, C₂₋₆ alkynyl, C₂₋₆ heteroalkyl, C₃₋₆ heteroalkenyl, C₃₋₆ heteroalkynyl, C₁₋₆ haloalkyl, C₂₋₆ haloalkenyl, C₂₋₆ haloalkynyl, C₃₋₁₄ cycloalkyl, C₃₋₁₄ heterocycloalkyl, C₅₋₁₄ cycloalkenyl, C₅₋₁₄ heterocycloalkenyl, C₃₋₁₄ halocycloalkyl, C₅₋₁₄ halocycloalkenyl, C₅₋₁₄ aryl, C₅₋₁₄ heteroaryl, haloC₅₋₁₄aryl, haloC₅₋₁₄heteroaryl, C₅₋₁₄aryl-C₁₋₆alkyl, C₅₋₁₄heteroaryl-C₁₋₆alkyl, haloC₅₋₁₄aryl-C₁₋₆alkyl, haloC₅₋₁₄heteroaryl-C₁₋₆alkyl, C₅₋₁₄aryl-C₂-heteroalkyl, C₅₋₁₄ heteroaryl-C₂₋₆heteroalkyl, haloC₅₋₁₄aryl-C₂-heteroalkyl or haloC₅₋₁₄heteroaryl-C₂₋₆ heteroalkyl, or R^{G} and R^{H} together with the nitrogen atom to which they are attached combine to form a heterocycle,
or a pharmaceutically acceptable salt, solvate, tautomer, stereoisomer or prodrug thereof, provided that the compound of Formula (I) is not:

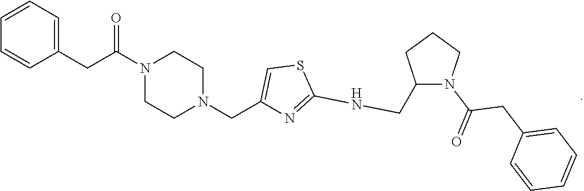 [SMILES: O=C(Cc1ccccc1)N1CCN(Cc2csc(NCC3CCCN3C(=O)Cc3ccccc3)n2)CC1]

2. The compound according to claim 1, or a pharmaceutically acceptable salt, solvate, tautomer, stereoisomer or prodrug thereof, wherein the compound is a compound of Formula (II): 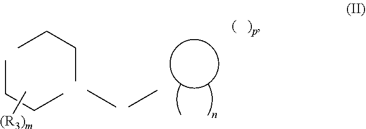
wherein:
X₂, X₃ and X₈ are independently —CH—, —CR^{A}— or —N—;
X₄, X₅ and X₆, and X₇ if present, are independently —CH—, —CR^{A}—, —C(O)—, —C(NR^{B})—, —C(S)—, —N—, —NR^{B}—, —O— or —S—, provided that the combination of X₄, X₅, X₆ and optionally X₇ together with the atoms to which they are attached forms an aromatic or heteroaromatic group;
m is 0 to 8;
n is 0 or 1;
p is 1 or 2; and
q is 0 to 9.

3. The compound according to claim 2, or a pharmaceutically acceptable salt, solvate, tautomer, stereoisomer or prodrug thereof, wherein:
X₁ is —CH₂—, —NH—, —N(C₁₋₆ alkyl)-, —O— or —S—; and/or
X₂, X₃ and X₈ are independently —CH—, —C(C₁₋₆ alkyl)- or —N—; and/or
Y₁ is —CH₂—, —CH(C₁₋₆ alkyl)-, —C(C₁₋₆ alkyl)₂-, —C(O)— or —S(O)₂—; and/or
Y₂ is —CR₅R₆—, —NR^{B}—, —O— or —S—, wherein R₅ and R₆ at each occurrence are independently —H or unsubstituted C₁₋₆ alkyl; or R₅ and R₆ together with the atom to which they are attached form a C₃₋₆ cycloalkyl; and R^{B} is —H or unsubstituted C₁₋₆ alkyl; and/or
Y₃ is —CH₂—, —CH(C₁₋₆ alkyl)-, —C(C₁₋₆ alkyl)₂- or —C(O)—; and/or
m is 0 to 4, and R₃, if present, at each occurrence is independently —F, —Cl, —Br, —I, —OH, C₁₋₆ alkyl or C₂₋₆ heteroalkyl, wherein each C₁₋₆ alkyl or C₂₋₆ heteroalkyl is unsubstituted two R₃ together with the atoms to which they are attached form a cyclic group; and/or two R₃ on the same atom form (═O); and/or
q is 0 to 4, and R₄, if present, at each occurrence is independently —F, —Cl, —Br, —I, —OH, C₁₋₆ alkyl, C₂₋₆ heteroalkyl or C₅₋₁₄aryl-C₂₋₆heteroalkyl, wherein each C₁₋₆ alkyl, C₂₋₆ heteroalkyl or C₅₋₁₄aryl-C₂₋₆heteroalkyl is unsubstituted.

4. The compound according to claim 1, or a pharmaceutically acceptable salt, solvate, tautomer, stereoisomer or prodrug thereof, wherein:
X₁ is —NH— or —N(C₁₋₆ alkyl)-; and/or
Y₁ is —CH₂—, —C(O)— or —S(O)₂—; and/or
Y₂ is —CH₂—, —CH(C₁₋₆ alkyl)-, —C(C₁₋₆ alkyl)₂-, or is as shown in the following structural fragment: 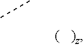
wherein z is 0 to 3; and/or
Y₃ is —CH₂— or —C(O)—.

5. The compound according to claim 2, or a pharmaceutically acceptable salt, solvate, tautomer, stereoisomer or prodrug thereof, wherein X₂ is —CH— and X₃ is —N—; X₂ is —N— and X₃ is —CH—; X₂ is —CH— and X₃ is —CH—; or X₂ is —N— and X₃ is —N—.

6. The compound according to claim 2, or a pharmaceutically acceptable salt, solvate, tautomer, stereoisomer or prodrug thereof, wherein m is 2, R₃ at each occurrence is independently C₁₋₆ alkyl, and Ring A is as shown in the following structural fragment:

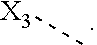

7. The compound according to claim 2, or a pharmaceutically acceptable salt, solvate, tautomer, stereoisomer or prodrug thereof, wherein m is 0.

8. The compound according to claim 2, or a pharmaceutically acceptable salt, solvate, tautomer, stereoisomer or prodrug thereof, wherein:
n is 1, and Ring B is defined as follows: 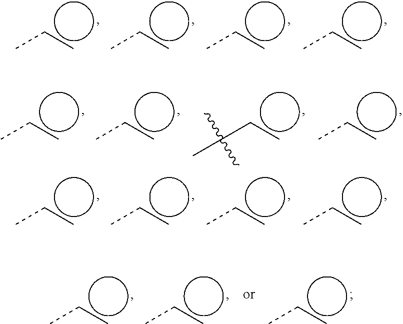
or
n is 0, and Ring B is defined as follows: 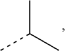
wherein X₄ is —CH—, —C(C₁₋₆ alkyl)- or —N—; X₅ is —NH—, —N(C₁₋₆ alkyl)-, —O— or —S—; and
X₆ is —CH—, —C(C₁₋₆ alkyl)- or —N—.

9. The compound according to claim 8, or a pharmaceutically acceptable salt, solvate, tautomer, stereoisomer or prodrug thereof, wherein Ring B is defined as follows:

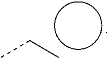

10. The compound according to claim 8, or a pharmaceutically acceptable salt, solvate, tautomer, stereoisomer or prodrug thereof, wherein Ring B is defined as follows:

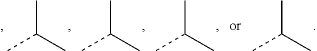

11. The compound according to claim 2, or a pharmaceutically acceptable salt, solvate, tautomer, stereoisomer or prodrug thereof, wherein:
q is 0 or 1;
R₄, if present, is —OH, —OC₁₋₆ alkyl, or —O—CH₂-phenyl; and
Ring C is as shown in the following structural fragment:

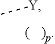

12. The compound according to claim 2, or a pharmaceutically acceptable salt, solvate, tautomer, stereoisomer or prodrug thereof, wherein p is 1.

13. The compound according to claim 2, or a pharmaceutically acceptable salt, solvate, tautomer, stereoisomer or prodrug thereof, wherein p is 2.

14. The compound according to claim 2, or a pharmaceutically acceptable salt, solvate, tautomer, stereoisomer or prodrug thereof, wherein X₈ is —N—.

15. The compound according to claim 1, or a pharmaceutically acceptable salt, solvate, tautomer, stereoisomer or prodrug thereof, wherein:
L₁ is absent, or L₁ is present and is C₁₋₄ alkyl or C₂₋₄ heteroalkyl; and/or
L₂ is present and is unsubstituted C₁₋₄ alkyl, —(CH(C₁₋₆ alkyl))-, —C(O)— or —C(O)O—.

16. The compound according to claim 1, or a pharmaceutically acceptable salt, solvate, tautomer, stereoisomer or prodrug thereof, wherein:
R₁ is C₁₋₆ alkyl, C₃₋₁₀ cycloalkyl, C₅₋₁₀ heterocycloalkenyl, C₆₋₁₀ aryl or C₆₋₁₀ heteroaryl, each of which is unsubstituted or substituted one or more times, independently, with —F, —Cl, —Br, —I, —NO₂, —OC₁₋₆ alkyl, unsubstituted C₁₋₆ alkyl, or C₁₋₆ alkyl substituted 1 to 3 times, independently, with —F, —Cl, —Br or —I; or C aryl or C₅₋₆ heteroaryl, wherein two adjacent atoms of the aryl or heteroaryl ring are bonded to the group —O—(CH₂)_{y}—O—, wherein y is 1 or 2; and/or
R₂ is C₁₋₆ alkyl, C₂₋₆ heteroalkyl, C₃₋₆ cycloalkyl, C₆₋₁₀ aryl or C₅₋₁₀ heteroaryl, each of which is unsubstituted or substituted one or more times, independently, with —F, —Cl, —Br, —I, —SO₂C₁₋₆ alkyl, —OC₁₋₆ alkyl, unsubstituted C₁₋₆ alkyl, unsubstituted C₃₋₆ cycloalkyl, or C₁₋₆ alkyl substituted 1 to 3 times, independently, with —F, —Cl, —Br or —I; or C aryl or C₅₋₆ heteroaryl, wherein two adjacent atoms of the aryl or heteroaryl ring are bonded to the group —O—(CH₂)_{y}—O—, wherein y is 1 or 2.

17. The compound according to claim 1, or a pharmaceutically acceptable salt, solvate, tautomer, stereoisomer or prodrug thereof, wherein:
R₁ is: 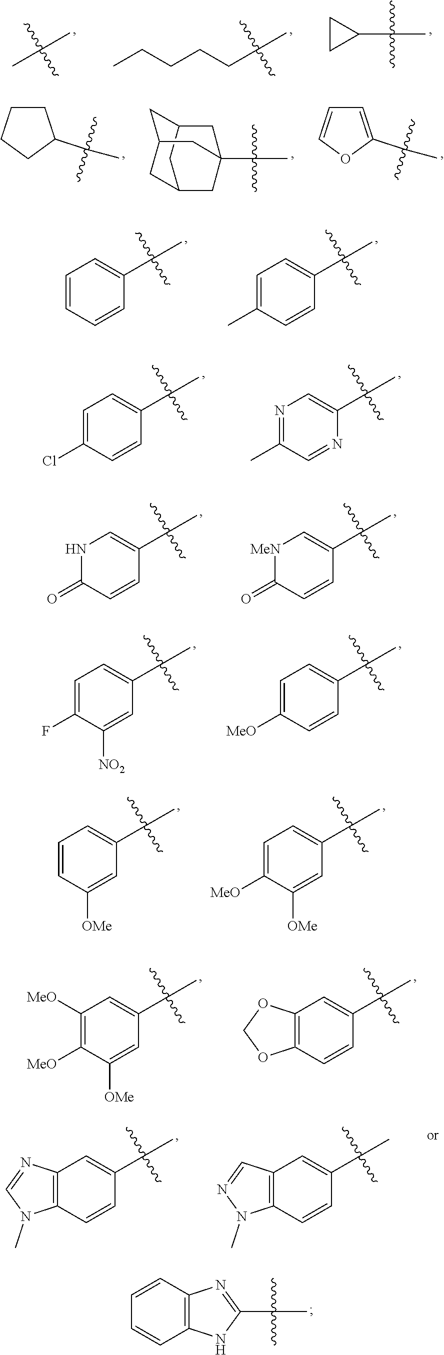
and/or
R₂ is:

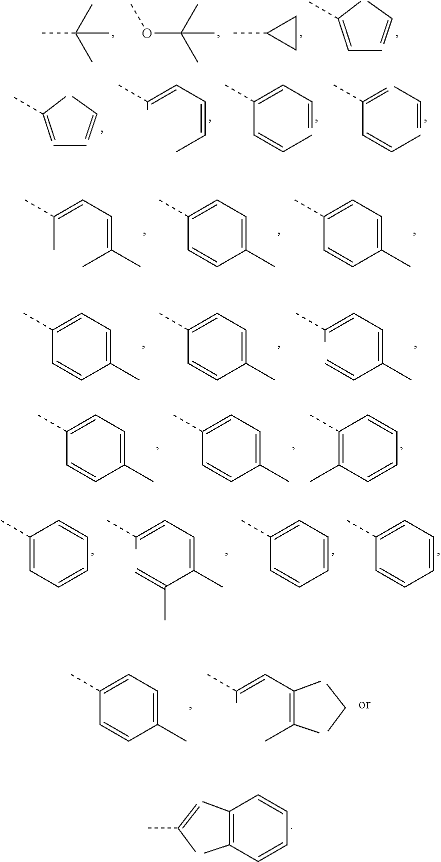

18. The compound according to claim 1, or a pharmaceutically acceptable salt, solvate, tautomer, stereoisomer or prodrug thereof, wherein L₁ and R₁ together are:

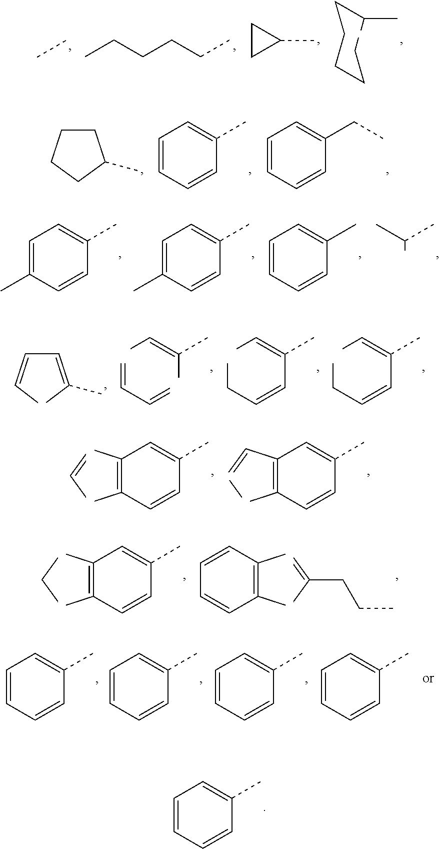

19. The compound according to claim 1, or a pharmaceutically acceptable salt, solvate, tautomer, stereoisomer or prodrug thereof, wherein L₂ and R₂ together are:

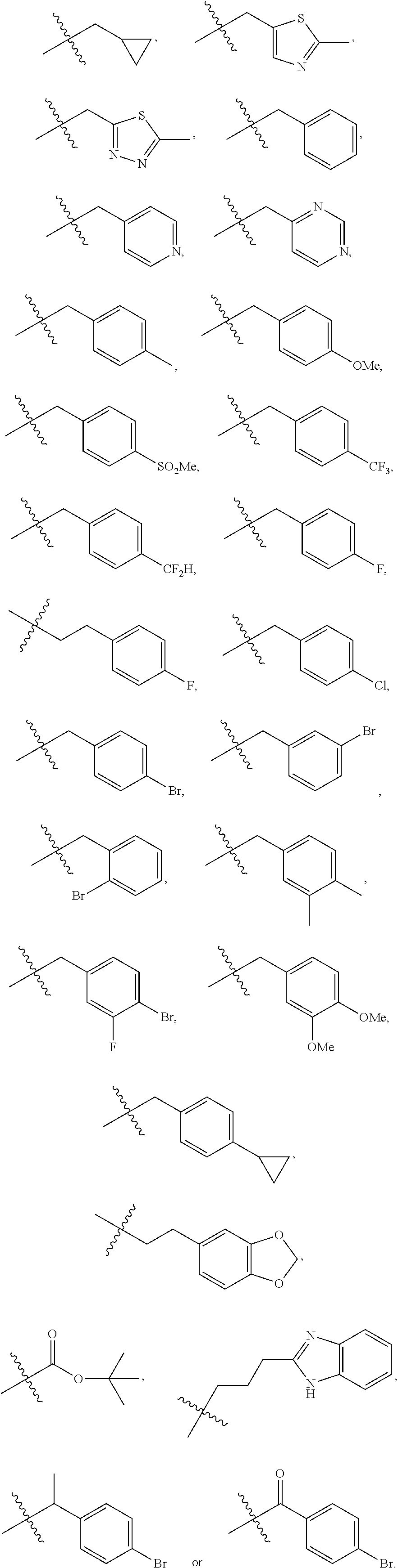

20. The compound according to claim 1, wherein the compound is as defined in the following table:

| R₁—L₁ | 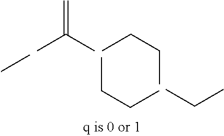 | R₄ | L₂—R₂ |
|---|---|---|---|
| 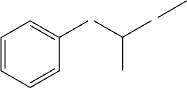 | 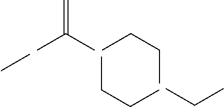 | absent | 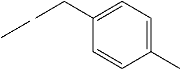 |
| 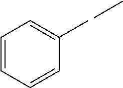 | 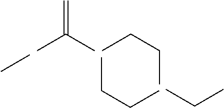 | OBzl | 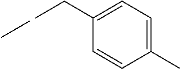 |
| 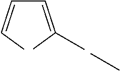 | 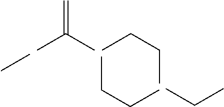 | absent | 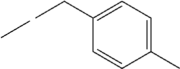 |
| 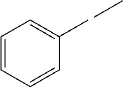 | 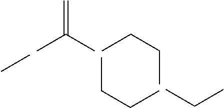 | absent | 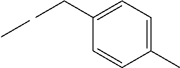 |
| 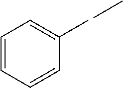 | 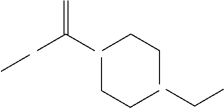 | absent | 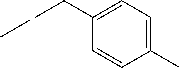 |
| 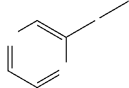 | 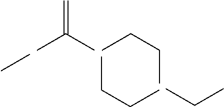 | absent | 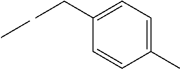 |
| 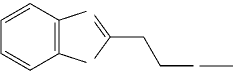 | 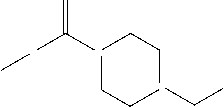 | absent | 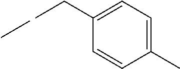 |
| 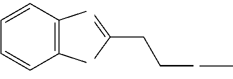 | 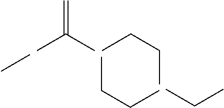 | OBzl | 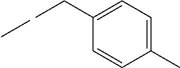 |
| 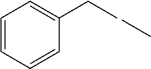 | 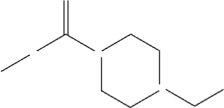 | absent | 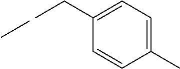 |
| 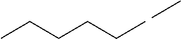 | 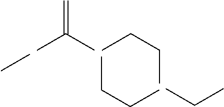 | absent | 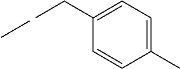 |
| 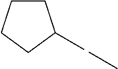 | 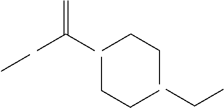 | absent | 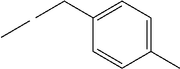 |
| 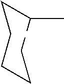 | 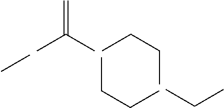 | absent | 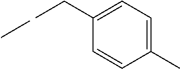 |
| 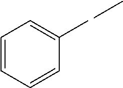 | 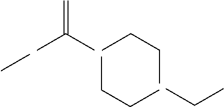 | absent | 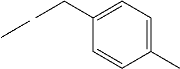 |
| 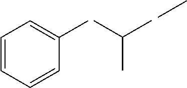 | 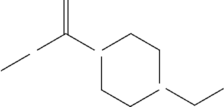 | absent | 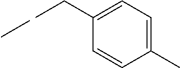 |
| 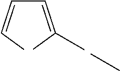 | 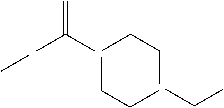 | absent | 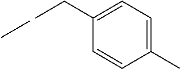 |
| 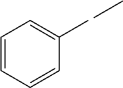 | 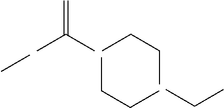 | absent | 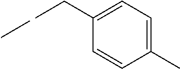 |
| 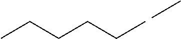 | 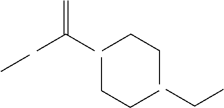 | absent | 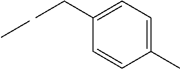 |
| 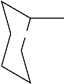 | 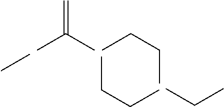 | absent | 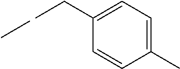 |
| 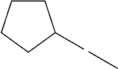 | 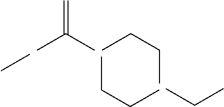 | absent | 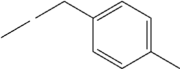 |
| 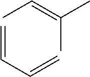 | 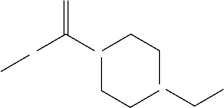 | absent | 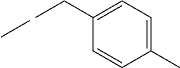 |
| 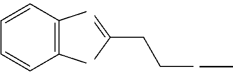 | 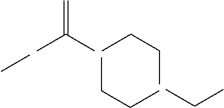 | absent | 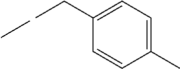 |
| 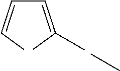 | 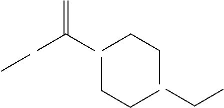 | absent | 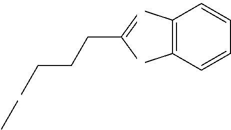 |
| 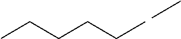 | 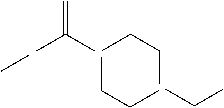 | absent | 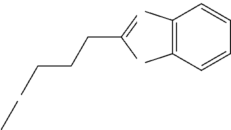 |
| 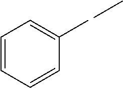 | 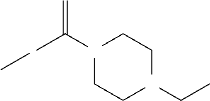 | absent | 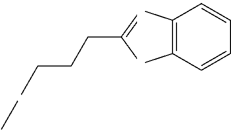 |
| 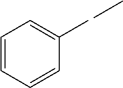 | 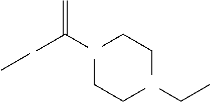 | absent | 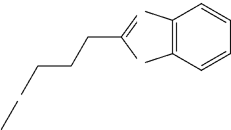 |
| 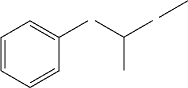 | 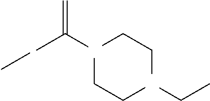 | absent | 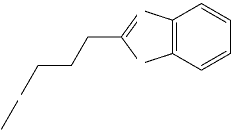 |
| 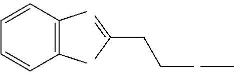 | 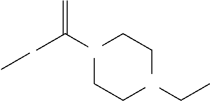 | absent | 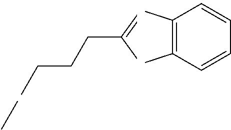 |
| 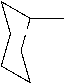 | 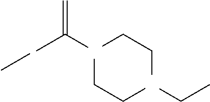 | absent | 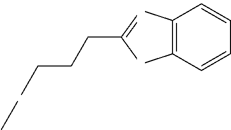 |
| 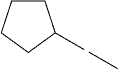 | 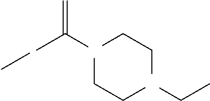 | absent | 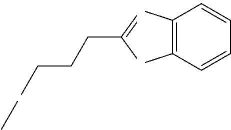 |
| 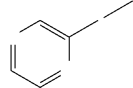 | 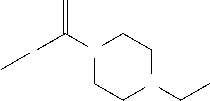 | absent | 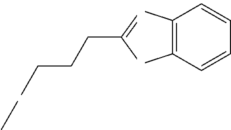 |
| 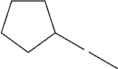 | 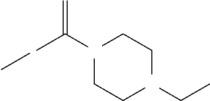 | absent | 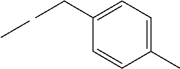 |
| 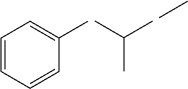 | 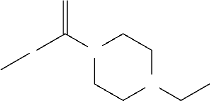 | absent | 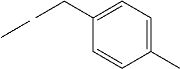 |
| 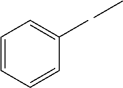 | 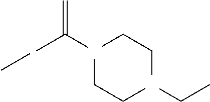 | absent | 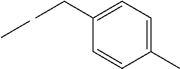 |
| 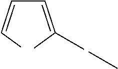 | 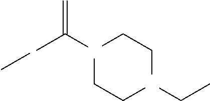 | absent | 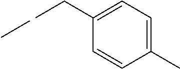 |
| 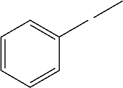 | 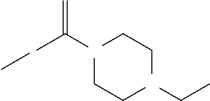 | absent | 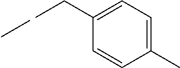 |
| 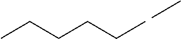 | 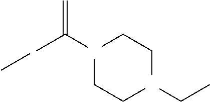 | absent | 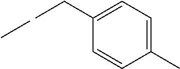 |
| 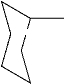 | 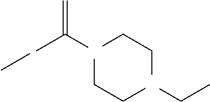 | absent | 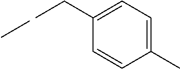 |
| 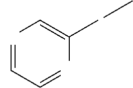 | 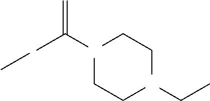 | absent | 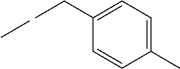 |
| 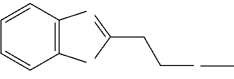 | 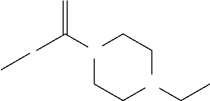 | absent | 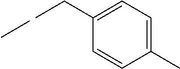 |
| 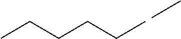 | 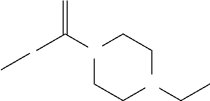 | absent | 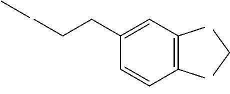 |
| 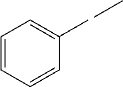 | 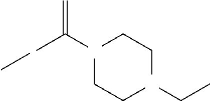 | absent | 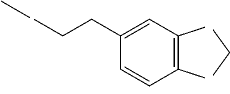 |
| 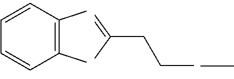 | 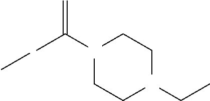 | absent | 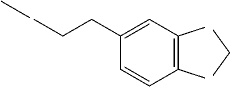 |
| 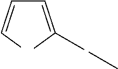 | 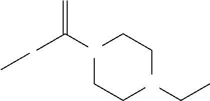 | absent | 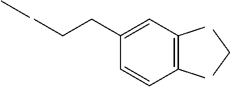 |
| 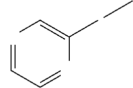 | 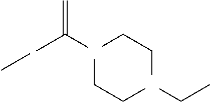 | absent | 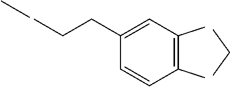 |
| 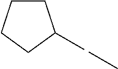 | 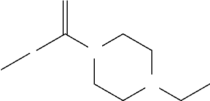 | absent | 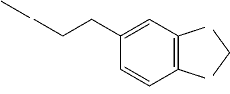 |
| 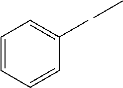 | 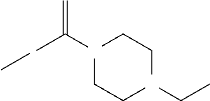 | absent | 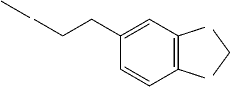 |
| 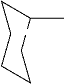 | 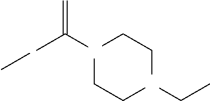 | absent | 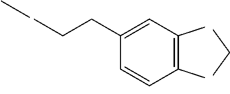 |
| 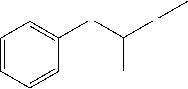 | 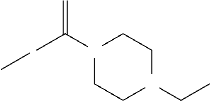 | absent | 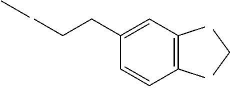 |
| 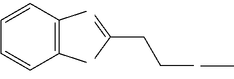 | 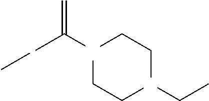 | OBzl | 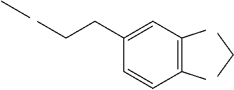 |
| 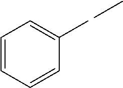 | 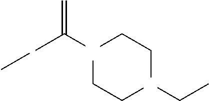 | OBzl | 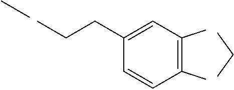 |
| 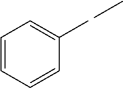 | 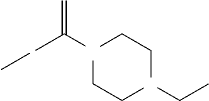 | absent | 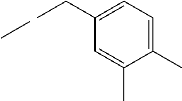 |
| 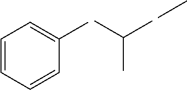 | 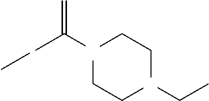 | absent | 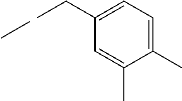 |
| 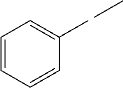 | 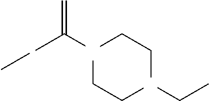 | absent | 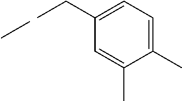 |
| 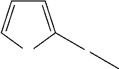 | 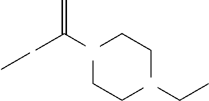 | absent | 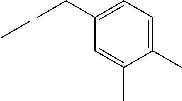 |
| 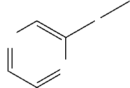 | 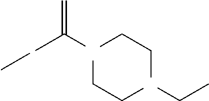 | absent | 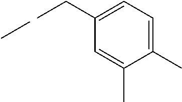 |
| 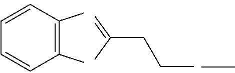 | 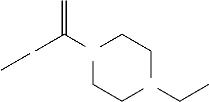 | absent | 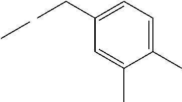 |
| 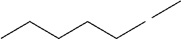 | 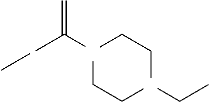 | absent | 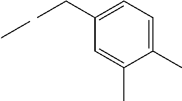 |
| 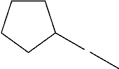 | 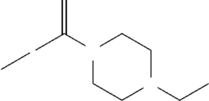 | absent | 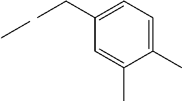 |
| 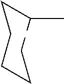 | 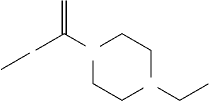 | absent | 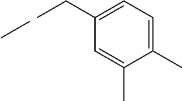 |
| 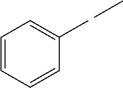 | 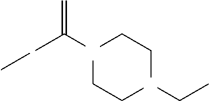 | absent | 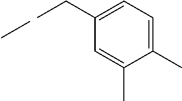 |
| 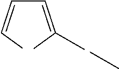 | 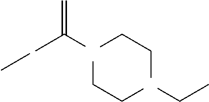 | absent | 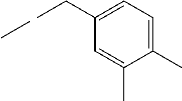 |
| 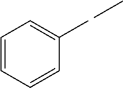 | 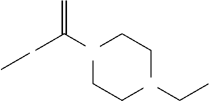 | absent | 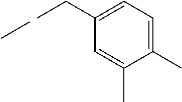 |
| 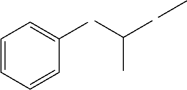 | 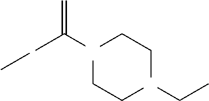 | absent | 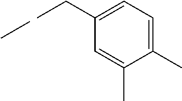 |
| 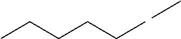 | 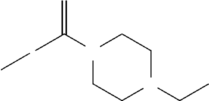 | absent | 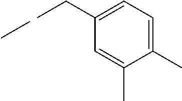 |
| 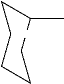 | 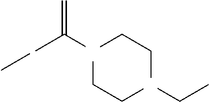 | absent | 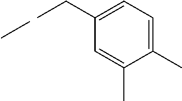 |
| 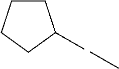 | 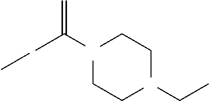 | absent | 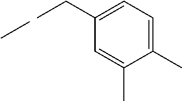 |
| 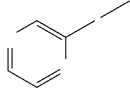 | 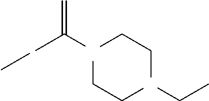 | absent | 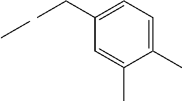 |
| 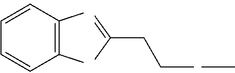 | 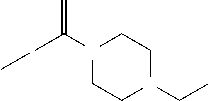 | absent | 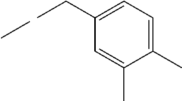 |
| 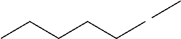 | 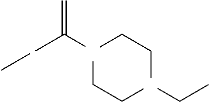 | absent | 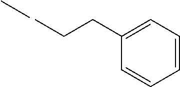 |
| 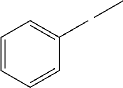 | 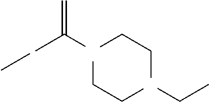 | absent | 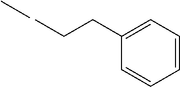 |
| 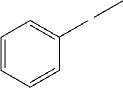 | 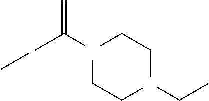 | absent | 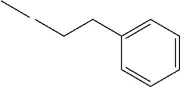 |
| 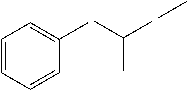 | 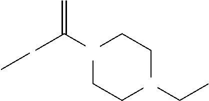 | absent | 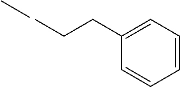 |
| 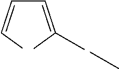 | 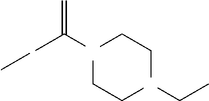 | absent | 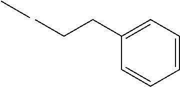 |
| 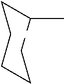 | 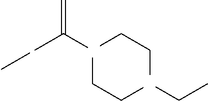 | absent | 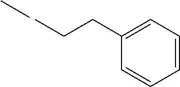 |
| 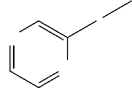 | 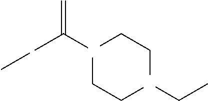 | absent | 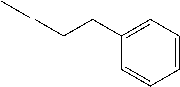 |
| 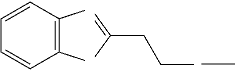 | 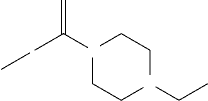 | absent | 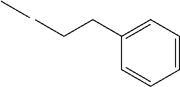 |
| 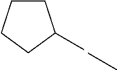 | 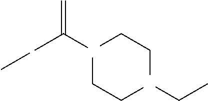 | absent | 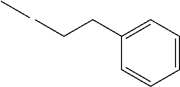 |
| 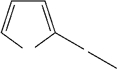 | 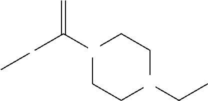 | absent | 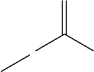 |
| 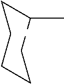 | 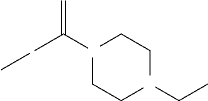 | absent | 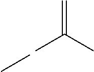 |
| 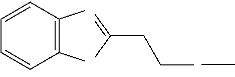 | 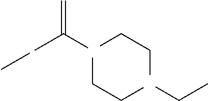 | absent | 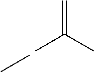 |
| 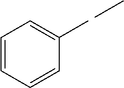 | 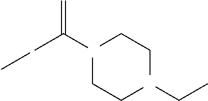 | absent | 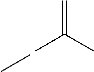 |
| 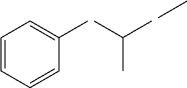 | 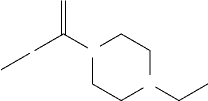 | absent | 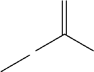 |
| 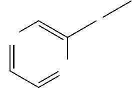 | 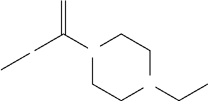 | absent | 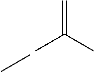 |
| 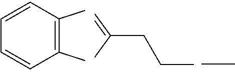 | 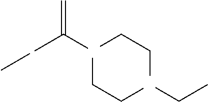 | absent | 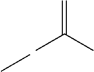 |
| 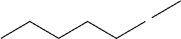 | 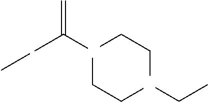 | absent | 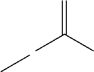 |
| 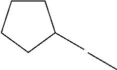 | 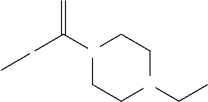 | absent | 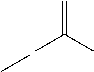 |
| 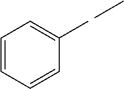 | 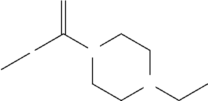 | absent | 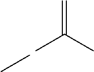 |

or a pharmaceutically acceptable salt, solvate, tautomer, stereoisomer or prodrug thereof.

21. The compound according to claim 1, wherein the compound is as defined in the following table:

| R₁—L₁ | 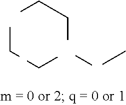 | R₄ | L₂—R₂ |
|---|---|---|---|
| 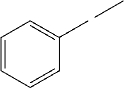 | 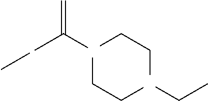 | absent | 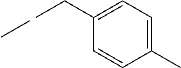 |
| 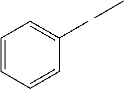 | 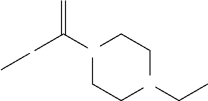 | absent | 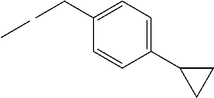 |
| 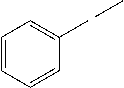 | 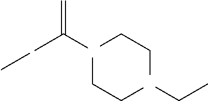 | absent | 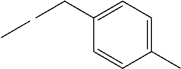 |
| 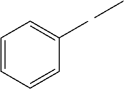 | 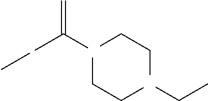 | absent | 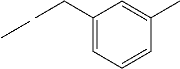 |
| 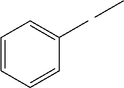 | 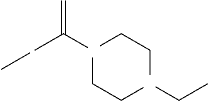 | absent | 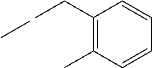 |
| 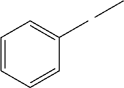 | 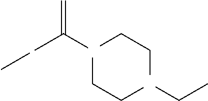 | absent | 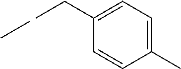 |
| 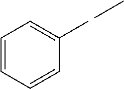 | 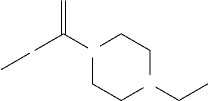 | absent | 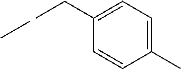 |
| 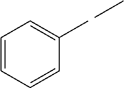 | 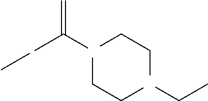 | absent | 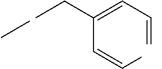 |
| 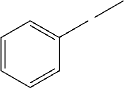 | 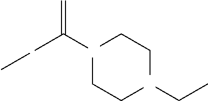 | absent | 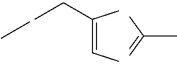 |
| 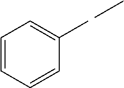 | 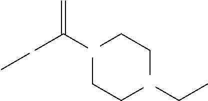 | absent | 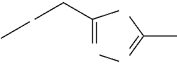 |
| 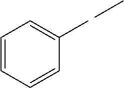 | 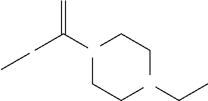 | absent | 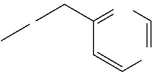 |
| 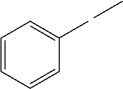 | 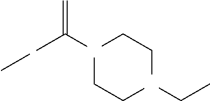 | absent | 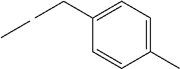 |
| 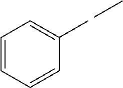 | 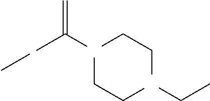 | absent | 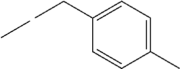 |
| 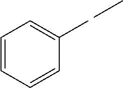 | 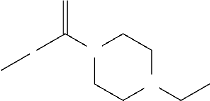 | absent | 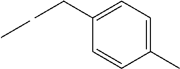 |
| 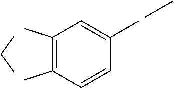 | 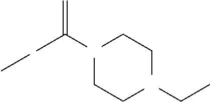 | absent | 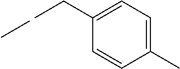 |
| 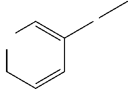 | 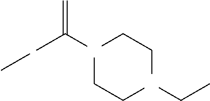 | absent | 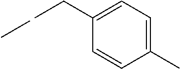 |
| 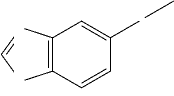 | 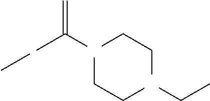 | absent | 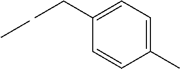 |
| 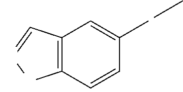 | 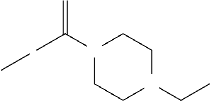 | absent | 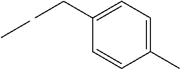 |
| 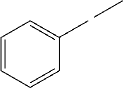 | 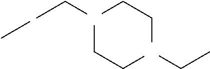 | absent | 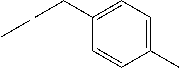 |
| 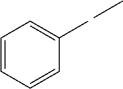 | 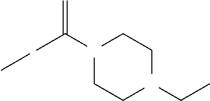 | absent | 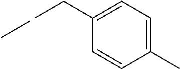 |
| 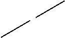 | 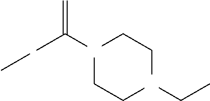 | absent | 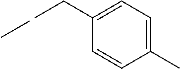 |
| 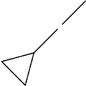 | 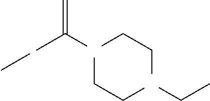 | absent | 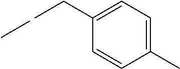 |
| 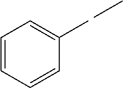 | 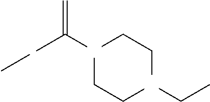 | absent | 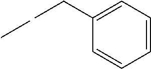 |
| 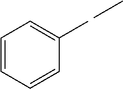 | 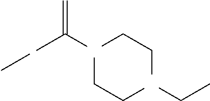 | absent | 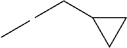 |
| 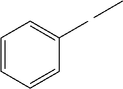 | 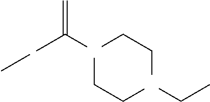 | absent | 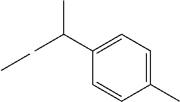 |
| 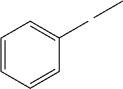 | 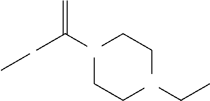 | absent | 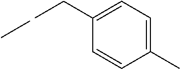 |
| 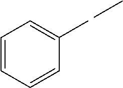 | 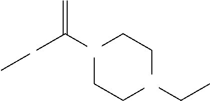 | absent | 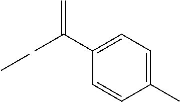 |
| 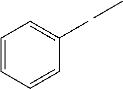 | 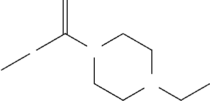 | absent | 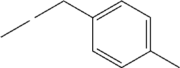 |
| 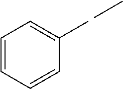 | 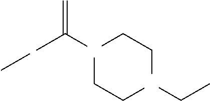 | absent | 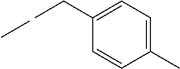 |
| 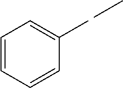 | 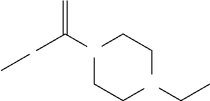 | absent | 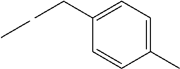 |
| 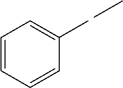 | 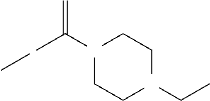 | OH | 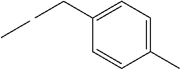 |
| 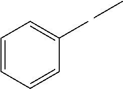 | 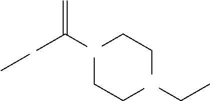 | OMe | 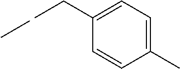 |
| 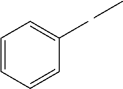 | 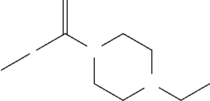 | absent | 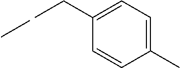 |
| 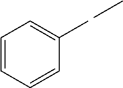 | 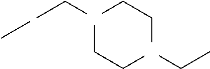 | absent | 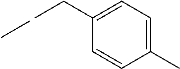 |
| 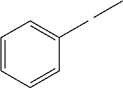 | 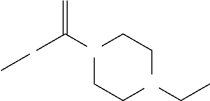 | absent | 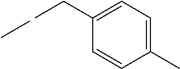 |
| 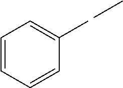 | 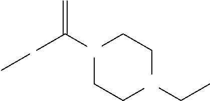 | absent | 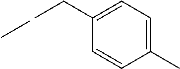 |
| 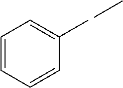 | 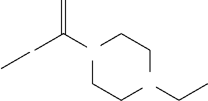 | absent | 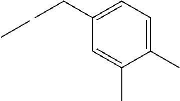 |
| 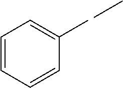 | 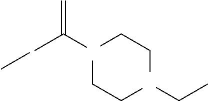 | absent | 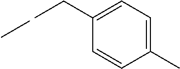 |
| 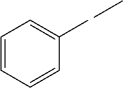 | 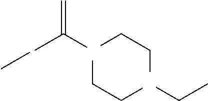 | absent | 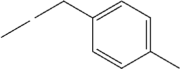 |
| 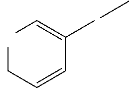 | 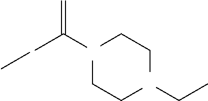 | absent | 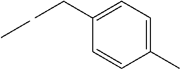 |

or a pharmaceutically acceptable salt, solvate, tautomer, stereoisomer or prodrug thereof.

22. The compound according to claim 1, wherein the compound is as defined in the following table:

| R₁—L₁ | 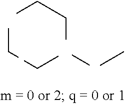 | L₂—R₂ |
|---|---|---|
| 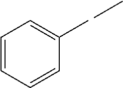 | 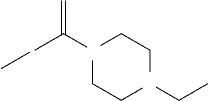 | 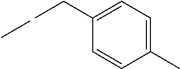 |
| 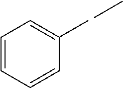 | 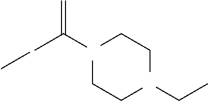 | 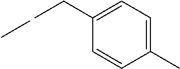 |
| 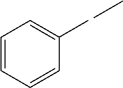 | 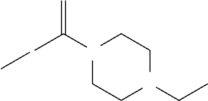 | 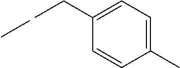 |
| 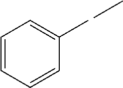 | 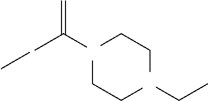 | 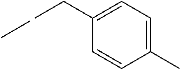 |
| 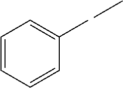 | 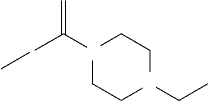 | 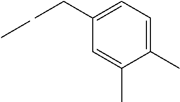 |
| 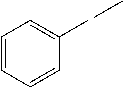 | 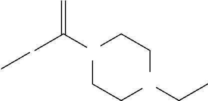 | 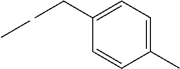 |
| 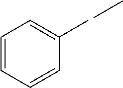 | 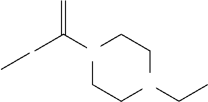 | 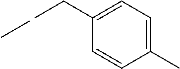 |
| 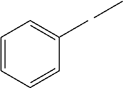 | 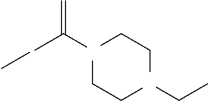 | 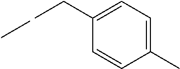 |
| 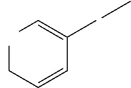 | 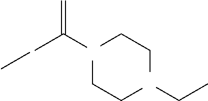 | 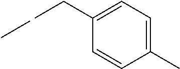 |
| 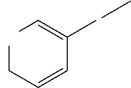 | 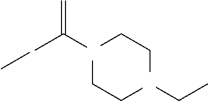 | 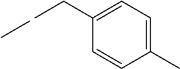 |
| 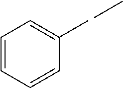 | 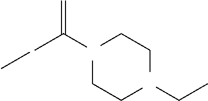 | 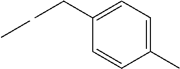 |
| 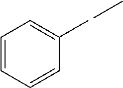 | 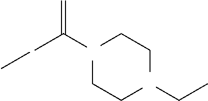 | 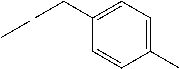 |
| 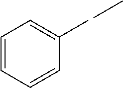 | 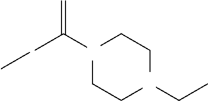 | 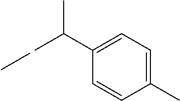 |
| 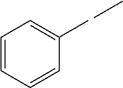 | 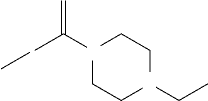 | 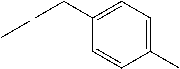 |
| 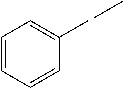 | 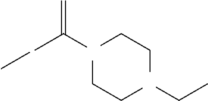 | 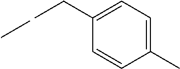 |
| 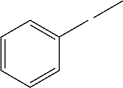 | 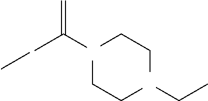 | 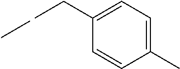 |
| 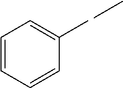 | 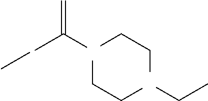 | 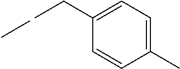 |
| 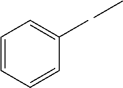 | 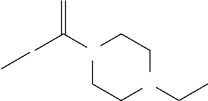 | 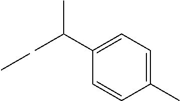 |
| 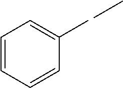 | 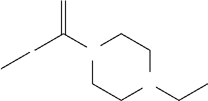 | 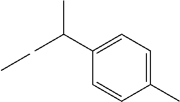 |
| 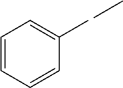 | 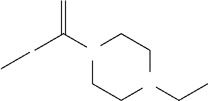 | 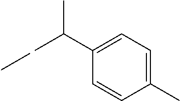 |
| 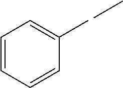 | 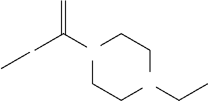 | 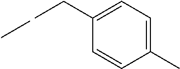 |

or a pharmaceutically acceptable salt, solvate, tautomer, stereoisomer or prodrug thereof.

23. The compound according to claim 1, wherein the compound is: 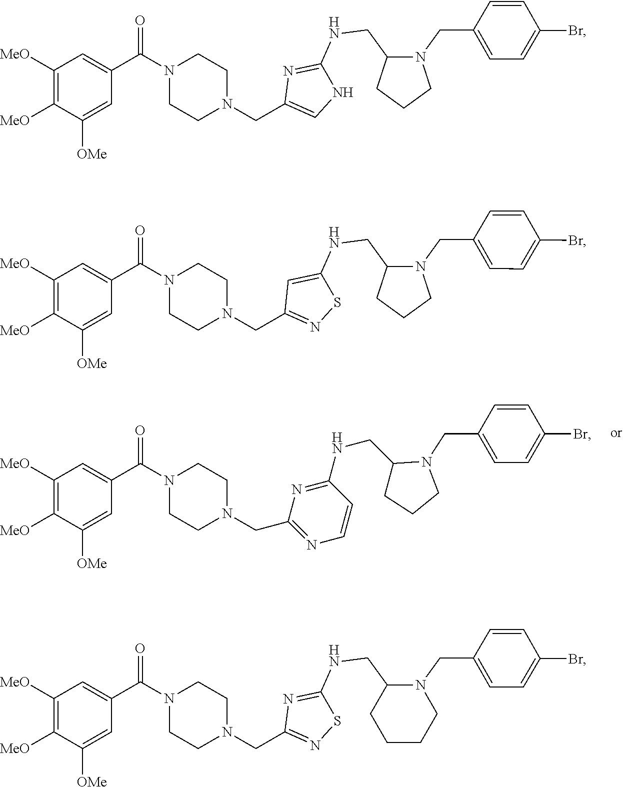 [SMILES: COc1cc(C(=O)N2CCN(Cc3c[nH]c(NCC4CCCN4Cc4ccc(Br)cc4)n3)CC2)cc(OC)c1OC ; COc1cc(C(=O)N2CCN(Cc3cc(NCC4CCCN4Cc4ccc(Br)cc4)sn3)CC2)cc(OC)c1OC ; COc1cc(C(=O)N2CCN(Cc3nccc(NCC4CCCN4Cc4ccc(Br)cc4)n3)CC2)cc(OC)c1OC ; "or" ; COc1cc(C(=O)N2CCN(Cc3nsc(NCC4CCCCN4Cc4ccc(Br)cc4)n3)CC2)cc(OC)c1OC]
or a pharmaceutically acceptable salt, solvate, tautomer, stereoisomer or prodrug thereof.

24. A pharmaceutical composition comprising the compound according to claim 1, or a pharmaceutically acceptable salt, solvate, tautomer, stereoisomer or prodrug thereof, and a pharmaceutically acceptable carrier, excipient and/or diluent.

25. A method of treating a disease or condition that is amenable to treatment by blocking PD-1, PD-L1 and/or the PD-1/PD-L1 interaction, wherein the method comprises administering a compound according to claim 1, or a pharmaceutically acceptable salt, solvate, tautomer, stereoisomer or prodrug thereof, or a compound which is 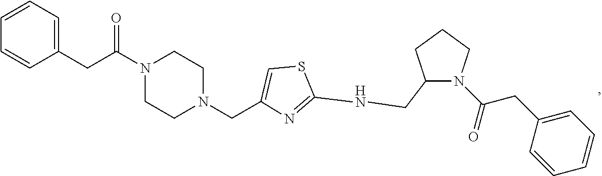 [SMILES: O=C(Cc1ccccc1)N1CCN(Cc2csc(NCC3CCCN3C(=O)Cc3ccccc3)n2)CC1]
or a pharmaceutically acceptable salt, solvate, tautomer, stereoisomer or prodrug thereof, to a subject in need thereof.
